SEQUENCE LISTING (37 sequences)

SEQ ID NO: 1 - DNA, 25 bp, synthetic construct
  source 1..25: mol_type = other DNA; note = primer binding site sequence
catgtaatgcacgtactttcagggt

SEQ ID NO: 2 - DNA, 20 bp, synthetic construct
  source 1..20: mol_type = other DNA; note = primer binding site sequence
aatgatacggcgaccaccga

SEQ ID NO: 3 - DNA, 35 bp, synthetic construct
  source 1..35: mol_type = other DNA; note = primer binding site sequence
cgtgctggattggctcaccagacaccttccgacat

SEQ ID NO: 4 - DNA, 33 bp, synthetic construct
  source 1..33: mol_type = other DNA; note = primer binding site sequence
acactctttccctacacgacgctcttccgatct

SEQ ID NO: 5 - DNA, 33 bp, synthetic construct
  source 1..33: mol_type = other DNA; note = primer binding site sequence
tcgtcggcagcgtcagatgtgtataagagacag

SEQ ID NO: 6 - DNA, 35 bp, synthetic construct
  source 1..35: mol_type = other DNA; note = primer binding site sequence
atgtcggaaggtgtgcaggctaccgcttgtcaact

SEQ ID NO: 7 - DNA, 34 bp, synthetic construct
  source 1..34: mol_type = other DNA; note = primer binding site sequence
agatcggaagagcacacgtctgaactccagtcac

SEQ ID NO: 8 - DNA, 34 bp, synthetic construct
  source 1..34: mol_type = other DNA; note = primer binding site sequence
ctgtctcttatacacatctccgagcccacgagac

SEQ ID NO: 9 - DNA, 23 bp, synthetic construct
  source 1..23: mol_type = other DNA; note = primer binding site sequence
agtcgtcgcagcctcacctgatc

SEQ ID NO: 10 - DNA, 21 bp, synthetic construct
  source 1..21: mol_type = other DNA; note = primer binding site seqence
tcgtatgccgtcttctgcttg

SEQ ID NO: 11 - DNA, 20 bp, synthetic construct
  source 1..20: mol_type = other DNA; note = left universal binding sequence
aatgatacggcgaccaccga

SEQ ID NO: 12 - DNA, 25 bp, synthetic construct
  source 1..25: mol_type = other DNA; note = left universal binding sequence
catgtaatgcacgtactttcagggt

SEQ ID NO: 13 - DNA, 33 bp, synthetic construct; identical to SEQ ID NO: 4
  source 1..33: mol_type = other DNA; note = left universal binding sequence

SEQ ID NO: 14 - DNA, 33 bp, synthetic construct; identical to SEQ ID NO: 5
  source 1..33: mol_type = other DNA; note = left universal binding sequence

SEQ ID NO: 15 - DNA, 35 bp, synthetic construct; identical to SEQ ID NO: 3
  source 1..35: mol_type = other DNA; note = left universal binding sequence

SEQ ID NO: 16 - DNA, 34 bp, synthetic construct; identical to SEQ ID NO: 7
  source 1..34: mol_type = other DNA; note = left universal binding sequence

SEQ ID NO: 17 - DNA, 34 bp, synthetic construct; identical to SEQ ID NO: 8
  source 1..34: mol_type = other DNA; note = left universal binding sequence

SEQ ID NO: 18 - DNA, 35 bp, synthetic construct; identical to SEQ ID NO: 6
  source 1..35: mol_type = other DNA; note = left universal binding sequence

SEQ ID NO: 19 - DNA, 21 bp, synthetic construct
  source 1..21: mol_type = other DNA; note = right universal binding sequence
tcgtatgccgtcttctgcttg

SEQ ID NO: 20 - DNA, 23 bp, synthetic construct
  source 1..23: mol_type = other DNA; note = right universal binding sequence
agtcgtcgcagcctcacctgatc

SEQ ID NO: 21 - DNA, 25 bp, synthetic construct
  source 1..25: mol_type = other DNA; note = first sub-region of the second splint strand
catgtaatgcacgtactttcagggt

SEQ ID NO: 22 - DNA, 23 bp, synthetic construct
  source 1..23: mol_type = other DNA; note = second sub-region of the second splint strand
agtcgtcgcagcctcacctgatc

SEQ ID NO: 23 - DNA, 48 bp, synthetic construct
  source 1..48: mol_type = other DNA; note = first and second sub-region of the second splint strand
agtcgtcgcagcctcacctgatccatgtaatgcacgtactttcagggt

SEQ ID NO: 24 - DNA, 20 bp, synthetic construct
  source 1..20: mol_type = other DNA; note = first surface primer
tcggtggtcgccgtatcatt

SEQ ID NO: 25 - DNA, 20 bp, synthetic construct
  source 1..20: mol_type = other DNA; note = short P5 surface primer
aatgatacggcgaccaccga

SEQ ID NO: 26 - DNA, 24 bp, synthetic construct
  source 1..24: mol_type = other DNA; note = long P5 surface primer
aatgatacggcgaccaccgagatc

SEQ ID NO: 27 - DNA, 21 bp, synthetic construct
  source 1..21: mol_type = other DNA; note = second region of the second surface primer
caagcagaagacggcatacga

SEQ ID NO: 28 - DNA, 21 bp, synthetic construct
  source 1..21: mol_type = other DNA; note = short P7 surface primer
caagcagaagacggcatacga

SEQ ID NO: 29 - DNA, 24 bp, synthetic construct
  source 1..24: mol_type = other DNA; note = long P7 surface primer
caagcagaagacggcatacgagat

SEQ ID NO: 30 - DNA, 25 bp, synthetic construct
  source 1..25: mol_type = other DNA; note = fourth sub-region of first splint strand
accctgaaagtacgtgcattacatg

SEQ ID NO: 31 - DNA, 23 bp, synthetic construct
  source 1..23: mol_type = other DNA; note = fifth sub-region of first splint strand
gatcaggtgaggctgcgacgact

SEQ ID NO: 32 - DNA, 89 bp, synthetic construct
  source 1..89: mol_type = other DNA; note = fourth and fifth sub-region of first splint strand
tcggtggtcgccgtatcattaccctgaaagtacgtgcattacatggatcaggtgaggctgcgacgactcaagcagaagacggcatacga

SEQ ID NO: 33 - DNA, 35 bp, synthetic construct; identical to SEQ ID NO: 3
  source 1..35: mol_type = other DNA; note = splint sequencing primer

SEQ ID NO: 34 - DNA, 33 bp, synthetic construct; identical to SEQ ID NO: 4
  source 1..33: mol_type = other DNA; note = primer binding site sequence

SEQ ID NO: 35 - DNA, 15 bp, synthetic construct
  source 1..15: mol_type = other DNA; note = barcode sequence
aaaaattgtcttttt

SEQ ID NO: 36 - DNA, 19 bp, synthetic construct
  source 1..19: mol_type = other DNA; note = Tn5 transposon-end sequence
ctgtctcttatacacatct

SEQ ID NO: 37 - DNA, 19 bp, synthetic construct
  source 1..19: mol_type = other DNA; note = Tn5 transposon-end sequence
agatgtgtataagagacag

(19) United States
(12) Patent Application Publication
(10) Pub. No.: US 2026/0103752 A1
(43) Pub. Date: Apr. 16, 2026

(54) INCREASING SEQUENCING THROUGHPUT IN NEXT GENERATION SEQUENCING OF THREE-DIMENSIONAL SAMPLES

(71) Applicant: Element Biosciences, Inc., San Diego, CA (US)

(72) Inventors: Connor THOMPSON, San Diego, CA (US); Sinan ARSLAN, San Diego, CA (US)

(21) Appl. No.: 19/086,849

(22) Filed: Mar. 21, 2025

Related U.S. Application Data
(63) Continuation of application No. PCT/US2023/074933, filed on Sep. 22, 2023.
(60) Provisional application No. 63/409,546, filed on Sep. 23, 2022.
(60) Provisional application No. 63/413,864, filed on Oct. 6, 2022.

(51) Int. Cl. C12Q 1/6874; G06V 10/141; G06V 20/69

(52) U.S. Cl. CPC C12Q 1/6874; G06V 10/141; G06V 20/695; G06V 20/698; G06V 2201/03

(57) ABSTRACT

Described herein are aspects for sequencing three-dimensional samples using flow cell images. An aspect begins by obtaining a plurality of subsets of flow cell images of a sample in a plurality of sequencing cycles from a subset of channels. The aspect then generates base calls for the sample based on the subsets of flow cell images.

### REFERENCE TO SEQUENCE LISTING SUBMITTED ELECTRONICALLY

The content of the electronically submitted Sequence Listing XML, File Name: 4585_0080003_Sequencelisting_ST26, Size: 41,149 bytes, and Date of Creation: Aug. 14, 2025, submitted herewith, is hereby incorporated by reference in its entirety.

### CROSS REFERENCE TO RELATED APPLICATIONS

This application is a continuation of PCT/US2023/074933 filed Sep. 22, 2023, which claims the benefit of U.S. Provisional Patent Application Nos. 63/409,546, filed Sep. 23, 2022, and 63/413,864, filed Oct. 6, 2022, which are hereby incorporated by reference in their entireties.

### TECHNICAL FIELD

This disclosure relates generally to three-dimensional sequencing, and particularly to sequencing high density 3D samples that spatially overlap during DNA sequencing.

### BACKGROUND

In next-generation sequencing (NGS) or NGS-like applications such as sequencing by synthesis, sequencing by binding, or sequencing by avidity, in order to identify the sequence of a target nucleic acid, a new strand is synthesized one nucleotide base at a time. During each sequencing cycle, one base attaches to any given strand. At the imaging step of each cycle, image(s) are recorded. A base-calling algorithm is applied to the image(s) to “read” the successive signals from each cluster or polony and convert the optical signals into an identification of the nucleotide base sequence added to each DNA fragment.

Traditional 3D sequencing may not be capable to generate reliable sequencing results when clusters or polonies are spatially overlapped. As a result, the sequencing throughput is limited by the number of clusters or polonies that can be spatially separated. Further, usage of different sequencing primers to sequence different clusters or polonies would require additional time and effort to hybridize and block certain polonies and hybridization of primers to other polonies.

### BRIEF SUMMARY

Provided herein are system, apparatus, method, and/or computer program product embodiments, and/or combinations and sub-combinations thereof which enables 3D sequencing of samples such as in situ cells or tissues with increased sequencing throughput than existing systems and methods.

As a particular application of such, embodiments of methods, systems, and media for 3D sequencing of in situ samples are disclosed herein, so that higher than traditional sequencing throughput can be achieved.

Other embodiments of these aspects include corresponding computer systems, apparatus, and computer program product recorded on computer storage device(s), which, alone or in combination, configured to perform the actions of the methods. For a computer system configured or to be configured to perform operations or actions, the computer system has installed on it software, firmware, hardware, or their combinations that in operation cause the computer system to perform the operations or actions. For a computer program product configured or to be configured to perform operations or actions, the computer program product includes instructions that, when executed, by a hardware processor, cause the hardware processor to perform the operations or actions.

Further embodiments, features, and advantages of the present disclosure, as well as the structure and operation of the various embodiments of the present disclosure, are described in detail below with reference to the accompanying drawings.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the art(s) to make and use the embodiments.

FIG. 1 illustrates a block diagram of a system for performing 3D base calling of flow cell images, according to some embodiments.

FIGS. 2A-2C show exemplary flow cell images, processed images, and filtered images for 3D base calling, according to some embodiments.

FIGS. 3A-3C show exemplary flow cell images, processed images, and filtered images for 3D base calling, according to some embodiments.

FIGS. 3D-3E show an exemplary flow cell image and its corresponding filtered image for 3D base calling, according to some embodiments.

FIG. 4 illustrates a block diagram of a computer system for performing sequencing analysis and/or base calling, according to some embodiments.

FIG. 5 show an exemplary projection image of flow cell images taken at different axial locations of a 3D sample, according to some embodiments.

FIG. 6A is a flow chart of an exemplar method of performing 3D base calling of flow cell images, according to some embodiments.

FIG. 6B is a flow chart of an exemplar method of performing 3D base calling of flow cell images, according to some embodiments.

FIGS. 7A-7B show exemplary registration of a sequencing image of polonies with a cell staining image.

FIG. 8A shows a schematic diagram of flow cell images, subtiles, and regions of polonies, according to some embodiments.

FIG. 8B shows a schematic diagram of a portion of a flow cell with multiple tiles, according to some embodiments.

FIG. 9 illustrates a flow chart of a method for performing image registration of flow cell images, according to some embodiments.

FIGS. 10A-10B show a schematic diagram of an image transformation and corresponding 2D shifts, according to some embodiments.

FIGS. 11A-11C show exemplary barcode sequences that each can be used to uniquely identify polonies corresponding to a DNA or RNA fragment, according to some embodiments.

FIGS. 12A-12C show schematic diagrams of contacting polonies in an exemplary sequencing cycle before imaging with 4 different types of avidites (FIG. 12A), three different types of avidites (FIG. 12B), and 4 different types of avidite including a type of avidite with “dark” fluorescent dye (FIG. 12C), according to some embodiments.

FIG. 13 show exemplary barcode sequences for 4 subsets of polonies and imaging of individual subset at different sequencing cycles, according to some embodiments.

FIG. 14 is a schematic showing of an exemplary linear single stranded library molecule (1101) which comprises: a surface pinning primer binding site (1121); an optional left unique identification sequence (1181); a left index sequence (1161); a forward sequencing primer binding site (1141); an insert region having a sequence of interest (1111); reverse sequencing primer binding site (1151); a right index sequence (1171); and a surface capture primer binding site (1131).

FIG. 15 is a schematic showing an exemplary linear single stranded library molecule (1101) which comprises: a surface pinning primer binding site (1121); a left index sequence (1161); a forward sequencing primer binding site (1141); an insert region having a sequence of interest (1111); a reverse sequencing primer binding site (1151); a right index sequence (1171); an optional right unique identification sequence (1190); and a surface capture primer binding site (1131).

FIG. 16 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 17 is a schematic of an exemplary multivalent molecule comprising a generic core attached to a plurality of nucleotide-arms.

FIG. 18 is a schematic of an exemplary multivalent molecule comprising a dendrimer core attached to a plurality of nucleotide-arms.

FIG. 19 shows a schematic of an exemplary multivalent molecule comprising a core attached to a plurality of nucleotide-arms, where the nucleotide arms comprise biotin, spacer, linker and a nucleotide unit.

FIG. 20 is a schematic of an exemplary nucleotide-arm comprising a core attachment moiety, spacer, linker and nucleotide unit.

FIG. 21 shows the chemical structure of an exemplary spacer (top), and the chemical structures of various exemplary linkers, including an 11-atom Linker, 16-atom Linker, 23-atom Linker and an N3 Linker (bottom).

FIG. 22 shows the chemical structures of various exemplary linkers, including Linkers 1-9.

FIG. 23 shows the chemical structures of various exemplary linkers joined/attached to nucleotide units.

FIG. 24 shows the chemical structures of various exemplary linkers joined/attached to nucleotide units.

FIG. 25 shows the chemical structures of various exemplary linkers joined/attached to nucleotide units.

FIG. 26 shows the chemical structure of an exemplary biotinylated nucleotide-arm. In this example, the nucleotide unit is connected to the linker via a propargyl amine attachment at the 5 position of a pyrimidine base or the 7 position of a purine base.

FIG. 27 provides a schematic illustration of one embodiment of the low binding solid supports of the present disclosure in which the support comprises a glass substrate and alternating layers of hydrophilic coatings which are covalently or non-covalently adhered to the glass, and which further comprises chemically-reactive functional groups that serve as attachment sites for oligonucleotide primers.

FIG. 28 is a schematic of a guanine tetrad (e.g., G-tetrad).

FIG. 29 is a schematic of an exemplary intramolecular G-quadruplex structure.

FIG. 30A(i) is a schematic of an exemplary support having a plurality of nucleic acid capture primers arranged on the support in a non-predetermined and random manner.

FIG. 30A(ii) is a schematic of the same support shown in FIG. 30A, where individual nucleic acid capture primers are attached to a nucleic acid template molecule having one of four different batch sequences. The different batch sequences of the template molecules are represented by horizontal stripes, vertical dashed, brick or solid black.

FIG. 30B(iii) is a schematic of an exemplary support having a plurality of nucleic acid template molecules immobilized to the support (e.g., via attachment to the capture primers) where the template molecules are arranged on the support in a predetermined manner.

FIG. 30B(iv) is a schematic of an exemplary support having a plurality of nucleic acid template molecules immobilized to the support (e.g., via attachment to the capture primers) where the template molecules are arranged on the support in a predetermined manner.

FIG. 31A is a schematic showing an exemplary workflow for generating circularized padlock probes, comprising hybridizing first and second target-specific padlock probes to the first and second target molecules (respectively) to generate first and second circularized padlock probes (respectively) having a nick or gap, and closing the nick or gap to generate circularized padlock probes. In some embodiments, the first padlock probe comprises: (i) a batch-specific barcode sequence which corresponds to the first target sequence (Batch BC-1); (ii) a batch-specific sequencing primer binding site sequence which corresponds to the first target sequence (e.g., Batch Seq-1); (iii) a capture primer binding site; and (iv) a compaction oligonucleotide binding site. In some embodiments, the second padlock probe comprises: (i) a batch-specific barcode sequence which corresponds to the second target sequence (Batch BC-2); (ii) a batch-specific sequencing primer binding site sequence which corresponds to the second target sequence (e.g., Batch Seq-2); (iii) a capture primer binding site; and (iv) a compaction oligonucleotide binding site.

FIG. 31B is a schematic showing an exemplary workflow in which the circularized padlock probes shown in FIG. 31A are subjected to rolling circle amplification (RCA) to generate first and second concatemer template molecules which are immobilized to a support having one type of immobilized capture primers.

FIG. 32 is a schematic of an exemplary workflow in which circularized padlock probes are subjected to rolling circle amplification and batch sequencing.

FIG. 33 is a schematic of an exemplary workflow in which circularized padlock probes are subjected to rolling circle amplification and batch sequencing.

FIG. 34 is a schematic of an exemplary workflow in which circularized padlock probes are subjected to rolling circle amplification and batch sequencing.

FIG. 35 is a schematic of an exemplary workflow in which circularized padlock probes are subjected to rolling circle amplification and batch sequencing.

FIG. 36 is a schematic of an exemplary workflow in which circularized padlock probes are subjected to rolling circle amplification and batch sequencing.

FIG. 37 is a schematic of an exemplary workflow of a linear single stranded library molecule (100) hybridizing with a single-stranded splint molecule/strand (200) thereby circularizing the library molecule to form a library-splint complex (300) with a nick.

FIG. 38 is a schematic of an exemplary workflow of a linear single stranded library molecule (100) hybridizing with a single-stranded splint molecule/strand (200) thereby circularizing the library molecule to form a library-splint complex (300) with a nick.

FIG. 39A is a schematic of an exemplary workflow of a linear single stranded library molecule-1 (100) hybridizing with a single-stranded splint molecule/strand (200) thereby circularizing the library molecule to form a library-splint complex (300) with a nick which is enzymatically ligatable.

FIG. 39B is a schematic of an exemplary workflow of a linear single stranded library molecule-2 (100) hybridizing with a single-stranded splint molecule/strand (200) thereby circularizing the library molecule to form a library-splint complex (300) with a nick.

FIG. 40A is a schematic of an exemplary workflow in which the nick in the library-splint complex (300) shown in FIG. 39A is ligated to generate a first covalently closed circular library molecule (400) which is shown in FIG. 40A.

FIG. 40B is a schematic of an exemplary workflow in which the nick in the library-splint complex (300) shown in FIG. 39B is ligated to generate a second covalently closed circular library molecule (400) which is shown in FIG. 40B.

FIG. 41A is a schematic of an exemplary workflow of a linear single stranded library molecule-1 (100) hybridizing with a single-stranded splint molecule/strand (200) thereby circularizing the library molecule to form a library-splint complex (300) with a nick which is enzymatically ligatable.

FIG. 41B is a schematic of an exemplary workflow of a linear single stranded library molecule-2 (100) hybridizing with a single-stranded splint molecule/strand (200) thereby circularizing the library molecule to form a library-splint complex (300) with a nick.

FIG. 42A is a schematic of an exemplary workflow in which the nick in the library-splint complex (300) shown in FIG. 41A is ligated to generate a first covalently closed circular library molecule (400) which is shown in FIG. 42A.

FIG. 42B is a schematic of an exemplary workflow in which the nick in the library-splint complex (300) shown in FIG. 41B is ligated to generate a second covalently closed circular library molecule (400) which is shown in FIG. 42B.

FIG. 43 is a schematic of an exemplary workflow of a linear single stranded library molecule (100) hybridizing with a double-stranded adaptor (500) thereby circularizing the library molecule to form a library-splint complex (800) with two nicks.

FIG. 44 is a schematic of an exemplary workflow of a linear single stranded library molecule (100) hybridizing with a double-stranded adaptor (500) thereby circularizing the library molecule to form a library-splint complex (800) with two nicks.

FIG. 45 is a schematic of an exemplary workflow of a linear single stranded library molecule (100) hybridizing with a double-stranded adaptor (500) thereby circularizing the library molecule to form a library-splint complex (800) with two nicks.

FIG. 46A is a schematic of an exemplary workflow of a linear single stranded library molecule-1 (100) hybridizing with a double-stranded adaptor (500) thereby circularizing the library molecule to form a library-splint complex (800) with two nicks that are enzymatically ligatable.

FIG. 46B is a schematic of an exemplary workflow of a linear single stranded library molecule-2 (100) hybridizing with a double-stranded adaptor (500) thereby circularizing the library molecule to form a library-splint complex (800) with two nicks that are enzymatically ligatable.

FIG. 47A is a schematic of an exemplary workflow in which the two nicks in the library-splint complex (800) shown in FIG. 46A are ligated to generate a first covalently closed circular library molecule (900) which is shown in FIG. 47A.

FIG. 47B is a schematic of an exemplary workflow in which the nick in the library-splint complex (800) shown in FIG. 46B is ligated to generate a second covalently closed circular library molecule (900) which is shown in FIG. 47B.

FIG. 48 is a schematic showing an exemplary linear single stranded library molecule (100) hybridizing with a single-stranded splint molecule/strand (200) thereby circularizing the library molecule to form a library-splint complex (300) with a nick.

FIG. 49 is a schematic showing an exemplary linear single stranded library molecule (100) hybridizing with a double-stranded splint molecule (200) thereby circularizing the library molecule to form a library-splint complex (500) with two nicks.

FIG. 50 shows sequencing images of polonies (e.g., DNA nanoballs) immobilized on a support at high density.

FIGS. 51A-51C show flow charts of different embodiments of the method for sequencing and analysis of high spatial density sample(s) in 3D.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, method, and/or computer program product embodiments, and/or combinations and sub-combinations thereof which enables sequencing run and sequencing analysis of high spatial density three-dimensional (3D) samples in which polonies or clusters may overlap. The techniques disclosed herein are useful for accurate and reliable base calling in next generation sequencing (NGS), and NGS will be used as the primary example herein for describing the application of these techniques. However, such techniques may also be useful in other applications.

Traditional two-dimensional (2D) flow cell images can show clusters or polonies from 2D samples and base callings can be performed using their corresponding image intensities. The optical system can be tuned to image the clusters or polonies while in-focus. However, in situ samples such as cells or tissue can have a thickness along the axial or z direction that may not be in-focus within a single 2D image. Further, 3D samples may have spatially overlapped polonies or clusters. The techniques disclosed herein can be used for acquiring a stack of flow cell images of a 3D sample and generating accurate and reliable base calls for polonies or clusters within the 3D sample based on the flow cell images, even when the spatial density of polonies or clusters is higher than what traditional 2D or 3D sequencing systems can typically handle.

Existing sequencing systems and methods may have limited sequencing throughput due to constraints including but not limited to the number of reagent cartridges that limits the available number of different primers, optical designs and structures, and/or chemistry features of the sequencing reactions. The optical system (e.g., design and structure) may control image resolution together with image quality, thus also limiting the spatial density of samples that the existing systems can accurately and reliably handle. The techniques disclosed herein enables sequencing of higher spatial density samples in 3D (e.g., 2×, 3×, 5×, 10×, 20×, 30×, or even higher spatial density than what the existing sequencing systems can handle) thereby advantageously increasing the sequencing throughput by multiple folds without the need to improve existing cartridges, optical designs, and/or chemistry in sequencing systems. The techniques disclosed herein also advantageously remove the need to hybridize and/or dehybridize different subset of polonies associated with usage of different primers, and can exceed sequencing throughputs that can be achieved by using only the different primers. The techniques disclosed herein may also advantageously reduce cost of goods sold (COGS) by using reagents, e.g., avidites, associated with only a subset but not all 4 types of nucleotides. The techniques disclosed herein may also save imaging time by acquiring image only from a subset of channels, e.g., 3 of the 4 channel sequencing system. The techniques herein may be used to sequence 3D samples that are of higher plexity (e.g., more than 20 or 30) and/or lower diversity than what existing sequencing systems can possibly sequence.

### Sequencing Systems

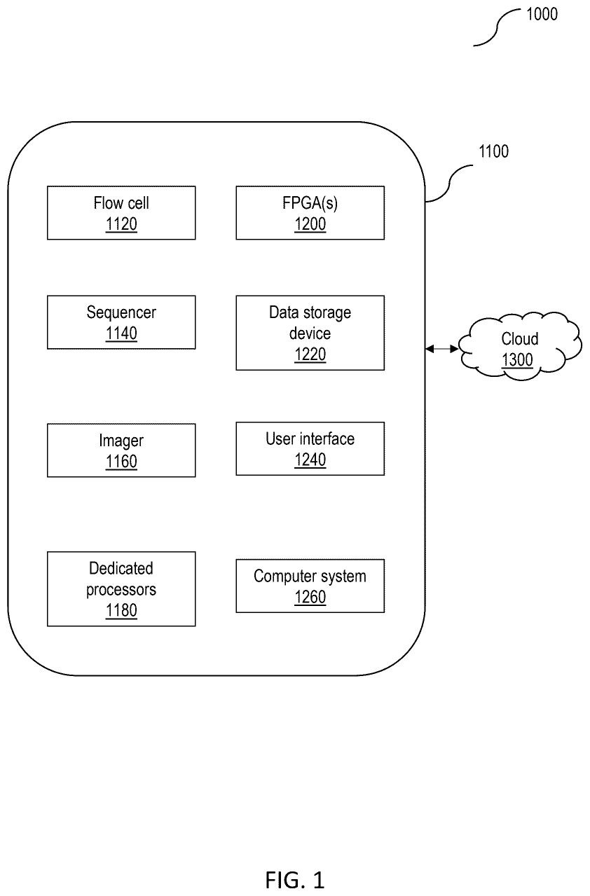

FIG. 1 illustrates a block diagram of a computer-implemented system 1000, according to one or more embodiments disclosed herein. The system 1000 has a sequencing system 1100 that includes a flow cell 1120, a sequencer 1140, an imager 1160, data storage device 1220, and user interface 1240. The sequencing system 1100 may be connected to a cloud 1300. The sequencing system 1100 may include one or more of dedicated processors 1180, Field-Programmable Gate Array(s) (FPGAs) 1200, and a computer system 1260.

In some embodiments, the flow cell 1120 is configured to capture DNA fragments and form DNA sequences for base-calling on the flow cell. The flow cell 1120 can include a support as disclosed herein. The support can be a solid support. The support can include a surface coating thereon as disclosed herein. The surface coating can be a polymer coating as disclosed herein.

A flow cell 1120 can include multiple tiles or imaging areas thereon, and each tile may be separated into a grid of subtiles. Each subtile can include a plurality of clusters or polonies thereon. As a nonlimiting example, a flow cell can have 424 tiles, and each tile can be divided into a 6×9 grid, therefore 54 subtiles. The flow cell image as disclosed herein can be an image including signals of a plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during an imaging or sequencing cycle using the imager 1160. In some embodiments, each tile may include millions of polonies or clusters. As a nonlimiting example, a tile can include about 1 to 10 million of clusters or polonies. Each polony can be a collection of many copies of DNA fragments.

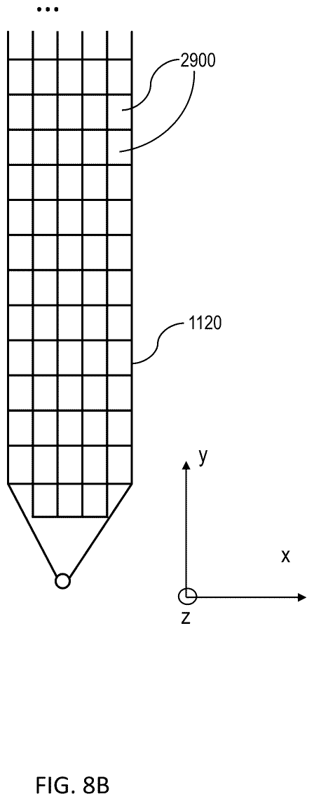

In cases where three-dimensional (3D) samples, e.g., cells or tissues are immobilized on the flow cell, are sequenced, the flow cell images may be acquired at multiple z levels which are orthogonal to the image plane of the flow cell images to cover the volume of the 3D sample. The z axis can extend from the objective lens of the optical system disclosed herein to the support, e.g., flow cell device. Each z level of flow cell images may be parallel to and separated from the adjacent z level(s) for a predetermined distance, for example, for about 0.1 um to about 15 ums. Each z level of flow cell images may be separated from the adjacent level(s) for 1 um to 10 ums. At each z-level, flow cell image(s) can be acquired from one or more sequencing cycles and/or one or more channels. Each flow cell image may include in its field of view at least part of one or more tiles or subtiles of the flow cell. FIG. 8B shows a portion of a flow cell 1120 with multiple tiles 2900. The image plane is defined by the x and y axis. And the z axis is orthogonal to the x-y plane. Although the flow cell images, samples, and the z axis are described in a Cartesian coordinate system, any other coordinate systems can be used to define spatial locations and relationships of the polonies or clusters and their images herein. Other coordinate systems can include but are not limited to the polar coordinate system, cylindrical, or spherical coordinate systems.

The sequencer 1140 may be configured to flow a nucleotide mixture onto the flow cell 1120, cleave blockers from the nucleotides in between flowing steps, and perform other steps for the formation of the DNA sequences on the flow cell 1120. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some embodiments, the sequencer 1140 and the flow cell 1120 may be configured to performing various sequencing methods disclosed herein, for example, sequencing-by-avidite.

For example, each nucleotide base may be assigned a color. Different types of nucleotides can have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

The imager 1160 may be configured to capture images of the flow cell 1120 after each flowing step. In an embodiment, the imager 1160 is a camera configured to capture digital images, such as a CMOS or a CCD camera. The camera may be configured to capture images at the wavelengths of the fluorescent elements bound to the nucleotides. The images can be called flow cell images.

In some embodiments, the imager 1160 can include one or more optical systems disclose herein. The optical system(s) can be configured to capture optical signals from the flow cell and generate corresponding digital images thereof. The digital images can then be used for base calling.

In an embodiment, the images of the flow cell may be captured in groups, where each image in the group is taken at a wavelength or in a spectrum that matches or includes only one of the fluorescent elements. In another embodiment, the images may be captured as single images that captures all of the wavelengths of the fluorescent elements.

The resolution of the imager 1160 can control the level of detail in the flow cell images, including pixel size. In existing systems, this resolution is very important, as it controls the accuracy with which a spot-finding algorithm identifies the polony centers. In some embodiments, the image resolution of flow cell images disclosed herein can be about 10 nanometers (nms) to a couple of hundreds of nms or greater. One way to increase the accuracy of spot finding is to improve the resolution of the imager 1160, or improve the processing performed on images taken by imager 1160. Detecting polony centers in pixels other than those detected by a spot-finding algorithm can be performed. These methods can allow for improved accuracy in detection of polony centers without increasing the resolution of the imager 1160. The resolution of the imager may even be less than existing systems with comparable performance, which may reduce the cost of the sequencing system 1100.

The image quality of the flow cell images can control the base calling quality. One way to increase the accuracy of base calling is to improve the imager 1160, or improve the processing performed on images taken by imager 1160 to result in a better image quality. The methods described herein register the flow cell images to a common coordinate system so that the base calling with respect to a cluster or polony can be more accurate than without such registration. These methods can allow for accurate and efficient base calling of 3D samples with higher system throughput than existing sequencing systems. Some or all of the operations disclosed herein can be advantageously performed by the FPGA(s) and data can be communicated between the CPU(s) and FPGA(s) to reduce the total operational time from methods operating without the FPGA(s). Further, instead of directly registering multiple flow cell images which may require saving the images before and/or after registration, image intensities and corresponding positions of selected polonies are extracted to estimate the transformation of the entire flow cell image.

The sequencing system 1100 may be configured to perform 3D sequencing runs and sequencing data analysis. The operations or actions disclosed herein may be performed by the dedicated processors 1180, the FPGA(s) 1200, the computer system 1260, or a combination thereof. One or more operations or actions in methods 6000 disclosed herein may be performed by the dedicated processors 1180, the FPGA(s) 1200, the computer system 1260, or a combination thereof. In some embodiments, which operations or actions are to be performed by performed by the dedicated processors 1180, the FPGA(s) 1200, the computer system 1260, or their combinations can be determined based on one or more of: a computation time for the specific operation(s), the complexity of computation in the specific operation(s), the need for data transmission between the hardware devices, or their combinations.

The computing system 1260 can include one or more general purpose computers that provide interfaces to run a variety of program in an operating system, such as Windows™ or Linux™. Such an operating system typically provides great flexibility to a user.

In some embodiments, the dedicated processors 1180 may be configured to perform operations in the methods disclosed herein. They may not be general-purpose processors, but instead custom processors with specific hardware or instructions for performing those steps. Dedicated processors directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the steps are performed and allow for real time processing.

In some embodiments, the dedicated processors 1180 or the computing system 1260 may comprise reconfigurable logic devices, such as artificial intelligence (AI) chips, neural processing units (NPUs), application specific integrated circuits (ASICs), or a combination there of. The reconfigurable logic devices may be configured to perform one or more operations herein. The reconfigurable logic devices may be configured to perform one or more operations herein and accelerate the operations by allowing parallel data processing in comparison to CPUs.

In some embodiments, the FPGA(s) 1200 may be configured to perform some or all of operations in the methods herein. An FPGA is programmed as hardware that will only perform a specific task. A special programming language may be used to transform software steps into hardware componentry. Once an FPGA is programmed, the hardware directly processes digital data that is provided to it without running software. The FPGA instead may use logic gates and registers to process the digital data. Because there is no overhead required for an operating system, an FPGA generally processes data faster than a general-purpose computer. Similar to dedicated processors, this is at the cost of flexibility.

The lack of software overhead may also allow an FPGA to operate faster than a dedicated processor, although this will depend on the exact processing to be performed and the specific FPGA and dedicated processor.

A group of FPGA(s) 1200 may be configured to perform the steps in parallel. For example, a number of FPGA(s) 1200 may be configured to perform a processing step for an image, a set of images, a subtile, or a select region in one or more images. Each FPGA(s) 1200 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over conventional systems may need to store the data before it may be processed, which may require more memory or accessing a computer system located in the cloud 1300.

In some embodiments, the data storage device 1220 is used to store information used in the methods herein. This information may include the flow cell images themselves or information and/or images derived from the flow images captured by the imager 1160. The DNA sequences determined from the base-calling may be stored in the data storage device 1220. The predetermined barcode sequences may be stored in the data storage. Parameters identifying polony locations may also be stored in the data storage device 1220. Raw and/or processed image intensities of each polony may be stored in the data storage. The region and/or subtile that each polony corresponds to may also be stored in the data storage device 1220. The transformation matrix of each region and/or subtile for different cycle(s) and/or channel(s) may also be stored in the data storage device 1220. Cell images may be stored in the data storage. The flow cell images, the processed images, and/or the filtered images may be stored in the data storage. Other information or images that can facilitate 3D base calling of the sample can be saved in the data storage.

The user interface 1240 may be used by a user to operate the sequencing system or access data stored in the data storage device 1220 or the computer system 1260.

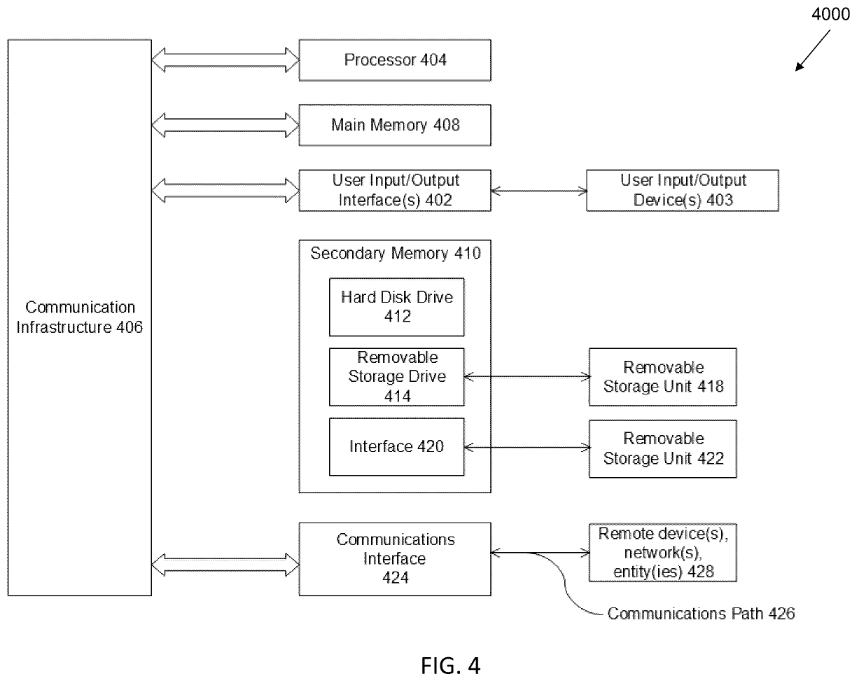

The computer system 1260 may control the general operation of the sequencing system and may be coupled to the user interface 1240. It may also perform steps in image processing, base calling, their preceding operations, and/or subsequent operations including but not limited to image registration. In some embodiments, the computer system 1260 is a computer system 4000, as described in more detail in FIG. 4. The computer system 1260 may store information regarding the operation of the sequencing system 1100, such as configuration information, instructions for operating the sequencing system 1100, or user information. The computer system 1260 may be configured to pass information between the sequencing system 1100 and the cloud 1300.

As discussed above, the sequencing system 1100 may have dedicated processors 1180, FPGA(s) 1200, or the computer system 1260. The sequencing system may use one, two, or all of these elements to accomplish necessary processing described above. In some embodiments, when these elements are present together, the processing tasks are split between them. For example, the FPGA(s) 1200 may be used to perform some or all of: the preprocessing operations, image processing, image registration, base calling, and any subsequent operations, while the computer system 1260 may perform other processing functions for the sequencing system 1100 such as registering images for base calling with cell staining image(s). Those skilled in the art will understand that various combinations of these elements will allow various system embodiments that balance efficiency and speed of processing with cost of processing elements.

The cloud 1300 may be a network, remote storage, or some other remote computing system separate from the sequencing system 1100. The connection to cloud 1300 may allow access to data stored externally to the sequencing system 1100 or allow for updating of software in the sequencing system 1100.

### Methods for Sequencing High Spatial Density Samples

During DNA sequencing of 3D samples, flow cell images of the sample may be acquired and bright spots in the flow cell images may represent different polonies or clusters with different light frequencies indicating different colors. When the spatial density of the sample is high, the bright spots representing different polonies and clusters may partially overlap with each other. As a result, it may be difficult to differentiate polonies and their corresponding colors apart using existing sequencing and analysis methods, thus errors may occur in base calling of high spatial density 3D samples using existing sequencing and analysis methods.

Disclosed herein are computer-implemented methods (e.g., 6000, 9000, 5200) for sequencing and/or performing 3D sequencing analysis on high spatial density samples in which polonies or clusters may overlap and cause errors in base calling using existing sequencing methods. The methods herein can include some or all of the operations disclosed herein. The operations may be performed in but is not limited to the order that is described herein.

The methods (e.g., 6000, 9000, 5200) herein can be performed by one or more processors disclosed herein. In some embodiments, the processor can include one or more of: a processing unit, an integrated circuit, or their combinations. For example, the processing unit can include a central processing unit (CPU), a graphic processing unit (GPU), and/or a neural processing unit (NPU). The integrated circuit can include a chip such as a field-programmable gate array (FPGA). In some embodiments, the processor can include the computer system 4000.

In some embodiments, some or all operations in the methods 6000, 9000, 5200 can be performed by the FPGA(s) and/or other devices, e.g., AI chips or NPUs. In embodiments when some operations are performed by FPGA(s), the data after an operation performed by the FPGA(s) can be communicated by the FPGA(s) s to other devices, e.g., the CPU(s) or NPUs, so that the other devices can perform subsequent operation(s) in the methods using such data. Similarly, data can also be communicated from the other devices to the FPGA(s) for processing by the FPGA(s). In some embodiments, all the operations in method 6000 can be performed by CPU(s). Alternatively, the operations performed by CPU(s) can be performed by other processors such as the dedicated processors, or NPU(s). In some embodiments, all the operations in method can be performed by FPGA(s). In some embodiments, some of the operations in methods 6000, 9000, and 5200 can be performed by FPGA(s) and some other operations in the methods are performed by AI chips or NPUs to improve energy consumption, heat dissipation, and/or computational time needed for sequencing analysis.

The flow cell images herein may be acquired using the optical system disclosed herein, from 1, 2, 3, 4, or more channels of the imager 1160. In some embodiments, the plurality of flow cell images are acquired in a single flow cycle or multiple flow cycles of a sequence run. Each flow cell image can include one or more tiles 2900 (imaging areas), and each tile can be divided into multiple subtiles. Each subtile can include a plurality of polonies or clusters. Each subtile can include multiple regions with each region including a number of polonies. For example, the polonies can be extracted or otherwise identified from corresponding regions of flow cell images from 4 different channels in a given cycle. As another example, the polonies can be extracted from flow cell images from a single channel. The flow cell image as disclosed herein can be an image that is acquired from imaging sample(s) immobilized on the flow cell 1120 0 as shown in FIG. 8B.

The flow cell 1120 may include sample(s) immobilized thereon. The sample(s) may include a plurality of nucleic acid template molecules. The sample(s) may include a two dimensional (2D) sample or a three-dimensional (3D) volumetric sample. The nucleic acid template molecules may be distributed randomly or in various patterns on the flow cell 1120. In some embodiments, the plurality of polonies or clusters herein may be extracted from specific regions of a tile, e.g., each subtile. With each subtile, the polonies may be extracted with a predetermined pattern or randomly.

In some embodiments, the polonies or clusters being sequenced in a flow cycle may have a certain nucleotide diversity, e.g., in base calling. The method may allow color correction of flow cell images even if the polonies or clusters are of low or unbalanced diversity in sequencing cycle(s). The nucleotide diversity of a population of nucleotide acid molecules, e.g., polonies or clusters, can refer to the relative proportion of nucleotides A, G, C, and T/U that are present in each flow cycle. The relative proportion of nucleotides may be within a region of the field of view or within the entire flow cell image. An optimally high or balanced diversity data can generally have approximately equal proportions of all four nucleotides represented in each flow cycle of a sequencing run. A low or unbalanced diversity data can generally include a high proportion of certain nucleotides and low proportion of other nucleotides in some flow cycles of a sequencing run, e.g., less than 10% of the total number of all 4 nucleotides. As a result, images corresponding to the high portion of certain nucleotides can have more signal spots (polonies or clusters) than images corresponding to the low portion of certain nucleotides. As an example of low or unbalanced diversity data, the bases A, T, C, G can be about 1%, about 2%, about 1%, and about 95%, respectively, of the total number of polonies, in a certain flow cycle. Subsequently, the flow cell images from channels corresponding to A, T, and C in this particular flow cycle are darker and with much fewer polonies or clusters than the flow cell image corresponding to nucleotide G. As another example of low or unbalanced diversity data, the bases A, T, C, G in polonies in multiple flow cycles can be about 2%, about 5%, about 10%, and about 83%, respectively. In embodiments where low or unbalanced diversity data is present in a particular cycle and is imaged for sequencing analysis, image registration using existing technologies may fail because image(s) from one or more channels are too dark (e.g., signal spots of polonies are too sparse and/or dim) comparing with images acquired from other channels thereby causing problems in subsequent color correction. Further, in embodiments where low or unbalanced diversity data is present in a particular cycle, correction of channel cross-talk using existing technologies may fail because image(s) from one or more channels are too dark (e.g., signal spots of polonies are too sparse and/or dim). In some embodiments, the methods (e.g., 6000, 9000, 5200) is configured to perform color correction of flow cell images even if the polonies or clusters are of low diversity.

The methods 6000, 9000, 5200 herein may enable sequencing and analysis of 3D samples, e.g., in situ samples like single cells or tissue with a spatial dense that is beyond what existing sequencing systems can handle with a predetermined quality level. For example, the 3D sample may have overlap of polonies or clusters, partially or completely, either in x-y plane or in 3D. The overlap, partially or completely, may cause inaccuracy in base calling of the overlapped polonies resulting in inaccurate DNA sequencing analysis. In some embodiments, such high density 3D sample(s) may be divided into groups as “batches,” and each batch may use a different sequencing primer. Details of exemplary embodiments of sequencing different batches of polonies or clusters are described in international patent application No. PCT/US23/65972 (where the contents are hereby incorporated by reference in its entirety). However, using different sequencing primers for binding polonies or clusters may require consumption of corresponding reagents and buffers, time and effort in hybridization and/or dehybridization of different “batches” of polonies, and the number of available primers may be limited by the number and volume of the cartridges in the sequencing system. The methods herein advantageously allow further increase of system throughput by 3×, 5×, 10×, or more than existing methods that sequence the samples in batches without changing the sequencing primers, cartridges, and the optical system.

In some embodiments, such high density 3D sample(s) being imaged can be divided into multiple subsets, e.g., the first, second, or third subset of polonies, by using different barcode sequence for identifying each different subset. For example, the overlapped polonies or clusters, e.g., of high spatial density sample(s), can be divided into different subsets. In some embodiments, the polonies in different subsets may use a same sequencing primer, thus the subsets are considered “sub-batches” within a single “batch.” During sequencing, one or more subsets can be turned “bright” and the other subset(s) that overlaps with the one or more subsets can be turned “dark,” so that “bright” polonies in flow cell images are of a lower spatial density than the spatial density of all the polonies. The different subsets can be “bright” and “dark” in different sequencing cycles such that each subset of polonies can be “bright” in at least some of the sequencing cycles, and optionally not in all the sequencing cycles. In some embodiments, the dark polonies or clusters are 5×, 8×, 10×, 12×, 15×, 20×, 30×, 40×, 50× dimer or more than the bright polonies. In some embodiments, the dark polonies are 5×, 8×, 10×, 12×, 15×, 20×, or 30× dimer than the bright polonies. In some embodiments, the dark polonies are about 0.5×, 0.8×, 1×, 1.2×, 1.5×, 2×, or 5× the intensity relative to an average background noise level of the corresponding flow cell image.

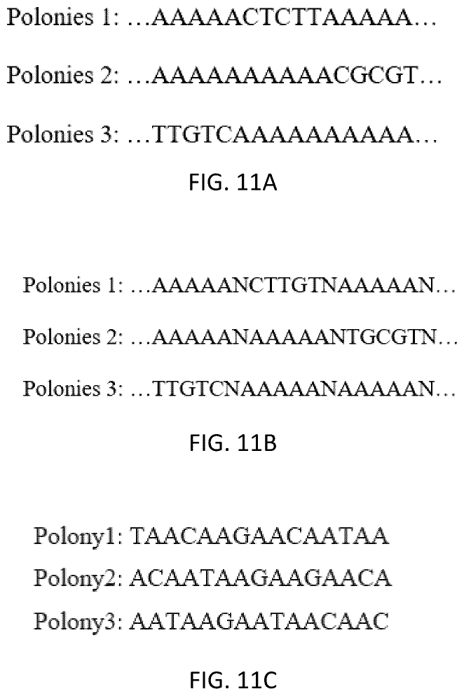

The total number of subsets may be customized based on the number of barcode sequences. Exemplary barcode sequences are shown in FIGS. 11A-11C. In some embodiment, the number of subsets can be customized to ensure that the spatial density of “bright” polonies does not deteriorate the accuracy and reliability in sequencing analysis. Each subset may have a spatial density that may be manageable by existing 3D sequencing systems. For example, each subset can have a spatial density that is equal to or less than the maximal spatial density that is manageable by existing 3D sequencing systems with a predetermined quality in base calling, e.g., Q40 or Q30. As another example, each subset may have a spatial density that is no less than about 0.01 to about 0.5 polonies/um³. As another example, each subset can have a spatial density that is no less than 0.01 to 0.5 polonies/um³. In some embodiments, each subset can have a spatial density of polonies or clusters that is no less than about 0.1 to about 1 polonies/um\hat{ }3. In some embodiments, each subset can have a spatial density of polonies or clusters that is no less than 0.1 to 1 polonies/um³. In some embodiments, each subset can have a spatial density of polonies or clusters that is no less than 0.002 to 50 polonies/um³. In some embodiments, each subset can have a spatial density of polonies or clusters that is no less than 0.005 to 30 polonies/um³. In some embodiments, the polony or cluster density is at least within the image plane, or the x-y plane. In some embodiments, the polony or cluster density is in 3D.

The total number of subsets may be customized based on spatial density of the sample(s). The total number of subsets may be an integer in the range from 3 to 8. The total number of subsets may be an integer in the range from 2 to 30. The total number of subsets may be an integer in the range from 2 to 100. For example, with 10 different sequencing primers and 4 different barcodes for each sequencing primer, the system throughput can be increased by 40 times using the methods disclosed herein. The spatial density of the sample that the sequencing system handles may be 40 times more than existing spatial density of 3D samples using existing sequencing systems and methods.

Sequencing without Imaging in the “Dark” Channel(s)

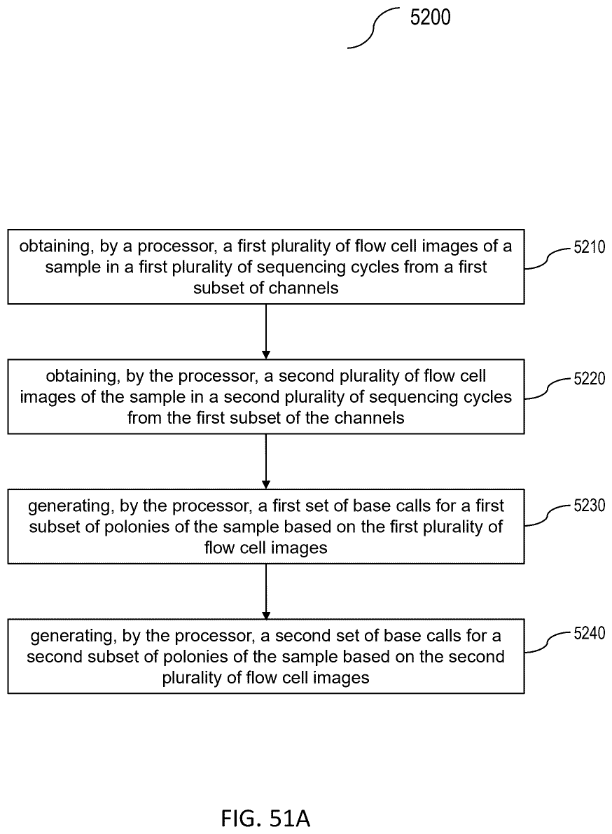

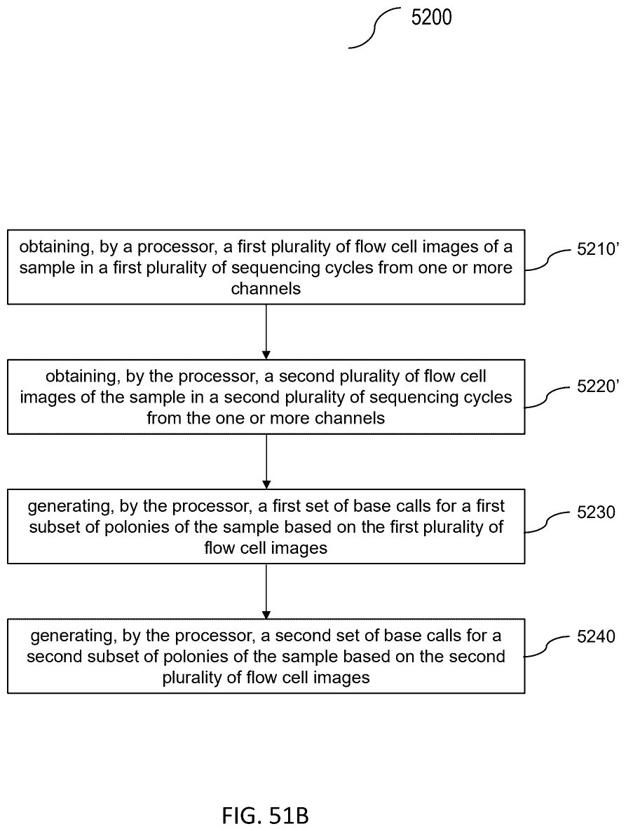

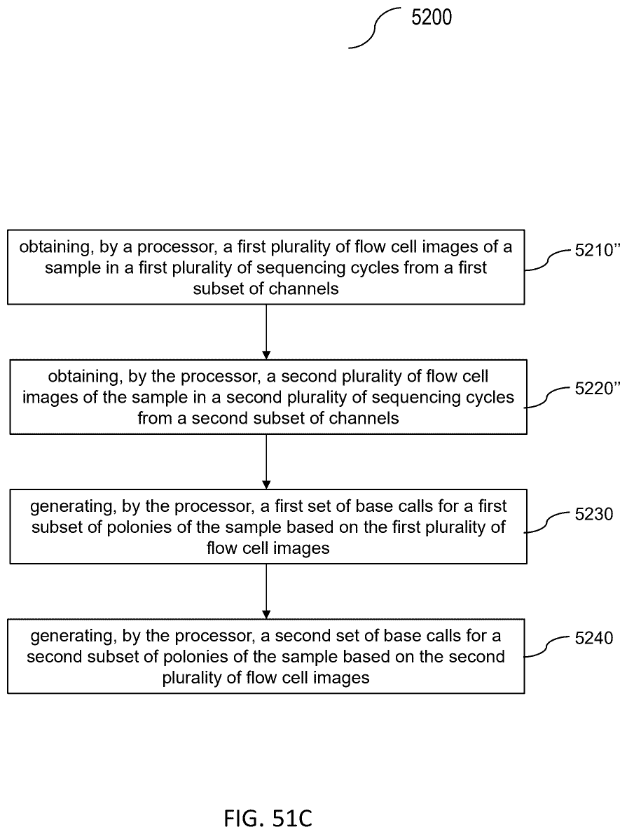

In some embodiments, the methods 5200 herein comprise obtaining flow cell images of sample(s) from some but not all of the channels, e.g., FIG. 51A. In some embodiments, the methods 5200 herein comprise obtaining flow cell images of sample(s) from the “bright” channels but not from the “dark” channels, e.g., FIG. 51A. In some embodiments, the flow cell images from the dark channels may be obtained/acquired but they are not used for generating base calls, e.g., as shown in FIG. 51B-51C. To save imaging time and cost for flowing reagents corresponding to the “dark” channel to the sample, e.g., avidite with “dark” dyes, it may be preferred to not acquire/obtain any flow cell images in the dark channel in some sequencing cycles and/or not contact the polonies with the avidite with dark fluorescent dyes or without dyes that corresponds to the dark channel in such sequencing cycles.

The sample(s) may be in 3D. The flow cell images can be acquired at different locations along an axial axis, i.e., the z axis shown in FIG. 8B. The flow cell images can be acquired in certain sequencing cycle(s) of a sequence run. The flow cell images can be acquired from one or more channels.

In some embodiments, the methods include imaging only from some but not all of the color channels of the sequencing system. The “dark” channel may not be used for imaging in at least some cycles, e.g., the cycles covering the barcode sequences. The “dark” channel may correspond to dark fluorescent dyes attached to a corresponding type of nucleotide bases, e.g., adenine (A). In some embodiments, the “dark” channel may correspond to lacking any fluorescent dyes that are attached to a corresponding type of nucleotide bases, e.g., adenine (A). In other words, the fluorescent dyes administered in such cycles may be some bright dyes and some dark dyes, or only bright dyes, and the number of different types of bright fluorescent dyes is less than the total number of channels.

There may be a single dark channel. In some embodiments, there may be multiple dark channels.

In some embodiments, the dark channel corresponds to a fluorescent dye attached to a nucleotide of adenine (A), thymine (T), guanine (G), or cytosine (C) that emits light below the predetermined threshold in the first, second, and/or third plurality of sequencing cycles. The predetermined threshold may be customized based on different sequencing applications. For example, the predetermined threshold may be a relative intensity level to the brightest image intensities within the same flow cell image(s). As another example, the predetermined threshold may be a relative intensity level to the noise intensities within the same flow cell image(s).

In some embodiments, the dark channel corresponds to lacking any fluorescent dye attached to a nucleotide of adenine (A), thymine (T), guanine (G), or cytosine (C) in the first plurality of sequencing cycles or the second plurality of sequencing cycles.

In some embodiments, the methods herein may use one or more identical dark channels corresponding to identical type(s) of nucleotide base(s) throughout the first, second, and/or third plurality of cycles flow cycles, e.g., throughout 15 cycles corresponding to sequencing the barcode sequence that has 15 bases. For example, the dark channels, e.g., the channel corresponding to nucleotide A, remain the same across the first, second, and/or third plurality of flow cycles. the dark channels, e.g., the channel corresponding to nucleotide A, remain the same across the first and second plurality of flow cycles.

In some embodiments, the method 5200 herein can comprise an operation 5210 of obtaining a first plurality of flow cell images of a sample in a first plurality of sequencing cycles from a first subset of channels; an operation 5220 of obtaining a second plurality of flow cell images of the sample in a second plurality of sequencing cycles from the first subset of the channels; an operation 5230 of generating, by the processor, a first set of base calls for the first subset of polonies of the sample based on the first plurality of flow cell images; and an operation 5240 of generating, by the processor, a second set of base calls for a second subset of polonies of the sample based on the second plurality of flow cell images. In some embodiments, the first subset of channels comprises only some but not all of the color channels of the sequencing system, and wherein the second plurality of sequencing cycles are subsequent to the first plurality of sequencing cycles.

In some embodiments, the image intensities of the second subset of polonies are below a predetermined threshold in the first plurality of flow cell images in the first plurality of sequencing cycles. The predetermined threshold can be customized based on different sequencing applications to be in various ranges. The second subset of polonies may appear 2×,5×, 8×, 10c, 12×, 15×, 18×, 20× darker or more than the first subset of polonies in the first plurality of cycles. In some embodiments, the image intensities of the second subset of polonies are 0.2×, 0.5×, 0.8×, 1×, 1.2×, 1.5×, 2×, 4×, 5× or more of the average background noise intensity in the corresponding flow cell images in the first plurality of sequencing cycles. In some embodiments, the image intensities of the second subset of polonies are less than 15%, 10%, 8%, 5%, 4%, 2%, or 1% of the highest signal intensities, e.g., an average of the top 1%, 2%, 3%, 4%, or 5% signal intensities, of the corresponding flow cell images in the first plurality of sequencing cycles. Individual intensities of each polony or average intensity of the polonies within the second subset of polonies may be used for comparison with the predetermined threshold, the average background noise, and/or the intensities of the first subset of polonies.

In some embodiments, the image intensities of the first subset of polonies are below a predetermined threshold in the second plurality of flow cell images. The predetermined threshold can be customized based on different sequencing applications to be in various ranges. In some embodiments, the first subset of polonies appear 5×, 10×, 15×, or 20× darker than the second subset of polonies in the second plurality of flow cell images. In some embodiments, the image intensities of the first subset of polonies are 0.2×, 0.5×, 0.8×, 1×, 1.2×, 1.5×, 2×, 4×, 5× or more of the average background noise intensity in the corresponding flow cell images in the second plurality of sequencing cycles. In some embodiments, the image intensities of the first subset of polonies are less than 15%, 10%, 8%, 5%, 4%, 2%, or 1% of the highest signal intensities, e.g., an average of the top 1%, 2%, 3%, 4%, or 5% signal intensities, of the corresponding flow cell images in the second plurality of sequencing cycles. Individual intensities of each polony or average intensity of the polonies within the first subset of polonies may be used for comparison with the predetermined threshold, the average background noise, and/or the intensities of the second subset of polonies.

In some embodiments, in the first plurality of flow cell images, image intensities of the second subset of polonies or any other polonies that are not in the first subset of polonies are below the predetermined threshold so that they appear “dark” in the first plurality of flow cell images. In some embodiments, the dark polonies are 5×, 8×, 10×, 12×, 15×, 20×, 30×, 40×, 50× dimer or more than the bright polonies, e.g., average intensity of the bright polonies. In some embodiments, the dark polonies are 5×, 8×, 10×, 12×, 15×, 20×, or 30× dimer than the bright polonies, e.g., average intensity of the bright polonies. In some embodiments, the dark polonies are about 0.5×, 0.8×, 1×, 1.2×, 1.5×, 2×, or 5× the intensity relative to an average background noise level of the corresponding flow cell image. Individual intensities of each polony or average intensity of the dark polonies may be used for comparison with the predetermined threshold, the average background noise, and/or the intensities of the bright polonies.

In some embodiments, the first subset of polonies is different from the second subset of polonies. In some embodiments, the first subset of polonies and the second subset of polonies are at least partly spatially overlapped either in the x-y plane or in 3D. In some embodiments, the first subset of polonies and the second subset of polonies comprise identical batch-specific sequencing binding sites configured to bind to identical sequencing primers, so that they are “sub-batches” within the same batch of polonies and clusters. In some embodiments, each polony of the first subset of polonies and the second subset of polonies comprises an identical batch-specific sequencing binding sites configured to bind to identical sequencing primers.

In some embodiments, each polony of the first subset of polonies is configured to bind to a first sequencing primer, and each polony of the second subset of polonies is configured to bind to a second sequencing primer different from the first sequencing primer. As such, the first and second subset of polonies are not within the same “batch.”

In some embodiments, the first set of base calls corresponds to the first subset of polonies in the first plurality of sequencing cycles. In some embodiments, the first set of base calls comprises only one, two, or three types of nucleotide bases but not all types of the nucleotide bases. In some embodiments, the second set of base calls corresponds to the second set of polonies in the second plurality of sequencing cycles. In some embodiments, the second set of base calls comprises only one, two, or three types of nucleotide bases but not all types of bases.

In some embodiments, the operation 5210 of obtaining the first plurality of flow cell images of the sample in the first plurality of sequencing cycles from the first subset of channels comprises: acquiring, by the optical system of the sequencing system, the first plurality of flow cell images of the sample in the first plurality of sequencing cycles only from the first subset of channels but not from any dark channel(s). In some embodiments, the operation 5210 of obtaining the first plurality of flow cell images of the sample in the first plurality of sequencing cycles from the first subset of channels comprises: controlling, by the processor, the optical system to avoid collecting data from one or more image sensors of the dark channel(s) in the first plurality of sequencing cycle. In some embodiments, the operation 5210 comprising: controlling, by the processor, the optical system to avoid illuminating light within a predetermined frequency range to the sample in the first plurality of sequencing cycle, the predetermined frequency range corresponding to the dark channel(s).

In some embodiments, the operation 5220 of obtaining the second plurality of flow cell images of the sample in the second plurality of sequencing cycles from the first subset of channels comprises: acquiring, by the optical system of the sequencing system, the second plurality of flow cell images of the sample in the second plurality of sequencing cycles only from the first subset of channels but not from any dark channel. In some embodiments, the operation 5220 of obtaining the second plurality of flow cell images of the sample in the second plurality of sequencing cycles from the first subset of channels comprises: controlling, by the processor, the optical system to avoid collecting any data from one or more image sensors of any dark channel(s) in the second plurality of sequencing cycle. In some embodiments, the operation 5220 of obtaining the second plurality of flow cell images of the sample in the second plurality of sequencing cycles from the first subset of channels comprises: controlling, by the processor, the optical system to avoid illuminating light within a predetermined frequency range to the sample in the second plurality of sequencing cycle, the predetermined frequency range corresponding to a dark channel.

In some embodiments, the operation 5230 of generating the first set of base calls for the first subset of polonies of the sample based on the first plurality of flow cell images comprises: generating the first set of base calls for the first subset of polonies of the sample only based on the first plurality of flow cell images obtained from the first subset of channels. In some embodiments, the operation 5240 of generating the second set of base calls for the second subset of polonies of the sample based on the second plurality of flow cell images comprises generating the second set of base calls for the second subset of polonies of the sample only based on the second plurality of flow cell images from the first subset of channels.

In some embodiments, the first plurality of sequencing cycles comprises an identical number of cycles as the second plurality of sequencing cycles. For example, the first or second plurality of cycles may each comprise 2, 3, 4, 5, 6, 7, 8, or more consecutive cycles in the sequencing run. In some embodiments, each of the first or second plurality of sequencing cycles comprises 2 to 50 consecutive cycles. In some embodiments, each of the first or second plurality of sequencing cycles comprises 2 to 20 consecutive cycles. In some embodiments, each of the first or second plurality of sequencing cycles comprises 3 to 8 consecutive cycles.

The sequence run may comprise any non-zero integer number of sequencing cycles, e.g., 150, 200, or 300. For example, the sequence run has 9, 10, 11, 12, 13, 14, 15, 18, 25 or more consecutive sequencing cycles that corresponds to the barcode sequences, e.g., 15 cycles as shown in FIGS. 11A-11C, that can be in any position of a sequencing run.

Referring to FIG. 11A, in a particular embodiment, the sample is divided into 3 subset of polonies. The first plurality of flow cell images can be from cycle n to cycle n+5 and obtained from a subset of channels corresponding to nucleotides T, G, and C, but not from the “dark” channel corresponding to nucleotide A (in polonies of subset 3), where n can be any non-zero integer, but smaller than the total number of cycles in the sequence run. The second plurality of flow cell images is from cycles n+6 to n+10 and obtained from a subset of channels corresponding to nucleotides T, and C, but not from the dark channel corresponding to nucleotide A (in polonies of subset 1). The second plurality of flow cell images may be obtained from a subset of channels corresponding to nucleotides T, C, and G.

Referring to FIG. 11A, in this particular embodiment, A is the “dark” base, and the channel corresponding to detecting fluorescent dyes attached to the type of avidite of A is a “dark” channel. The other three channels corresponding to bases T, C, and G are bright channels. In this embodiment, the first plurality of flow cell images are the flow cell images including bright signals from the first subset of polonies, i.e., “polonies 3,” in cycles n to n+5. In this particular embodiment, n equals one. Polonies in other subsets, i.e., “polonies 2” and “polonies 1” appear dark in the channels corresponding to nucleotides T, G, and C in the cycles n to n+5, so that the spatial density of the sample may be reduced in the first plurality of flow cell images by turning polonies 1 and polonies 2 dark in the cycles n to n+5. The first plurality of flow cell images may be from channels corresponding to bases T, C, and G. The other polonies in “polonies 2” and “polonies 1” may also appear dark in the dark channel corresponding to nucleotide A, as well.

Similarly, in the second plurality of flow cell images, image intensities of the first subset of polonies or any other polonies that are not in the second subset of polonies are below a predetermined threshold so that they appear dark. Continuing referring to FIG. 11A, the second plurality of flow cell images are the flow cell images from the subset “polonies 1” in cycles n+6 to n+10. Other polonies in “polonies 2” and “polonies 3” appear dark in channels corresponding to nucleotides T and C in these sequencing cycles. Although channel corresponding to nucleotide G is a “bright” channel in this embodiments, there may be no bright signal in the cycles n+6 to n+10, but there may be signals in cycles n+1 to n+5. In comparison, the dark channel corresponding to A remains dark throughout cycles n+1 to n+15.

In some embodiments, the method 5200 herein further comprises an operation of obtaining a third plurality of flow cell images of the sample in a third plurality of sequencing cycles from the first subset of channels.

Continuing referring to FIG. 11A, the third plurality of flow cell images corresponds the subset of polonies labeled as “polonies 2” in cycles n+11 to n+15. The first subset of channels, in this embodiments, includes 3 channels corresponding to bases C, G, and T, but not A.

The first subset of channels may not include the dark channels. The first subset of channels may not include all the channels, e.g., 4, of the sequencing system. Operations 5210, 5220, and the operation of acquiring the third plurality of flow cell images may use some or all channels of the first subset of channels. The channels from which one of the first, second, third or other pluralities of flow cell images are obtained may or may not be identical as the channels from which another one of the first, second, third or other pluralities of flow cell images are obtained, as shown in FIGS. 11A-11B. For example, the first plurality of flow cell images are acquired from channels corresponding to T, G, and C, while the second plurality of flow cell images are acquired from channels corresponding to T, G, and C, but more particularly, from channels of C and T only.

In some embodiments, the subset of channels from which the first plurality of flow cell images are obtained in the first plurality of sequencing cycles may not include any dark channel(s). Similarly, the subsets of channels from which the second or third plurality of flow cell images are obtained in their corresponding sequencing cycles may not include any dark channels. As shown in FIGS. 11A-11B, the subsets of channels for acquiring the first, second, and third plurality of flow cell images do not include the dark channel corresponding to “A.” The dark channel herein can be a channel that detects emitted light from fluorescent dye(s) attached a specific nucleotide, e.g., A, but the emitted light is below a predetermined threshold so that the flow cell images from the dark channel appear dark.

In some embodiments, operations 5230 and 5240 are based on only the flow cell images from the first subset of channels. The time and computation for acquiring flow cell images in the dark channel and/or performing base calling using the flow cell images from the dark channel may be saved thereby reducing the total time needed for sequencing and analysis.

### Sequencing Using all Channels

In some embodiments, the methods 6000, 9000, 5200 may include operations that obtains flow cell images from all the channels of the sequencing system including the “dark” channels.

In some embodiments, as shown in FIG. 51B, the methods herein include an operation of obtaining flow cell images from all the channels of the sequencing system. In some embodiments, the method comprises an operation 5210′ of obtaining, by a processor, a first plurality of flow cell images of a sample in a first plurality of sequencing cycles from one or more channels; an operation 5220′ of obtaining, by the processor, a second plurality of flow cell images of the sample in a second plurality of sequencing cycles from the one or more channels; an operation 5230 of generating, by the processor, a first set of base calls for a first subset of polonies of the sample based on the first plurality of flow cell images; and an operation 5240 of generating, by the processor, a second set of base calls for a second subset of polonies of the sample based on the second plurality of flow cell images. In some embodiments, the one or more channels comprise a dark channel in which image intensities of some of the first or second plurality of flow cell images are below a predetermined threshold, and wherein the second plurality of sequencing cycles are subsequent to the first plurality of sequencing cycles.

In some embodiments, the operations 5210′ and 5220′ are similar to the operations 5210 and operation 5220 except that the operation 5210′ and 5220′ are from the one or more channels.

In some embodiments, the operation 5210′ comprises: acquiring, by an optical system of a sequencing system, the first plurality of flow cell images of the sample in the first plurality of sequencing cycles from the one or more channels. In some embodiments, the operation 5220′ comprises acquiring, by an optical system of a sequencing system, the second plurality of flow cell images of the sample in the second plurality of sequencing cycles from the one or more channels. In some embodiments, the one or more channels comprises at least a dark channel and at least a channel that is not a dark channel. In some embodiments, the one or more channels comprises only a single dark channel and two or three channels different from the dark channel.

As shown in FIG. 51B, the methods 5200 may include operations 5230 and 5240 as shown in FIG. 51A and disclosed herein in relation to FIG. 51A.

In some embodiments, the image intensities of the second subset of polonies are below a predetermined threshold in the first plurality of flow cell images in the first plurality of sequencing cycles. The second subset of polonies may appear 2×,5×, 8×, 10c, 12×, 15×, 18×, 20× darker or more than the first subset of polonies in the first plurality of cycles. In some embodiments, the image intensities of the second subset of polonies are 0.2×, 0.5×, 0.8×, 1×, 1.2×, 1.5×, 2×, 4×, 5× or more of the average background noise intensity in the corresponding flow cell images in the first plurality of sequencing cycles. In some embodiments, the image intensities of the second subset of polonies are less than 15%, 10%, 8%, 5%, 4%, 2%, or 1% of the highest signal intensities, e.g., an average of the top 1%, 2%, 3%, 4%, or 5% signal intensities, of the corresponding flow cell images in the first plurality of sequencing cycles.

In some embodiments, the image intensities of the first subset of polonies are below a predetermined threshold in the second plurality of flow cell images. In some embodiments, the first subset of polonies appear 5×, 10×, 15×, or 20× darker than the second subset of polonies in the second plurality of flow cell images. In some embodiments, the image intensities of the first subset of polonies are 0.2×, 0.5×, 0.8×, 1×, 1.2×, 1.5×, 2×, 4×, 5× or more of the average background noise intensity in the corresponding flow cell images in the second plurality of sequencing cycles. In some embodiments, the image intensities of the first subset of polonies are less than 15%, 10%, 8%, 5%, 4%, 2%, or 1% of the highest signal intensities, e.g., an average of the top 1%, 2%, 3%, 4%, or 5% signal intensities, of the corresponding flow cell images in the second plurality of sequencing cycles.

In some embodiments, in the first plurality of flow cell images, image intensities of the second subset of polonies or any other polonies that are not in the first subset of polonies are below the predetermined threshold so that they appear “dark” in the first plurality of flow cell images. In some embodiments, the dark polonies are 5×, 8×, 10×, 12×, 15×, 20×, 30×, 40×, 50× dimer or more than the bright polonies, e.g., average intensity of the bright polonies. In some embodiments, the dark polonies are 5×, 8×, 10×, 12×, 15×, 20×, or 30× dimer than the bright polonies, e.g., average intensity of the bright polonies. In some embodiments, the dark polonies are about 0.5×, 0.8×, 1×, 1.2×, 1.5×, 2×, or 5× the intensity relative to an average background noise level of the corresponding flow cell image.

In some embodiments, the first subset of polonies is different from the second subset of polonies. In some embodiments, the first subset of polonies and the second subset of polonies are at least partly overlapped spatially either in the x-y plane or in 3D. In some embodiments, the first subset of polonies and the second subset of polonies comprise identical batch-specific sequencing binding sites configured to bind to identical sequencing primers, so that they are “sub-batches” within the same batch of polonies and clusters. In some embodiments, each polony of the first subset of polonies and the second subset of polonies comprises an identical batch-specific sequencing binding sites configured to bind to identical sequencing primers.

In some embodiments, each polony of the first subset of polonies is configured to bind to a first sequencing primer, and each polony of the second subset of polonies is configured to bind to a second sequencing primer different from the first sequencing primer. As such, the first and second subset of polonies are not within the same “batch.”

In some embodiments, the first set of base calls corresponds to the first set of polonies in the first plurality of sequencing cycles. In some embodiments, the first set of base calls comprises all four types of the nucleotide bases. In some embodiments, the second set of base calls corresponds to the second set of polonies in the second plurality of sequencing cycles. In some embodiments, the second set of base calls comprises all four types of nucleotide bases.

In some embodiments, the operation 5210′ of obtaining the first plurality of flow cell images of the sample in the first plurality of sequencing cycles from the one or more channels comprises: acquiring, by the optical system of the sequencing system, the first plurality of flow cell images of the sample in the first plurality of sequencing cycles from the one or more channels, which may include the dark channel(s). In some embodiments, the operation 5210′ of obtaining the first plurality of flow cell images of the sample in the first plurality of sequencing cycles from the one or more channels comprises: controlling, by the processor, the optical system to collecting data from one or more image sensors of the one or more channel(s) in the first plurality of sequencing cycle; controlling, by the processor, the optical system to illuminate light within a predetermined frequency range to the sample in the first plurality of sequencing cycle, the predetermined frequency range corresponding to the dark channel(s).

In some embodiments, the operation 5220′ of obtaining the second plurality of flow cell images of the sample in the second plurality of sequencing cycles from the one or more channels comprises: acquiring, by the optical system of the sequencing system, the second plurality of flow cell images of the sample in the second plurality of sequencing cycles from the one or more channels, which may or may not include the dark channels. In some embodiments, the operation 5220′ of obtaining the second plurality of flow cell images of the sample in the second plurality of sequencing cycles from the one or more channels comprises: controlling, by the processor, the optical system to collect data from one or more image sensors corresponding to the one or more channel(s) in the second plurality of sequencing cycle. In some embodiments, the operation 5220′ of obtaining the second plurality of flow cell images of the sample in the second plurality of sequencing cycles from the one or more channels comprises: controlling, by the processor, the optical system to illuminate light within a predetermined frequency range to the sample in the second plurality of sequencing cycle, the predetermined frequency range corresponding to a dark channel(s).

In some embodiments, the operation 5230 of generating the first set of base calls for the first subset of polonies of the sample based on the first plurality of flow cell images comprises: generating the first set of base calls for the first subset of polonies of the sample only based on the first plurality of flow cell images obtained from the one or more channels. In some embodiments, the operation 5240 of generating the second set of base calls for the second subset of polonies of the sample based on the second plurality of flow cell images comprises generating the second set of base calls for the second subset of polonies of the sample only based on the second plurality of flow cell images from the one or more channels.

In some embodiments, the first plurality of sequencing cycles comprises an identical number of cycles as the second plurality of sequencing cycles. For example, the first or second plurality of cycles may each comprise 2, 3, 4, 5, 6, 7, 8, or more consecutive cycles in the sequencing run. In some embodiments, each of the first or second plurality of sequencing cycles comprises 2 to 60 cycles. In some embodiments, the first or second plurality of sequencing cycles comprises 2 to 40 cycles. In some embodiments, the first or second plurality of sequencing cycles comprises 3 to 15 cycles.

The sequence run may comprise any non-zero integer number of sequencing cycles. For example, the sequence run has 10, 12, 15, 24 or more consecutive sequencing cycles that corresponds to the barcode sequences, e.g., 15 cycles as shown in FIGS. 11A-11C, that can be in any position of a sequencing run.

In some embodiments, the method herein further comprises an operation of obtaining a third plurality of flow cell images of the sample in a third plurality of sequencing cycles from the one or more channels.

The one or more channels may include some or all of the dark channel(s). The one or more channels may not include all the channels, e.g., 4, of the sequencing system. Operations 5210′, 5220′, and the operation of acquiring the third plurality of flow cell images may use one or more channels. The channels from which one of the first, second, third or other pluralities of flow cell images are obtained may or may not be identical as the channels from which another one of the first, second, third or other pluralities of flow cell images are obtained, as shown in FIGS. 11A-11B. For example, the first plurality of flow cell images are acquired from channels corresponding to T, G, and C, while the second plurality of flow cell images are acquired from channels corresponding to T, G, and C, but more particularly, from channels of C and T only.

In some embodiments, the channels from which that the first plurality of flow cell images are obtained in the first plurality of sequencing cycles may include one or more dark channel(s). Similarly, the channels from which the second or third plurality of flow cell images are obtained in their corresponding sequencing cycles may include one or more dark channels. For example, the channels for acquiring the first, second, and third plurality of flow cell images include all four channels. Each dark channel herein can be a channel that detects emitted light from fluorescent dye(s) attached a specific nucleotide, e.g., A, but the emitted light is below a predetermined threshold so that the flow cell images from the dark channel appear dark.

In some embodiments, the dark channel corresponds to a channel from which no emission of fluorescent light above a predetermined threshold and in a frequency range corresponding to the dark channel is generated from the sample in the first plurality of sequencing cycles. In some embodiments, the signal intensity of the flow cell images obtained from the dark channel may be much dimmer, e.g., 10× less than the bright spots in flow cell images from other channels. In some embodiments, the signal intensity of the flow cell images obtained from the dark channel may be comparable with background noise so that the flow cell images acquired or obtained from the dark channel may not be used for base calling to avoid possible errors in base calls. Further, the time and computation for performing base calling using the flow cell images may be reduced. In other words, base calling may be performed using flow cell images from the bright channels, e.g., 2 or 3 bright channels.

Sequencing with Different “Dark” Channel(s)

In some embodiments, the methods 6000, 9000, 5200 may include operations that obtain or acquire flow cell images from one or more channels of the sequencing system. The one or more channels may not include the dark channel(s). In some embodiments, the one or more channels may include all the channels including the dark channel(s). The dark channels in the first plurality of sequencing cycles may be different from the dark channels in the second plurality of sequencing cycles. In other words, the dark channel(s) may be alternating but not consistent in multiple consecutive cycles of a sequencing run, for example, the consecutive cycles covering the nucleotide bases of barcodes.

In some embodiments, the methods 5200 herein include an operation 5210″ of obtaining, by the processor, the first plurality of flow cell images of the sample in the first plurality of sequencing cycles from a first subset of channels; an operation 5220″ of obtaining, by the processor, a second plurality of flow cell images of the sample in a second plurality of sequencing cycles from a second subset of the channels; an operation 5230 of generating, by the processor, a first set of base calls for a first subset of polonies of the sample based on the first plurality of flow cell images; and an operation 5240 of generating, by the processor, a second set of base calls for a second subset of polonies of the sample based on the second plurality of flow cell images. In some embodiments, the first and second subsets of channels are at least partly different and the one or more channels does not comprise a first dark channel, and the second subset of channels does not comprise a second dark channel different from the first dark channel.

In embodiments where flow cell images are only obtained or acquired from bright channels but not dark channel(s), the first and second subsets of channels are at least partly different and the one or more channels does not comprise a first dark channel, e.g., corresponding to nucleotide A in the first plurality of cycles, and the second subset of channels does not comprise a second dark channel different from the first dark channel, e.g., corresponding to nucleotide C in the second plurality of cycles.

In embodiments where flow cell images are obtained or acquired from the dark channels, the first and second subsets of the channels are identical and the one or more channels may include the dark channel(s). In such embodiments, the first and second subsets of the one or more channels may each include all of the one or more channels, e.g. all four channels of the sequencing system.

Alternating the dark channel(s), e.g., from the channel corresponding to nucleotide A in the first plurality of cycles to nucleotide C in the second plurality of cycles, may be compatible with operations in methods 5200 when the operations include obtaining flow cell images from the dark channel(s). In some embodiments, alternating the dark channel may be compatible in methods 5200 when the operations include obtaining flow cell images from some or all of the bright channels but not the dark channel(s).

In some embodiments, the operations 5210″ and 5220″ are similar to the operations 5210 and operation 5220 except that the operation 5210″ and 5220″ correspond to different subsets of the one or more channels, i.e., the first and second subsets of channels. The first subset may not include a first dark channel while the second subset may not include a second dark channel different from the first channel.

As shown in FIG. 51C, the methods 5200 may include operations 5230 and 5240 as shown in FIGS. 52A-52B and disclosed herein in relation to FIGS. 51A-51B.

In some embodiments, the image intensities of the second subset of polonies are below a predetermined threshold in the first plurality of flow cell images in the first plurality of sequencing cycles. The predetermined threshold can be customized based on different sequencing applications to be within various ranges. The second subset of polonies may appear 2×, 5×, 8×, 10c, 12×, 15×, 18×, 20× darker or more than the first subset of polonies in the first plurality of cycles. In some embodiments, the image intensities of the second subset of polonies are 0.2×, 0.5×, 0.8×, 1×, 1.2×, 1.5×, 2×, 4×, 5× or more of the average background noise intensity in the corresponding flow cell images in the first plurality of sequencing cycles. In some embodiments, the image intensities of the second subset of polonies are less than 15%, 10%, 8%, 5%, 4%, 2%, or 1% of the highest signal intensities, e.g., an average of the top 1%, 2%, 3%, 4%, or 5% signal intensities, of the corresponding flow cell images in the first plurality of sequencing cycles. Individual intensities of each polony or average intensity of the polonies within the second subset of polonies may be used for comparison with the predetermined threshold, the average background noise, and/or the intensities of the first subset of polonies.

In some embodiments, the image intensities of the first subset of polonies are below a predetermined threshold in the second plurality of flow cell images. The predetermined threshold can be customized based on different sequencing applications to various ranges. In some embodiments, the first subset of polonies appear 5×, 10×, 15×, or 20× darker than the second subset of polonies in the second plurality of flow cell images. In some embodiments, the image intensities of the first subset of polonies are 0.2×, 0.5×, 0.8×, 1×, 1.2×, 1.5×, 2×, 4×, 5× or more of the average background noise intensity in the corresponding flow cell images in the second plurality of sequencing cycles. In some embodiments, the image intensities of the first subset of polonies are less than 15%, 10%, 8%, 5%, 4%, 2%, or 1% of the highest signal intensities, e.g., an average of the top 1%, 2%, 3%, 4%, or 5% signal intensities, of the corresponding flow cell images in the second plurality of sequencing cycles. Individual intensities of each polony or average intensity of the polonies within the first subset of polonies may be used for comparison with the predetermined threshold, the average background noise, and/or the intensities of the second subset of polonies.

In some embodiments, in the first plurality of flow cell images, image intensities of the second subset of polonies or any other polonies that are not in the first subset of polonies are below the predetermined threshold so that they appear “dark” in the first plurality of flow cell images. In some embodiments, the dark polonies are 5×, 8×, 10×, 12×, 15×, 20×, 30×, 40×, 50× dimer or more than the bright polonies, e.g., average intensity of the bright polonies. In some embodiments, the dark polonies are 5×, 8×, 10×, 12×, 15×, 20×, or 30× dimer than the bright polonies, e.g., average intensity of the bright polonies. In some embodiments, the dark polonies are about 0.5×, 0.8×, 1×, 1.2×, 1.5×, 2×, or 5× the intensity relative to an average background noise level of the corresponding flow cell image.

In some embodiments, the first subset of polonies is different from the second subset of polonies. In some embodiments, the first subset of polonies and the second subset of polonies are at least partly overlapped spatially either in the x-y plane or in 3D. In some embodiments, the first subset of polonies and the second subset of polonies comprise identical batch-specific sequencing binding sites configured to bind to identical sequencing primers, so that they are “sub-batches” within the same batch of polonies and clusters. In some embodiments, each polony of the first subset of polonies and the second subset of polonies comprises an identical batch-specific sequencing binding sites configured to bind to identical sequencing primers.

In some embodiments, each polony of the first subset of polonies is configured to bind to a first sequencing primer, and each polony of the second subset of polonies is configured to bind to a second sequencing primer different from the first sequencing primer. As such, the first and second subset of polonies are not within the same “batch.”

In some embodiments, the first set of base calls corresponds to the first set of polonies in the first plurality of sequencing cycles. In some embodiments, the first set of base calls comprises only 1, 2, or 3 types of the nucleotide bases. In some embodiments, the second set of base calls corresponds to the second set of polonies in the second plurality of sequencing cycles. In some embodiments, the second set of base calls comprises only 1, 2, or 3 types of nucleotide bases. For example, when the alternating dark channels are not imaged, the first set of base calls may include A, T, C, but not G which corresponds to the first dark channel while the second set of base calls may include T, C, and G but not A, which correspond to the second dark channel. In some embodiments, the first set of base calls comprises all four types of the nucleotide bases. In some embodiments, the second set of base calls corresponds to the second set of polonies in the second plurality of sequencing cycles. In some embodiments, the second set of base calls comprises all four types of nucleotide bases. For examples, each of the first and second set of base calls may include all four types of nucleotide bases with G being the dark channel in the first plurality of cycles and A being the second dark channel in the second plurality of cycles.

In some embodiments, the operation 5210″ of obtaining the first plurality of flow cell images of the sample in the first plurality of sequencing cycles from the one or more channels comprises: acquiring, by the optical system of the sequencing system, the first plurality of flow cell images of the sample in the first plurality of sequencing cycles the first subset of channels, which may or may not include a first dark channel(s). In some embodiments, the operation 5210″ of obtaining the first plurality of flow cell images of the sample in the first plurality of sequencing cycles from the first subset of channels comprises: controlling, by the processor, the optical system to avoid collecting data from one or more image sensors of the dark channel(s) in the first plurality of sequencing cycle; controlling, by the processor, the optical system to avoid illuminating light within a predetermined frequency range to the sample in the first plurality of sequencing cycle, the predetermined frequency range corresponding to the first dark channel(s).

In some embodiments, the operation 5220″ of obtaining the second plurality of flow cell images of the sample in the second plurality of sequencing cycles from the second subset of channels comprises: acquiring, by the optical system of the sequencing system, the second plurality of flow cell images of the sample in the second plurality of sequencing cycles from the second subset of channels, which may or may not include a second dark channel(s). In some embodiments, the operation 5220″ of obtaining the second plurality of flow cell images of the sample in the second plurality of sequencing cycles from the one or more channels comprises: controlling, by the processor, the optical system to avoid collecting any data from one or more image sensors of any dark channel(s) in the second plurality of sequencing cycle. In some embodiments, the operation 5220″ of obtaining the second plurality of flow cell images of the sample in the second plurality of sequencing cycles from the second subset of channels comprises: controlling, by the processor, the optical system to avoid illuminating light within a predetermined frequency range to the sample in the second plurality of sequencing cycle, the predetermined frequency range corresponding to the second dark channel(s).

In some embodiments, the operation 5230 of generating the first set of base calls for the first subset of polonies of the sample based on the first plurality of flow cell images comprises: generating the first set of base calls for the first subset of polonies of the sample based on the first plurality of flow cell images obtained from the first subset of channels. In some embodiments, the operation 5240 of generating the second set of base calls for the second subset of polonies of the sample based on the second plurality of flow cell images comprises generating the second set of base calls for the second subset of polonies of the sample only based on the second plurality of flow cell images from the second subset of channels.

In some embodiments, the first plurality of sequencing cycles comprises an identical number of cycles as the second plurality of sequencing cycles. For example, the first or second plurality of cycles may each comprise 2, 3, 4, 5, 6, 7, 8, or more consecutive cycles in the sequencing run. In some embodiments, each of the first or second plurality of sequencing cycles comprises 2 to 60 cycles. In some embodiments, the first or second plurality of sequencing cycles comprises 2 to 40 cycles. In some embodiments, the first or second plurality of sequencing cycles comprises 3 to 15 cycles.

The sequence run may comprise any non-zero integer number of sequencing cycles. For example, the sequence run has 12, 15, 24, 30 or more consecutive sequencing cycles that corresponds to the barcode sequences, e.g., 15 cycles as shown in FIGS. 11A-11C, that can be in any position of a sequencing run.

In some embodiments, the method 5200 herein further comprises an operation of obtaining a third plurality of flow cell images of the sample in a third plurality of sequencing cycles from a third subset of channels.

The one or more channels may include some or all of the first, second, and/or third dark channel(s). The one or more channels may not include all the channels, e.g., 4, of the sequencing system. Operations 5210″, 5220″, and the operation of acquiring the third plurality of flow cell images may use different subsets of channels. The channels from which one of the first, second, third or other pluralities of flow cell images are obtained may or may not be identical as the channels from which another one of the first, second, third or other pluralities of flow cell images are obtained. For example, in the first 5 cycles, the first subset of channels corresponding to nucleotide bases A, T, and C are used for acquiring the flow cell images and the dark channel corresponds nucleotide base G. In the next 5 cycles, the second subset of channels corresponds to A, G, C while the dark channel may be altered, and correspond nucleotide base T. In the third 5 cycles, the third subset of channels corresponds to nucleotide bases A, G, and T, while the dark channel may be altered, and correspond nucleotide base C.

In some embodiments, the channels from which that the first plurality of flow cell images are obtained in the first plurality of sequencing cycles may or may not include one or more dark channel(s). Similarly, the channels from which the second or third plurality of flow cell images are obtained in their corresponding sequencing cycles may or may not include one or more dark channels.

In some embodiments, the signal intensity of the flow cell images may be much dimmer, e.g., 10× less than the bright spots in flow cell images from other channels, and comparable with background noise so that the flow cell images acquired or obtained from the dark channel may not be used for base calling to avoid possible errors in base calls. Further, the time and computation for performing base calling using the flow cell images may be reduced. In other words, base calling may be performed using flow cell images from the bright channels, e.g., 2 or 3 bright channels.

In some embodiments, there is only 1 dark channel. In some embodiments, there can be multiple dark channels, each dark channel or each combination of dark channels corresponds to a specific set of different cycles. In some embodiment, the dark channel(s) are alternating in different cycles. In other words, a first dark channel in the first plurality of cycles may not be dark in the second plurality of cycles while a second dark channel that is not dark in the first plurality of cycles may be dark in the second plurality of channels. In some embodiments, a first dark channel corresponds to a fluorescent dye attached to a first nucleotide of adenine (A), thymine (T), guanine (G), or cytosine (C) that emits light below a predetermined threshold in the first plurality of sequencing cycles, and the second dark channel corresponds to a second fluorescent dye attached to a second nucleotide of A, T, G, or C that emits light below a predetermined threshold in the second plurality of sequencing cycles, and the first and second nucleotides are different. The emitted light from the sample corresponding to the first nucleotide may be zero, when there is no administration of the fluorescent dye corresponding to the first nucleotide to the sample. The emitted light corresponding to the first nucleotide may be dimmer than other dyes corresponding to the “bright” nucleotides when there is administration of the fluorescent dye corresponding to the dark nucleotide, but the concentration of the fluorescent dye is less than other dyes per volume or the emitted light may be darker or dimer from the dark nucleotides with a same concentration among different dyes.

In some embodiments, the first dark channel corresponds to a first channel from which no flow cell images or only dark flow cell images (e.g., image intensities from polonies therewithin are all below the predetermined threshold) are obtained in the first plurality of sequencing cycles. The second dark channel can correspond to a second channel from which no flow cell images or only dark flow cell images (e.g., image intensities from polonies therewithin are all below the predetermined threshold) are obtained in the second plurality of sequencing cycles. For example, a barcode sequence can be AAAAATTGTCTTTTT (SEQ ID NO: 35). In the first 5 cycles, channel corresponding to nucleotide A is dark, while in the last 5 cycles, channel corresponding to nucleotide T is dark. The other barcode sequences within the same set will have “bright” bases that are not A in the first 5 cycles and different “bright” bases that are not T in the last 5 cycles.

In some embodiments, two different subset of channels, e.g., among the first, second, or third subset of channels, are at least partly different from each other. For example, the first subset may corresponds to channels of T, C, and G, while the second subset of channel may corresponds to T and C. In some embodiments, two different subset of channels, e.g., the first, second, or third subset of channels, are identical to each other. The channels herein may include 3 or 4 different channels. In some embodiments, two of the 3 or 4 channels can be of a same first fluorescent color, while the other one or two channels can be of a same second fluorescent color.

In some embodiments, each of the first or second set of base calls comprises a sequence of base calls corresponding to the first plurality of sequencing cycles or the second plurality of sequencing cycles. For example, one entry in the first set of base calls can be “TTGTC,” and one entry in the second set of base calls can be “CTCTT,” as shown in FIG. 11A.

After the first and/or second set of base calls are determined, the methods 5200 (e.g., in FIGS. 52A-52C) herein can further include an operation of determining whether one or more sequences of base calls of the first and/or second set of base calls matches at least a part of a barcode sequence. For example, a sequence of “TGGTC” may be matched the barcode for polonies from subset 3, which is “TTGTC” with 1 mismatched base. The mismatch rate of ⅕ may or may not satisfy an error tolerance rate. The error tolerance rate can be preset. If the error tolerance rate is met, and in response to the determination, the methods can include an operation of assigning the one or more sequences of the first and/or second set of base calls to the corresponding barcode sequence, thereby linking the polony to the DNA or RNA fragment that the barcode sequence uniquely identifies. In some cases, the determined sequence from base calls only matches a portion of the barcode but not the entire barcode. The portion of barcode that is matched can be a fragment with consecutive bases, for example, bases 1-5 or bases 10-15 of the barcode sequence.

In some embodiments, each barcode sequence is used to uniquely identifies a DNA or RNA fragment of the sample. The barcode sequence(s) can be predetermined. The barcode sequences can be saved and retrieved later when need as reference for matching the base calls. The number of barcode sequences may determine how many subsets can the polonies be divided into.

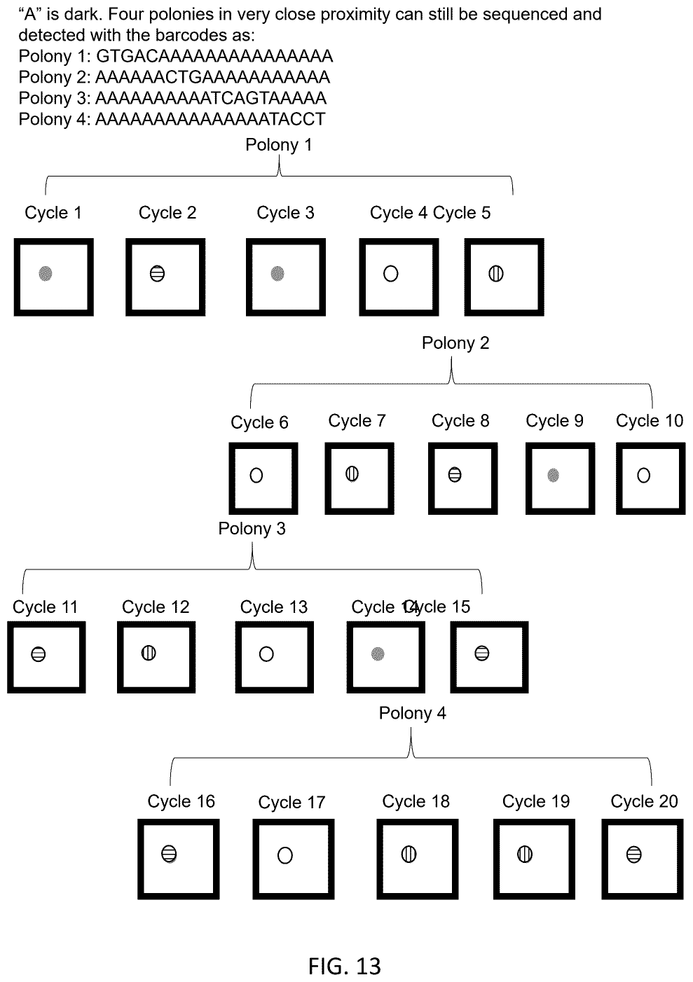

FIG. 13 shows exemplary barcode sequences that can be used to enable accurate and reliable sequencing and analysis with samples of a spatial density that is 4× greater than typical 3D samples that existing systems can sequence using method 5200. The polonies are divided into 4 subsets, each subset sharing a barcode sequence that can uniquely identify a DNA or RNA fragment of the sample. The same barcode sequence can be shared within the same subset of polonies. The first subset of polonies are imaged in cycles 1 to 5 while the other polonies are dark. Similarly, polonies in subsets 2, 3, and 4 are “bright” in sequence in later sequencing cycles. Images from the channel that corresponds to nucleotide A are not obtained to save imaging time. So that the system throughput is higher than imaging the sample of the same polonies using existing systems.

In some embodiments, the different barcode sequences are determined so that only a certain number of barcode sequences have “bright” bases in a specific sequencing cycle. As shown in FIGS. 11A-11C and FIG. 13, at the first sequencing cycle, n+1, only one barcode sequence among its own barcode sequences set has a nucleotide base that is “bright,” the rest of the barcode sequences in the corresponding set, at cycle n+1, are “dark.” FIG. 11C shows an exemplary set of barcode sequences including 3 different barcode sequences with a “dark” channel corresponding to A. In some embodiments, when there is relatively large number of barcode sequences, e.g., 10 or 40, more than one barcode sequence may have a “bright” base in a same sequencing cycle.

In some embodiments, a fragment of the barcode sequence comprises only 1, 2, or 3 bases, and wherein the rest of the barcode sequence comprises all four different bases. In some embodiments, the one or more reference cycles, e.g., first 5 cycles, correspond to the barcode sequence and correspond to only three bases, and wherein the rest of the barcode sequence corresponds to subsequent cycles and comprises all four different bases. In some embodiments, the barcode sequence(s) comprises only three bases. In some embodiments, the barcode sequence comprises all four bases. The barcode sequence may have about 2 to about 100 nucleotide bases. In some embodiments, the barcode sequence has about 3 to 60 nucleotide bases. In some embodiments, the barcode sequence has about 3 to 30 nucleotide bases.

In some embodiments, the barcode sequence used in correspondence with method 5200 (e.g., in FIGS. 52A-52C) can have some random base(s). For example, to avoid homopolomers, which is a same base repeating consecutively for n times, n is greater than 4, 5, 6, or even larger, the barcode sequence can have the one or more nucleotides after a preset of number of consecutive repeat of an identical nucleotide base is randomly selected from 3 nucleotide bases other than the identical nucleotide base. For example, FIG. 11B shows “N” as random bases, which can be randomly selected from bases T, G, and C. In some embodiments, the barcode sequence comprises an identical unique base repeating consecutively of no more than 3, 4, 5, 6, 7, 8, 9, or 10 times.

In the sequencing cycle(s) corresponding to the randomized base, e.g., base N in FIG. 11B, all the polonies in all the subsets, e.g., first, second, and third subsets, are not dark. The polonies can be of a spatial density that they may overlap with each other and base calling from the flow cell images may not be reliable. In some embodiments, no flow cell images in the sequencing cycle(s) corresponding to the randomized base is acquired. Alternatively, the trapping step that traps specific avidite for imaging may also be skipped to further save the sequencing run time.

In some embodiments, the method 5200 (e.g., in FIGS. 52A-52C) herein can include an operation of determining, by the processor and for a sequencing cycle, whether image intensities from both the first and second sets of polonies are above a predetermined threshold. If the determination is yes, both subset of polonies are emitting light, and they may be overlapping spatially that may cause base calling problems. Therefore, in response to the determination, the method can include an operation of obtaining no flow cell images in the sequencing cycle, e.g., the n+6, and n+12 cycle as shown in FIG. 11B, which is the randomized base that breaks up homopolomers in the barcode sequence. This particular sequencing cycle, with a randomized base, e.g., N, can be before and/or after the first or second plurality of sequencing cycles. In some embodiments, this particular sequencing cycle is before and after the first plurality of sequencing cycles. In some embodiments, this particular sequencing cycle is before and after the second plurality of sequencing cycles.

In some embodiments, the method 5200 (e.g., in FIGS. 52A-52C) herein advantageously allow sequencing of high spatial density 3D samples. Existing technologies may be limited by cartridge designs and optical system characteristics so that when the spatial density of 3D sample increases, polonies and clusters overlap with other and base callings and subsequent sequencing analysis may not be accurate or reliable. The methods herein, without changing the cartridge design or optical system of the sequencing system, enable sequencing of 3D samples with a spatial density that is n fold higher than what existing systems and methods can handle. The number n can depend on different existing systems and sample. In some embodiments, n is in the range of 2 to 100. In some embodiments, n is in the range of 2 to 20. In some embodiments, n is in the range of 2 to 15. In some embodiments, n is the total number of barcode sequences that are used.

In some embodiments, the method 5200 (e.g., in FIGS. 52A-52C) herein can include an operation of preparing the sample for imaging in each sequencing cycle. In some embodiments, the method 5200 herein can include an operation of contacting at least one subset of the first, second, and third subset of polonies of the sample with a plurality of sequencing primers, a first plurality of polymerases and a first mixture of different types of avidites. The avidites are disclosed in more details below. The individual avidites in the first mixture can comprise a core attached with multiple nucleotide arms and each arm of the individual avidite comprises the same type of nucleotide unit, e.g., A, T, C, or G.

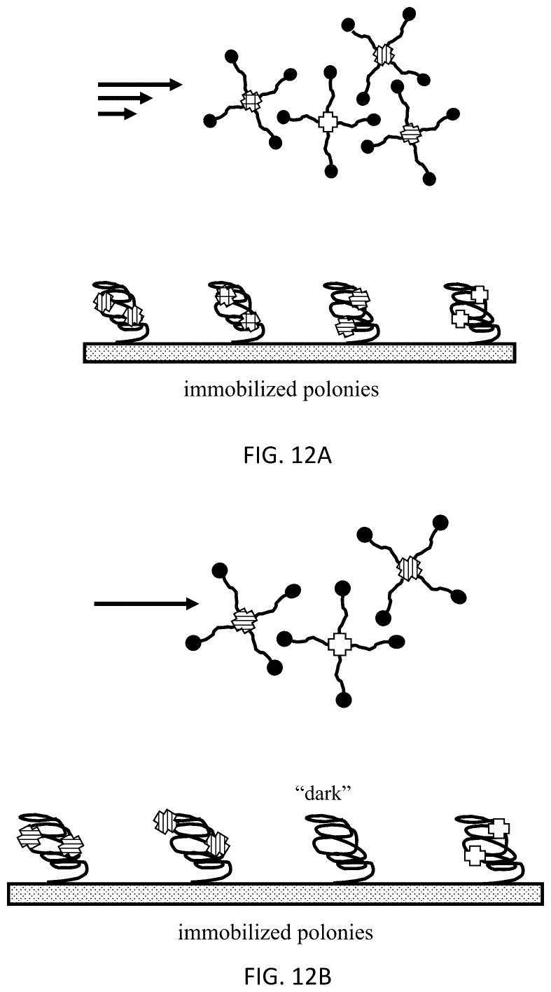

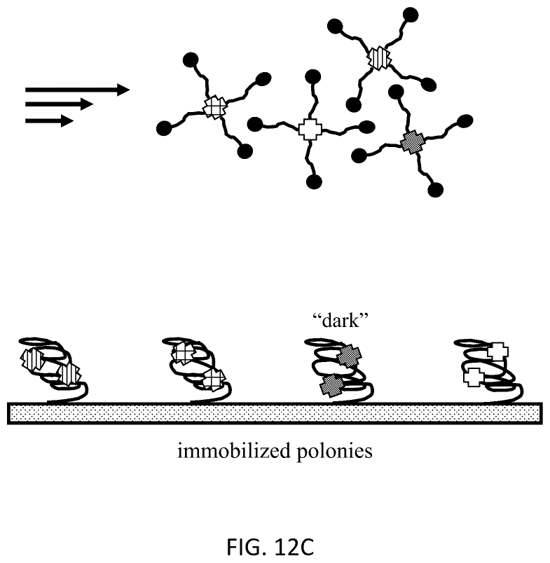

FIGS. 12A-12C show schematic diagrams of steps in an exemplary sequencing cycle using 4 different types of avidites (FIG. 12A), three different types of avidites (FIG. 12B), and 4 different types of avidite including a type of avidite with “dark” fluorescent dye(s) (FIG. 12C). The operation of flowing of a mixture of different labeled avidites can be performed by the sequencing system 110. Either consecutive flows or a single flow can be used to flow the different types of labeled avidites into the flow cell. Imaging can either follow each flow in consecutive flows or the single flow to capture emitted light from the polonies. The operation of flowing the avidites and obtain the flow cell images may be performed by the sequencing system in various manners. The disclosure herein do not limit how specifically the reagents including avidites are flowed into contact with polonies, and timing imaging in relation to the flowing operation.

As shown in FIG. 12B, the flow(s) lack one type of avidite that correspond to nucleotide A, so that at least ¼ of the total COGS and/or avidite can be saved. FIG. 12B, in some embodiments, the flow(s) may include 4 different type of avidite include one type of avidite that is not labeled with any florescent dye(s). Alternatively, the one type of avidite is labeled with “dark” fluorescent dye(s) that are not emitting any fluorescent light when exited or only emitting fluorescent light beyond a detectable level by the optical system of the sequencing system 110. Either consecutive flows or a single flow containing a mixture of 3 different labeled avidites may be administered to the sample(s) on the support. Imaging the sample may follow either each separate flow or the single flow and imaging may include detecting 3 different labeled avidites bound to polonies so that at least one subset of polonies are “dark.”

In some embodiments, the first mixture of different types of avidities comprise 4 different types of avidites corresponding to 4 different nucleotide bases. Each type of different types of avidites in the first mixture can be labeled with one type of a fluorescent dye that corresponds to the nucleotide units to distinguish the different types of avidites in the first mixture. In some embodiments, the fluorescent dye of each type of avidite in the first mixture emits light at a different wavelength when excited. In some embodiments, one type of avidite is labeled with one type of a dark fluorescent dye that emits light below a predetermined threshold in the channels while the fluorescent dye of other types of avidite emit light above a second predetermined threshold that can be different from the first threshold.

In some embodiments, the first mixture of different types of avidities comprise only 2 or 3 different types of avidites. The 2 or 3 types of different types of avidites in the first mixture is labeled with one corresponding type of a fluorescent dye that corresponds to the nucleotide units to distinguish 2 or 3 types of avidites among the different types of avidites in the first mixture. The first mixture may lack a 4th type of avidite, so that one type of nucleotide bases, e.g., A, may not be contacted with its corresponding avidite. As a result, during imaging, the one type of nucleotide does not emit light, if any, and appear “dark.”

In some embodiments, the operation of generating the first set of base calls for a first, second, third, or other subset of polonies of the sample based on the first plurality of flow cell images comprises one or more operations of the 3D base calling method 6000 disclosed herein. In some embodiments, such base calling operations include: generating, by the processor, a first plurality of processed images of the first plurality of flow cell images; filtering, by the processor, the first plurality of flow cell images based on the first plurality of processed images thereby generating a first plurality of filtered images; obtaining, by the processor, a 3D polony map of the sample; extracting, by the processor, image intensity of polonies based on the 3D polony map from: a second plurality of flow cell images; a second plurality of processed images; a second plurality of filtered images; or their combinations; and performing, by the processor, 3D base callings of the first subset of polonies of the sample based on the extracted image intensity of the polonies.

In some embodiments, the operation of generating the first set of base calls for a first, second, third, or other subset of polonies of the sample based on the first plurality of flow cell images comprises one or more operations of the image registration method 9000 disclosed herein.

In some embodiments, the operation of obtaining multiple flow cell images comprises actively retrieving or passively receiving multiple flow cell images of a sample to be processed.

In some embodiments, the operation comprises acquiring the flow cell images using the imager 1160 of the sequencing system.

The sample can be in situ. The sample can be a 3D sample. The sample can be a volumetric sample that may contain different biological information at the same x-y location but different z location. The sample can include multiple cells, tissue, or their combination. The 3D sample can be any biological sample that has a thickness that is greater than a predetermined threshold along the axial axis. For example, the thickness can be greater than 2 um, 3 um, 4 um, 5 um, 10 um, 20 um, or more. The z axis (e.g., axial axis) is orthogonal to the image plane defined by x and y axes.

The flow cell images can be acquired using the optical system disclosed herein, from the 1, 2, 3, 4, or more channels of the imager 1160. Each flow cell image can include one or more tiles (e.g., imaging areas), and each tile can be divided into multiple subtiles. Each tile or subtile can include a plurality of polonies. Each subtile can include multiple regions with each region including a number of polonies. For example, the polonies can be extracted from corresponding regions of flow cell images from 4 different channels in a given cycle. As another example, the polonies can be extracted from flow cell images from a single channel. The flow cell image as disclosed herein can be an image that is acquired from a flow cell 1120 as shown in FIG. 1.

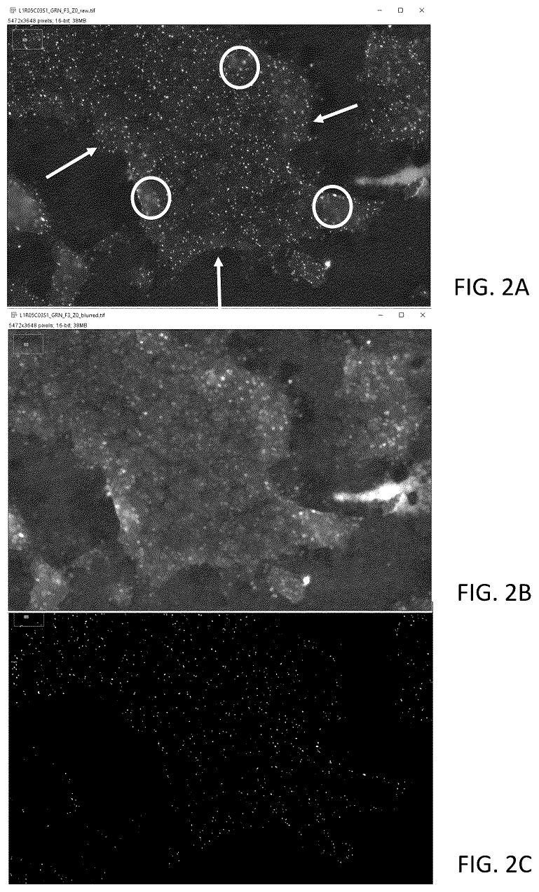

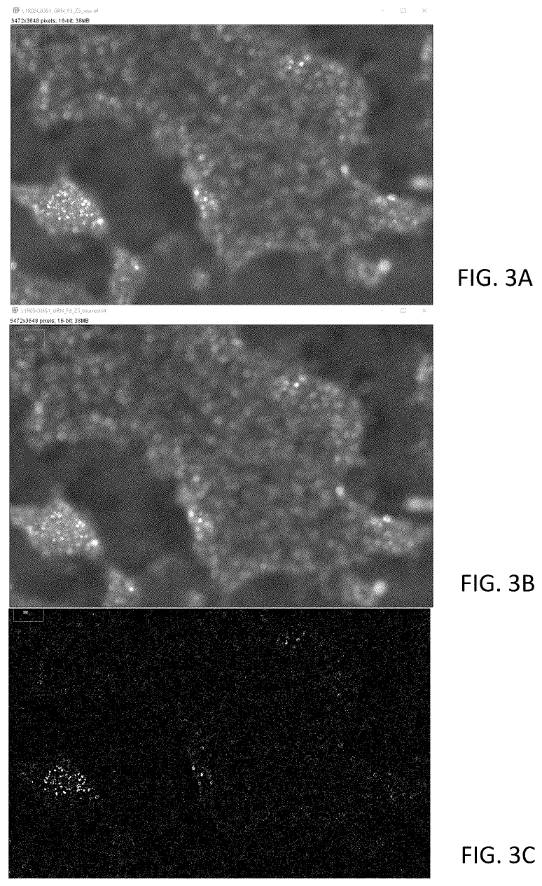

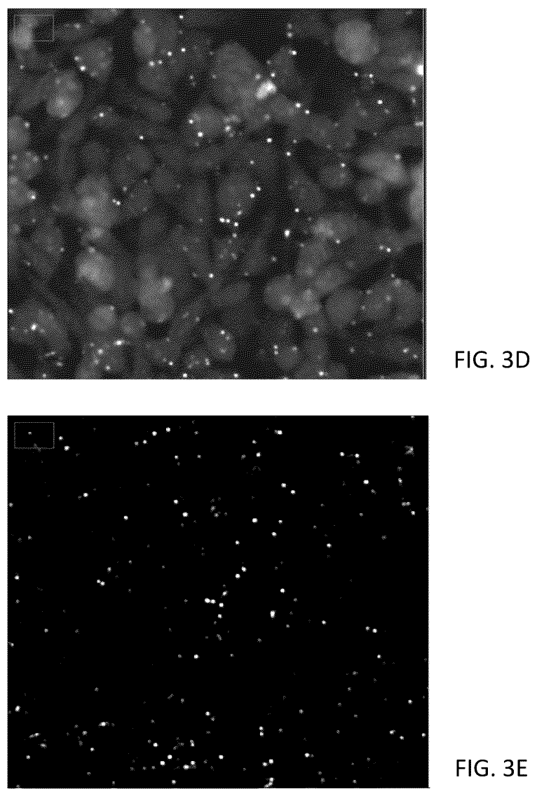

In some embodiments, a flow cell image herein can be an image of one or more tiles, one or more subtiles, one or more segmented regions within tile(s) or subtile(s), or their combinations. Each flow cell image can comprise a field of view (FOV). The FOV can be orthogonal to the axial axis. The FOV can be within the x-y plane. The FOV of different flow cell images at different axial locations can be identical within the x-y plane. The FOV of different flow cell images at different axial locations can have at least an overlapping portion within the x-y plane. The image resolution of different flow cell images at different axial locations can be about identical or exactly identical. In some embodiments, the image resolution of different flow cell images at different axial locations is different. FIGS. 2A and 3A show two exemplary flow cell images acquired at two different z locations along the axial axis of a same 3D sample within a same sequencing cycle.

Each flow cell image at a specific z location includes intensities generated by polonies and clusters at the corresponding z location. As shown in FIGS. 2A-3A, signals from polonies and clusters are small bright spots within the images. Each bright spot can be of various sizes that is less than a couple of pixels, e.g., less than a pixel, about a pixel, about 2 pixels, 3 pixels, 4, pixels, or 5 pixels. In some embodiments, each signal spot of the polonies or clusters can be any number of pixels in the range from 0.01 pixel to about 72 pixels. In some embodiments, each signal spot of the polonies or clusters can be any number of pixels in the range from 0.1 pixel to about 16 pixels.

Each flow cell image can also include intensities generated by the cell and its structural elements. Such structural elements can be background objects or components.

In some embodiments, when the depth of field the optical system includes a range, e.g., 0.1 um, 0.2 um, 0.3 um, 0.5 um, 0.6 um, 0.8 um, 1 um, 2 um, 3, um, 4 um, 5 um, etc. expanding along z axis. Polonies and clusters that are within the range of depth of field can appear in-focus or about in-focus in the flow cell image. Flow cell images at a specific z location can also include signals from polonies and clusters that are not within the focus range of the image. Such polonies or clusters are out-of-focus. As shown in FIG. 3A, bigger and blurry signal spots represent out-of-focus polonies or clusters. Some of the out-of-focus polonies or clusters are circled in FIG. 3A.

Each flow cell image at a specific z location can also include noises caused by the optical system and/or undesired signal from the sample. The undesired signal can be signal coming from components of the sample such as membrane, cytosol, and mitochondria. Such background objects can be any objects, relatively larger in size than the polonies or clusters. As shown in FIG. 3A, there is a blurry cellular contour (at the arrows) in the flow cell image, and most of the signal spots are contained within the blurry contour. FIG. 3D shows multiple cells and the polonies or clusters as small bright spots are generally within the contours of different cells. In some embodiments, background objects can include any objects within the 3D sample but are not polonies or clusters.

### 3D Base Callings

In some embodiments, the operation 2530 of generating, by the processor, a first set of base calls for a first subset of polonies of the sample based on the first plurality of flow cell images; or the operation 2540 of generating, by the processor, a second set of base calls for a second subset of polonies of the sample based on the second plurality of flow cell images may include one or more base calling methods (e.g., method 6000) disclosed herein.

In some embodiments, the method 6000 may include operations comprising one or more of: generating, by the processor, a first plurality of processed images of the first plurality of flow cell images; filtering, by the processor, the first plurality of flow cell images based on the first plurality of processed images thereby generating a first plurality of filtered images; obtaining, by the processor, a 3D polony map of the sample; extracting, by the processor, image intensity of polonies based on the 3D polony map from: a second plurality of flow cell images; a second plurality of processed images; a second plurality of filtered images; or their combinations; and performing, by the processor, 3D base callings of the first subset of polonies of the sample based on the extracted image intensity of the polonies.

In some embodiments, the method 6000 is performed during a cycle N that is different from a reference cycle. The template image(s) can be generated in the reference cycle(s) and polonies from one or more channels within the reference cycle(s) can be included in the template image in a reference coordinate system, while base calling of cycle N is yet to be performed. In some embodiments, cycle N is the current cycle. N can be any non-zero integer. For example, for short read sequencing, N can be any integer from 1 to 150.

FIG. 8B shows a schematic diagram of one or more template images generated in the reference cycle in a reference coordinate system. The template image 2100, in some embodiments, include a size that is about identical to a single tile 2900 that includes a 5×5 grid of subtiles. In some embodiments, the template image disclosed herein can be individual regions within a subtile. Each template image can include a plurality of polonies therein.

In some embodiments, the template image can be of about the same size of a flow cell image so that all the polonies, from different tiles, 2900 in FIG. 8B, and from multiple channels, can be registered to the same template image. However, such template image may contain polonies that will not be used in at least some operations described herein to reduce computational burden without sacrificing accuracy.

In some embodiments, more than one template images can be generated, and each template image corresponds to at least part of a subtile of a flow cell image from a channel.

The template image herein can be initialized as a virtual image that has a black or dark background with no signals from polonies. For example, the template image can be initialized to be zero or include otherwise minimal image intensity at all pixels.

After the coordinates of a polony is determined by image registration of flow cell images, e.g., across different channels, the intensity of the polony can be added to the template image at the location determined by the coordinates and with the size and shape determined based on registration. The template image can be a virtual image that combines image intensity from polonies obtained from 2, 3, 4, or even more channels at the reference cycle. The pixels of the template containing no polonies in them remains to be black or dark so that the template image can have a cleaner background without noise that appear in actual flow cell images.

The polonies can be from a subtile of flow cell images within a reference cycle, and more specifically, from one or more selected regions of the subtile. The flow cell images can be from different channels of 1, 2, 3, 4, or more channels of the system 1000. As a nonlimiting example, a reference cycle can be any cycle of the first 5 or 6 cycles. In some embodiments, the reference cycle can be any cycle that is greater than 0. In some embodiments, the reference cycle is the first cycle.

In some embodiments, the method 6000 comprises an operation of generating 2D flow cell images with intensities of polonies or clusters of the 3D sample so that the intensities can be used for performing 3D base calling for different sequencing cycles. The operation of generating the flow cell images can be performed using the sequencing system 1100 here.

The method 6000 can comprise an operation of obtaining processed images of the follow cell images. In some embodiments, the operation of obtaining processed images can comprise processing the flow cell images with one or more predetermined processing methods.

In some embodiments, the one or more processing methods can include selecting a kernel and generating the processed images by performing an operation on the flow cell images using the selected kernel. For example, the operation performed can be an opening operation, which can be expressed as f ok, where f is the flow cell image, and k is the kernel. The opening operation can be performed in spatial domain. Alternatively, to make computation fast or less complex, the opening operation may be performed in a different domain, such as the Fourier domain. The opening operation can be the dilation of the erosion of image f by a kernel k. The opening operation can remove objects that are smaller than the kernel, and the subsequent dilation operation may restore the size and shape of the remaining objects. FIGS. 2B and 3B show exemplary processed images after an opening operation. The processed images can also be called opened images in which most of the bright spots of polonies or clusters are removed, and other background objects are retained.

As another example, the operation performed can be a convolution, and the flow cell image can be convoluted with the selected kernel. As yet another example, obtaining the plurality of processed images further comprises: selecting a first kernel and a second kernel; generating first images by convolving the first or second plurality of flow cell images flow cell images using the first kernel; and generating second images by convolving the first or second plurality of flow cell images flow cell images using the second kernel. The first images and the second images can be different blurred images after the convolution with different blurring kernel.

The kernel may take any size that is smaller than the size of the flow cell image. For example, with an opening operation, the kernel can be 2 by 2, 3 by 3, 4 by 4, 5 by 5, 6 by 6. In some embodiments, the kernel size can be customized to remove at least some of the noise and unwanted signal that are larger than the kernel size. In some embodiments, the kernel can be circular. The kernel can be other various shapes.

In some embodiments, the kernel is a Gaussian kernel. In embodiments where 2 different kernels are used, the first kernel and the second kernel can be different Gaussian kernels.

In some embodiments, the method 6000 can comprise an operation 6200 of filtering the flow cell images. The operation of filtering 6200 can be based on the processed images generated in its preceding operation. The operation of filtering 6200 can generate multiple filtered images, each filtered image corresponding to a flow cell image at a corresponding z location along the axial axis.

In some embodiment, the operation 6200 can include subtracting the processed images from a corresponding flow cell image, thereby generating the filtered image. The filtered image can be obtained as fi=f−f∘k, where fi represents the filtered image, f represents the flow cell image, and k represent the kernel, and ∘ represents the opening operation.

In some embodiments, the filtered image is an image of the corresponding flow cell image filtered by a top-hat filter. In some embodiments, the filtered image is an image of the corresponding flow cell image filtered by a difference of Gaussian (DoG) filter. In some embodiments, the filtered image is an image of the corresponding flow cell image filtered by other filters configured to extract small elements and details (e.g., in-focus polonies) from the flow cell images

After filtering, at least part of the noise and undesired signal in the flow cell images are removed, which can include the cell components and out-of-focus polonies. Removal of such noise and undesired signal which are inevitable in 3D flow cell images can advantageously facilitate generating intensities that are attributed to polonies or clusters but not other background objects or noises. Intensities after filtration can be used for more accurate and reliable base calling than those without filtering.

FIGS. 2C and 3C shows two filtered images from two different axial locations. FIG. 2C indicates that a larger number of polonies or clusters are in-focus at the first axial location of z=0, and a much smaller number of polonies or clusters are in-focus at a second axial location of z=5, which is about 10 um away from the first axial location.

FIG. 3E shows another exemplary filtered image of the flow cell image in FIG. 3D. The background objects in the flow cell image are filtered out in the filtered image. The out-of-focus polonies in the flow cell image are also removed from the filtered image. The filtered image shows in-focus polonies or clusters.

In some embodiments, the method 6000 can include an operation of adding an offset to the filtered image(s). In some embodiments, the offset can be predetermined so that after the offset, the range of image intensity can be within a predetermined range. In some embodiments, different offset can be used to bring different filtered images with various intensities ranges to be within a predetermined range that is common to all the filtered images.

The method 6000 can comprise an operation of 6300 of generating a maximum intensity projection (MIP) image based on the plurality of filtered images. The operation of 6300 can include computing a maximum intensity for each pixel of the MIP image using intensities of the plurality of filtered images at the corresponding pixels. The MIP image can have the same image size, resolution, and/or FOV as the flow cell images. The MIP image can be a flattened 2D image of a stack of flow cell images from different axial locations.

As an example, an axial slide deck of 10 flow cell images is acquired at 10 different z locations. Each z location is about 0.1 um to about 20 um away from its adjacent z location. The first z location is at z=0, and the 10^{th} z location is at z=9. 10 filtered images are generated for each of the different z locations. A MIP image can be initialized to be the same size as the flow cell images, e.g., 1028 by 1028. All intensity in the initial MIP image can be 0 or any other minimal intensity. For each pixel at pixel (i, j), image intensities at pixel (i,j) from 10 filtered images are extracted, and a maximum intensity among the 10 different image intensity is selected for pixel (i,j) in the MIP image. In some embodiments, a MIP image can be generated for each cycle. In some embodiments, a MIP image can be generated for each different channel of 1, 2, 3, 4, 5, 6, or more channels.

In some embodiments, the image intensities in the filtered images are normalized to a predetermined range before the MIP image is obtained using the filtered images. In some embodiments, the MIP images can be normalized across different cycles. In some embodiments, the MIP images for different FOV within the x-y plane, e.g., covering different tiles can be normalized. The normalization can be to a predetermined range.

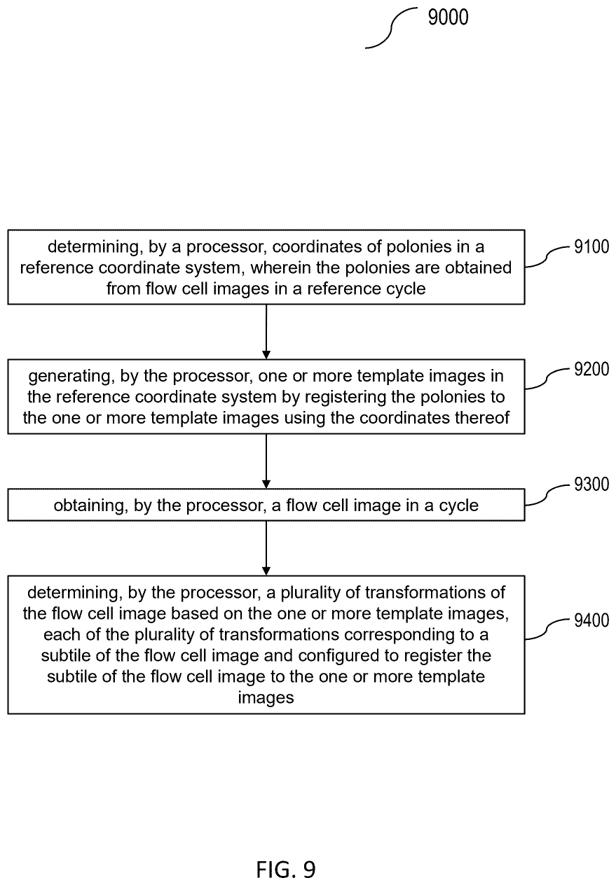

In some embodiments, the method 6000 include an operation of registering the MIP images, e.g., 9000 in FIG. 9. In some embodiments, the MIP images are registered across channels and different cycles before any base calling is performed. Various image registration techniques can be used to register the MIP images. The MIP images can be registered using 2D registration techniques for example, by treating the MIP images as flow cell images acquired from the sequencing system 110. In some embodiments, the MIP images can be registered, e.g., across different channels and/or different cycles, using the image registration method 9000 as disclosed herein by treating each MIP image as a flow cell image. In some embodiments, the MIP images can be registered after one or more preprocessing operations disclosed herein are performed.

The method 6000 can comprise an operation 6400 of performing 3D base calling using the MIP image(s). The MIP image(s) can be 2D, and the base calling can be performed for MIP images from different channels per cycle per z location.

In some embodiments, the operation 6400 of performing base callings using the MIP image comprises performing primary analysis step(s) herein to adjust image intensities of polonies in the MIP; and making base calls for the polonies based on the adjusted image intensities in the MIP. In some embodiments, the primary analysis steps comprise one or more of the following: background subtraction; image sharpening; intensity offset adjustment; color correction; intensity normalization; phasing and prephasing correction; image registration; quality score estimation. In some embodiments, the image registration as a primary analysis step herein is configured to align images from different cycles and different channels, for example, with respect to a template image or a reference coordinate system. In some embodiments, the image registration as a primary analysis step herein is configured to register polonies or clusters from different cycles and different channels, e.g., in the MIP image, to a template image or a reference coordinate system.

For example, the base calling can be performed using MIP images from different channels in cycle N, after the MIP images from different channels are registered relative to the template image disclosed herein. Various existing 2D base calling algorithms can be used. The base calling results can be saved with its 3D coordinates. Such 3D coordinates can be used to register the base calling across different cycles and at different z locations.

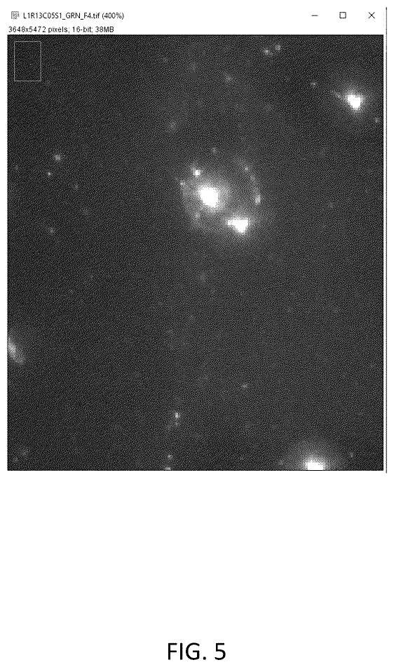

In some embodiments, the method 6000 comprise an operation 6500 of obtaining a second MIP image based on the plurality of flow cell images. The second MIP is a flattened 2D image of an axial stack of the flow cell images. In some embodiments, the flow cell image are raw images acquired by the sequencing system 110. The operation of obtaining the second MIP can be different from that of the first MIP image. In the flow cell images, e.g., raw images, polonies out-of-focus can have larger full width half maximum (FWHM), so, the signal of the out-of-focus polonies is more spread out than in-focus polonies or clusters. In some embodiments, the larger FWHM can cause a white ring around a polony. FIG. 5 shows an exemplary second MIP image generated directly from the flow cell images, and there is a ring or halo round the polony in the center of the image. The ring or halo can be image artifacts that may cause error in base calling. The second MIP can also retain some background information, e.g., from undesired background objects, that may interfere with base calling if the second MIP is used for base calling. As such, the second MIP includes artifacts and/or undesired background objects that require additional processing before accurate and reliable base calling can be make based on the intensities in the second MIP.

In some embodiments, the second MIP can be used for registration of the first MIP and/or polonies or clusters since it shares the same FOV, resolution, image size, etc. The artifacts and/or undesired background objects in second MIP may interfere with base calling if the second MIP is used for base calling, but the same artifacts and/or undesired background objects (that are not in the first MIP) can facilitate registration of first MIP by using the second MIP, for example, for registration to cell images with staining.

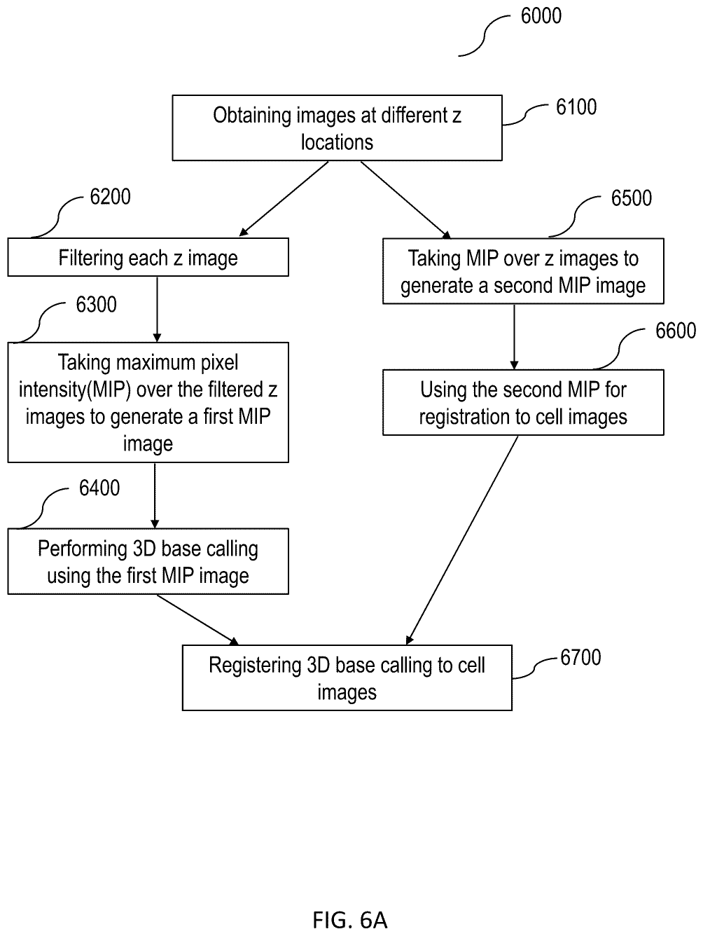

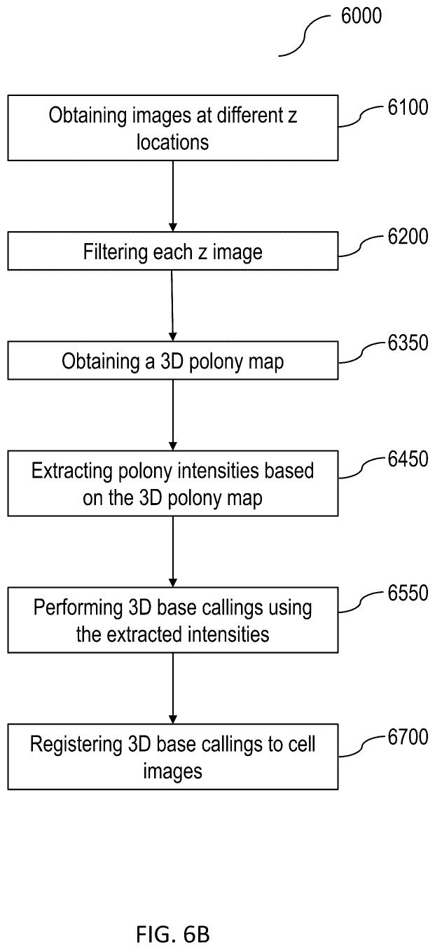

FIG. 6B shows a flow chart of a computer-implemented method 6000 for performing 3D base calling from flow cell images. The method 6000 can include some or all of the operations disclosed herein. The operations may be performed in but is not limited to the order that is described herein.

The method 6000 can be performed by one or more processors disclosed herein. In some embodiments, the processor can include one or more of: a processing unit, an integrated circuit, or their combinations. For example, the processing unit can include a central processing unit (CPU), a graphic processing unit (GPU), or NPU. The integrated circuit can include a chip such as a field-programmable gate array (FPGA). In some embodiments, the processor can include the computer system 4000.

The method 6000 can be performed based on flow cell images from a current sequencing cycle alone or in combination with information from preceding cycle(s) of the current sequencing cycle.

In some embodiments, some or all operations in method 6000 can be performed by the FPGA(s). In embodiments when some operations are performed by FPGA(s), the data after an operation performed by the FPGA(s) can be communicated by the FPGA(s) s to the CPU(s) so that CPU(s) can perform subsequent operation(s) in method 6000 using such data. Similarly, data can also be communicated from the CPU(s) to the FPGA(s) for processing by the FPGA(s). In some embodiments, all the operations in method 6000 can be performed by CPU(s).

Alternatively, the operations performed by CPU(s) can be performed by other processors such as the dedicated processors, or GPU(s). In some embodiments, all the operations in method 6000 can be performed by FPGA(s).

The method 6000 can comprise an operation 6100 of obtaining multiple flow cell images of a sample. The flow cell images can be acquired at different locations along an axial axis, i.e., z axis. In some embodiments, the operation 6100 comprises actively retrieving or passively receiving multiple flow cell images of a sample to be processed. In some embodiments, the operation 6100 comprises acquiring the flow cell images using the imager 1160 of the sequencing system. In some embodiments, the flow cell images are acquired by a NGS sequencing system.

The sample can be in 3D. The sample can be a volumetric sample that may contain different biological information at the same x-y location but different z location. The sample can be an in situ sample. The sample can include multiple cells, tissue, or their combination. The sample can be any biological sample that has a thickness that is greater than a predetermined threshold (e.g., depth of field) along the axial axis. The sample can be any biological sample that has a thickness that is greater than the depth of field of the optical system. For example, the thickness can be greater than 1 um, 2 um, 3 um, 4 um, 5 um, 8 um, 10 um, 12 um, 15 um, 20 um, or more. The z axis (e.g., axial axis) can be orthogonal to the image plane defined by x and y axes, as show in FIG. 8B.

In some embodiments, the flow cell images can include a first plurality of flow cell images and a second plurality of flow cell images. Each of the first plurality of flow cell images and the second plurality of flow cell images can be acquired at a corresponding location along an axial axis or a z axis. The axial axis can extend from an objective lens of the sequencing system 1100 to the sample located on the flow cell positioned on the sequencing system. As a nonlimiting example, the first plurality of flow cell images can be obtained from the reference cycles and the second plurality of flow cell images can be obtained from one or more cycles different from the reference cycles. As another example, the first plurality of flow cell images can be identical to the second plurality of flow cell images.

The flow cell images can be acquired using the optical system disclosed herein, from the 1, 2, 3, 4, or more channels of the imager 1160. Each flow cell image can include one or more tiles (imaging areas), and each tile can be divided into multiple subtiles. Each subtile can include a plurality of polonies or clusters. Each subtile can include multiple regions with each region including a number of polonies. For example, the polonies can be extracted from corresponding regions of flow cell images from 4 different channels in a given cycle. As another example, the polonies can be extracted from flow cell images from a single channel. The flow cell image as disclosed herein can be an image that is acquired from a flow cell 1120 as shown in FIG. 1.

In some embodiments, a flow cell image herein can be an image of one or more tiles, one or more subtiles, one or more segmented regions with tile(s) or subtile(s), or their combinations. Each flow cell image can comprise a field of view (FOV). The FOV can be orthogonal to the axial axis. The FOV can be within the x-y plane. The FOV of different flow cell images at different axial locations can be identical within the x-y plane. The FOV of different flow cell images at different axial locations can have at least an overlapping portion within the x-y plane.

The image resolution of different flow cell images at different axial locations can be about identical or exactly identical. In some embodiments, the image resolution of different flow cell images at different axial locations is different.

FIGS. 2A and 3A show two exemplary flow cell images acquired at two different z locations along the axial axis of a same 3D sample within the same sequencing cycle.

Each flow cell image at a specific z location includes intensities generated by polonies and clusters at the corresponding z location. As shown in FIGS. 2A-3A, signals from polonies and clusters are small bright spots within the images. Each bright spot can be of various sizes that is less than a couple of pixels, e.g., less than a pixel, about a pixel, about 2 pixels, 3 pixels, 4, pixels, 5 pixels, or more. In some embodiments, each signal spot of the polonies or clusters can be any number of pixels in the range from 0.01 pixel to about 72 pixels. In some embodiments, each signal spot of the polonies or clusters can be any number of pixels in the range from 0.1 pixel to about 16 pixels.

The polonies can be from a subtile of flow cell images within a reference cycle, and more specifically, from one or more selected regions of the subtile. The flow cell images can be from different channels of 1, 2, 3, 4, or more channels of the system 1000. As a nonlimiting example, a reference cycle can be any cycle of the first 5 or 6 cycles. In some embodiments, the reference cycle can be any cycle that is greater than 0. In some embodiments, the reference cycle is the first cycle.

In some embodiments, the flow cell images are acquired at one or more reference cycles or a cycle different from the one or more reference cycles. As a nonlimiting example, cycles 1-5 or 2-5 can be reference cycles. The flow cell images can be acquired within a single cycle or a couple of cycles.

Each flow cell image can include intensities generated by the cell and its structural elements. Such structural elements can be background objects or components. FIG. 3D shows multiple cells and the polonies or clusters as small bright spots are generally within the contours of different cells.

In some embodiments, when the focus of the optical system includes a range, e.g., 0.1 um, 0.2 um, 0.3 um, 0.5 um, 0.6 um, 0.8 um, 1 um, 2 um, 3, um, 4 um, 5 um, etc. expanding along z axis. Polonies and clusters that are within the range of focus can appear in-focus or about in-focus in the flow cell image. Flow cell images at a specific z location can also include signals from polonies and clusters that are not within the focus range of the image, but at different z locations. So, such polonies or clusters are out-of-focus. As shown in FIG. 3A, bigger and blurred signal spots represent out-of-focus polonies or clusters. Some of the out-of-focus polonies or clusters are circled in FIG. 3A.

Each flow cell image at a specific z location can also include noises caused by the optical system and/or undesired signal from the sample. The undesired signal can be signal coming from components of the sample such as membrane, cytosol, and mitochondria. Such background objects can be any objects, relatively larger in size than the polonies or clusters. As shown in FIG. 3A, there is a blurry cellular contour (at the arrows) in the flow cell image, and most of the signal spots are contained within the blurry contour. In some embodiments, background objects can include any objects within the 3D sample but are not polonies or clusters.

In some embodiments, bases calls from the polonies include 4 different bases, and percentage of polonies for each of the 4 different bases can be greater than about 10% so that the data are relatively diverse. In some other embodiments, bases called from the plurality of polonies includes 4 or less different bases, and percentage of polonies for one or more bases can be less than about 10%, and such data can be considered as low diversity data. In some embodiments, bases called from the plurality of polonies include 4 or less different bases, and percentage of polonies for some of the bases can be less than about 5%, about 2%, or even about 1%, and such data can be considered as low diversity data. As an example, the base called for bases A, T, C, G in the plurality of polonies can be about 1%, about 2%, about 1%, and about 95%. As another example, the base called for bases A, T, C, G in the plurality of polonies can be about 10%, about 10%, about 10%, and about 70%, respectively. In addition to the base biases affecting diversity, plexity can also be a factor that when plexity is lower than a number, e.g., 8 or 16, the signal could be of low diversity. The method 6000 is configured to process flow cell images, e.g., including intensity processing, registration, and base calling, even if the polonies are low diversity data.

In some embodiments, the method 6000 is performed at least partly during a cycle N that is different from a reference cycle. The template image(s) and/or the 3D polony map can be generated in the reference cycle(s) and polonies from one or more channels within the reference cycle(s) can be included in the template image(s) and/or the 3D polony map in a reference coordinate system, while base calling of cycle N is yet to be performed. In some embodiments, cycle N is the current cycle. N can be any non-zero integer. For example, for short read sequencing, N can be any integer from 1 to 150.

FIG. 8B shows a schematic diagram of one or more template images generated in the reference cycle in a reference coordinate system. The template image 2100, in some embodiments, include a size that is about identical to a single tile 2900 that includes a 5×5 grid of subtiles. In some embodiments, the template image disclosed herein can be individual regions within a subtile. Each template image can include a plurality of polonies therein.

In some embodiments, the template image can be of about the same size of a flow cell image so that all the polonies, from different tiles, 2900 in FIG. 8B, and from multiple channels, can be registered to the same template image. However, such template image may contain polonies that will not be used in at least some operations described herein to reduce computational burden without sacrificing accuracy.

In some embodiments, more than one template images can be generated for the same axial location, and each template image corresponds to at least part of a subtile of a flow cell image from a channel.

The template image herein can be initialized as a virtual image that has a black or dark background with no signals from polonies. For example, the template image can be initialized to be zero or include otherwise minimal image intensity at all pixels.

After the coordinates of a polony is determined by image registration of flow cell images, e.g., across different channels, the intensity of the polony can be added to the template image at the location determined by the coordinates and with the size and shape determined based on registration. The template image can be a virtual image that combines image intensity from polonies obtained from 2, 3, 4, or even more channels at the reference cycle. The pixels of the template containing no polonies in them remains to be black or dark so that the template image can have a cleaner background without noise that appear in actual flow cell images.

In some embodiments, the method 6000 comprises an operation of generating an axial stack of 2D flow cell images. The flow cell images can include intensities of polonies or clusters from a 3D sample. The intensities can be used for performing 3D base calling for different sequencing cycles. The operation of generating 2D flow cell image can be performed by the sequencing system 1100 herein.

The method 6000 can comprise an operation of generating the processed images of the flow cell images. In some embodiments, the operation of generating the processed images can comprise processing the flow cell images with one or more predetermined processing methods.

In some embodiments, the one or more processing methods can include selecting a kernel and generating the processed images by performing an operation on the flow cell images using the selected kernel. For example, the operation performed can be an opening operation, which can be expressed as f∘k, where f is the flow cell image, and k is the kernel. The opening operation can be performed in spatial domain. Alternatively, to make computation fast or less complex, the opening operation may be performed in a different domain, such as the Fourier domain. The opening operation can be the dilation of the erosion of image f by a kernel k. The opening operation can remove objects that are smaller than the kernel, and the subsequent dilation operation may restore the size and shape of the remaining objects. FIGS. 2B and 3B show exemplary processed images after an opening operation. The processed images can also be called opened images in which most of the bright spots of polonies or clusters are removed, and other background objects are retained.

As another example, the operation performed can be a convolution, and the flow cell image can be convoluted with the selected kernel. As yet another example, obtaining the plurality of processed images further comprises: selecting a first kernel and a second kernel; generating first images by convolving the plurality of flow cell images using the first kernel; and generating second images by convolving the plurality of flow cell images using the second kernel. The first images and the second images can be different blurred images after the convolution with different blurring kernel.

The kernel may take any size that is smaller than the size of the flow cell image. For example, with an opening operation, the kernel can be 2 by 2, 3 by 3, 4 by 4, 5 by 5, 6 by 6 pixels. In some embodiments, the kernel size can be customized to remove at least some of the noise and unwanted signal that are larger than the kernel size. In some embodiments, the kernel can be circular. The kernel can be other various shapes.

In some embodiments, the kernel is a Gaussian kernel. In embodiments where 2 different kernels are used, the first kernel and the second kernel can be different Gaussian kernels.

In some embodiments, the method 6000 can comprise an operation 6200 of filtering the flow cell images. The operation of filtering 6200 can be based on the processed images generated in its preceding operation. The operation of filtering 6200 can generate multiple filtered images, each filtered image corresponding to a flow cell image at a corresponding z location along the axial axis.

In some embodiment, the operation 6200 can include subtracting the processed images from a corresponding flow cell image, thereby generating the filtered image. The filtered image can be obtained as fi=f−f∘k, where fi represents the filtered image, f represents the flow cell image, and k represent the kernel, and ∘ represents the opening operation.

In some embodiments, the filtered image is an image of the corresponding flow cell image filtered by a top-hat filter. In some embodiments, the filtered image is an image of the corresponding flow cell image filtered by a difference of Gaussian (DoG) filter. In some embodiments, the filtered image is an image of the corresponding flow cell image filtered by various filters that extracts small element and details (e.g., in-focus polonies).

After filtering, at least part of the noise and undesired signal in the flow cell images are removed, which can include the cell components and out-of-focus polonies. Removal of such noise and undesired signal advantageously facilitate generating intensities that are attributed to polonies or clusters but not other background objects or noises. Intensities after filtration can be used for more accurate and reliable base calling than those without filtering.

FIGS. 2C and 3C shows two filtered images from two different axial locations. FIG. 2C indicates that a larger number of polonies or clusters are in-focus at the first axial location of z=0, and a much smaller number of polonies or clusters are in-focus at a second axial location of z=5, which is about 10 um away from the first axial location.

FIG. 3E shows another exemplary filtered image of the flow cell image in FIG. 3D. The background objects in the flow cell image are filtered out in the filtered image. The out-of-focus polonies in the flow cell image are also removed from the filtered image. The filtered image shows in-focus polonies or clusters.

In some embodiments, the method 6000 can include an operation of adding an offset to the filtered image(s). In some embodiments, the offset can be predetermined so that after the offset, the range of image intensity can be within a predetermined range. In some embodiments, different offset can be used to bring different filtered images with various intensities ranges to be within a predetermined range that is similar to all the filtered images.

In some embodiments, the processed images include the first and second pluralities of processed images, and the filtered images include the first and second pluralities of filtered images. In some embodiments, the first plurality of processed images and the first plurality of filtered images are from the one or more reference cycles and different channels. In some embodiments, the second plurality of flow cell images, the second plurality of processed images and the second plurality of filtered images are from the one or more reference cycles and the different channels. In some embodiments, the second plurality of flow cell images, the second plurality of processed images and the second plurality of filtered images are from one or more cycles different from the one or more reference cycles and from different channels. In some embodiments, the first and second plurality of flow cell images are identical, the first and second plurality of processed images are identical, and the first and second plurality of filtered images are identical.

The method 6000 can comprise an operation 6350 of obtaining the 3D polony map. The 3D polony map may be determined in one or more reference cycles. During a cycle different from the reference cycle(s), the 3D polony map may have been pre-generated in the reference cycle(s) and can be obtained by actively requesting or retrieving or passively receiving the 3D polony map. In some embodiments, a single 3D polony map is used during sequencing analysis of the same 3D sample so that in cycles different from the reference cycles, no new 3D polony map needs to be generated.

The method 6000 can comprise an operation of generating the 3D polony map. The operation of generating the 3D polony map can but is not limited to occur during one or more reference cycles. As a nonlimiting example, cycles 1-5 or 2-5 can be reference cycles. In some embodiments, the 3D polony map generated in the one or more reference cycles can be used in any cycles different from the reference cycles so that additional computational complexity, cost, and time for generating 3D polony maps in every cycle can be avoided.

The 3D polony map can be generated based on the one or more 2D template images. The 2D template image is used herein equivalent as the 2D polony map. Each of the 2D template image or polony map corresponds to the flow cell image(s) at a specific z location. Each of the 2D template image corresponds to the flow cell image(s) at the specific z location and at a specific tile or subtile. Each of the 2D template image corresponds to a specific sequencing cycle. Each template image may correspond to one or multiple channels. For example, if there are 10 different z locations for the 3D sample to be sequenced, there can be ten 2D template images corresponding to the different z locations for a single subtile during cycle N.

A polony map herein, either 2D or 3D, can be saved a list of coordinates. Each entry in the list of coordinates can correspond to a polony, for example, a center of the polony. Instead of saving a 2D or 3D matrix as the polony map, the list of coordinates can be stored with much less storage space, and can be utilized more efficiently in computations.

In some embodiments, the operation of generating the 3D polony map can comprise obtaining the 2D template images(s). Obtaining the template images can comprise generating the template images or receiving or retrieving the template images. The 2D template images can be generated using the methods and operations described herein. The 2D template images can be generated after filtered, e.g., by the top hat filter or the DOG filter. The 2D template images can be generated after filtering the flow cell images and registering the filtered images to a reference coordinate system. The 2D template images can be generated in one or more reference cycles and the same template images can be used across different cycles and channels. The 2D template image can be a list of coordinates of polonies. For example, each entry in the list can be 2D or 3D coordinates of the polonies.

In some embodiments, the operation of generating the 3D polony map can comprise combining the one or more 2D template images into a candidate 3D polony map. For example, the lists of coordinates in the 2D template images can be added together.

In some embodiments, when the 3D polony map is saved as a 3D matrix instead of a list of entries, the operation of generating the 3D polony map can comprise extracting polonies in the one or more template images. Instead of directly combining the 2D template images, the polonies in the one or more template images can be extracted, and the extracted polonies can be included in the candidate 3D polony map based on their coordinates in the template images. The candidate 3D polony map can be initialized with 0 or other minimal intensity values in all its pixels at different z locations. The extracted intensities can be used to replace the initialized value in the candidate 3D polony map to indicate the pixels or voxels that is at least part of a polony. As a nonlimiting example, the 3D polony map can be a 3D matrix of 0 and 1, where each pixel with 1 indicates that pixel that is part of a polony.

A single polony may appear in one, two, or even more z locations so that the same polony may be included in multiple flow cell images at different z locations. For example, a polony at (x1, y1) at the location z1 may be included again at (x1-1, y1-1) at the location z2. As such, the candidate 3D polony map can include duplicate polonies. The duplicate polonies need to be removed for accurate and reliable 3D base calling. The operation 6350 of generating the 3D polony map can comprise removing duplicate polonies from the candidate 3D polony map.

To remove duplicate polonies, preliminary base callings can be performed. The location of polonies for base callings can be determined by the 2D template image(s) while the intensities for performing base callings can be extracted from the filtered images. The filtering herein can advantageously remove intensity interferences from out-of-focus polonies and background objects, so that the intensities can be used for more accurate and reliable base calling than the unfiltered flow cell images. In some embodiments, the 2D template images contains coordinates of polonies in a reference coordinate system so that even the polonies may have shifted across cycles, the base callings can still be attributed to the same polonies.

After the preliminary base callings are obtained, the operation 6350 can include a repetitive operation of removing the duplicate polonies until a stopping criteria is met. The repetitive operation can include identifying candidate polonies with an identical base call. In response to identifying candidate polonies with an identical base call, the candidate polonies may contain zero, one, two, or more duplicate polonies. The operation 6350 can further comprise an operation of determining 3D distance between each pair of polonies among the candidate polonies. For each pair of polonies (non-repetitive pair), the 3D distance can be calculated based on the coordinates of the polonies. The coordinates can be 3D. The coordinates can include the 2D coordinates of the polonies, e.g., after registration in the reference coordinate system, and the z location of polonies. The 3D distance can be in pixels. The 3D distance can be in other units, e.g., um. The 3D distance can be used to determine if the two polonies with identical base calls are in proximity to each other or not.

In response to determining that the 3D distance between the two polonies is within a predetermined distance threshold, the operation 6350 can include an operation of determining the image intensity for each of the two polonies from the filtered images or the template images. In some embodiments, determining the image intensity for each of the polonies can include normalizing and/or offsetting the image intensity at different z locations to a predetermined range. For example, the normalization and/or offsetting can be based on intensities of fiducial markers at the different z locations. Subsequently, the operation 635 can include removing a polony of the two with a smaller image intensity. The two polonies within the predetermined distance threshold can be considered as a same polony that is duplicated. The duplicate with smaller intensity can be more out-of-focus than the one with greater intensity, and can be removed to ensure accurate and reliable base calling. The predetermined distance threshold can be customized based on the characteristics of the sample, the imaging parameters, the polonies, etc. For example, the predetermined distance threshold can be based on a depth of field of an optical system, a distance between two adjacent flow cell images along an axial direction, or a combination thereof. The distance threshold alone or combination with the stopping criteria, can be adjusted to balance the true duplicates and the false duplicates that are removed. As an example, polony p1 has a preliminary base calling of A determined using its image intensity after filtering, and its location after registration to the reference coordinate system can be at (xp1, yp1). Candidate polonies can be determined to be those with the same base calling of A. 3D distance from each candidate polony to polony p1 can be calculated and compared to a predetermined threshold, e.g., of 0.5 um. Polony p2 that satisfies the distance threshold can be considered as a possible duplicate of p1. Intensities of p1 and p2 are compared and polony p2 with a greater intensity is retained while the coordinates of polony p1 is removed as a duplicate.

After the duplicate polonies are removed, the 3D polony map is generated which includes all the polonies and corresponding coordinates that base calling can be performed on. The 3D polony map can include location information of such polonies. For example, the 3D polony map may include coordinates of each polony in the corresponding filtered images. The 3D polony map may further include the z location of each polony. The 3D polony map may include size and/or shape of the polonies. The 3D polony map may include a unique identification of each polony. The 3D polony map may include image intensity of polonies. Such image intensity may be filtered intensities obtained from the filtered images disclosed herein.

The stopping criteria can be customized. For example, the stopping criteria can be based on different type of samples, imaging parameters, size and shape of polonies, etc. The stopping criteria, the distance threshold, or both may be adjusted to balance the true duplicates and the false duplicates that are removed. As a nonlimiting example, the stopping criteria can be removing the first 1000 duplicate polonies. As a nonlimiting example, the stopping criteria can be a selected time window. As yet another example, the stopping criteria can be there is no duplicate which satisfies the predetermined distance threshold.

In some embodiments, the method 6000 include an operation of registering the flow cell images, the processed images, and/or the filtered images. In some embodiments, the images are registered across channels and different cycles. In some embodiments, the images are registered before any base calling are performed. In some embodiments, the images are registered across channels and different cycles before generating the template images or obtaining the 3D polony maps. In some embodiments, the images are registered across channels and different cycles before generating the filtered images or the processed images.

Various image registration techniques can be used to register the images. The images can be registered using 2D registration techniques. When registering the processed images or the filtered images, they can be treated as flow cell images during registration. In some embodiments, the images can be registered, e.g., across different channels and/or different cycles, using the image registration method 9000 as disclosed herein by treating each image to be registered as a flow cell image that can be acquired using the sequencing system 1100. In some embodiments, the images can be registered after one or more preprocessing operations disclosed herein are performed. In some embodiments, the operation of registering the flow cell images, the processed images, and/or the filtered images may occur before the operation 6350 of obtaining a 3D polony map.

In some embodiments, the operation of registering the flow cell images, the processed images, and/or the filtered images is with respect to a reference coordinate system. In some embodiments, the operation of registering the flow cell images, the processed images, and/or the filtered images is with respect to one or more template images. The operation of registering the images can comprise generating the one or more template images in a reference coordinate system. In some embodiments, the operation of registering the images can comprise registering polonies to template polonies in the one or more template images. The operation of registering the images can comprise determining a plurality of transformations based on the one or more template images. Each of the plurality of transformations can corresponds to a corresponding subtile of the flow cell images, the processed images, or the filtered images and configured to register the subtile to the one or more template images. Each transformation can be used to register a corresponding subtile or tile to the one or more template images. The plurality of transformations can comprise one or more affine transformations.

In some embodiments, the operation of registering the images can comprise performing image registration of the polonies based on fiducial markers. The fiducial markers can be located on the flow cell. Alternatively, the fiducial markers can be external to the flow cell.

In some embodiments, the image registration as a primary analysis step herein is configured to align images from different cycles and/or different channels, for example, with respect to a template image or a reference coordinate system. In some embodiments, the image registration as a primary analysis step herein is configured to register polonies or clusters from different cycles and different channels, e.g., in the filtered image, to a template image or a reference coordinate system.

For example, the base calling can be performed using the filtered images from different channels in cycle N after the filtered images from different channels are registered relative to the corresponding template image disclosed herein.

The method 6000 can comprise an operation 6450 of extracting polony intensities based on the 3D polony map. For each polony in the 3D polony map, the location information of such polony can be obtained from the 3D polony map, e.g., 2D coordinates of the polony and the z location. Using the 2D coordinates and the z location, the corresponding filtered image and its pixel(s) can be determined. Image intensity of such pixels can be extracted from the corresponding filtered image as intensity of such pixel for performing base calling.

The method 6000 can comprise an operation 6550 of performing 3D base calling using the extracted image intensities. Various existing 2D base calling algorithms can be used. The base calling results can be saved with its 3D location information, e.g., coordinates. Such 3D coordinates can be used to register the base callings across different cycles and at different z locations, and/or to register the base callings to cell images herein.

In some embodiments, the operation 6550 of performing base callings comprises performing primary analysis step(s) herein to adjust image intensities of polonies in the filtered images; and making base calls for the polonies based on the adjusted image intensities in the filtered images. The adjustment to image intensity in the filtered images can occur before or after filtering.

In some embodiments, the primary analysis steps comprise one or more of the following: background subtraction; image sharpening; intensity offset adjustment; color correction; intensity normalization; phasing and prephasing correction; quality score estimation.

In some embodiments, the method 6000 comprise an operation of obtaining a second MIP image based on the plurality of flow cell images. The second MIP can be a flattened 2D image of an axial stack of the flow cell images. In some embodiments, the flow cell image are raw images acquired by the sequencing system 1100. The operation of obtaining the second MIP can be different from that of the first MIP image. In the flow cell images, e.g., raw images, polonies out-of-focus can have larger full width half maximum (FWHM), so, the signal of the out-of-focus polonies is more spread out than in-focus polonies or clusters. In some embodiments, the larger FWHM can cause a white ring around a polony. FIG. 5 shows an exemplary second MIP image generated directly from the flow cell images, and there is a ring or halo round the polony in the center of the image. The ring or halo can be image artifacts that may cause error in base calling. The second MIP can also retain some background information, e.g., from undesired background objects, that may interfere with base calling if the second MIP is used for base calling. As such, the second MIP includes artifacts and/or undesired background objects that require additional processing before accurate and reliable base calling can be make based on the intensities in the second MIP.

In some embodiments, the second MIP can be used for registration of the filtered images and/or polonies or clusters since it shares the same FOV, resolution, image size, etc. The artifacts and/or undesired background objects in second MIP may interfere with base calling if the second MIP is used for base calling, but the same artifacts and/or undesired background objects (that are not in the filtered image) can facilitate registration of filtered image to cell images with staining.

In some embodiments, instead of the second MIP, the flow cell images can be directly used for registering the filtered images to the cell images. The flow cell images also contains noise and undesired background objects for base calling purpose but can facilitate registering the flow cell images and the filtered images to cell images.

### Cell Images

In some embodiments, the methods herein advantageously utilize the second MIP for registering the flow cell images to the cell images. The out-of-focus polonies and background objects which may interfere with correct base calling can be used to provide information for registering the flow cell images to the cell images.

The method 6000 can further comprise an operation 6500 of performing image registration of the flow cell images based on the second MIP image. In some embodiments, the image registration of the flow cell images to the one or more cell images, e.g., staining images, is in addition to the image registration as part of the primary analysis, e.g., image registration across cycles and/or channels. The image registration of the flow cell images is configured to align the polonies or clusters relative to the cell structures so that base calling can be assigned to the nuclei, membrane, or other regions of the cell. In some embodiments, registering the flow cell images based on the second MIP image comprises: registering or aligning the background objects in the second MIP to corresponding objects in the one or more cell images. For example, membrane information can be obtained from the second MIP image and aligned with the membrane(s) in the cell images.

In some embodiments, the operation 6600 of performing image registration of the plurality of flow cell images based on the second MIP image comprises registering the second MIP image to the template image or the reference coordinate system. In some embodiments, registering the second MIP image to the template image or the reference coordinate system can rely on the image registration information of the first MIP, since the second MIP and the first MIP are taken from the same FOV.

In some embodiments, the method 6000 can comprise an operation 6700 of registering the first MIP image and/or the 3D base calling based on the first MIP image and the second MIP image to one or more cell images. The background objects in the second MIP can be used to align the second MIP and the first MIP to the cell images. The cell images can also have identical background objects as those in the second MIP, but with transformation. The transformation may be represented by a single transformation of the whole image or be separated into multiple transformations, each transformation representing a portion of the whole image. After finding the transformation(s) of the background objects between the second MIP and the cell images, the polonies and clusters can be registered to the cell images.

In some embodiments, the operation 6700 of registering the MIP image and/or the 3D base calling to the cell images may not relied on the second MIP image(s) obtained directly from the flow cell image but only on the first MIP image(s) of the filtered images. In these embodiments, background information can be obtained from one or more of: the first MIP image(s), the opened images/processed images, the filtered images, and the flow cell images. The background objects can be used to align the first MIP to the cell images by using one or more transformation(s). The transformation may be represented by a single transformation of the whole image or be separated into multiple transformations, each representing a portion of the whole image. After finding the transformation(s) of the background objects between the first MIP and the cell images, the polonies and clusters can be registered to the cell images.

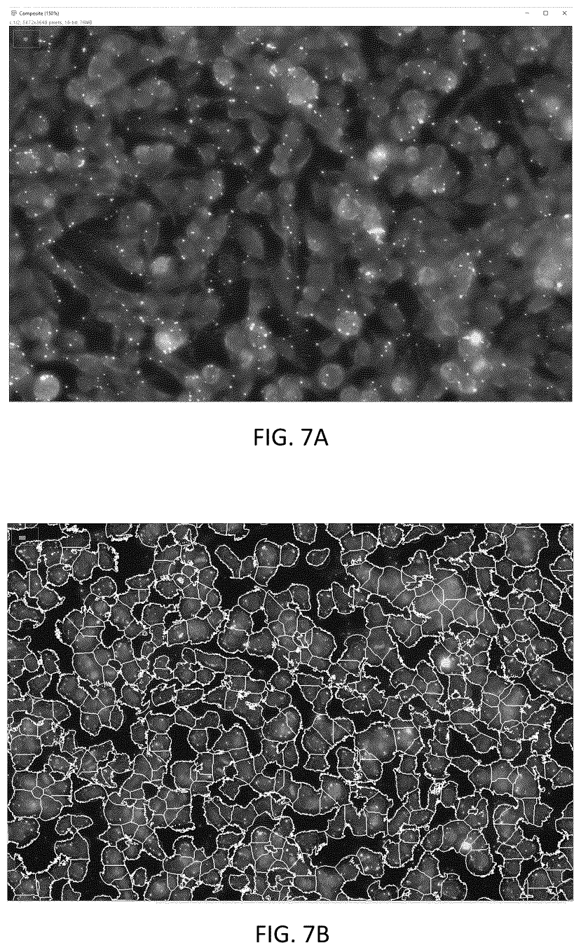

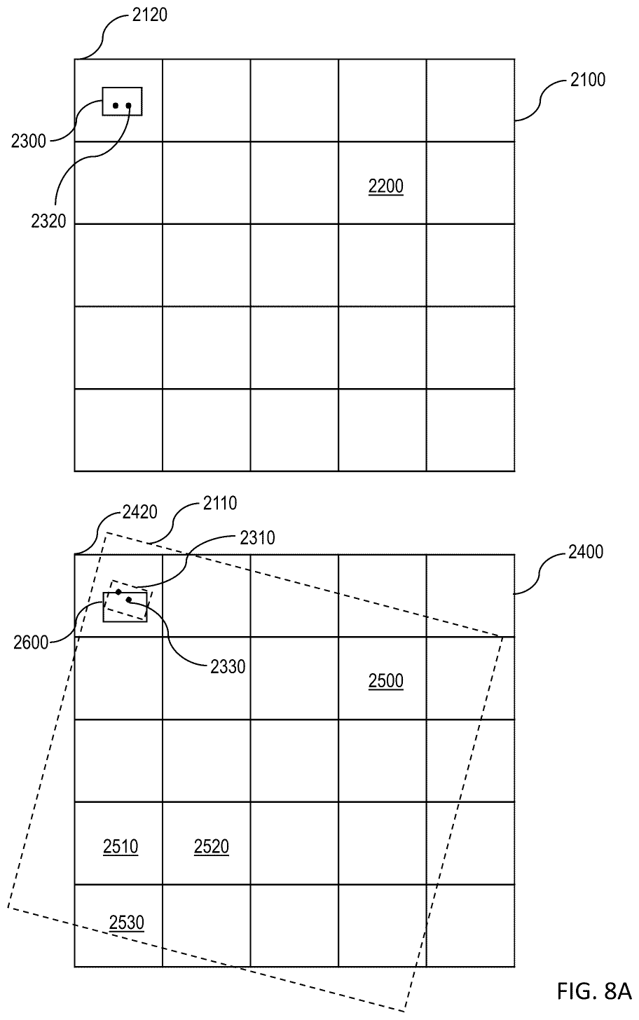

In some embodiments, the operation 6700 of registering the MIP image and/or the 3D base calling to the cell images may not relied on the second MIP image(s) obtained directly from the flow cell image but on fiducial markers. Signal from same fiducial markers can exist in one or more of: the first MIP image(s), the opened images/processed images, the filtered images, and the flow cell images. Such fiducial markers can also be included in the cell images. Aligning the fiducial markers can generate the transformation(s) between the sequencing images, e.g., the first MIP image(s) to the cell images. The transformation(s) can be used to register or align polonies or clusters between the sequencing images and the cell images. FIGS. 7A-7B show exemplary registered MIP image overlayed on the corresponding cell image. The MIP image includes bright spots representing polonies or clusters. Some of the bright spots overlap with the stained nuclei, while some other bright spots occur within the cell membrane but outside of the nuclei. FIG. 7B shows segmentation of individual cells so that polonies or clusters can be grouped with respect to each individual cell.

In some embodiments, the one or more cell images are images of the cell and/or tissue with one or more staining, e.g., fluorescent staining. In some embodiments, the one or more images can comprise staining of cellular structures that help locating polonies or clusters relative to the stained structures. For example, staining can be of cellular structures or components including but not limited to membranes, nuclei, and mitochondria.

In some embodiments, the cell membrane after sequencing analysis and imaging using the sequencing system and reactions can be permeabilized. In some embodiments, the one or more cell images can comprise staining of lipids, such as lipids comprised in the cell membrane. In some embodiments, instead of labeling the lipids, the one or more cell images can comprise staining of one or more transmembrane proteins. The transmembrane proteins can be proteins embedded in the permeabilized membrane.

In some embodiments, the one or more cell images comprises fluorescence signals from cell membranes. The one or more cell images can be microscopic images. The one or more images can be fluorescent images. In some embodiments, different fluorescent colors can be included in the cell images. For example, the nuclei and the cell membrane can be stained with different colors.

In some embodiments, the one or more images can comprise segmentation of: cells, membranes, nuclei, or their combinations. FIG. 7B shows an exemplary cell image with segmentation of individual cells. In some embodiments, the edge(s) of each segment encompass the entire membrane of the cell within the segment. There can be only one cell in each segment. Some segments may not have any cell in them. In some embodiments, adjacent segments do not overlap with each other. In some embodiments, adjacent segments only overlap with each other by sharing one or more edges. In some embodiments, various segmentation algorithms can be used for segmenting the cells.

In some embodiments, the cell images disclosed herein are stained. The staining can occur after acquiring sequencing images using the sequencing system 110. In some embodiments, the staining can occur before acquiring sequencing images. The methods of staining the 3D sample such as the cells, tissue can include one or more operations disclosed herein. The staining of the 3D sample can use various methods that can specifically label one or more cell protein(s) that are located mostly in the membrane but with neglectable occurrence in other regions of the cell (e.g., less than 10%, 5%, 2% in amount or concentration).

The operations can include selecting one or more primary antibodies, each of the one or more primary antibody binding specifically to a corresponding protein. The corresponding protein can be a transmembrane protein of one or more cells. In some embodiments, the corresponding transmembrane protein does not exist in other cellular areas such as the cytosol or nuclei at a predetermined concentration so that staining of the transmembrane protein does not create perceivable signals in cellular areas other than the membrane. In some embodiments, one or more different transmembrane protein can be labeled by a primary antibody. For example, if there are 5 different types of transmembrane proteins, 5 different primary antibodies can be used and each primary antibody specifically binds to one of the transmembrane protein, but not the other transmembrane proteins. In some embodiments, a same type of primary antibody can non-specifically bind different proteins.

The staining methods can comprise an operation selecting one or more secondary antibodies that binds to the one or more primary antibodies. The staining methods can further comprise an operation of labeling the one or more secondary antibodies with a fluorescent label.

In some embodiments, the staining methods can further comprise an operation of linking the secondary antibody and the fluorescent label using a scaffold element or a tertiary probe, e.g., a hydrogel. The scaffold element can be used to retain mRNA of the membrane to facilitate binding and generation of fluorescent signal. In some embodiments, the mRNA can be any mRNAs in the cell. In some embodiments, the staining methods can comprise tissue clearing using various methods to remove some or all of parts of the cells to reduce the background fluorescence coming from portions of the cell that is not the membrane. In some embodiments, the fluorescent label comprises a fluorophore that re-emit light within a specific wavelength range upon light excitation. FIG. 8 shows an exemplary staining of the transmembrane protein using the staining method disclosed herein.

The staining methods can further comprise an operation of generating one or more cell images of the corresponding proteins. The one or more images contains fluorescent signal emitted from the fluorescent labels.

In some embodiments, the methods for base calling in sequencing data analysis can comprise generating a 3D polony map based on the plurality of filtered images. The 3D polony map can include a stack of 2D polony maps, each 2D polony map corresponding to a flow cell image acquired at the corresponding axial location. The 3D polony map can include some or all of polonies that can be identified in the axial stack of flow cell images.

In some embodiment, each 2D polony map can be generated based on the filtered image at the same axial location. In some embodiments, the 2D polony map can be generated from the filtered image similarly as the template image from flow cell images. In some embodiments, a 2D polony map is equivalent to a template image because both of them are virtual images that includes all the polonies identified in one or more cycles at a specific axial location.

As disclosed herein, the filtered image can advantageously exclude background objects that may interfere with signals from polonies. The filtered image can also remove some of the out-of-focus polonies or clusters. The parameters of the filtering, e.g., size and shape of the kernel, can be customized to balance between removing out-of-focus polonies and retaining relatively larger polonies or clusters that assemble out-of-focus polonies.

In some embodiments, each 2D polony map is registered to a template image (3D) or a reference coordinate system (in 3D). The template image or the reference coordinate system can be determined in a reference cycle. For each cycle different than the reference cycle, a 2D polony map can be generated and polony maps from different cycles can be registered relative to each other, using the template image or the reference coordinate system. In some embodiments, a single polony map can be generated for all channels with the same cycle. In some embodiments, the polony map is generated per channel per cycle.

In some embodiments, the 3D polony map is a volumetric polony map stacking all 2D polony maps at different axial locations. In some embodiments, the methods herein include removing duplicates of polonies from the stacked 2D polony maps to generate the 3D polony map. The 3D polony map without duplicates can be used as a reference to locate individual polonies in the sample. In some embodiments, the 3D polony map can be saved as a list of 3D coordinates that indicates the center of polonies.

In some embodiments, the methods herein further comprise an operation of extracting image intensity of polonies based on the 3D polony map. The image intensity can be extracted from one or more of: the flow cell images; the processed images; the filtered images. In some embodiments, image intensity can be extracted from filtered image. In some embodiments, the image intensity can be extracted from the filtered image after processing the filtered image with one or more primary analysis steps disclosed herein. For example, the filtered image can go through phasing and prephasing correction before the image intensity can be extracted.

In some embodiments, the 3D polony map can include duplicates of polonies, and such duplicates can be removed after base calling. For example, all the polonies in the 3D polony map including the duplicates can be used to extract image intensities for base calling. Candidate duplicates of polonies can be identified as polonies at different z locations, e.g., adjacent z locations, and at same x,y locations. If the base calling of such candidate duplicates are identical, one of them can be removed as duplicates. Alternatively, both of them can be removed, and a new polony representing both can be added at the z location as an average of the two and at the same x and y locations.

In some embodiments, the methods herein further comprise an operation of performing 3D base callings based on the extracted image intensity of the polonies. Various 2D base calling algorithms can be used here. For example, base calling of a polony can be made by comparing image intensity of the same polony from different channels, and call the base that corresponds to the largest image intensities among all the channels.

In some embodiments, the operation of filtering flow cell images thereby generating a plurality of filtered images can include performing deconvolution of the plurality of flow cell images. The deconvolution can be at least along the axial direction. In some embodiments, the deconvolution can be 3D. The deconvolution can be in the spatial domain or performing its equivalent in a transformed domain, e.g., the Fourier domain. The deconvolution is configured to reduce or remove the spreading or blurring effect of the optical system on the polonies so that the size and shape of the polonies can appear more accurate in flow cell images. In some embodiments, the deconvolution operation can be used alone as the filtering operation or in combination with other filtering operations, such as the top hat filtering.

### Image Registration to Cell Images

Various methods can be used for registering flow cell images based on fiducial markers. The fiducial markers can be internal or external to the sample. For example, internal fiducial markers can include at least some of the polonies or clusters or background objects in the sample. As another example, external fiducial markers can be microspheres coated on the flow cell so that the signal from the microspheres can function similarly as internal fiducial markers for registration. The same fiducial markers can appear in sequencing images, e.g., the MIP image(s), the flow cell image(s), the filtered images, as well as the cell images so that transformation(s) can be derived from aligning the fiducial markers in different images. The transformation(s) can be used for registering or aligning the sequencing image(s) and cell image(s) and objects that appear in them. Exemplary embodiment of image registration methods are described in PCT patent application No. PCT/US2023/067931 (where the contents of the patent are hereby incorporated by reference in its entirety).

For example, a polony or other object, e.g., background objects, with image intensity I centers at location (x1,y1) in a sequencing image can appear at location (x2, y2) with intensity I′ in a cell image, where (x2,y2)˜Mr*(x1,y1), and Mr is the transformation matrix. Similarly, the inverse transformation matrix Mr⁻¹ can be determined such that (x1,y1)˜Mr⁻¹*(x2,y2). The registration of images can be in 2D and can include translation, scaling, rotation, and/or shearing of flow cell images among different channels. Multiple points in the sequencing image and their corresponding points in the cell image can be used to determine the transformation. The minimum number of points that is needed can be determined by the degree of freedom in the transformation. In some embodiments, the image registration can be 3D with coordinates in x, y, and z axes.

In some embodiments, a sequencing image can be divided into multiple subtitles, and a transformation can be determined for each subtile to represent the transformation of the whole image. In some embodiments, the image transformation of each subtile can be uniquely represented by a transformation matrix. The transformation matrix can be determined as below:

$$( \begin{matrix}a 1 & a 2 & & an \\ b 1 & b 2 & … & bn \\ 1 & 1 & & 1\end{matrix} ) ≃ ( \begin{matrix}M 11 & M 12 & M 13 \\ M 21 & M 22 & M 23 \\ M 31 & M 32 & M 33\end{matrix} ) ( \begin{matrix}x 1 & x 2 & & x n \\ y 1 & y 2 & … & y n \\ 1 & 1 & & 1\end{matrix} ) \qquad ( 1 )$$

$$M = ( \begin{matrix}M 11 & M 12 & M 13 \\ M 21 & M 22 & M 23 \\ M 31 & M 32 & M 33\end{matrix} ) \qquad ( 2 )$$

where n is the number of subtiles, a1=x1+dx1, b1=y1+dy1, a2=x2+dx2, b2=y2+dy2, . . . an=xn+dxn, bn=yn+dyn, d1 . . . dn are 2D shifts corresponding to the subtiles, and where dxn and dyn are shift components of the 2D shift, dn, in the x and y axis, respectively, and wherein M is the 3×3 transformation matrix of the subtile.

In some embodiments, the transformation matrix can be defined as the inverse matrix of M, i.e., M⁻¹, so that equation (1) can be expressed differently as

$${M}^{- 1} ( \begin{matrix}a 1 & a 2 & & a n \\ b 1 & b 2 & … & b n \\ 1 & 1 & & 1\end{matrix} ) ≃ ( \begin{matrix}x 1 & x 2 & & x n \\ y 1 & y 2 & … & y n \\ 1 & 1 & & 1\end{matrix} ) \qquad ( 3 )$$

In some embodiments, the transformation matrix M is an estimation in equations (1) and (3) based on the 2D shifts. In some embodiments, the value of n may affect the accuracy of the estimation.

In some embodiments, more than one region can be selected within a subtile for cross correlation calculation, and more than one 2D shift can be calculated for each subtile and used for estimating the transformation of the subtile. In these embodiments, n in equation (1) can be replaced by a larger number, e.g., 2*n when 2 regions are selected per subtile, and the transformation matrix M can be estimated using equations (1) and (2).

In some embodiments, (a1,b1) . . . (an, bn) in equations (1)-(3) are coordinates for selected region(s) (e.g., coordinates of a center pixel of the corresponding region(s)) after transformation, (x1, y1) . . . (xn, yn) are coordinates of the selected region(s) before transformation, e.g., coordinates of a center pixel.

In some embodiments, n is a number that is no less than 3. The larger the n, the more information can be used to estimate the transformation matrix M. In some embodiments, n is not greater than 9.

In some embodiments, the transformation of one or more subtiles is linear. In some embodiments, the transformation of all subtiles is linear. In some embodiments, the transformation matrix is a matrix in which M31 and M32 is equal to 0, and M33 is 1. In some embodiments, one or more of the transformations per subtile is an affine transformation and the transformation matrix of the entire flow cell image is an affine matrix.

In some embodiments, the transformation matrix M is an estimation in equations (1) and (3) based on the size of the selected region(s). In some embodiments, the size of the selected region may affect the accuracy of the estimation. In some embodiments, the size of the select region can be about 128×128. In some embodiments, the size of the selected region can be about 32×32, 48×48, 64×64, 96×96, 160×160, 196×196, 256×256, or of various different sizes. The transformations per subtile as disclosed herein can be calculated using a selected region within a subtile, the selected region can be equal to or smaller than the subtile. In either case, the transformation estimated using the region can be used to estimate the transformation of the entire subtile given the intrinsic characteristics of image transformation across sequencing cycles. The image transformation between cycles and/or between neighboring pixels can be relatively small, e.g., with less than about 8%, 5%, or less than about 1% of scaling, rotation, and/or shearing. In some embodiments, the transformations disclosed herein can include an image translation with greater than about 5% difference between cycles and/or between neighboring pixels.

After the plurality of transformations are determined for individual subtiles, the transformation of the entire flow cell image can be accurately and reliably estimated by transforming individual subtiles using the plurality of transformations and combining the transformed subtiles into a transformed flow cell. The techniques disclosed herein advantageously estimate the transformation of the flow cell image by determining a plurality of transformations of its individual subtiles. The plurality of transformations can be linear and yet accurately and reliably estimate the transformation of the flow cell image even if the transformation is non-linear. The techniques disclosed herein advantageously eliminate the need to calculate the transformation of the entire flow cell image which can be more computationally intensive and time-consuming and prone to failure than estimating a plurality of transformations for the subtiles.

### Image Registration Across Channels and Cycles

In some embodiments, the method include an operation of aligning or registering the flow cell images across different sequencing cycles, from different channels, and/or at different z levels to a common coordinate system before base calling. The common coordinate system can be the reference coordinate system disclosed herein. The common coordinate system can be predetermined. The common coordinate system can be the reference coordinate system disclosed herein. The common coordinate system can be predetermined. The common coordinate system may be a Cartesian coordinate system. Various other coordinate systems may be used. Other coordinate systems can include but are not limited to the polar coordinate system, cylindrical, or spherical coordinate systems.

Exemplary embodiments of image registration methods are described in PCT patent application No. PCT/US2023/067931 (where the contents of the patent are hereby incorporated by reference in its entirety).

Prior to registering to the cell images, the flow cell images can be registered relative to each other so that polonies or clusters in different cycles and/or channels can be aligned and base calling can be accurate and reliable respect to specific polonies or clusters.

Various methods can be used to register the sequencing images, e.g., flow cell images, filtered images, or MIP images, of different cycles and/or channels.

In some embodiments, the method 6000 include an operation of registering the MIP images, e.g., 9000 in FIG. 9. In some embodiments, the MIP images are registered across channels and different cycles before any base calling are performed. The MIP images can be registered using 2D registration techniques for example, by treating the MIP images as flow cell images acquired from the sequencing system 110. In some embodiments, the MIP images can be registered, e.g., across different channels and/or different cycles, using the image registration method 9000 as disclosed herein by treating each MIP image as a flow cell image. In some embodiments, the MIP images can be registered after one or more preprocessing operations disclosed herein are performed.

For example, the sequencing images can be registered to the reference coordinate system that is common to all the flow cell images so that sequencing images from different cycles and/or channels can be aligned relative to each other. The reference coordinate system can be determined in the reference cycle or any other predetermined cycle. For example, a reference coordinate system can be the coordinate system of the flow cell image from one channel. As another example, the reference coordinate system can be based on the external fiducial markers or other objects external to the flow cell images.

In some embodiments, the method 6000 includes an operation of generating one or more template images in the reference coordinate system by registering the polonies to the one or more template images using the coordinates thereof. FIG. 8A shows a schematic diagram of one or more template images generated in the reference cycle in a reference coordinate system. The template image 2100, in some embodiments, include a size that is about identical to a single tile 2900 that includes a 5×5 grid of subtiles 2200. A region 2300 is selected in each subtile and includes center pixels of the corresponding subtile. The reference coordinate system in this embodiment has an origin 2120 at its top left pixel. In some embodiments, the template image disclosed herein can be individual regions such as region 2300. Each template image can include a plurality of polonies 2320 therein.

In some embodiments, the template image can be of about the same size of a flow cell image so that all the polonies, from different tiles, 2900 in FIGS. 8A-8B, and from multiple channels, can be registered to the same template image. However, such template image may contain polonies that will not be used in at least some operations described herein to reduce computational burden without sacrificing accuracy.

In some embodiments, more than one template images can be generated, and each template image 2300 corresponds to at least part of a subtile of a flow cell image from a channel.

The template image herein can be initialized as a virtual image that has a black or dark background with no signals from polonies. For example, the template image can be initialized to be zero or include otherwise minimal image intensity at all pixels.

After the coordinates of a polony is determined in operation 9100 by image registration of flow cell images across different channels, the intensity of the polony can be added to the template image at the location determined by the coordinates and with the size and shape determined based on registration. The template image can be a virtual image that combines image intensity from polonies obtained from 2, 3, 4, or even more channels at the reference cycle. The pixels of the template containing no polonies in them remains to be black or dark so that the template image can have a cleaner background without noise that appear in actual flow cell images.

In some embodiments, the method 9000 includes an operation of obtaining image intensities, sizes, shapes, or their combinations of the polonies from at least a portion of one or more subtiles in the reference cycle so that such information can be used to include the polonies in the template image. In some embodiments, polonies can have a fixed shape and/or size. In some embodiments, a point spread function determined by the optical system herein is used to determine the fixed shape and/or size of polonies. In some embodiments, the polonies has a fixed spot size that is based on the sigma of a Gaussian point spread function. In some embodiments, one or more polonies have a size of 1-9 pixels. In some embodiments, one or more polonies have a size of 1-3 pixels.

The template image can include polonies from different channels along with the channel information. As an example, the channel information can be provided as a label or a specific order of how the polonies are included.

In some embodiments with multiple template images, each template image 2300 can cover a region within a subtile, and such template image may but is not required to include all the polonies within the subtile.

In some embodiments, the method 9000 includes an operation 9300 of obtaining a flow cell image in a cycle after the reference cycle. The operation 9300 can include passively receiving or actively requesting the flow cell image from an optical system disclosed herein after the flow cell image is generated by the optical system. The optical system can be included in the imager 1160 in FIG. 1.

The flow cell image can include some or all of the same polonies in the template image(s) of the reference cycle. In particular, the flow cell image can include some or all of the same polonies in regions corresponding to the selected region in the reference cycle.

FIG. 8A shows the flow cell image acquired in a cycle different from the reference cycle at the bottom. The flow cell image 2400 is acquired with multiple subtiles 2500. The selected region 2600 in this cycle can be at the same location relative to the new origin 2420 in this cycle as the selected region 2300 to the origin 2120 in the reference cycle. In this cycle, the flow cell image 2100 in the reference may have transformed to the transformed image 2110, and the selected region 2300 correspondingly transformed to region 2310 with some overlap to region 2600. The image transformation herein can be 2D, and can include translation, scaling, rotation, and/or shearing.

In some embodiments, the method 9000 is configured to align template image 2100 or 2300 in the reference cycle and the transformed image 2110 or 2310 in another cycle to the reference coordinate system.

In some embodiments, instead of using region 2310 or 2110 directly in image registration, the method 9000 can include an operation of selecting region 2300 and 2600 for simpler and more convenient determination of image registration. The region 2600 can include at least part of the polonies 2320 that were in the template image 2300 as polonies 2330.

In some embodiments, the method 9000 comprises an operation 9400 of determining a plurality of transformations of the flow cell image 2400 based on the one or more template images 2100 or 2300. As shown in FIG. 8A, each of the plurality of transformations can correspond to a subtile 2500 of the flow cell image 2400 and is configured to register the subtile 2500 of the flow cell 2400 image to a corresponding portion of the template image 2100 (if the template image includes the entire tile) or a corresponding template image 2300 (if there are multiple template images within the tile).

In some embodiments, the operation 6400 may include determining each transformation corresponding to a subtile of the flow cell image. More particularly, each transformation can correspond to a selected region in each of some or all the subtiles. A region can be selected in various ways from a subtile to include at least part of the subtile. The region may be a predetermined two-dimensional shape, e.g., rectangle, circle, or square. As a non-limiting example, the selected region can include one or more center pixels of the subtile as shown in FIG. 8A at 2600. The size of the region can be determined to balance the trade-off between computational complexity and accuracy of image registration. For example, selecting a 64×64 region can be computationally simpler than selecting a 128×128 region but may not be as accurate. In some embodiments, the selected region includes some or all of the polonies 2320 registered in the template image(s) in the reference cycle so that the same polonies and their relative locations in the template image(s) and the flow cell image can be used for determining the transformation. In some embodiments, the size of the template image, e.g., 2300 and the region 2600 can be identical or about identical. In some embodiments, the size of the template image 2100 or 2200 and the selected region 2600 can be different.

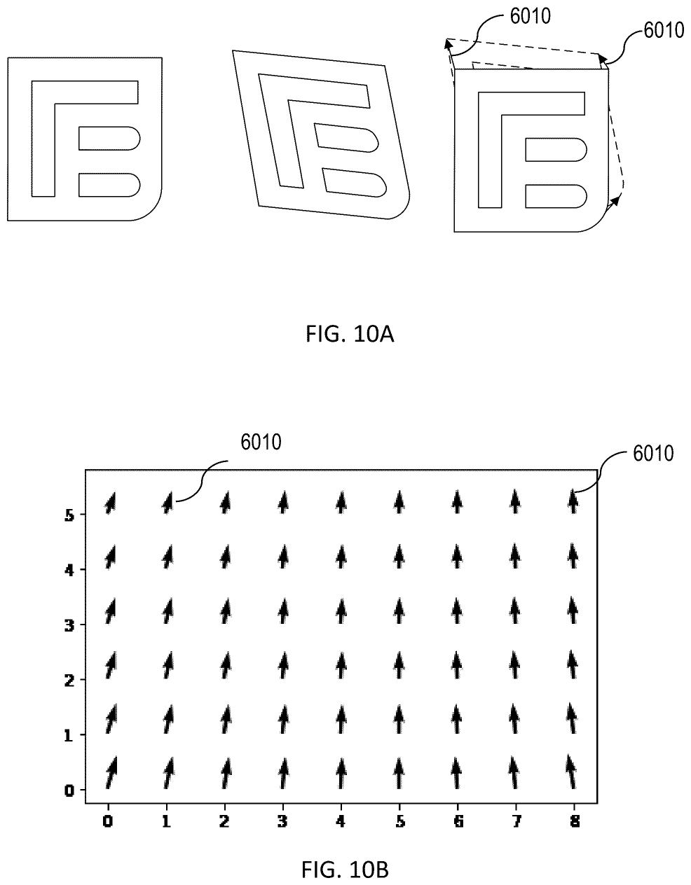

In some embodiments, cross correlation of the selected region and the template image can be computed for determining a 2D shift of the region relative to the template image. FIG. 10A shows a reference image (left) that is transformed with 2D shear, scaling, and rotation into a different image (middle). 2D shifts 6010 at the four corners of the reference image can be determined, for example, using the methods disclosed herein using cross correlation. And the 2D shifts at four corners can be used to estimate the transformation between the two images.

In some embodiments, cross correlation can be calculated in the spatial domain. In some embodiments, cross correlation can be calculated in the spatial frequency domain after Fourier transform (FT). The method 9000 may comprise generating a corresponding Fourier Transformed Image (FTI) of a template image and a Fourier transform of the selected region. The Fourier transformation herein can be calculated using discrete FT (DFT), fast FT (FFT), or the like. The cross correlation can be determined based on the FTI and the Fourier transform of the selected region. As a nonlimiting example, the cross correlation can be the elementwise multiplication of the FTI with the FT of the selected region, with a complex conjugate or rotation of the one of them. Then, an inverse FT of the elementwise multiplication can be obtained. In some embodiments, the cross correlation can be a 2D image with a peak intensity at its coordinate [xp, yp]. In some embodiments, a 2D shift can be determined based the coordinates [xp, yp] in comparison to the coordinates of a peak obtained from cross-correlation of two original images without transformation. The 2D shift of the selected region 2600 can be used to estimate the 2D shift for the entire subtile. In some embodiments, results of calculating the cross correlation in the spatial domain or Fourier domain can be equivalent. In some embodiments, calculation in the Fourier domain can be simpler and more efficient than calculation in the spatial domain.

In some embodiments, the image transformation of the subtile can be determined from 2D shifts from some or all neighboring subtiles with or without the 2D shift from itself. In some embodiments, 2D shifts from all immediate neighbors can be used. For example, to determining transformation of subtile 2530, 2D shifts from 3 neighboring subtiles and the 2D shift from itself can be used. For subtile 2510, a total number of 6 2D shifts including immediate neighboring subtiles and itself can be used. For subtile 2520, a total number of 9 2D shifts including neighboring subtiles and itself can be used. In some embodiments, 2D shifts from some but not all neighboring subtiles can be used. In some embodiments, 2D shifts from all neighboring subtiles except 1-2 outliers can be used to determine the transformation. The outlier(s) can be excluded using a predetermined criterium, e.g., more than 30% or 50% different from other 2D shifts.

FIG. 10B is an image showing 2D shifts within a tile of a flow cell image. In this embodiment, the tile has a 6×9 grid of subtiles, and each subtile has a 2D shift 6010 that is determined using the technologies disclosed herein. Each shift has a magnitude of less than about 5 pixels along x or y axis. A pixel size may vary depending on imaging parameters, an exemplary pixel can be from 0.01 um to 0.9 um. The 2D shifts 6010 can be used to calculate transformation, e.g., affine matrix, for the tile, by individually calculating a transformation for each subtile. In this embodiment, an affine matrix can be calculated using the methods disclosed herein.

In some embodiments, subpixel resolution, e.g., about 0.01, 0.02, 0.03, or 0.05 pixel, of the 2D shifts 6010 can be achieved using various methods including interpolation, upsampling, etc. In some embodiments, subpixel resolution can be achieved by fitting the peak with a selected filter, e.g., a 3×3 or 5×5 Gaussian filter.

In some embodiments, the image transformation of a subtile can be uniquely represented by a transformation matrix. The transformation matrix can be determined as below:

$$( \begin{matrix}a 1 & a 2 & & an \\ b 1 & b 2 & … & bn \\ 1 & 1 & & 1\end{matrix} ) ≃ ( \begin{matrix}M 11 & M 12 & M 13 \\ M 21 & M 22 & M 23 \\ M 31 & M 32 & M 33\end{matrix} ) ( \begin{matrix}x 1 & x 2 & & x n \\ y 1 & y 2 & … & y n \\ 1 & 1 & & 1\end{matrix} ) \qquad ( 4 )$$

$$M = ( \begin{matrix}M 11 & M 12 & M 13 \\ M 21 & M 22 & M 23 \\ M 31 & M 32 & M 33\end{matrix} ) \qquad ( 5 )$$

where n is the number of subtiles, a1=x1+dx1, b1=y1+dy1, a2=x2+dx2, b2=y2+dy2, . . . an=xn+dxn, bn=yn+dyn, d1 . . . dn are 2D shifts corresponding to the subtiles, and where dxn and dyn are shift components of the 2D shift, dn, in the x and y axis, respectively, and wherein M is the 3×3 transformation matrix of the subtile.

In some embodiments, the transformation matrix can be defined as the inverse matrix of M, i.e., M⁻¹, so that equation (1) can be expressed differently as

$${M}^{- 1} ( \begin{matrix}a 1 & a 2 & & a n \\ b 1 & b 2 & … & b n \\ 1 & 1 & & 1\end{matrix} ) ≃ ( \begin{matrix}x 1 & x 2 & & x n \\ y 1 & y 2 & … & y n \\ 1 & 1 & & 1\end{matrix} ) \qquad ( 6 )$$

In some embodiments, the transformation matrix M is an estimation in equations (4) and (6) based on the 2D shifts. In some embodiments, the value of n may affect the accuracy of the estimation. In some embodiments, more than one region can be selected within a subtile for cross correlation calculation, and more than one 2D shift can be calculated for each subtile and used for estimating the transformation of the subtile. In these embodiments, n in equation (1) can be replaced by a larger number, e.g., 2*n when 2 regions are selected per subtile, and the transformation matrix M can be estimated using equations (4) and (5).

In some embodiments, n is a number that is no less than 3. The larger the n, the more information can be used to estimate the transformation matrix M. In some embodiments, n is not greater than 9.

In some embodiments, the transformation of one or more subtiles is linear. In some embodiments, the transformation of all subtiles is linear. In some embodiments, the transformation matrix is a matrix in which M31 and M32 is equal to 0, and M33 is 1. In some embodiments, one or more of the transformations per subtile is an affine transformation and the transformation matrix is an affine matrix.

In some embodiments, the transformation matrix M is an estimation in equations (4) and (6) based on the size of the selected region(s). In some embodiments, the size of selected region may affect the accuracy of the estimation. In some embodiments, the size of the select region can be about 128×128. In some embodiments, the size of the selected region can be about 32×32, 48×48, 64×64, 96×96, 160×160, 196×196, or 256×256. The transformations per subtile as disclosed herein can be calculated using a selected region within a subtile, the selected region can be equal to or smaller than the subtile. In either case, the transformation estimated using the region can be used to estimate the transformation of the entire subtile given the intrinsic characteristics of image transformation across sequencing cycles. The image transformation between cycles and/or between neighboring pixels can be relatively small, e.g., with less than about 5% or less than about 1% of scaling, rotation, and/or shearing. In some embodiments, the transformations disclosed herein can include an image translation with greater than about 5% difference between cycles and/or between neighboring pixels.

After the plurality of transformations are determined for individual subtiles, the transformation of the flow cell image can be accurately and reliably estimated by the plurality of transformations. The techniques disclosed herein advantageously estimate the transformation of the flow cell image by determining a plurality of transformations of its individual subtiles. The plurality of transformations can be linear and yet accurately and reliably estimate the transformation of the flow cell image even if the transformation is non-linear. The techniques disclosed herein advantageously eliminate the need to calculate the transformation of the entire flow cell image which can be more computationally intensive and time-consuming than estimating a plurality of transformations for the subtiles.

In some embodiments, the computer-implemented method 9000 further include an operation of saving the plurality of transformation by the processor disclosed herein. In some embodiments, the computer-implemented method 9000 further include an operation of communicating the plurality of transformations to a processing unit such as a CPU for subsequent operations.

In some embodiments, the computer-implemented method 9000 further include registering subtitles to the one or more template images using the plurality of transformations. This operation can be performed by the processing unit such as the CPU(s). In any given cycle different from the reference cycle, each subtile can be registered or transformed to the one or more template images by multiplying the subtile by the transformation matrix corresponding to the subtile.

In some embodiments, the computer-implemented method 9000 may include an operation of performing one or more preprocessing steps on the flow cell images of the reference cycle and/or other cycles before registration of images from that cycle.

In some embodiments, this operation of performing one or more preprocessing steps can be performed by the FPGA(s). In some embodiments, the data after the operation can be communicated by the FPGA(s) to the CPU(s) so that CPU(s) can perform subsequent operation(s) in method 6000 and 9000 using such data.

In some embodiments, the one or more preprocessing steps of flow cell images in the reference cycle can be performed before operation 9100 or 9200 or after operation 9200. In some embodiments, the one or more preprocessing steps of flow cell images in the reference cycle can be performed after the operation of receiving the flow cell images in the reference cycle from the optical system disclosed herein. In some embodiments, the one or more preprocessing steps of flow cell images in the reference cycle can be performed before the operation of obtaining image intensities, sizes, shapes, or their combinations of the polonies from the plurality of subtiles of the flow cell images in the reference cycle.

In some embodiments, the one or more preprocessing steps of flow cell images in cycles other than the reference cycle can be performed after operation 9300 or 9400. In some embodiments, the one or more preprocessing steps of flow cell images in cycles other than the reference cycle can be performed after the operation of registering the subtiles of flow cell image to the one or more template images. In some embodiments, the one or more preprocessing steps of flow cell images in cycles other than the reference cycle can be before the operation of extracting image intensities of a plurality of polonies from the subtiles of the flow cell image. In some embodiments, the one or more preprocessing steps of flow cell images in cycles other than the reference cycle can be before the operation of making base calls using image intensities of the subtiles of the flow cell image.

The one or more preprocessing steps can comprise background subtraction. The background subtraction is configured to remove at least some background signal that may interfere with the signal of interest, i.e., image intensities of the polonies. The background signal can be noise caused by multiple sources including the flow cell 1120, the imager 1160, the sequencer 1140, and other sources. The background subtraction can be adjusted to avoid over subtraction.

The one or more preprocessing steps can include image sharpening so that image intensities of polonies can be optimized in consideration of their surroundings in the flow cell images. For example, a Laplacian of Gaussian (LoG) filter can be used for sharpening.

The one or more preprocessing steps can include image registration so that image intensities of polonies can be registered relative to each other. For example, the image intensities can be registered to the template as disclosed herein.

The one or more preprocessing steps can include intensity offset adjustment that can remove the offset in the intensity that has not been removed during background subtraction.

The one or more preprocessing steps can include color correction to remove interference of one channel from other channels or colors.

The one or more preprocessing steps can include phasing and prephasing correction which is configured to correct image intensities within a specific cycle by removing intensity biases caused by sequencing of DNA fragments that are out of synchronization from other fragments by either falling behind or getting ahead.

The one or more preprocessing steps can include intensity normalization so that the image intensity of polonies from different channels can be normalized to be within a predetermined range.

The one or more preprocessing steps can comprise: background subtraction; image sharpening; or a combination thereof.

In some embodiments, the computer-implemented method 9000 further include extracting image intensities of a plurality of polonies, from the subtiles registered to the template image(s). This operation can be performed by the processing unit such as the CPU(s) or FPGA(s). In some embodiments, polonies with their corresponding intensities are extracted from the flow cell image(s) into a different data format that is simpler and more efficient to handle. For example, each polony can have 4 different intensities, each intensity from a different channel. Such intensities can be extracted into a list, with each entry of the list corresponding to a polony. The list can be generated after image registration to reflect location information of the same polonies in different cycles. As such, image intensities of the same polony in different cycles can be located in different lists each corresponding to a cycle.

In some embodiments, the computer-implemented method 9000 further include making base calls using image intensities of the subtiles of the flow cell image after the registration so that base calling can be made accurately relative to the same polonies across different channels and in different cycles.

In some embodiments, the method 9000 includes an operation 9400 of determining a plurality of transformations of the flow cell image. The operation 9400 can include determining each of the transformations without using any neighboring subtiles as disclosed herein. Instead, more than 2 regions can be selected within the subtile, and 2D shift can be determined for each of the regions. The transformation of the subtile can be determined using the 2D shifts obtained from regions within the same subtile using equations (1) and (2). The regions within a subtile can be smaller in size than the region 2600 in neighboring subtiles. For example, the region 2600 can be about 128×128, and the regions within a subtile can be 3, 4, 5, or even more regions, and each region include a about 64×64 matrix. The other operations of method 9000 can remain the same for image registration with or without using neighboring subtiles in generating the transformations.

As used herein, the terms “dark,” refer to image intensity or signal intensity that is below a predetermined threshold so that no base calls can be performed with a predetermined quality. The predetermined threshold can be customized based on different samples, sequencing parameters, and/or other factors that may influence signal intensity. In other words, base calls performed with “dark” intensity may be erroneous and not reliable. Alternatively, no base calls can be made with “dark” intensity. The image intensity or signal intensity are in the flow cell images. A “dark” flow cell image herein refer to a flow cell image in which substantially all the pixels or voxels have intensity that are below a predetermined intensity threshold. A “dark” base herein refer to a nucleotide base that is attached to “dark” fluorescent dyes and the flow cell images of a channel corresponding to the “dark” base can be “dark” flow cell images.

As used herein, the terms “bright,” refer to image intensity or signal intensity that is above a predetermined threshold so that no base calls can be performed with a predetermined quality. The predetermined threshold can be customized to be various numbers or ranges absolute image intensities, percentage of image intensities, and/or relative image intensities. The predetermined thresholds for determining “bright” and “dark” may not be the same. For example, the lowest 5% of the image intensities within the flow cell image may be “dark” and the highest 15% of the image intensities within the flow cell image may be “bright.”

### Computer Systems

Various embodiments of the methods 6000, 9000, 5200 may be implemented, for example, using one or more computer systems, such as computer system 4000 shown in FIG. 4. One or more computer systems 4000 may be used, for example, to implement any of the embodiments discussed herein, as well as combinations and sub-combinations thereof.

Computer system 4000 may include one or more hardware processors 404. The hardware processor 404 can be central processing unit (CPU), graphic processing units (GPU), or their combination. Processor 404 may be connected to a bus or communication infrastructure 406.

Computer system 4000 may also include user input/output device(s) 403, such as monitors, keyboards, pointing devices, etc., which may communicate with communication infrastructure 406 through user input/output interface(s) 402. The user input/output devices 403 may be coupled to the user interface 1240 in FIG. 1.

One or more of processors 404 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, and/or producing results of other proof-of-work computations for some blockchain-based applications, for example. With capabilities of general-purpose computing on graphics processing units (GPGPU), the GPU may be particularly useful in at least the image recognition and machine learning aspects described herein.

Additionally, one or more of processors 404 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors and/or other logic to facilitate such acceleration.

Computer system 4000 may also include a data storage device such as a main or primary memory 408, e.g., random access memory (RAM). Main memory 408 may include one or more levels of cache. Main memory 408 may have stored therein control logic (i.e., computer software) and/or data.

Computer system 4000 may also include one or more secondary data storage devices or secondary memory 410. Secondary memory 410 may include, for example, a main storage drive 412 and/or a removable storage device or drive 414. Main storage drive 412 may be a hard disk drive or solid-state drive, for example. Removable storage drive 414 may be a floppy disk drive, a magnetic tape drive, a compact disk drive, an optical storage device, tape backup device, and/or any other storage device/drive.

Removable storage drive 414 may interact with a removable storage unit 418.

Removable storage unit 418 may include a computer usable or readable storage device having stored thereon computer software and/or data. The software can include control logic. The software may include instructions executable by the hardware processor(s) 404. Removable storage unit 418 may be a floppy disk, magnetic tape, compact disk, DVD, optical storage disk, and/any other computer data storage device. Removable storage drive 414 may read from and/or write to removable storage unit 418.

Secondary memory 410 may include other means, devices, components, instrumentalities or other approaches for allowing computer programs and/or other instructions and/or data to be accessed by computer system 4000. Such means, devices, components, instrumentalities or other approaches may include, for example, a removable storage unit 422 and an interface 420. Examples of the removable storage unit 422 and the interface 420 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 4000 may further include a communication or network interface 424. Communication interface 424 may enable computer system 4000 to communicate and interact with any combination of external devices, external networks, external entities, etc. (individually and collectively referenced by reference number 428). For example, communication interface 424 may allow computer system 4000 to communicate with external or remote devices 428 over communication path 426, which may be wired and/or wireless (or a combination thereof), and which may include any combination of LANs, WANs, the Internet, etc. Control logic and/or data may be transmitted to and from computer system 4000 via communication path 426. In some embodiments, communication path 426 is the connection to the cloud 130, as depicted in FIG. 1. The external devices, etc. referred to by reference number 428 may be devices, networks, entities, etc. in the cloud 1300.

Computer system 4000 may also be any of a personal digital assistant (PDA), desktop workstation, laptop or notebook computer, netbook, tablet, smart phone, smart watch or other wearable, appliance, part of the Internet of Things (IoT), and/or embedded system, to name a few non-limiting examples, or any combination thereof.

It should be appreciated that the framework described herein may be implemented as a method, process, apparatus, system, or article of manufacture such as a non-transitory computer-readable medium or device. For illustration purposes, the present framework may be described in the context of distributed ledgers being publicly available, or at least available to untrusted third parties. One example as a modern use case is with blockchain-based systems. It should be appreciated, however, that the present framework may also be applied in other settings where sensitive or confidential information may need to pass by or through hands of untrusted third parties, and that this technology is in no way limited to distributed ledgers or blockchain uses.

Computer system 4000 may be a client or server, accessing or hosting any applications and/or data through any delivery paradigm, including but not limited to remote or distributed cloud computing solutions; local or on-premises software (e.g., “on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); and/or a hybrid model including any combination of the foregoing examples or other services or delivery paradigms.

Any applicable data structures, file formats, and schemas may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

Any pertinent data, files, and/or databases may be stored, retrieved, accessed, and/or transmitted in human-readable formats such as numeric, textual, graphic, or multimedia formats, further including various types of markup language, among other possible formats. Alternatively or in combination with the above formats, the data, files, and/or databases may be stored, retrieved, accessed, and/or transmitted in binary, encoded, compressed, and/or encrypted formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform and/or unique identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C#, Objective-C, Java, Scala, Clojure, Elixir, Swift, Go, Perl, PHP, Python, Ruby, JavaScript, WebAssembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some embodiments, a tangible, non-transitory apparatus or article of manufacture comprising a tangible, non-transitory computer usable or readable medium having control logic (software) stored thereon may also be referred to herein as a computer program product or program storage device. This includes, but is not limited to, computer system 4000, main memory 408, secondary memory 410, and removable storage units 418 and 422, as well as tangible articles of manufacture embodying any combination of the foregoing. Such control logic, when executed by one or more data processing devices (such as computer system 4000), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 4. In particular, embodiments may operate with software, hardware, and/or operating system implementations other than those described herein.

### Optical Systems

The imager 1160 in FIG. 1 can include one or more optical systems. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus enable higher throughput image acquisition and analysis.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being images.

In some instances, improvements in imaging performance, e.g., for dual-side (flow cell) imaging applications comprising the use of thick flow cell walls (e.g., wall (or coverslip) thickness >700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls and/or intervening fluid layer in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Exemplary embodiments disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 μm, at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm², at least 0.2 mm², at least 0.5 mm², at least 0.7 mm², at least 1 mm², at least 2 mm², at least 3 mm², at least 5 mm², or at least 10 mm², or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some embodiments, the numerical aperture may be at least 0.75. In some embodiments, the numerical aperture is at least 1.0. In some embodiments, the working distance is at least 850 μm. In some embodiments, the working distance is at least 1,000 μm. In some embodiments, the field-of-view may have an area of at least 2.5 mm². In some embodiments, the field-of-view may have an area of at least 3 mm². In some embodiments, the spatial sampling frequency may be at least 2.5 times the optical resolution of the fluorescence imaging system. In some embodiments, the spatial sampling frequency may be at least 3 times the optical resolution of the fluorescence imaging system. In some embodiments, the system may further comprise an X-Y-Z translation stage such that the system is configured to acquire a series of two or more fluorescence images in an automated fashion, wherein each image of the series is or can be acquired for a different field-of-view. In some embodiments, a position of the sample plane may be simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some embodiments, the system further comprises an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm or greater. In some embodiments, the specified error threshold is 50 nm or less. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also discloser herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens may be a commercially-available microscope objective. In some embodiments, the commercially-available microscope objective may have a numerical aperture of at least 0.3. In some embodiments, the objective lens may have a working distance of at least 700 μm. In some embodiments, the objective lens may be corrected to compensate for a cover slip thickness (or flow cell wall thickness) of 0.17 mm or of greater or lesser thickness than 0.17 mm. In some embodiments, the optical system may be corrected to compensate for cover slip thickness, flow cell thickness, or distance between desired focal planes. In some embodiments, said correction may be made by inserting a corrective optic, such as a lens or optical assembly into the light path of the optical system. In some embodiments, said correction may be made without inserting a corrective optic, such as a lens or optical assembly into the light path of the optical system. In some embodiments, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some embodiments, the at least one tube lens may be a compound lens comprising three or more optical components. In some embodiments, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side imaging compared to that for a conventional system comprising an objective lens, a motion-actuated compensator, and an image sensor. In some embodiments, the use of the at least one tube lens provides for an at least 10% improvement in the imaging performance metric for dual-side imaging compared to that for a conventional system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm². In some embodiments, the light delivered to the specified field-of-illumination is of uniform intensity across a specified field-of-view for an imaging system used to acquire images of the support surface. In some embodiments, the specified field-of-view has an area of at least 2 mm². In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

### Imaging Modules and Systems

It will be understood by those of skill in the art that the disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality and/or image processing functionality. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical and/or optomechanical components, such as an X-Y translation stage, an X-Y-Z translation stage, a piezoelectric focusing mechanism, and the like. In some instances, they may function as modules, components, sub-assemblies, or sub-systems of larger systems designed for genomics applications (e.g., genetic testing and/or nucleic acid sequencing applications). For example, in some instances, they may function as modules, components, sub-assemblies, or sub-systems of larger systems that further comprise light-tight and/or other environmental control housings, temperature control modules, fluidics control modules, fluid dispensing robotics, pick-and-place robotics, one or more processors or computers, one or more local and/or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

The present disclosure provides methods for sequencing immobilized or non-immobilized template molecules. The methods can be operated in system 1000, for example, in sequencer 1140. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. In some embodiments, the sequencing reactions employ phosphate chain labeled nucleotides.

In some embodiments, the immobilized concatemers each comprise tandem repeat units of the sequence-of-interest (e.g., insert region) and any adaptor sequences. For example, the tandem repeat unit comprises: (i) a left universal adaptor sequence having a binding sequence for a first surface primer (1121) (e.g., surface pinning primer), (ii) a left universal adaptor sequence having a binding sequence for a first sequencing primer (1141) (e.g., forward sequencing primer), (iii) a sequence-of-interest (1111), (iv) a right universal adaptor sequence having a binding sequence for a second sequencing primer (1151) (e.g., reverse sequencing primer), (v) a right universal adaptor sequence having a binding sequence for a second surface primer (1131) (e.g., surface capture primer), and (vii) a left sample index sequence (1161) and/or a right sample index sequence (1171). In some embodiments, the tandem repeat unit further comprises a left unique identification sequence (1181) and/or a right unique identification sequence (1191). In some embodiments, the tandem repeat unit further comprises at least one binding sequence for a compaction oligonucleotide. In some embodiments, FIGS. 11 and 12 show linear library molecules or a unit of a concatemer molecule.

The immobilized concatemer can self-collapse into a compact nucleic acid nanoball. Inclusion of one or more compaction oligonucleotides during the RCA reaction can further compact the size and/or shape of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yields an increased signal intensity for each concatemer. Multiple portions of a given concatemer can be simultaneously sequenced. Furthermore, a plurality of binding complexes can form along a particular concatemer molecule, each binding complex comprising a sequencing polymerase bound to a template/primer duplex and bound to a multivalent molecule, wherein the plurality of binding complexes remain stable without dissociation resulting in increased persistence time which increases signal intensity and reduces imaging time.

### Methods for Sequencing Using Nucleotide Analogs

The present disclosure provides methods for sequencing any of the immobilized template molecules described herein, the methods comprising step (a): contacting a sequencing polymerase to (i) a nucleic acid template molecule and (ii) a nucleic acid sequencing primer, wherein the contacting is conducted under a condition suitable to bind the sequencing polymerase to the nucleic acid template molecule which is hybridized to the nucleic acid primer, wherein the nucleic acid template molecule hybridized to the nucleic acid primer forms the nucleic acid duplex. In some embodiments, the sequencing polymerase comprises a recombinant mutant sequencing polymerase that can bind and incorporate nucleotide analogs.

In some embodiments, in the methods for sequencing template molecules, the sequencing primer comprises a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemers). In some embodiments, the plurality of nucleic acid template molecules comprise the same target sequence of interest or different target sequences of interest. In some embodiments, the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., fluorophore) that emits a detectable signal. The detectable reporter moiety comprises a fluorophore. In some embodiments, the fluorophore is attached to the nucleo-base. In some embodiments, the fluorophore is attached to the nucleo-base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleo-base. When the incorporated chain terminating nucleotide is detectably labeled, step (b) further comprises detecting the emitted signal from the incorporated chain terminating nucleotide. In some embodiments, step (b) further comprises identifying the nucleo-based of the incorporated chain terminating nucleotide.

In some embodiments, the methods for sequencing further comprise step (c): removing the chain terminating moiety from the incorporated chain terminating nucleotide to generate an extendible 3′OH group. In some embodiments, step (c) further comprises removing the detectable label from the incorporated chain terminating nucleotide. In some embodiments, the sequencing polymerase remains bound to the template molecule which is hybridized to the sequencing primer which is extended by one nucleo-base.

In some embodiments, the methods for sequencing further comprise step (d): repeating steps (b) and (c) at least once.

### Two-Stage Methods for Nucleic Acid Sequencing

The present disclosure provides a two-stage method for sequencing any of the immobilized template molecules described herein. In some embodiments, the first stage generally comprises binding multivalent molecules to complexed polymerases to form multivalent-complexed polymerases, and detecting the multivalent-complexed polymerases.

In some embodiments, the first stage comprises step (a): contacting a plurality of a first sequencing polymerase to (i) a plurality of nucleic acid template molecules and (ii) a plurality of nucleic acid sequencing primers, wherein the contacting is conducted under a condition suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex wherein the nucleic acid duplex comprises a nucleic acid template molecule hybridized to a nucleic acid primer. In some embodiments, the first polymerase comprises a recombinant mutant sequencing polymerase.

In some embodiments, in the methods for sequencing template molecules, the sequencing primer comprises an oligonucleotide having a 3′ extendible end or a 3′ non-extendible end. In some embodiments, the plurality of nucleic acid template molecules comprise amplified template molecules (e.g., clonally amplified template molecules). In some embodiments, the plurality of nucleic acid template molecules comprise one copy of a target sequence of interest. In some embodiments, the plurality of nucleic acid molecules comprise two or more tandem copies of a target sequence of interest (e.g., concatemers). In some embodiments, the nucleic acid template molecules in the plurality of nucleic acid template molecules comprise the same target sequence of interest or different target sequences of interest. In some embodiments, the plurality of nucleic acid template molecules and/or the plurality of nucleic acid primers are in solution or are immobilized to a support. In some embodiments, when the plurality of nucleic acid template molecules and/or the plurality of nucleic acid primers are immobilized to a support, the binding with the first sequencing polymerase generates a plurality of immobilized first complexed polymerases. In some embodiments, the plurality of nucleic acid template molecules and/or nucleic acid primers are immobilized to 10²-10¹⁵ different sites on a support. In some embodiments, the binding of the plurality of template molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

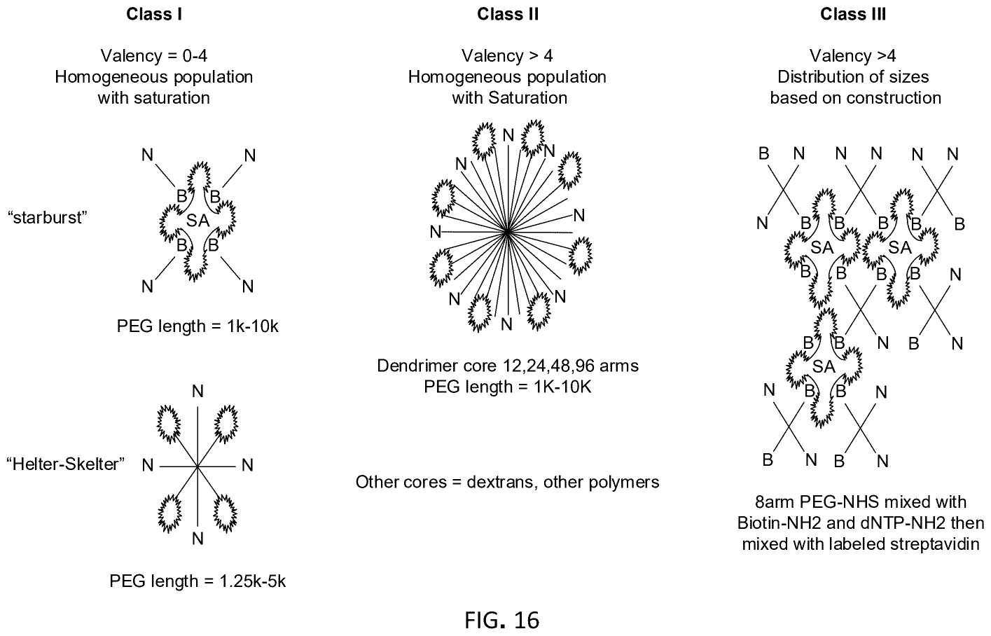

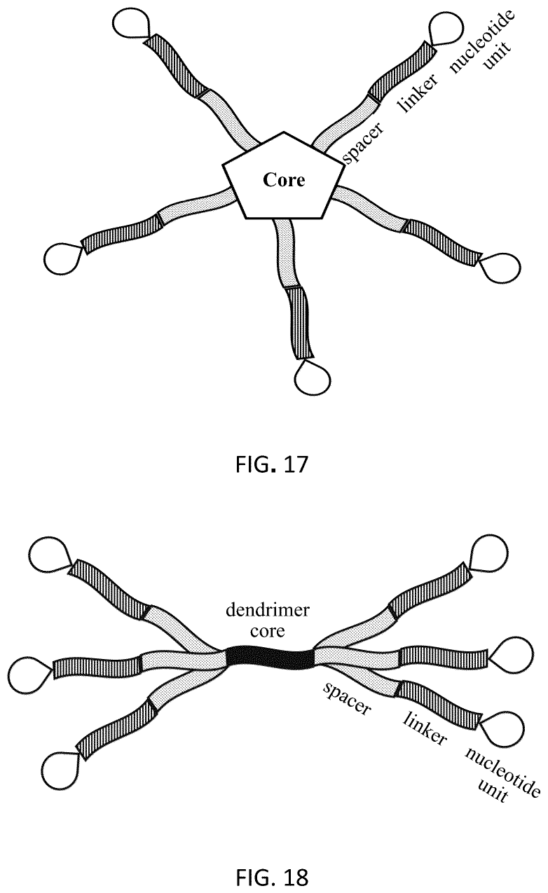

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., nucleotide unit) (e.g., FIGS. 16-20). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprise at least one multivalent molecule having multiple nucleotide arms (e.g., FIGS. 16-19) each attached with a nucleotide analog (e.g., nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety that emits a signal. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the signals emitted by the multivalent molecules that are bound to the complexed polymerases, where the complementary nucleotide units of the multivalent molecules are bound to the primers but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing further comprise step (d): identifying the nucleo-base of the complementary nucleotide units that are bound to the plurality of first complexed polymerases, thereby determining the sequence of the template molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some embodiments, the second stage of the two-stage sequencing method generally comprises nucleotide incorporation. In some embodiments, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) have an amino acid sequence that is 100% identical to the amino acid sequence as the plurality of the second sequencing polymerases of step (f). In some embodiments, the plurality of first sequencing polymerases of step (a) have an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases thereby extending the sequencing primer by one nucleo-base. In some embodiments, the incorporating the nucleotide into the 3′ end of the sequencing primer in step (g) comprises a primer extension reaction. In some embodiments, the contacting of step (g) is conducted in the presence of at least one catalytic cation comprising magnesium and/or manganese. In some embodiments, the plurality of nucleotides comprise native nucleotides (e.g., non-analog nucleotides) or nucleotide analogs. In some embodiments, the plurality of nucleotides comprise a 2′ and/or 3′ chain terminating moiety which is removable or is not removable. In some embodiments, at least one of the nucleotides in the plurality is not labeled with a detectable reporter moiety. In some embodiments, the plurality of nucleotides are non-labeled. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, when the plurality of nucleotides in step (g) are detectably labeled, the methods for sequencing further comprise step (h): detecting the complementary nucleotides which are incorporated into the primers of the nucleotide-complexed polymerases. In some embodiments, the plurality of nucleotides are labeled with a detectable reporter moiety to permit detection. In some embodiments, when the plurality of nucleotides in step (g) are non-labeled, the detecting of step (h) is omitted.

In some embodiments, when the plurality of nucleotides in step (g) are detectably labeled, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid template molecules. In some embodiments, when the plurality of nucleotides in step (g) are non-labeled, the identifying of step (i) is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once. In some embodiments, the sequence of the nucleic acid template molecules can be determined by detecting and identifying the multivalent molecules that bind the sequencing polymerases but do not incorporate into the 3′ end of the primer at steps (c) and (d). In some embodiments, the sequence of the nucleic acid template molecule can be determined (or confirmed) by detecting and identifying the nucleotide that incorporates into the 3′ end of the primer at steps (h) and (i).

In some embodiments, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 16-19.

In some embodiments, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. Exemplary multivalent molecules are shown in FIGS. 16-19.

The present disclosure provides methods for sequencing any of the immobilized template molecules described herein, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

Methods for Sequencing using Phosphate-Chain Labeled Nucleotides

The present disclosure provides methods for sequencing using immobilized sequencing polymerases which bind non-immobilized template molecules, wherein the sequencing reactions are conducted with phosphate-chain labeled nucleotides. In some embodiments, the sequencing methods comprise step (a): providing a support having a plurality of sequencing polymerases immobilized thereon. In some embodiments, the sequencing polymerase comprises a processive DNA polymerase. In some embodiments, the sequencing polymerase comprises a wild type or mutant DNA polymerase, including for example a Phi29 DNA polymerase. In some embodiments, the support comprise a plurality of separate compartments and a sequencing polymerase is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

In some embodiments, the sequencing method further comprises step (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises step (c): contacting the plurality of polymerase/template/primer complexes with a plurality of phosphate chain labeled nucleotides each comprising an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes are contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiment, the polymerase-catalyzed nucleotide incorporation reaction cleaves between the alpha and beta phosphate groups thereby releasing a multi-phosphate chain linked to a fluorophore.

In some embodiments, the sequencing method further comprises step (d): detecting the fluorescent signal emitted by the phosphate chain labeled nucleotide that is bound by the sequencing polymerase, and incorporated into the terminal end of the sequencing primer. In some embodiments, step (d) further comprises identifying the phosphate chain labeled nucleotide that is bound by the sequencing polymerase, and incorporated into the terminal end of the sequencing primer.

In some embodiments, the sequencing method further comprises step (e): repeating steps (c)-(d) at least once. In some embodiments, sequencing methods that employ phosphate chain labeled nucleotides can be conducted according to the methods described in U.S. Pat. Nos. 7,170,050; 7,302,146; and/or 7,405,281.

### Sequencing Polymerases

The present disclosure provides methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a template molecule. In some embodiments, the sequencing polymerase(s) is/are capable of binding a complementary nucleotide unit of a multivalent molecule opposite a nucleotide in a template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Nucleotides

The present disclosure provides methods for sequencing nucleic acid molecules, where any of the sequencing methods described herein employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride. In some embodiments, the chain terminating moiety may be cleavable/removable with nitrous acid. In some embodiments, a chain terminating moiety may be cleavable/removable using a solution comprising nitrite, such as, for example, a combination of nitrite with an acid such as acetic acid, sulfuric acid, or nitric acid. In some further embodiments, said solution may comprise an organic acid.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as, for example, formic acid, acetic acid, propionic acid, butyric acid, isobutyric acid, or the like. In some embodiments, the chain terminating moiety comprises a 3′-acetal moiety which can be cleaved with a palladium deblocking reagent (e.g., Pd(0)).

In some embodiments, in any of the methods for sequencing nucleic acid molecules described herein, the nucleotide comprises a chain terminating moiety which is selected from a group consisting of 3′-deoxy nucleotides, 2′, 3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety or derivatives thereof.

In some embodiments, in any of the methods for sequencing nucleic acid molecules described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

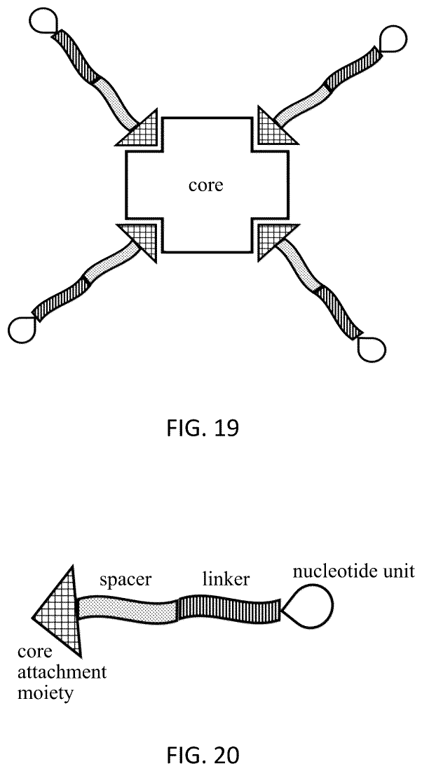

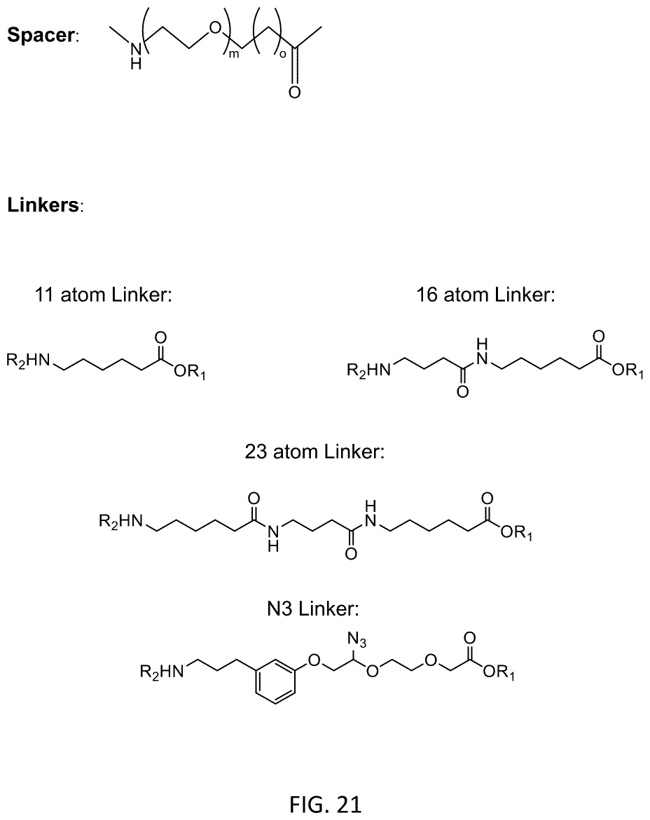

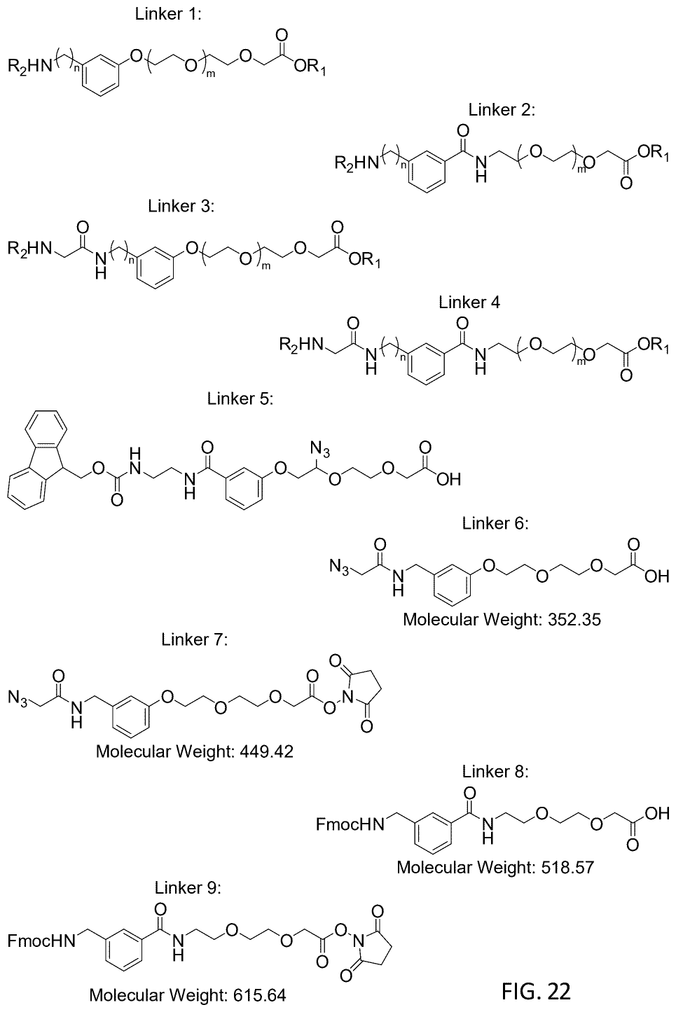

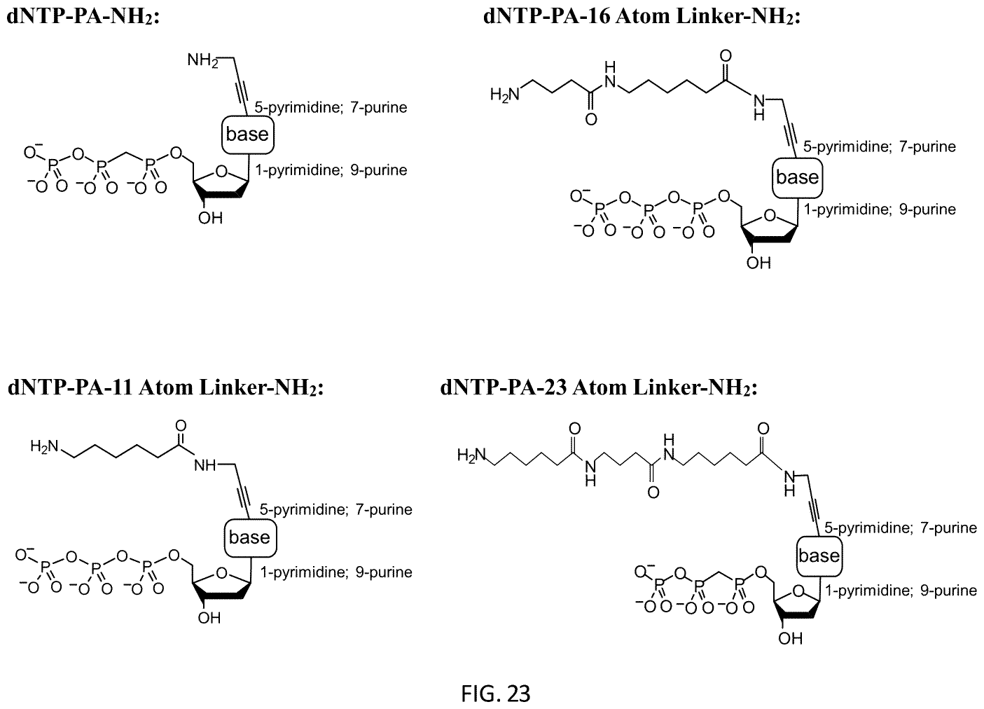

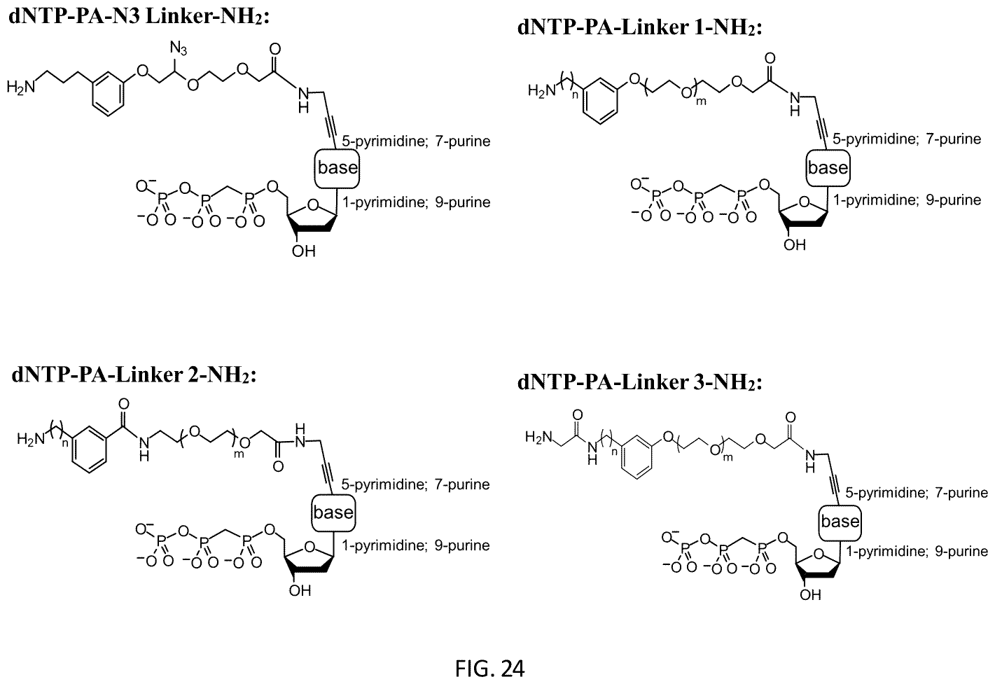

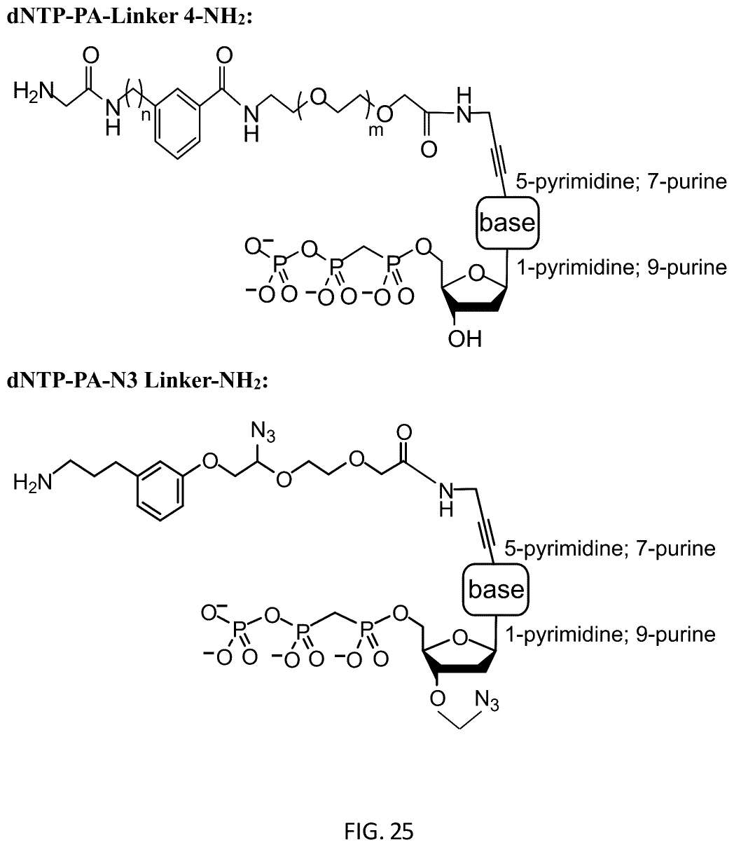

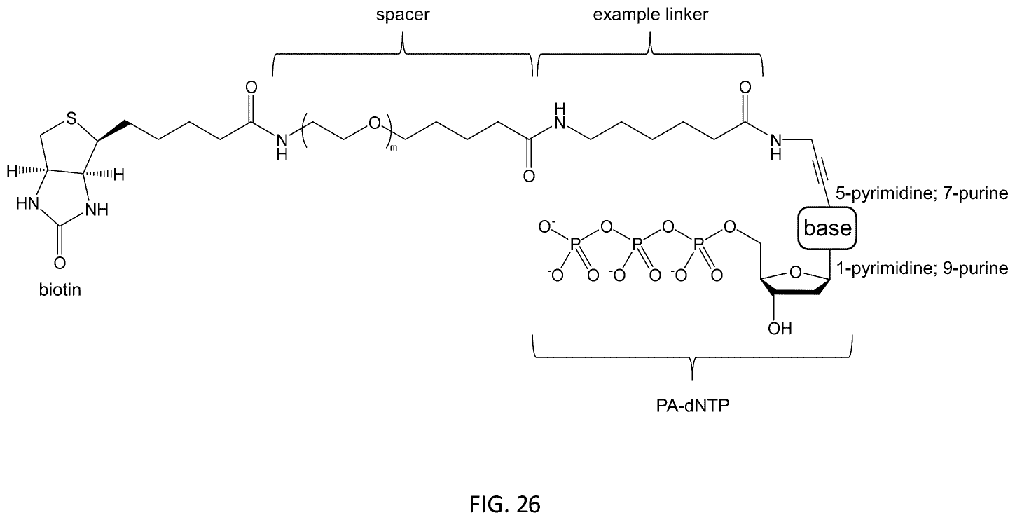

The present disclosure provides methods for sequencing nucleic acid molecules, where any of the sequencing methods described herein employ at least one multivalent molecule. In some embodiments, the multivalent molecule comprises a plurality of nucleotide arms attached to a core and having any configuration including a starburst, helter skelter, or bottle brush configuration (e.g., FIG. 16). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 subunits. In some embodiments, the linker also includes an aromatic moiety. An exemplary nucleotide arm is shown in FIG. 20. Exemplary multivalent molecules are shown in FIGS. 16-19 An exemplary spacer is shown in FIG. 18 (top) and exemplary linkers are shown in FIG. 21 (bottom) and FIG. 19. Exemplary nucleotides attached to a linker are shown in FIGS. 23-26. An exemplary biotinylated nucleotide arm is shown in FIG. 26.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′, 3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the methods for sequencing nucleic acid molecules described herein can include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

A compaction oligonucleotide comprises a single-stranded linear oligonucleotide having a 5′ region that can hybridize to a first portion of a concatemer molecule and the compaction oligonucleotide having a 3′ region that can hybridize to a second portion of the concatemer molecule (e.g., the same concatemer molecule). In some embodiments, hybridization of the compaction oligonucleotides to individual concatemer molecules causes the concatemer molecule to collapse or fold into a DNA nanoball which is more compact in shape and size compared to a non-collapsed DNA molecule. A spot image of a DNA nanoball can be represented as a Gaussian spot and the size can be measured as a full width half maximum (FWHM). A smaller spot size as indicated by a smaller FWHM typically correlates with an improved image of the spot. In some embodiments, the FWHM of a DNA nanoball spot can be about 10 um or smaller. The DNA nanoball can be a compact nucleic acid structure having a full width half maximum (FWHM) that is smaller compared to a concatemer that is not collapsed/folded into a DNA nanoball.

In some embodiments, compaction oligonucleotides comprise a single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprises a 5′ region and a 3′ region, and optionally an intervening region between 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer molecule. The 3′ region of the compaction oligonucleotides can be wholly complementary or partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule. The 5′ and 3′ regions of the compaction oligonucleotide can hybridize to the concatemer to pull together distal portions of the concatemer causing compaction of the concatemer to form a DNA nanoball.

The 5′ region of the compaction oligonucleotide can have the same sequence as 3′ region. The 5′ region of the compaction oligonucleotide can have a sequence that is different from 3′ region. The 3′ region of the compaction oligonucleotide can have a sequence that is a reverse sequence of 5′ region.

In some embodiments sequence data may be derived through nanopore sequencing, which comprises sequencing of a nucleic acid by translocating said nucleic acid across a membrane, such as through a pore, and wherein sequence reads or base calls are made by measuring one or more signals during the translocation event, such as impedance, current, voltage, or capacitance. In some embodiments, the identity of a nucleotide may be determined by distinctive electrical signatures, such as the timing, duration, extent, or lineshape of a current block, impedance change, voltage change, or capacitance change. Sequencing of nucleic acids by translocation across a membrane and/or through a pore does not foreclose alternative detection methods, such as optical, chemical, biochemical, fluorescent, luminescent, magnetic, electromagnetic, acoustic, or electroacoustic detection.

### Supports and Low Non-Specific Coatings

In some embodiments, the flow cell 1120 in FIG. 1 can include a support, e.g., a solid support as disclosed herein. The present disclosure provides pairwise sequencing compositions and methods which employ a support comprising a plurality of oligonucleotide surface primers immobilized thereon. In some embodiments, the support is passivated with a low non-specific binding coating. The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

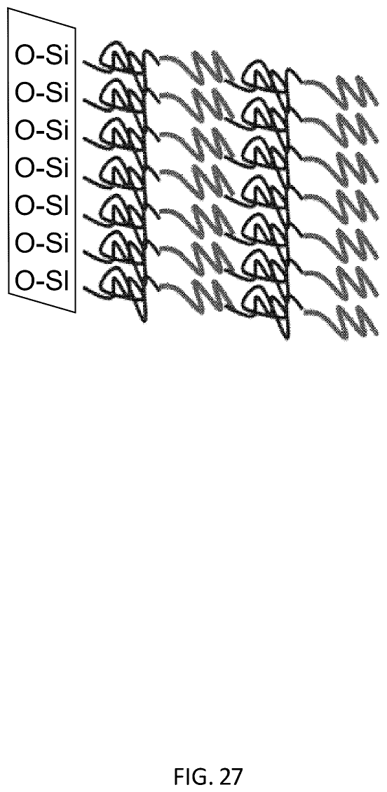

The low non-specific binding coating comprises one layer or multiple layers (FIG. 27). In some embodiments, the plurality of surface primers are immobilized to the low non-specific binding coating. In some embodiments, at least one surface primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein. Amplification levels suitable for detection are achieved in no more than 30, 40, 50, 60, 70, 80, 90, 100, 110, 120, 130, 140, 150, or more than 150 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl)triethoxysilane (APTES), any of a variety of PEG-silanes (e.g., comprising molecular weights of 1K, 2K, 5K, 10 K, 20 K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, surface primers with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a desired surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl) acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N. Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 900 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 900,000/μm², while also comprising at least a second region having a substantially different local density.

In some embodiments, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (Signal)-B(interstial) in the FOV) yields a discernable feature that can be classified. In some embodiments, the intrastitial background (B (intrastitial)) can contribute a confounding fluorescence signal that is not specific to the target of interest, but is present in the same ROI thus making it far more difficult to average and subtract.

Nucleic acid amplification on the low-binding coated supports described herein may decrease the B (interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization and/or amplification reaction formulations, may lead to improvements in CNR by a factor of 2, 5, 10, 100, 250, 900 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, i.e., the limitations of the measurement system. For example, “about,” “approximately,” or “substantially” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” “substantially” should be assumed to be within an acceptable error range for that particular value or composition. Also, where ranges and/or subranges of values are provided, the ranges and/or subranges can include the endpoints of the ranges and/or subranges.

The term “polony” used herein refers to a nucleic acid library molecule can be clonally amplified in-solution or on-support to generate an amplicon that can serve as a template molecule for sequencing. In some embodiments, a linear library molecule can be circularized to generate a circularized library molecule, and the circularized library molecule can be clonally amplified in-solution or on-support to generate a concatemer. In some embodiments, the concatemer can serve as a nucleic acid template molecule which can be sequenced. The concatemer is sometimes referred to as a polony. In some embodiments, a polony includes nucleotide strands. Although “polony” is used embodiments herein for describing the application of the methods disclosed herein. Such methods may also be useful in other applications that works with clusters that may be generated using various sequencing reactions in NGS.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, comprises any enzyme that can catalyze polymerization of nucleotides (including analogs thereof) into a nucleic acid strand. Typically but not necessarily such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, a nucleotide unit of a multivalent molecule or a free (e.g., unconjugated) nucleotide. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “primer” and related terms used herein refers to an oligonucleotide, either natural or synthetic, that is capable of hybridizing with a DNA and/or RNA polynucleotide template to form a duplex molecule. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-mediated primer extension reaction. Alternatively, 3′ end of the primer can lack a 3′ OH moiety, or can include a terminal 3′ blocking group that inhibits nucleotide polymerization in a polymerase-mediated reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for generating a complementary nucleic acid strand. The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog. The template nucleic acid can be linear, circular, or other forms. The template nucleic acids can include an insert region having an insert sequence which is also known as a sequence of interest. The template nucleic acids can also include at least one adaptor sequence. The template nucleic acid can be a concatemer having two or tandem copies of a sequence of interest and at least one adaptor sequence. The insert region can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid can be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7 mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′, 3′-dideoxyribosyl; 2′, 3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6Iohexyl)-2-((1E,3E)-5-((E)-1-(6-((2,5-dioxopyrrolidin-1-yl)oxy)-6-oxohexyl)-3,3-dimethyl-5-indolin-2-ylidene) penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene) penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene) hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene) hepta-1,3,5-trien-1-yl]-3H-indolium-5-sulfon'the), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure can include but are not limited to: nucleotide transient-binding; nucleotide incorporation; de-blocking; washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The term “adaptor” and related terms refers to oligonucleotides that can be operably linked (appended) to a target polynucleotide, where the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, or a capture primer (e.g., soluble or immobilized capture primers). Adaptors can include a random sequence or degenerate sequence. Adaptors can include at least one inosine residue. Adaptors can include at least one phosphorothioate, phosphorothiolate and/or phosphoramidate linkage. Adaptors can include a barcode sequence which can be used to distinguish polynucleotides (e.g., insert sequences) from different sample sources in a multiplex assay. Adaptors can include a unique identification sequence (e.g., unique molecular index, UMI; or a unique molecular tag) that can be used to uniquely identify a nucleic acid molecule to which the adaptor is appended. In some embodiments, a unique identification sequence can be used to increase error correction and accuracy, reduce the rate of false-positive variant calls and/or increase sensitivity of variant detection. Adaptors can include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence can be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support is coated with one or more compounds to produce a passivated layer on the support. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

The present disclosure provides a plurality (e.g., two or more) of nucleic acid templates immobilized to a support. In some embodiments, the immobilized plurality of nucleic acid templates have the same sequence or have different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support. In some embodiments, the support comprises a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 10²-10¹⁵ sites per mm², or more, to form a nucleic acid template array. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid templates to form a nucleic acid template array. In some embodiments, the nucleic acid templates that are immobilized at a plurality of pre-determined sites by hybridization to immobilized surface capture primers, or the nucleic acid templates are covalently attached to the surface capture primers. In some embodiments, the nucleic acid templates that are immobilized at a plurality of pre-determined sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of pre-determined sites. In some embodiments, individual immobilized nucleic acid template molecules comprise one copy of a target sequence of interest, or comprise concatemers having two or more tandem copies of a target sequence of interest.

In some embodiments, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) are immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates are covalently attached to the surface capture primer. In some embodiments, the nucleic acid templates that are immobilized at a plurality of randomly located sites, for example immobilized at 10²-10¹⁵ sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of randomly located sites. In some embodiments, individual immobilized nucleic acid template molecules comprise one copy of a target sequence of interest, or comprise concatemers having two or more tandem copies of a target sequence of interest.

In some embodiments, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations and/or buffers and the like) onto the support so that the plurality of immobilized nucleic acid template molecules on the support can be reacted with the reagents in a massively parallel manner. In some embodiments, the fluid communication of the plurality of immobilized nucleic acid template molecules can be used to conduct nucleotide binding assays and/or conduct nucleotide polymerization reactions (e.g., primer extension or sequencing) on the plurality of immobilized nucleic acid template molecules, and to conduct detection and imaging for massively parallel sequencing. In some embodiments, the term “immobilized” and related terms refer to nucleic acid molecules or enzymes (e.g., polymerases) that are attached to the support at pre-determined or random locations, where the nucleic acid molecules or enzymes are attached directly to a support through covalent bond or non-covalent interaction, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 900, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and it variants refers to a nucleic acid template molecule that has been subjected to one or more amplification reactions either in-solution or on-support. In the case of in-solution amplified template molecules, the resulting amplicons are distributed onto the support. Prior to amplification, the template molecule comprises a sequence of interest and at least one universal adaptor sequence. In some embodiments, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

As used herein, the term “sequencing” and its variants comprise obtaining sequence information from a nucleic acid strand, typically by determining the identity of at least some nucleotides (including their nucleobase components) within the nucleic acid template molecule. While in some embodiments, “sequencing” a given region of a nucleic acid molecule includes identifying each and every nucleotide within the region that is sequenced, in some embodiments “sequencing” comprises methods whereby the identity of only some of the nucleotides in the region is determined, while the identity of some nucleotides remains undetermined or incorrectly determined. Any suitable method of sequencing may be used. In an exemplary embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299 (5607): 682-686; Eid, et al., 2009 Science 323 (5910): 133-138; U.S. Pat. Nos. 7,170,050; 7,302,146; and 7,405,281). An example of sequence-by-hybridization includes SOLID sequencing (e.g., from Life Technologies; WO 2006/084132). An example of sequence-by-binding includes Omniome sequencing (e.g., U.S. Pat. No. 10,246,744).

As used herein, the term “strand displacing” refers to the ability of a polymerase to locally separate strands of double-stranded nucleic acids and synthesize a new strand in a template-based manner. Strand displacing polymerases displace a complementary strand from a template strand and catalyze new strand synthesis. Strand displacing polymerases include mesophilic and thermophilic polymerases. Strand displacing polymerases include wild type enzymes, and variants including exonuclease minus mutants, mutant versions, chimeric enzymes and truncated enzymes. Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi from Expedeon), or variant EquiPhi29 DNA polymerase (e.g., from Thermo Fisher Scientific), or chimeric QualiPhi DNA polymerase (e.g., from 4basebio).

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

When used in reference to nucleic acids, the terms “amplify”, “amplifying”, “amplification”, and other related terms include producing multiple copies of an original polynucleotide template molecule, where the copies comprise a sequence that is complementary to the template sequence, or the copies comprise a sequence that is the same as the template sequence. In some embodiments, the copies comprise a sequence that is substantially identical to a template sequence, or is substantially identical to a sequence that is complementary to the template sequence.

The term “support” as used herein refers to a substrate that is designed for deposition of biological molecules or biological samples for assays and/or analyses. Examples of biological molecules to be deposited onto a support include nucleic acids (e.g., DNA, RNA), polypeptides, saccharides, lipids, a single cell or multiple cells. Examples of biological samples include but are not limited to saliva, phlegm, mucus, blood, plasma, serum, urine, stool, sweat, tears and fluids from tissues or organs.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The support can have a plurality (e.g., two or more) of nucleic acid templates immobilized thereon. The plurality of immobilized nucleic acid templates have the same sequence or have different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support.

The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid templates to form a nucleic acid template array. In some embodiments, the nucleic acid templates that are immobilized at a plurality of pre-determined sites by hybridization to immobilized surface capture primers, or the nucleic acid templates are covalently attached to the surface capture primer. In some embodiments, the nucleic acid templates that are immobilized at a plurality of pre-determined sites, for example immobilized at 10²-10¹⁵ sites or more. In some embodiments, the immobilized nucleic acid templates are clonally-amplified to generate immobilized nucleic acid clusters at the plurality of pre-determined sites. In some embodiments, individual immobilized nucleic acid clusters comprise linear clusters, or comprise single-stranded or double-stranded concatemers.

In some embodiments, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) are immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates are covalently attached to the surface capture primer. In some embodiments, the nucleic acid templates that are immobilized at a plurality of randomly located sites, for example immobilized at 10²-10¹⁵ sites or more. In some embodiments, the immobilized nucleic acid templates are clonally-amplified to generate immobilized nucleic acid clusters at the plurality of randomly located sites. In some embodiments, individual immobilized nucleic acid clusters comprise linear clusters, or comprise single-stranded or double-stranded concatemers.

In some embodiment, the plurality of immobilized surface capture primers on the support are in fluid communication with each other to permit flowing a solution of reagents (e.g., nucleic acid template molecules, soluble primers, enzymes, nucleotides, divalent cations, buffers, and the like) onto the support so that the plurality of immobilized surface capture primers on the support can be essentially simultaneously reacted with the reagents in a massively parallel manner. In some embodiments, the fluid communication of the plurality of immobilized surface capture primers can be used to conduct nucleic acid amplification reactions (e.g., RCA, MDA, PCR and bridge amplification) essentially simultaneously on the plurality of immobilized surface capture primers.

In some embodiment, the plurality of immobilized nucleic acid clusters on the support are in fluid communication with each other to permit flowing a solution of reagents (e.g., enzymes, nucleotides, divalent cations, and the like) onto the support so that the plurality of immobilized nucleic acid clusters on the support can be essentially simultaneously reacted with the reagents in a massively parallel manner. In some embodiments, the fluid communication of the plurality of immobilized nucleic acid clusters can be used to conduct nucleotide binding assays and/or conduct nucleotide polymerization reactions (e.g., primer extension or sequencing) essentially simultaneously on the plurality of immobilized nucleic acid clusters, and optionally to conduct detection and imaging for massively parallel sequencing.

When used in reference to immobilized enzymes, the term “immobilized” and related terms refer to enzymes (e.g., polymerases) that are attached to a support through covalent bond or non-covalent interaction, or attached to a coating on the support, or buried within a matrix formed by a coating on the support.

When used in reference to immobilized nucleic acids, the term “immobilized” and related terms refer to nucleic acid molecules that are attached to a support through covalent bond or non-covalent interaction, or attached to a coating on the support, or buried within a matrix formed by a coating on the support, where the nucleic acid molecules include surface capture primers, nucleic acid template molecules and extension products of capture primers. Extension products of capture primers includes nucleic acid concatemers (e.g., nucleic acid clusters).

In some embodiments, one or more nucleic acid templates are immobilized on the support, for example immobilized at the sites on the support. In some embodiments, the one or more nucleic acid templates are clonally-amplified. In some embodiments, the one or more nucleic acid templates are clonally-amplified off the support (e.g., in-solution) and then deposited onto the support and immobilized on the support. In some embodiments, the clonal amplification reaction of the one or more nucleic acid templates is conducted on the support resulting in immobilization on the support. In some embodiments, the one or more nucleic acid templates are clonally-amplified (e.g., in solution or on the support) using a nucleic acid amplification reaction, including any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, and/or single-stranded binding (SSB) protein-dependent amplification.

The term “persistence time” and related terms refers to the length of time that a binding complex, which is formed between the target nucleic acid, a polymerase, a conjugated or unconjugated nucleotide, remains stable without any binding component dissociates from the binding complex. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label.

For massively parallel sequencing, the limit of optical resolution impedes the ability to perform highly multiplex sequencing. Batch-specific sequencing enables sequencing a desired subset (e.g., a batch) of the template molecules immobilized to the same flowcell using selected batch-specific sequencing primers to reduce over-crowding signals and images. The use of batch-specific sequencing primers produces optical images that are intense and resolvable. The batch-specific sequencing methods described herein have many uses. For example, the number of spots that are imaged and associated with sequencing can be counted. The counted spots can be used as a measure for target nucleic acid levels in a sample.

The present disclosure provides compositions, apparatus and methods for conducting separate sequencing batches on a support having nucleic acid template molecules immobilized thereon, where the separate sequencing batches can be conducted using any massively parallel sequencing technology. In some embodiments, a plurality of sub-populations of nucleic acid template molecules are immobilized to the support including at least a first and second sub-population. In some embodiments, the first sub-population of template molecules undergo first sequencing reactions (e.g., first batch sequencing) and a region of the support is imaged to detect the first sequencing reactions, wherein the second sub-population of template molecules do not undergo sequencing reactions. In some embodiments, the second sub-population of template molecules undergo second sequencing reactions (e.g., second batch sequencing) and the same region of the support is imaged to detect the second sequencing reactions, wherein the first sub-population of template molecules do not undergo sequencing reactions. Thus, the first and second sub-populations of template molecules undergo batch sequencing.

In some embodiments, the plurality of sub-populations of nucleic acid template molecules are immobilized to the support at a high density where at least some of the immobilized template molecules in the first and second sub-populations comprise nearest neighbor template molecules that touch each other and/or overlap each other when viewed from any angle of the support including above, below or side views of the support.

In some embodiments, the support comprises a plurality of template molecules immobilized at pre-determined positions on the support (e.g., a patterned support). In some embodiments, the support comprises a plurality of template molecules immobilized at random and non-pre-determined positions on the support. In some embodiments, the support comprises a mixture of at least two sub-populations of template molecules immobilized at random and non-pre-determined positions on the support. In some embodiments, the support lacks any contours (e.g., wells, protrusions, and the like) arranged in a pre-determined pattern. In some embodiments, the support lacks contours which include features as sites for attachment of the nucleic acid template molecules. In some embodiments, the support lacks interstitial regions arranged in a pre-determined pattern where the interstial regions are sites designed to have no attached template molecules. In some embodiments, the support lacks features that can be prepared using photo-chemical, photo-lithography, or micron-scale or nano-scale printing.

In some embodiments, individual template molecules in a given sub-population of template molecules comprise a sequence of interest, a batch barcode sequence that corresponds to the sequence of interest, and a batch sequencing primer binding site sequence that corresponds to the sequence of interest. In some embodiments, a pre-determined batch barcode sequence can be linked to a given sequence of interest, thus the pre-determined batch barcode sequence corresponds to a given sequence of interest. In some embodiments, a pre-determined batch sequencing primer binding site sequence can be linked to a given sequence of interest, thus the pre-determined batch sequencing primer binding site sequence corresponds to a given sequence of interest. In some embodiments, template molecules within a given sub-population have the same or different sequence of interest. In some embodiments, template molecules within a given sub-population have the same target barcode sequence. In some embodiments, template molecules within a given sub-population have the same sequencing primer binding site sequence. Thus, the different sub-populations of template molecules can undergo batch sequencing using a batch-specific sequencing primer.

In some embodiments, the sequence of interest need not undergo sequencing. Instead, the target barcode can be sequenced by conducting a small number of sequencing cycles to reveal the target barcode which corresponds to its sequence of interest.

In some embodiments, individual template molecules in a given sub-population of template molecules further comprise a sample index sequence that can be used to distinguish sequences of interest obtained from different sample sources in a multiplex assay. In some embodiments, template molecules within a given sub-population have the same or different sample index sequences.

In some embodiments, the sequence of interest need not undergo sequencing. Instead, the target barcode and the sample index can be sequenced by conducting a small number of sequencing cycles to reveal the target barcode which corresponds to its sequence of interest and to reveal the sample index which corresponds to the sample source of the sequence of interest. In some embodiments, the template molecules lack a sample index and the target barcode can serve as a sample index.

In some embodiments, the same portion of individual template molecules can be re-sequenced (e.g., reiterative sequencing) from the same start position to generate overlapping sequencing reads that can be aligned to a reference sequence. For example, the same portion of individual template molecules can be sequenced at least two, three, four, five, or up to 50 times, or up to 100 times, or more than 100 times. The start sequencing site can be any location of the template molecule and is dictated by the sequencing primers which are designed to anneal to a selected position within the template molecule. In some embodiments, the target barcodes (or the target barcodes and sample indexes) can be reiteratively sequenced by repeatedly conducting a short number of sequencing cycles of the target barcode region (or the target barcode and sample index regions) of a given template molecule. The reiterative sequencing reads increase the redundancy of sequencing information for individual bases in the template molecule. Reiteratively sequencing one strand of the template molecule provides enough base coverage so that pairwise sequencing of the complementary strand is not necessary.

In some embodiments, after sequencing the first and/or second sub-populations of template molecules, the support can be re-seeded at least once with additional sub-populations of nucleic acid template molecules (e.g., a third sub-population) which can undergo additional batch sequencing. In some embodiments, an ongoing batch sequencing run can be stopped prior to completion (e.g., interrupted) to permit re-seeding the support with an additional sub-population of immobilized template molecules (e.g., third sub-population) and then the interrupted batch sequencing can be resumed. Thus, the support can be re-seeded any time and/or before a previous sequencing batch is completed.

In some embodiments, the support comprises a plurality of template molecules immobilized at an initial low density where most of the nearest neighbor template molecules do not touch each other and/or do not overlap each other. In some embodiments, the initial low density support comprises a plurality of immobilized template molecules having interstitial space between the immobilized template molecules.

In some embodiments, the same support can undergo a first re-seeding with additional template molecules so that the first re-seeded density has some nearest template molecules (e.g., 10-30% of the first immobilized re-seeded template molecules) that touch each other and/or overlap each other. In some embodiments, the resulting first re-seeded support comprises a plurality of immobilized template molecules having a reduced number of interstitial space (and/or having a reduced size of interstitial space) between the immobilized template molecules compared to the initial low density support.

In some embodiments, the same support can undergo a second re-seeding with additional template molecules so that the second re-seeded density has an increase in nearest neighbor template molecules (e.g., 25-50% or more of the first immobilized re-seeded template molecules) that touch each other and/or overlap each other. In some embodiments, the resulting second re-seeded support comprises a plurality of immobilized template molecules having a further reduced number of interstitial space (and/or having a further reduced size of interstitial space) between the immobilized template molecules compared to the first re-seeded density support. In some embodiments, the support can undergo multiple re-seeding workflows to generate increasing nearest neighbor template molecules that touch each other and/or overlap each other.

In some embodiments, individual template molecules comprise nucleic acid concatemer molecules. In some embodiments, a concatemer molecule can be generated by conducting rolling circle amplification of a circularized nucleic acid library molecule. In some embodiments, a concatemer molecule comprises a single-stranded nucleic acid strand carrying numerous tandem copies of a polynucleotide unit, where each polynucleotide unit comprises a sequence of interest and at least one batch sequencing primer binding site. In some embodiments, each polynucleotide unit further comprises at least one batch barcode sequence. In some embodiments, each polynucleotide unit further comprises at least one sample index sequence. Individual polynucleotide units can bind a sequencing primer, a sequencing polymerase and a detectably-labeled nucleotide reagent (e.g., detectably labeled multivalent molecules or nucleotide analogs), to form a detectable sequencing complex. In some embodiments, individual concatemers can collapse into a compact DNA nanoball, where individual nanoballs carry numerous tandem copies of a polynucleotide unit along their lengths. During batch sequencing, individual nanoballs carry numerous detectable sequencing complexes. Thus, the compact nature of the nanoballs increases the local concentration of detectably-labeled nucleotide reagents that are used during batch sequencing which increases the signal intensity emitted from a nanoball to give a discrete detectable signal which can be imaged as a fluorescent spot. In some embodiments, a spot corresponds to a concatemer and each concatemer corresponds to a sequence of interest. Multiple spots can be detected and imaged simultaneously on a support having high density concatemers immobilized thereon.

The methods described herein employ batch sequencing on high density immobilized template molecules which offers the advantage of maximizing space on a support (e.g., flowcell). Furthermore, the same seeded support can be re-used by re-seeding the support with additional immobilized template molecules and conducting additional sequencing reactions on the re-seeded template molecules.

Batch sequencing can be conducted using template molecules arranged in a pre-determined manner on the support (e.g., a patterned support). Alternatively, batch sequencing can be conducted using template molecules arranged in a random manner on the support which obviates the need to fabricate a support having organized and pre-determined features for attaching template molecules (e.g., fabrication via lithography is not needed).

By conducting short sequencing reads of the target barcode regions of the template molecules, batch sequencing also significantly reduces sequencing run times, reagent use, and reagent costs.

When short sequencing reads of the target barcode regions are conducted in a reiterative manner, it is not necessary to assemble the sequencing reads or to obtain a full length sequence of the sequence of interest which reduces the need for long assembly computations. Also, the redundant sequencing information obtained from the short sequencing reads obviates the need to sequence the complementary strand of the template molecules, thus pairwise sequencing is not necessary.

Batch sequencing also offers the flexibility of re-seeding the support any time between sequencing different batches, or an ongoing sequencing batch can be interrupted to permit re-seeding then the ongoing batch sequencing can be resumed. The ability to re-seed the support any time increases throughput and efficiency.

Conducting batch sequencing with immobilized concatemer template molecule offers advantages over one-copy template molecules (e.g., one-copy template molecule generated via bridge amplification). For example concatemers carry multiple sequencing primer binding sites along the same concatemer molecule. The multiple sequencing primer binding sites can be used to generate multiple sequencing reads for increased sequencing depth. Together, reiteratively sequencing one strand of the concatemer templates increases sequencing base coverage and sequencing depth compared to sequencing a one-copy template molecule.

Batch sequencing has many uses including but not limited to detecting specific nucleic acids of interest, mutant nucleic acid sequences, splice variants, and their abundance levels thereof.

### Batch Sequencing

The present disclosure provides methods for sequencing comprising step (a): providing a support comprising a plurality of nucleic acid template molecules immobilized to the support, wherein the plurality of nucleic acid template molecules comprises a plurality of sub-populations of template molecules including at least a first and a second sub-population of template molecules, wherein the first sub-population of template molecules comprises a first batch sequencing primer binding site and at least one first sequence-of-interest and, wherein the second sub-population of template molecules comprises a second batch sequencing primer binding site and at least one second sequence-of-interest. In some embodiments, template molecules within the first sub-population have the same first batch sequencing primer binding site, and have the same sequence of interest or different sequences of interest. In some embodiments, the sequence of the first batch sequencing primer binding site sequence corresponds to the first sequence of interest, or the first batch sequencing primer binding site sequence corresponds to one of the first sequences of interest in the first sub-population. In some embodiments, a pre-determined first batch sequencing primer binding site sequence can be linked to a given sequence of interest in the first sub-population (or can be linked to different sequences of interest in a first sub-population), thus the pre-determined first batch sequencing primer binding site sequence corresponds to a given sequence of interest in the first sub-population.

In some embodiments, the sequences of interest in the first sub-population are about 50-250 bases in length, or about 250-500 bases in length, or about 500-800 bases in length, or about 800-1200 bases in length, or up to 2000 bases in length.

In some embodiments, template molecules within the second sub-population have the same second batch sequencing primer binding site, and have the same sequence of interest or different sequences of interest. In some embodiments, the sequence of the second batch sequencing primer binding site sequence corresponds to the second sequence of interest, or the sequence of the second batch sequencing primer binding site sequence corresponds to one of the second sequences of interest in the second sub-population. In some embodiments, a pre-determined second batch sequencing primer binding site sequence can be linked to a given sequence of interest in the second sub-population (or can be linked to different sequences of interest in a second sub-population), thus the pre-determined second batch sequencing primer binding site sequence corresponds to a given sequence of interest in the second sub-population.

In some embodiments, the sequences of interest in the second sub-population are about 50-250 bases in length, or about 250-500 bases in length, or about 500-800 bases in length, or about 800-1200 bases in length, or up to 2000 bases in length.

In some embodiments, the first and second batch sequencing primer binding sites have different sequences.

In some embodiments, the plurality of nucleic acid template molecules can be immobilize to the support at random and non-pre-determined positions on the support, or at pre-determined positions on the support (e.g., a patterned support).

In some embodiments, in the methods for sequencing of step (a), the support comprises a plurality of nucleic acid template molecules immobilized thereon at a density of about 10²-10¹⁵ template molecules per mm², wherein the immobilized template molecules comprise a mixture of at least two sub-populations of template molecules including at least a first and second sub-population of template molecules. In some embodiments, the plurality of sub-populations of template molecules are immobilized to the support at a high density where at least some of the immobilized template molecules in the first and second sub-populations comprise nearest neighbor template molecules that touch each other and/or overlap each other when viewed from any angle of the support including above, below or side views of the support. In some embodiments, the support comprises up to 500 million concatemers immobilized thereon, or up to 1 billion concatemers immobilized thereon, up to 2 billion concatemers immobilized thereon, up to 5 billion concatemers immobilized thereon, or up to 10 billion concatemers immobilized thereon, or up to 20 billion concatemers immobilized thereon.

In some embodiments, in the methods for sequencing of step (a), the support comprises features on the support that are located in a random and non-prep-determined manner, where the features are sites for attachment of the template molecules.

In some embodiments, the support is passivated with at least one polymer coating layer comprising a plurality of surface capture primers covalently tethered to the at least one polymer layer.

In some embodiments, the plurality of surface capture primers comprise a plurality of sub-populations of surface capture primers including at least a first and second sub-population of surface capture primers. In some embodiments, the surface capture primers in the at least first and second sub-population have different sequences. In some embodiments, the surface capture primers in the at least first and second sub-population can hybridize/capture different circularized library molecules carrying different surface capture primer binding site sequences.

In some embodiments, the plurality of surface capture primers are randomly distributed throughout and embedded within the at least one polymer layer.

In some embodiments, the support lacks any contours (e.g., wells, protrusions, and the like) arranged in a pre-determined pattern where the contours have features that are sites for attachment of the nucleic acid template molecules. In some embodiments, the support lacks interstitial regions arranged in a pre-determined pattern where the interstial regions are sites designed to have no attached template molecules.

In some embodiments, in the methods for sequencing of step (a), the support lacks partitions/barriers that would create separate regions of the support. Thus, the immobilized template molecules are in fluid communication with each other in a massively parallel manner with no barriers to physically separate different batches of template molecules.

In some embodiments, in the methods for sequencing of step (a), individual template molecules in the first sub-population further comprise a first batch barcode sequence which corresponds to the first sequence of interest, or the first batch barcode sequence corresponds to one of the first sequences of interest in the first sub-population. In some embodiments, a pre-determined first batch barcode sequence can be linked to a given sequence of interest in the first sub-population (or can be linked to different sequences of interest in a first sub-population), thus the pre-determined first batch barcode sequence corresponds to a given sequence of interest in the first sub-population.

In some embodiments, individual template molecules in the second sub-population further comprise a second batch barcode sequence which corresponds to the second sequence of interest, or the second batch barcode sequence corresponds to one of the second sequences of interest in the second sub-population. In some embodiments, a pre-determined second batch barcode sequence can be linked to a given sequence of interest in the second sub-population (or can be linked to different sequences of interest in a second sub-population), thus the pre-determined second batch barcode sequence corresponds to a given sequence of interest in the second sub-population.

In some embodiments, in the methods for sequencing of step (a), individual template molecules in the first sub-population further comprises at least one sample index sequence that can be used in a multiplex assay to distinguish the first sequences of interest obtained from different sample sources. In some embodiments, individual template molecules in the second sub-population further comprises at least one sample index sequence that can be used in a multiplex assay to distinguish the second sequences of interest obtained from different sample sources.

In some embodiments, in the methods for sequencing of step (a), the plurality of template molecules comprise nucleic acid concatemer template molecules, including first and second sub-populations of concatemer template molecules. In some embodiments, the concatemer template molecules can be generated by conducting rolling circle amplification using circularized library molecules and amplification primers. In some embodiments, a concatemer molecule comprises numerous tandem copies of a polynucleotide unit, where each polynucleotide unit comprises a sequence of interest and at least one sequencing primer binding site. In some embodiments, concatemer template molecules immobilized to a support can be generated using circularized library molecules and conducting rolling circle amplification. In some embodiments, the circularized library molecules can be generated using padlock probes, single-stranded splint strands, or double-stranded adaptors. In some embodiments, the circularized library molecules comprise a mixture of any combination of circularized padlock probes, linear library molecules circularized using single-stranded splint strands, and/or linear library molecules circularized using double-stranded adaptors. Methods for generating circularized library molecules are described herein.

In some embodiments, individual concatemers in the first sub-population comprise a plurality of tandem polynucleotide units, where each polynucleotide unit comprises a first sequence of interest and a first batch sequencing primer binding site sequence which corresponds to the first sequence of interest. In some embodiments, the polynucleotide unit further comprises a first batch barcode sequence which corresponds to the first sequence of interest. In some embodiments, the polynucleotide unit further comprises at least one sample index sequence that can be used in a multiplex assay to distinguish sequences of interest obtained from different sample sources. In some embodiments, concatemer molecules in the first sub-population have the same first batch sequencing primer binding site, and have the same sequence of interest or different sequences of interest.

In some embodiments, individual concatemers in the second sub-population comprise a plurality of tandem polynucleotide units, where each polynucleotide unit comprises a second sequence of interest and a second batch sequencing primer binding site sequence which corresponds to the second sequence of interest. In some embodiments, the polynucleotide unit further comprises a second batch barcode sequence which corresponds to the second sequence of interest. In some embodiments, the polynucleotide unit further comprises at least one sample index sequence that can be used in a multiplex assay to distinguish sequences of interest obtained from different sample sources. In some embodiments, concatemer molecules in the second sub-population have the same second batch sequencing primer binding site, and have the same sequence of interest or different sequences of interest.

In some embodiments, the methods for sequencing further comprise step (b): sequencing the first sub-population of template molecules using a plurality of first batch sequencing primers thereby generating a plurality of first batch sequencing read products. In some embodiments, the sequencing of step (b) comprises imaging a region of the support to detect the sequencing reactions of the first sub-population of template molecules.

In some embodiments, the methods for sequencing further comprises step (b1): conducting short read sequencing by performing up to 1000 sequencing cycles of the first sub-population of template molecules to generate a plurality of first batch sequencing read products that comprise up to 1000 bases in length. In some embodiments, the methods for sequencing further comprises step (b1): conducting short read sequencing by performing no more than 150 sequencing cycles of the first sub-population of template molecules to generate a plurality of first batch sequencing read products that comprise no more than 150 bases in length. In some embodiments, the first batch sequencing read products comprise the first batch barcode sequence. In some embodiments, the first batch sequencing read products comprise the first batch barcode sequence and the sample index sequence. In some embodiments, the first batch sequencing read products comprise the first batch barcode sequence and at least a portion of the first sequence of interest. In some embodiments, the first batch sequencing read products comprise the first batch barcode sequence, the sample index sequence, and at least a portion of the first sequence of interest. In some embodiments, the short read sequencing comprises hybridizing sequencing primers to sequencing primer binding sites on concatemer template molecules and conducting up to 1000 cycles of polymerase-catalyzed sequencing reactions using nucleotide reagents.

In some embodiments, the methods for sequencing further comprises step (b2): stopping/blocking the short read sequencing of step (b1). In some embodiments, the stopping/blocking comprises incorporating a chain terminating nucleotide to 3′ terminal end of the first batch sequencing read products to inhibit further sequencing reactions. Exemplary chain terminating nucleotides include dideoxynucleotide or a nucleotide having a 2′ or 3′ chain terminating moiety.

In some embodiments, the methods for sequencing further comprise step (b3): removing the plurality of first batch sequencing read products from the template molecules of the first sub-population, and retaining the template molecules of the first sub-population.

In some embodiments, the methods for sequencing further comprise step (b4): reiteratively sequencing the template molecules of the first sub-population by repeating steps (b1)-(b3) at least once. In some embodiments, the reiterative sequencing can be conducted 1-10 times, or 10-25 times, or 25-50 times or more. For example, the reiterative sequencing can be conducted up to 100 times.

In some embodiments, the sequences of all of the first batch sequencing read products can be determined and aligned with a first reference sequence to confirm the presence of the first sequence of interest. The first reference sequence can be the first batch barcode and/or the first sequence of interest.

In some embodiments, hybridizing the sequencing primers to the concatemer template molecules of step (b1) can be conducted with a hybridization reagent comprising an SSC buffer (e.g., 2× saline-sodium citrate) buffer with formamide (e.g., 10-20% formamide).

In some embodiments, in step (b3) the plurality of plurality of first batch sequencing read products can be removed from the template molecules and the plurality of template molecules can be retained using a de-hybridization reagent comprising an SSC buffer (e.g., saline-sodium citrate) buffer, with or without formamide, at a temperature that promotes nucleic acid denaturation.

In some embodiments, the methods for sequencing further comprise step (c): sequencing the second sub-population of template molecules using a plurality of second batch sequencing primers thereby generating a plurality of second batch sequencing read products and imaging the same region of the support to detect the sequencing reactions of the second sub-population of template molecules.

In some embodiments, the sequencing reactions of the first sub-population of template molecules is stopped before initiating the sequencing reactions of the second sub-population of template molecules.

In some embodiments, the methods for sequencing further comprise step (c1): conducting short read sequencing by performing up to 1000 sequencing cycles of the second sub-population of template molecules to generate a plurality of second batch sequencing read products that comprise up to 1000 bases in length. In some embodiments, the methods for sequencing further comprise step (c1): conducting short read sequencing by performing no more than 150 sequencing cycles of the second sub-population of template molecules to generate a plurality of second batch sequencing read products that comprise no more than 150 bases in length. In some embodiments, the second batch sequencing read products comprise the second batch barcode sequence. In some embodiments, the second batch sequencing read products comprise the second batch barcode sequence and the sample index sequence. In some embodiments, the second batch sequencing read products comprise the second batch barcode sequence and at least a portion of the second sequence of interest. In some embodiments, the second batch sequencing read products comprise the second batch barcode sequence, the sample index sequence, and at least a portion of the second sequence of interest. In some embodiments, the short read sequencing comprises hybridizing sequencing primers to sequencing primer binding sites on concatemer template molecules and conducting up to 1000 cycles of polymerase-catalyzed sequencing reactions using nucleotide reagents.

In some embodiments, the methods for sequencing further comprise step (c2): stopping/blocking the short read sequencing of step (c1). In some embodiments, the stopping/blocking comprises incorporating a chain terminating nucleotide to 3′ terminal end of the first batch sequencing read products to inhibit further sequencing reactions. Exemplary chain terminating nucleotides include dideoxynucleotide or a nucleotide having a 2′ or 3′ chain terminating moiety.

In some embodiments, the methods for sequencing further comprise step (c3): removing the plurality of second batch sequencing read products from the template molecules of the second sub-population, and retaining the template molecules of the second sub-population.

In some embodiments, the methods for sequencing further comprise step (c4): reiteratively sequencing the template molecules of the second sub-population by repeating steps (c1)-(c3) at least once. In some embodiments, the reiterative sequencing can be conducted 1-10 times, or 10-25 times, or 25-50 times or more.

In some embodiments, the sequences of all of the second batch sequencing read products can be determined and aligned with a second reference sequence to confirm the presence of the second sequence of interest. The second reference sequence can be the second batch barcode and/or the second sequence of interest.

In some embodiments, hybridizing the sequencing primers to the concatemer template molecules of step (c1) can be conducted with a hybridization reagent comprising an SSC buffer (e.g., 2× saline-sodium citrate) buffer with formamide (e.g., 10-20% formamide).

In some embodiments, in step (c3) the plurality of plurality of first batch sequencing read products can be removed from the template molecules and the plurality of template molecules can be retained using a de-hybridization reagent comprising an SSC buffer (e.g., saline-sodium citrate) buffer, with or without formamide, at a temperature that promotes nucleic acid denaturation.

Re-Seeding a Support with Interrupted Sequencing

The present disclosure provides methods for re-seeding a support comprising step (a): providing a support comprising a plurality of surface capture primers immobilized to the support. In some embodiments, the plurality of immobilized capture primers have the same sequence. In some embodiments, the plurality of immobilized capture primers comprise at least two sub-populations of capture primers including at least a first sub-population of capture primers having a first sequence and a second sub-population of capture primers having a second sequence. In some embodiments, the plurality of surface capture primers comprise single-stranded oligonucleotides. In some embodiments, the plurality of surface capture primers can be used to generate nucleic acid concatemer template molecules immobilized to the support. In some embodiments, the density of the plurality of surface capture primers is about 10²-10¹⁵ per um².

In some embodiments, the plurality of surface capture primers can be immobilized to the support at random and non-pre-determined positions. In some embodiments, the plurality of surface capture primers can be immobilized to the support at pre-determined positions (e.g., a patterned support).

In some embodiments, the support is passivated with at least one polymer coating layer comprising a plurality of surface capture primers covalently tethered to the at least one polymer layer. In some embodiments, the plurality of surface capture primers are randomly distributed throughout and embedded within the at least one polymer layer.

In some embodiments, the support lacks any contours (e.g., wells, protrusions, and the like) arranged in a pre-determined pattern where the contours have features that are sites for attachment (e.g., immobilization) of the nucleic acid template molecules.

In some embodiments, the support lacks partitions/barriers that would create separate regions of the support.

In some embodiments, the methods for re-seeding a support further comprise step (b): distributing on the support a first plurality of circularized nucleic acid library molecules under a condition suitable for hybridizing individual circularized library molecules to individual surface capture primers to generate a first plurality of primed circularized library molecules and conducting a rolling circle amplification reaction, in a template-dependent manner using individual circularized library molecules in the first plurality, thereby generating a first plurality of nucleic acid concatemer template molecules immobilized to the support, wherein a subset of the surface capture primers hybridize individual circularized library molecules to generate the first plurality of concatemer template molecules. In some embodiments, the number of surface capture primers immobilized to the support exceeds the number of first plurality of circularized nucleic acid library molecules distributed onto the support. In some embodiments, individual concatemer template molecules in the first plurality comprise a plurality of tandem copies of a polynucleotide unit, where each polynucleotide unit comprises a sequence of interest and a batch seeding sequencing primer binding site sequence. In some embodiments, the first plurality of circularized library molecules can be generated using padlock probes, single-stranded splint strands, or double-stranded adaptors. In some embodiments, the first plurality of circularized library molecules comprise a mixture of any combination of circularized padlock probes, linear library molecules circularized using single-stranded splint strands, and/or linear library molecules circularized using double-stranded adaptors. Methods for generating circularized library molecules are described herein.

In some embodiments, in the methods for re-seeding a support of step (b), individual circularized library molecules in the first plurality comprise a sequence of interest, a seeding batch sequencing primer binding site sequence which corresponds to the sequence of interest, and a surface capture primer binding site. In some embodiments, a pre-determined first seeding batch sequencing primer binding site sequence can be linked to a given sequence of interest in the first plurality of circularized library molecules (or can be linked to different sequences of interest in a first plurality of circularized library molecules), thus the pre-determined first seeding batch sequencing primer binding site sequence corresponds to a given sequence of interest in the first plurality of circularized library molecules.

In some embodiments, individual circularized library molecules in the first plurality further comprise a seeding batch barcode sequence which corresponds to the sequence of interest. In some embodiments, a pre-determined first seeding batch barcode sequence can be linked to a given sequence of interest in the first plurality of circularized library molecules (or can be linked to different sequences of interest in a first plurality of circularized library molecules), thus the pre-determined first seeding batch barcode sequence corresponds to a given sequence of interest in the first plurality of circularized library molecules.

In some embodiments, individual circularized library molecules in the first plurality comprise a sequence of interest, the same seeding batch sequencing primer binding site sequence which corresponds to the sequence of interest, and individual circularized library molecules further comprise a surface capture primer binding site, and a first seeding batch barcode sequence which corresponds to the sequence of interest.

In some embodiments, the sequences of interest in the first plurality of circularized nucleic acid library molecules are about 50-250 bases in length, or about 250-500 bases in length, or about 500-800 bases in length, or about 800-1200 bases in length, or up to 2000 bases in length.

In some embodiments, the concentration of the first plurality of circularized nucleic acid library molecules that are distributed onto the support can be about 1-5 pM, or about 5-10 pM, or about 10-50 pM.

In some embodiments, in the methods for re-seeding a support of step (b), the first plurality of circularized nucleic acid library molecules comprise a plurality of sub-populations of circularized library molecules including at least a first and second sub-population of circularized library molecules.

In some embodiments, individual circularized library molecules in the first sub-population comprise the same first sub-population seeding batch sequencing primer binding site sequence and have the same sequence of interest or different sequences of interest. In some embodiments, the first sub-population seeding batch sequencing primer binding site sequence corresponds to the first sequence of interest, or the first sub-population seeding batch sequencing primer binding site sequence corresponds to one of the sequences of interest in the first sub-population. In some embodiments, a pre-determined first sub-population seeding batch sequencing primer binding site sequence can be linked to a given sequence of interest in the first sub-population of circularized library molecules (or can be linked to different sequences of interest in a first sub-population of circularized library molecules), thus the pre-determined first sub-population seeding batch sequencing primer binding site sequence corresponds to a given sequence of interest in the first sub-population of circularized library molecules.

In some embodiments, individual circularized library molecules in the first sub-population further comprise a first sub-population seeding batch barcode sequence which corresponds to the first sequence of interest, or the first sub-population seeding batch barcode sequence corresponds to one of the sequences of interest in the first sub-population. In some embodiments, a pre-determined first sub-population seeding batch barcode sequence can be linked to a given sequence of interest in the first sub-population of circularized library molecules (or can be linked to different sequences of interest in a first sub-population of circularized library molecules), thus the pre-determined first sub-population seeding batch barcode sequence corresponds to a given sequence of interest in the first sub-population of circularized library molecules.

In some embodiments, individual circularized library molecules in the first sub-population further comprise a sample index sequence that can be used in a multiplex assay to distinguish sequences of interest obtained from different sample sources. In some embodiments, individual circularized library molecules in the first sub-population further comprise a surface capture primer binding site. In some embodiments, individual circularized library molecules in the first sub-population further comprise a surface pinning primer binding site. In some embodiments, individual circularized library molecules in the first sub-population further comprise a compaction oligonucleotide binding site.

In some embodiments, the sequences of interest in the first sub-population of circularized nucleic acid library molecules are about 50-250 bases in length, or about 250-500 bases in length, or about 500-800 bases in length, or about 800-1200 bases in length, or up to 2000 bases in length.

In some embodiments, in the methods for re-seeding a support of step (b), the method comprises conducting a rolling circle amplification reaction, in a template-dependent manner using individual circularized library molecules in the first sub-population, thereby generating a plurality of first sub-population concatemer template molecules immobilized to the support, wherein a subset of the surface capture primers hybridize to individual circularized library molecules to generate the plurality of first sub-population concatemer template molecules.

In some embodiments, the plurality of first sub-population concatemer template molecules can be immobilized to the support at random and non-predetermined positions on the support, or at pre-determined positions on the support (e.g., patterned support).

In some embodiments, in the methods for re-seeding a support of step (b), individual circularized library molecules in the second sub-population comprise the same second sub-population seeding batch sequencing primer binding site sequence and have the same sequence of interest or different sequences of interest. In some embodiments, the second sub-population seeding batch sequencing primer binding site sequence corresponds to the second sequence of interest, or the second sub-population seeding batch sequencing primer binding site sequence corresponds to one of the sequences of interest in the second sub-population. In some embodiments, a pre-determined second sub-population seeding batch sequencing primer binding site sequence can be linked to a given sequence of interest in the second sub-population of circularized library molecules (or can be linked to different sequences of interest in a second sub-population of circularized library molecules), thus the pre-determined second sub-population seeding batch sequencing primer binding site sequence corresponds to a given sequence of interest in the second sub-population of circularized library molecules.

In some embodiments, individual circularized library molecules in the second sub-population further comprise a second sub-population seeding batch barcode sequence which corresponds to the second sequence of interest, or the second sub-population seeding batch barcode sequence corresponds to one of the sequences of interest in the second sub-population. In some embodiments, a pre-determined second sub-population seeding batch barcode sequence can be linked to a given sequence of interest in the second sub-population of circularized library molecules (or can be linked to different sequences of interest in a second sub-population of circularized library molecules), thus the pre-determined second subs-population seeding batch barcode sequence corresponds to a given sequence of interest in the second sub-population of circularized library molecules.

In some embodiments, individual circularized library molecules in the second sub-population further comprise a sample index sequence that can be used in a multiplex assay to distinguish sequences of interest obtained from different sample sources. In some embodiments, individual circularized library molecules in the second sub-population further comprise a surface capture primer binding site. In some embodiments, individual circularized library molecules in the second sub-population further comprise a surface pinning primer binding site. In some embodiments, individual circularized library molecules in the second sub-population further comprise a compaction oligonucleotide binding site.

In some embodiments, the sequences of interest in the second sub-population of circularized nucleic acid library molecules are about 50-250 bases in length, or about 250-500 bases in length, or about 500-800 bases in length, or about 800-1200 bases in length, or up to 2000 bases in length.

In some embodiments, the first sub-population seeding batch sequencing primer binding site sequence and second sub-population seeding batch sequencing primer binding site sequence have different sequences.

In some embodiments, in the methods for re-seeding a support of step (b), the method comprises conducting a rolling circle amplification reaction, in a template-dependent manner using individual circularized library molecules in the second sub-population, thereby generating a plurality of second sub-population concatemer template molecules immobilized to the support, wherein a subset of the surface capture primers hybridize to individual circularized library molecules to generate the plurality of second sub-population concatemer template molecules.

In some embodiments, the plurality of second sub-population concatemer template molecules can be immobilized to the support at random and non-predetermined positions on the support, or at pre-determined positions on the support (e.g., patterned support).

In some embodiments, in the methods for re-seeding a support of step (b), the rolling circle amplification reaction comprises contacting the primed circularized library molecules with a plurality of a strand displacing polymerase, and a plurality of nucleotides which include dATP, dCTP, dGTP, dTTP.

In some embodiments, the plurality of nucleotide further comprises a plurality of a nucleotide having a scissile moiety (e.g., uracil).

In some embodiments, the rolling circle amplification reaction of step (b) can be conducted in the presence, or in the absence, of a plurality of compaction oligonucleotides.

In some embodiments, the methods for re-seeding a support further comprise step (c): sequencing at least a subset of the first plurality of immobilized concatemer template molecules thereby generating a first plurality of sequencing read products. In some embodiments, the sequencing of step (c) comprises imaging a region of the support to detect the sequencing reactions of the first plurality of template molecules.

In some embodiments, the immobilized concatemer template molecules in the first plurality are sequenced. For example, at least 30-50%, or at least 50-70%, or at least 70-90% of the immobilized concatemer template molecules in the first plurality are sequenced.

In some embodiments, the full length of the immobilized concatemer template molecules in the first plurality are sequenced. In some embodiments, only a partial length of the immobilized concatemer template molecules in the first plurality are sequenced.

In some embodiments, the immobilized concatemer template molecules in the first plurality are subjected to up to 1000 sequencing cycles. In some embodiments, the immobilized concatemer template molecules in the first plurality are subjected to no more than 150 sequencing cycles.

In some embodiments, a partial length of the immobilized concatemer template molecules in the first plurality are reiteratively sequenced.

In some embodiments, in the methods for re-seeding a support of step (c), a first sub-population of the immobilized concatemer template molecules in the first plurality are sequenced using the first batch sequencing primer binding sites in the first sub-population of immobilized concatemer template molecules.

In some embodiments, the full length of the immobilized concatemer template molecules in the first sub-population are sequenced. In some embodiments, only a partial length of the immobilized concatemer template molecules in the first sub-population are sequenced.

In some embodiments, the immobilized concatemer template molecules in the first sub-population are subjected to up to 1000 sequencing cycles. In some embodiments, the immobilized concatemer template molecules in the first sub-population are subjected to no more than 150 sequencing cycles.

In some embodiments, a partial length of the immobilized concatemer template molecules in the first sub-population are reiteratively sequenced.

In some embodiments, in the methods for re-seeding a support of step (c), a second sub-population of the immobilized concatemer template molecules in the second plurality are sequenced using the second batch sequencing primer binding sites in the second sub-population of immobilized concatemer template molecules.

In some embodiments, the full length of the immobilized concatemer template molecules in the second sub-population are sequenced. In some embodiments, only a partial length of the immobilized concatemer template molecules in the second sub-population are sequenced.

In some embodiments, the immobilized concatemer template molecules in the second sub-population are subjected to up to 1000 sequencing cycles. In some embodiments, the immobilized concatemer template molecules in the second sub-population are subjected to no more than 150 sequencing cycles.

In some embodiments, a partial length of the immobilized concatemer template molecules in the second sub-population are reiteratively sequenced.

In some embodiments, the methods for re-seeding a support further comprise step (d): distributing on the support a second plurality of circularized nucleic acid library molecules under a condition suitable for hybridizing individual circularized library molecules of the second plurality to individual surface capture primers to generate a second plurality of primed circularized library molecules and conducting a second rolling circle amplification reaction, in a template-dependent manner using individual circularized library molecules in the second plurality, thereby generating a second plurality of nucleic acid concatemer template molecules immobilized to the support. In some embodiments, individual concatemer template molecules in the second plurality comprise a plurality of tandem copies of a polynucleotide unit, where each polynucleotide unit comprises a sequence of interest and a batch seeding sequencing primer binding site sequence. In some embodiments, the first plurality of concatemer template molecules of step (c) can be completely sequenced or the sequencing can interrupted at any time prior to distributing the second plurality of circularized nucleic acid library molecules onto the support of step (d). In some embodiments, the second plurality of circularized library molecules can be generated using padlock probes, single-stranded splint strands, or double-stranded adaptors. In some embodiments, the second plurality of circularized library molecules comprise a mixture of any combination of circularized padlock probes, linear library molecules circularized using single-stranded splint strands, and/or linear library molecules circularized using double-stranded adaptors. Methods for generating circularized library molecules are described herein.

In some embodiments, in the methods for re-seeding the support of step (d), individual circularized library molecules in the second plurality comprise a sequence of interest, a seeding batch sequencing primer binding site sequence which corresponds to the sequence of interest, and a surface capture primer binding site. In some embodiments, a pre-determined second seeding batch sequencing primer binding site sequence can be linked to a given sequence of interest in the second plurality of circularized library molecules (or can be linked to different sequences of interest in a second plurality of circularized library molecules), thus the pre-determined second seeding batch sequencing primer binding site sequence corresponds to a given sequence of interest in the second plurality of circularized library molecules.

In some embodiments, individual circularized library molecules in the second plurality further comprise a seeding batch barcode sequence which corresponds to the sequence of interest.

In some embodiments, a pre-determined second seeding batch barcode sequence can be linked to a given sequence of interest in the second plurality of circularized library molecules (or can be linked to different sequences of interest in a second plurality of circularized library molecules), thus the pre-determined second seeding batch barcode sequence corresponds to a given sequence of interest in the second plurality of circularized library molecules.

In some embodiments, individual circularized library molecules in the second plurality comprise a sequence of interest, the same seeding batch sequencing primer binding site sequence which corresponds to the sequence of interest, and individual circularized library molecules further comprise a surface capture primer binding site, and a second seeding batch barcode sequence which corresponds to the sequence of interest.

In some embodiments, the sequences of interest in the second plurality of circularized nucleic acid library molecules are about 50-250 bases in length, or about 250-500 bases in length, or about 500-800 bases in length, or about 800-1200 bases in length, or up to 2000 bases in length.

In some embodiments, the concentration of the second plurality of circularized nucleic acid library molecules that are distributed onto the support can be about 1-5 pM, or about 5-10 pM, or about 10-50 pM.

In some embodiments, in the methods for re-seeding a support of step (d), the second plurality of circularized nucleic acid library molecules comprise a plurality of sub-populations of circularized library molecules including at least a third and fourth sub-population of circularized library molecules.

In some embodiments, individual circularized library molecules in the third sub-population comprise the same third sub-population seeding batch sequencing primer binding site sequence and have the same sequence of interest or different sequences of interest. In some embodiments, the third sub-population seeding batch sequencing primer binding site sequence corresponds to the third sequence of interest, or the third sub-population seeding batch sequencing primer binding site sequence corresponds to one of the sequences of interest in the third sub-population. In some embodiments, a pre-determined third sub-population seeding batch sequencing primer binding site sequence can be linked to a given sequence of interest in the third sub-population of circularized library molecules (or can be linked to different sequences of interest in a third sub-population of circularized library molecules), thus the pre-determined third sub-population seeding batch sequencing primer binding site sequence corresponds to a given sequence of interest in the third sub-population of circularized library molecules.

In some embodiments, individual circularized library molecules in the third sub-population further comprise a third sub-population seeding batch barcode sequence which corresponds to the third sequence of interest, or the third sub-population seeding batch barcode sequence corresponds to one of the sequences of interest in the third sub-population. In some embodiments, a pre-determined third sub-population seeding batch barcode sequence can be linked to a given sequence of interest in the third sub-population of circularized library molecules (or can be linked to different sequences of interest in a third sub-population of circularized library molecules), thus the pre-determined third sub-population seeding batch barcode sequence corresponds to a given sequence of interest in the third sub-population of circularized library molecules.

In some embodiments, individual circularized library molecules in the third sub-population further comprise a sample index sequence that can be used in a multiplex assay to distinguish sequences of interest obtained from different sample sources. In some embodiments, individual circularized library molecules in the third sub-population further comprise a surface capture primer binding site. In some embodiments, individual circularized library molecules in the third sub-population further comprise a surface pinning primer binding site. In some embodiments, individual circularized library molecules in the third sub-population further comprise a compaction oligonucleotide binding site.

In some embodiments, the sequences of interest in the third sub-population of circularized nucleic acid library molecules are about 50-250 bases in length, or about 250-500 bases in length, or about 500-800 bases in length, or about 800-1200 bases in length, or up to 2000 bases in length.

In some embodiments, in the methods for re-seeding a support of step (d), the method comprises conducting a rolling circle amplification reaction, in a template-dependent manner using individual circularized library molecules in the third sub-population, thereby generating a plurality of third sub-population concatemer template molecules immobilized to the support, wherein a subset of the surface capture primers hybridize to individual circularized library molecules to generate the plurality of third sub-population concatemer template molecules.

In some embodiments, the plurality of third sub-population concatemer template molecules can be immobilize to the support at random and non-predetermined positions, or at pre-determined positions (e.g., patterned support).

In some embodiments, in the methods for re-seeding a support of step (d), individual circularized library molecules in the fourth sub-population comprise the same fourth sub-population seeding batch sequencing primer binding site sequence and have the same sequence of interest or different sequences of interest. In some embodiments, the fourth sub-population seeding batch sequencing primer binding site sequence corresponds to the fourth sequence of interest, or the fourth sub-population seeding batch sequencing primer binding site sequence corresponds to one of the sequences of interest in the fourth sub-population. In some embodiments, a pre-determined fourth sub-population seeding batch sequencing primer binding site sequence can be linked to a given sequence of interest in the fourth sub-population of circularized library molecules (or can be linked to different sequences of interest in a fourth sub-population of circularized library molecules), thus the pre-determined fourth sub-population seeding batch sequencing primer binding site sequence corresponds to a given sequence of interest in the fourth sub-population of circularized library molecules.

In some embodiments, individual circularized library molecules in the fourth sub-population further comprise a fourth sub-population seeding batch barcode sequence which corresponds to the fourth sequence of interest, or the fourth sub-population seeding batch barcode sequence corresponds to one of the sequences of interest in the fourth sub-population. In some embodiments, a pre-determined fourth sub-population seeding batch barcode sequence can be linked to a given sequence of interest in the fourth sub-population of circularized library molecules (or can be linked to different sequences of interest in a fourth sub-population of circularized library molecules), thus the pre-determined fourth subs-population seeding batch barcode sequence corresponds to a given sequence of interest in the fourth sub-population of circularized library molecules.

In some embodiments, individual circularized library molecules in the fourth sub-population further comprise a sample index sequence that can be used in a multiplex assay to distinguish sequences of interest obtained from different sample sources. In some embodiments, individual circularized library molecules in the fourth sub-population further comprise a surface capture primer binding site. In some embodiments, individual circularized library molecules in the fourth sub-population further comprise a surface pinning primer binding site. In some embodiments, individual circularized library molecules in the fourth sub-population further comprise a compaction oligonucleotide binding site.

In some embodiments, the sequences of interest in the fourth sub-population of circularized nucleic acid library molecules are about 50-250 bases in length, or about 250-500 bases in length, or about 500-800 bases in length, or about 800-1200 bases in length, or up to 2000 bases in length.

In some embodiments, the third sub-population seeding batch sequencing primer binding site sequence and fourth sub-population seeding batch sequencing primer binding site sequence have different sequences.

In some embodiments, in the methods for re-seeding a support of step (d), the method comprises conducting a rolling circle amplification reaction, in a template-dependent manner using individual circularized library molecules in the fourth sub-population, thereby generating a plurality of fourth sub-population concatemer template molecules immobilized to the support, wherein a subset of the surface capture primers hybridize to individual circularized library molecules to generate the plurality of fourth sub-population concatemer template molecules.

In some embodiments, the plurality of fourth sub-population concatemer template molecules can be immobilize to the support at random and non-predetermined positions, or at pre-determined positions (e.g., patterned support).

In some embodiments, in the methods for re-seeding a support of step (d), the rolling circle amplification reaction comprises contacting the primed circularized library molecules with a plurality of a strand displacing polymerase, and a plurality of nucleotides which include dATP, dCTP, dGTP, dTTP.

In some embodiments, the plurality of nucleotide further comprises a plurality of a nucleotide having a scissile moiety (e.g., uracil).

In some embodiments, the rolling circle amplification reaction of step (d) can be conducted in the presence, or in the absence, of a plurality of compaction oligonucleotides.

In some embodiments, the methods for re-seeding a support further comprise step (e): sequencing at least a subset of the second plurality of immobilized concatemer template molecules thereby generating a second plurality of sequencing read products. In some embodiments, the sequencing of step (e) comprises imaging a region of the support to detect the sequencing reactions of the second plurality of template molecules. In some embodiments, the same region of the support is sequenced in steps (c) and (e). In some embodiments, different regions of the support are sequenced in steps (c) and (e).

In some embodiments, the immobilized concatemer template molecules in the second plurality are sequenced. For example, at least 30-50%, or at least 50-70%, or at least 70-90% of the immobilized concatemer template molecules in the second plurality are sequenced.

In some embodiments, the full length of the immobilized concatemer template molecules in the second plurality are sequenced. In some embodiments, only a partial length of the immobilized concatemer template molecules in the second plurality are sequenced.

In some embodiments, the immobilized concatemer template molecules in the second plurality are subjected to up to 1000 sequencing cycles. In some embodiments, the immobilized concatemer template molecules in the second plurality are subjected to no more than 150 sequencing cycles.

In some embodiments, a partial length of the immobilized concatemer template molecules in the second plurality are reiteratively sequenced.

In some embodiments, in the methods for re-seeding a support of step (e), the third sub-population of the immobilized concatemer template molecules in the second plurality are sequenced using the third batch sequencing primer binding sites in the third sub-population of immobilized concatemer template molecules.

In some embodiments, the full length of the immobilized concatemer template molecules in the third sub-population are sequenced. In some embodiments, only a partial length of the immobilized concatemer template molecules in the third sub-population are sequenced.

In some embodiments, the immobilized concatemer template molecules in the third sub-population are subjected to up to 1000 sequencing cycles. In some embodiments, the immobilized concatemer template molecules in the third sub-population are subjected to no more than 150 sequencing cycles.

In some embodiments, a partial length of the immobilized concatemer template molecules in the third sub-population are reiteratively sequenced.

In some embodiments, in the methods for re-seeding a support of step (e), the fourth sub-population of the immobilized concatemer template molecules in the second plurality are sequenced using the fourth batch sequencing primer binding sites in the fourth sub-population of immobilized concatemer template molecules.

In some embodiments, the full length of the immobilized concatemer template molecules in the fourth sub-population are sequenced. In some embodiments, only a partial length of the immobilized concatemer template molecules in the fourth sub-population are sequenced.

In some embodiments, the immobilized concatemer template molecules in the fourth sub-population are subjected to up to 1000 sequencing cycles. In some embodiments, the immobilized concatemer template molecules in the fourth sub-population are subjected to no more than 150 sequencing cycles.

In some embodiments, a partial length of the immobilized concatemer template molecules in the fourth sub-population are reiteratively sequenced.

In some embodiments, the methods for re-seeding a support further comprise reiteratively sequencing the first sub-population of concatemer template molecules, which comprises step (c1): conducting short read sequencing by performing up to 1000 sequencing cycles of the first sub-population of concatemer template molecules to generate a plurality of first sub-population batch sequencing read products that comprise up to 1000 bases in length. In some embodiments, the methods for re-seeding a support further comprise reiteratively sequencing the first sub-population of concatemer template molecules, which comprises step (c1): conducting short read sequencing by performing no more than 150 sequencing cycles of the first sub-population of concatemer template molecules to generate a plurality of first sub-population batch sequencing read products that comprise no more than 150 bases in length.

In some embodiments, the first sub-population batch sequencing read products comprise the first sub-population seeding batch barcode sequence.

In some embodiments, the first sub-population batch sequencing read products comprise the first sub-population seeding batch barcode sequence and the sample index sequence.

In some embodiments, the first sub-population batch sequencing read products comprise the first sub-population seeding batch barcode sequence and at least a portion of the first sequence of interest.

In some embodiments, the first sub-population batch sequencing read products comprise the first sub-population seeding batch barcode sequence, the sample index sequence, and at least a portion of the first sequence of interest.

In some embodiments, the methods for re-seeding a support further comprise step (c2): stopping/blocking the short read sequencing of step (c1). In some embodiments, the stopping/blocking comprises incorporating a chain terminating nucleotide to 3′ terminal end of the first sub-population batch sequencing read products to inhibit further sequencing reactions. Exemplary chain terminating nucleotides include dideoxynucleotide or a nucleotide having a 2′ or 3′ chain terminating moiety.

In some embodiments, the methods for re-seeding a support further comprise step (c3): removing the plurality of first sub-population batch sequencing read products from the concatemer template molecules of the first sub-population, and retaining the concatemer template molecules of the first sub-population. In some embodiments, step (c3) is optional.

In some embodiments, the methods for re-seeding a support further comprise step (c4): reiteratively sequencing the concatemer template molecules of the first sub-population by repeating steps (c1)-(c3) at least once. In some embodiments, the reiterative sequencing can be conducted 1-10 times, or 10-25 times, or 25-50 times or more.

In some embodiments, the sequences of the first sub-population batch sequencing read products can be determined and aligned with a first reference sequence to confirm the presence of the first sequence of interest. The first reference sequence can be the first sub-population seeding batch barcode and/or the first sequence of interest.

In some embodiments, the methods for re-seeding a support further comprise reiteratively sequencing the third sub-population of concatemer template molecules in a manner similar to steps (c1)-(c4) as described above for the first sub-population of concatemer template molecules.

In some embodiments, the methods for re-seeding a support further comprise reiteratively sequencing the second sub-population of concatemer template molecules, which comprises step (c1): conducting short read sequencing by performing up to 1000 sequencing cycles of the second sub-population of concatemer template molecules to generate a plurality of second sub-population batch sequencing read products that comprise up to 1000 bases in length. In some embodiments, the methods for re-seeding a support further comprise reiteratively sequencing the second sub-population of concatemer template molecules, which comprises step (c1): conducting short read sequencing by performing no more than 150 sequencing cycles of the second sub-population of concatemer template molecules to generate a plurality of second sub-population batch sequencing read products that comprise no more than 150 bases in length.

In some embodiments, the second sub-population batch sequencing read products comprise the second sub-population seeding batch barcode sequence.

In some embodiments, the second sub-population batch sequencing read products comprise the second sub-population seeding batch barcode sequence and the sample index sequence.

In some embodiments, the second sub-population batch sequencing read products comprise the second sub-population seeding batch barcode sequence and at least a portion of the second sequence of interest.

In some embodiments, the second sub-population batch sequencing read products comprise the second sub-population seeding batch barcode sequence, the sample index sequence, and at least a portion of the second sequence of interest.

In some embodiments, the methods for re-seeding a support further comprise step (c2): stopping/blocking the short read sequencing of step (c1). In some embodiments, the stopping/blocking comprises incorporating a chain terminating nucleotide to 3′ terminal end of the second sub-population batch sequencing read products to inhibit further sequencing reactions. Exemplary chain terminating nucleotides include dideoxynucleotide or a nucleotide having a 2′ or 3′ chain terminating moiety.

In some embodiments, the methods for re-seeding a support further comprise step (c3): removing the plurality of second sub-population batch sequencing read products from the concatemer template molecules of the second sub-population, and retaining the concatemer template molecules of the second sub-population. In some embodiments, step (c3) is optional.

In some embodiments, the methods for re-seeding a support further comprise step (c4): reiteratively sequencing the concatemer template molecules of the second sub-population by repeating steps (c1)-(c3) at least once. In some embodiments, the reiterative sequencing can be conducted 1-10 times, or 10-25 times, or 25-50 times or more.

In some embodiments, the sequences of the second sub-population batch sequencing read products can be determined and aligned with a second reference sequence to confirm the presence of the second sequence of interest. The second reference sequence can be the second sub-population seeding batch barcode and/or the second sequence of interest.

In some embodiments, the methods for re-seeding a support further comprise reiteratively sequencing the fourth sub-population of concatemer template molecules in a manner similar to steps (c1)-(c4) as described above for the second sub-population of concatemer template molecules.

Generating Circularized Library Molecules with Padlock Probes

The present disclosure provides methods for generating circularized library molecules comprising step (a): providing a plurality of target nucleic acid molecules comprising at least a first and second target nucleic acid molecule. In some embodiments, the target nucleic acid molecules comprise RNA, DNA, cDNA or chimeric RNA/DNA. In some embodiments, the target nucleic acid molecules are present in a mixture of non-target nucleic acid molecules.

In some embodiments, methods generating circularized library molecules further comprise step (b): contacting the plurality of target nucleic acid molecules with a plurality of target-specific padlock probes, wherein individual target-specific padlock probes comprise a first and second end (e.g., first and second padlock binding arms) and an internal region having at least one adaptor sequence, wherein the first end of individual padlock probes selectively hybridize to a first region of a target molecule and the second end selectively hybridizes to a second region of the same target molecule, wherein the first and second ends of the first target-specific padlock probes hybridize to proximal positions on the target molecule to form a circular target-specific padlock probe having a nick or gap between the hybridized first and second ends. In some embodiments, the plurality of target-specific padlock probes includes at least a first plurality of target-specific padlock probes and a second plurality of target-specific padlock probes.

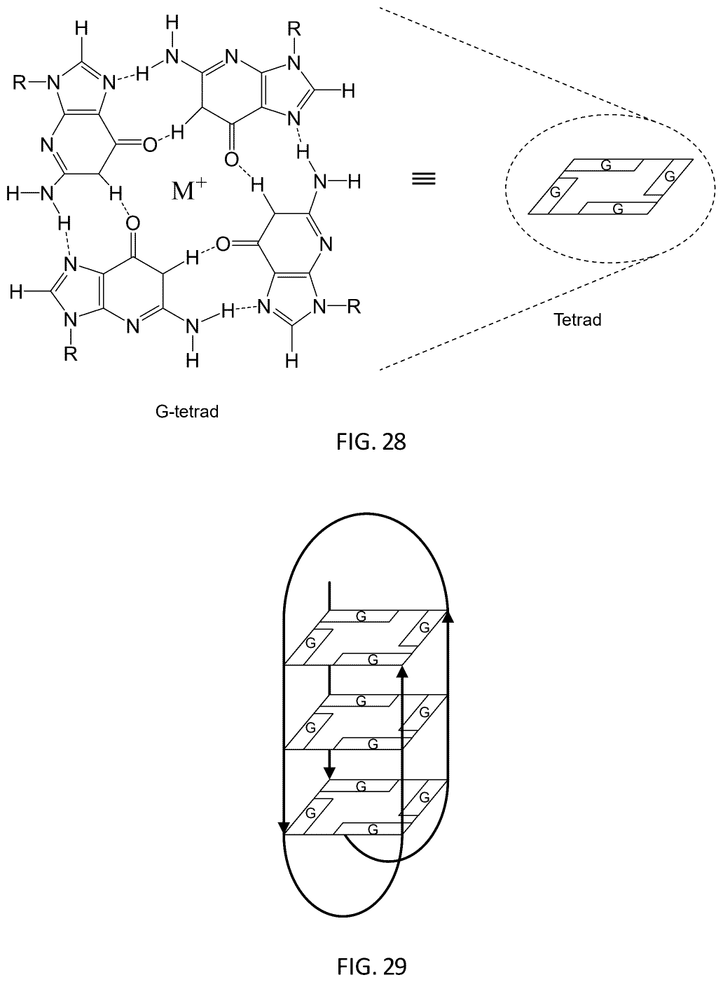

In some embodiments, individual target-specific padlock probes of step (b) comprise a first and second end (e.g., first and second padlock binding arms) and an internal region having at least one adaptor sequence, wherein the first end selectively hybridizes to a first region of a target nucleic acid molecule and the second end selectively hybridizes to a second region of the same nucleic acid target molecule. In some embodiments, the internal region of individual target-specific padlock probes comprise any one or any combination of adaptor sequence that are organized in any order including: (i) a batch-specific sequencing primer binding site sequence which corresponds to the target sequence (e.g., sequence of interest); (ii) a batch-specific barcode sequence which corresponds to the target sequence (e.g., sequence of interest); (iii) a sample index sequence that can be used in a multiplex assay to distinguish sequences of interest obtained from different sample sources; (iv) a capture primer binding site; (v) a surface pinning primer binding site; and/or (vi) a compaction oligonucleotide binding site. An exemplary embodiment of target specific padlock probes are shown in FIGS. 28A, 28B, 29-33.

In some embodiments, a pre-determined batch-specific sequencing primer binding site sequence can be linked to a given first and second padlock binding arms, thus the pre-determined batch-specific sequencing primer binding site sequence corresponds to a given target region of a target nucleic acid molecule.

In some embodiments, a pre-determined batch-specific barcode sequence can be linked to a given first and second padlock binding arms, thus the pre-determined batch-specific barcode sequence corresponds to a given target region of a target nucleic acid molecule.

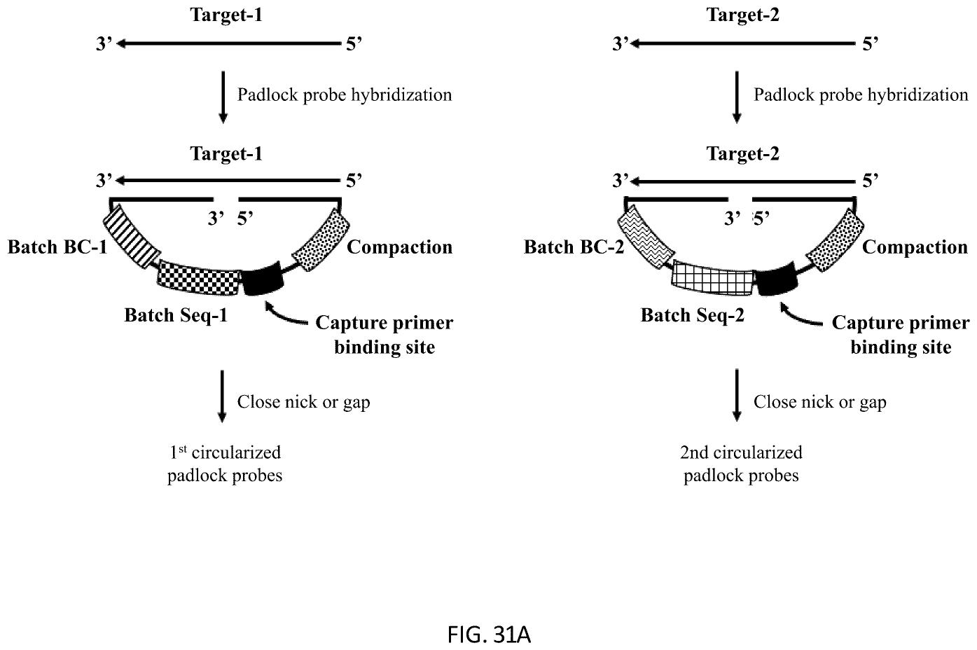

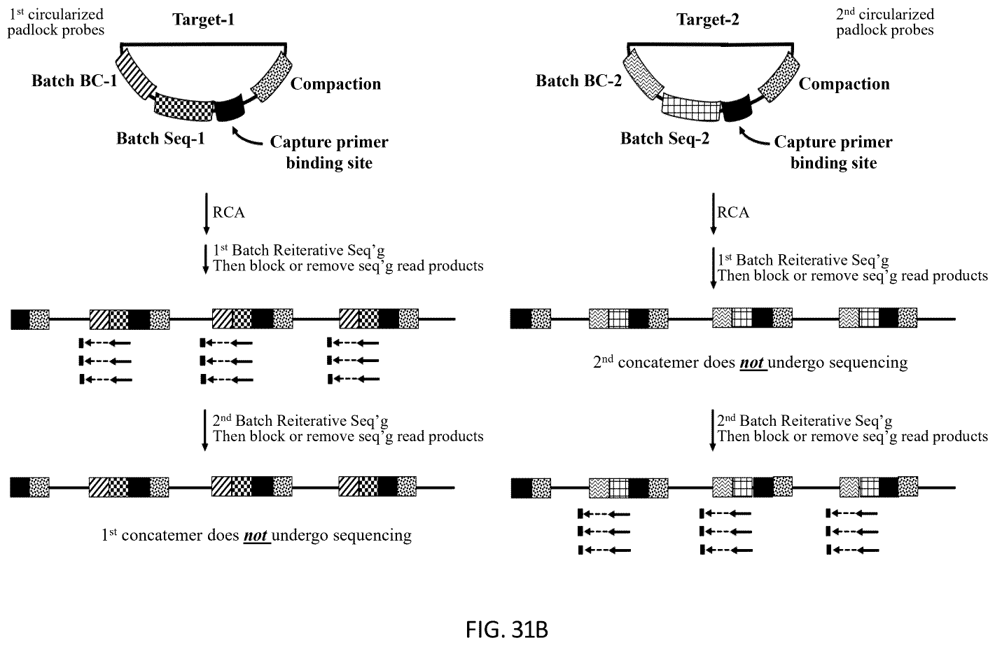

In some embodiments, individual padlock probes in the plurality of first target-specific padlock probes comprise a first and second end (e.g., first and second padlock binding arms) and an internal region, wherein the first end selectively hybridizes to a first region of the first target molecule (Target-1; e.g., see FIG. 31A) and the second end selectively hybridizes to a second region of the first target molecule. In some embodiments, the contacting of step (b) comprises: hybridizing the first and second ends of the first target-specific padlock probes to proximal positions on the first target molecule to form a circular first target-specific padlock probe having a nick or gap between the hybridized first and second ends (e.g., FIG. 31A). In some embodiments, the internal region of the first target-specific padlock probe comprises a first batch barcode sequence (Batch BC-1; see e.g., FIG. 31A) that corresponds to the first target sequence. In some embodiments, the first batch barcode sequence is located adjacent to one of the regions of the first target-specific padlock probe that selectively hybridizes to the first target molecule. In some embodiments, the first target-specific padlock probe comprises a first batch sequencing primer binding site sequence (Batch Seq-1; e.g., see FIG. 31A) (or a complementary sequence thereof). In some embodiments, the first target-specific padlock probe comprises a primer binding site for a rolling circle amplification primer (surface primer binding site; e.g., see FIG. 31A) (or a complementary sequence thereof). In some embodiments, the first target-specific padlock probe comprises a compaction oligonucleotide binding site (compaction; e.g., see FIG. 31A) (or a complementary sequence thereof). In some embodiments, the first target-specific padlock probes comprise a first batch sequencing primer binding site and a first batch barcode sequence that are adjacent to each other so that the first batch barcode region of the concatemer is sequenced first. The first batch barcode sequence can be any length, for example 3-15 bases, or 15-25 bases, or 25-40 bases, or longer. Other examples of first target-specific padlock probes are shown in FIGS. 31B, 32, 34 and 35.

In some embodiments, individual padlock probes in the plurality of second target-specific padlock probes comprise a first and second end (e.g., first and second padlock binding arms) and an internal region, wherein the first end selectively hybridizes to a first region of the second target molecule (Target-2; e.g., see FIG. 31A) and the second end selectively hybridizes to a second region of the second target molecule. In some embodiments, the contacting of step (b) comprises: hybridizing the first and second ends of the second target-specific padlock probes to proximal positions on the second target molecule to form a circular second target-specific padlock probe having a nick or gap between the hybridized first and second ends (e.g., FIG. 31A). In some embodiments, the internal region of the second target-specific padlock probe comprises a second batch barcode sequence (Batch BC-2; see e.g., FIG. 31A) that corresponds to the second target sequence. In some embodiments, the second batch barcode sequence is located adjacent to one of the regions of the second target-specific padlock probe that selectively hybridizes to the second target molecule. In some embodiments, the second target-specific padlock probe comprises a second batch sequencing primer binding site sequence (Batch Seq-2; e.g., see FIG. 31A) (or a complementary sequence thereof). In some embodiments, the second target-specific padlock probe comprises a primer binding site for a rolling circle amplification primer (surface primer binding site; e.g., see FIG. 31A) (or a complementary sequence thereof). In some embodiments, the second target-specific padlock probe comprises a compaction oligonucleotide binding site (compaction; e.g., see FIG. 31A) (or a complementary sequence thereof). In some embodiments, the second target-specific padlock probes comprise a second batch sequencing primer binding site and a second batch barcode sequence that are adjacent to each other so that the second batch barcode region of the concatemer is sequenced first. The second batch barcode sequence can be any length, for example 3-15 bases, or 15-25 bases, or 25-40 bases, or longer. Other examples of first target-specific padlock probes are shown in FIGS. 31B, 32, 34 and 35.

In some embodiments, methods generating circularized library molecules further comprise step (c): closing the nick or gap in the at least first and second circular target-specific padlock probes by conducting an enzymatic reaction, thereby generating a plurality of circularized library molecule including at least a first covalently closed circularized padlock probe and a second covalently closed circularized padlock probe (e.g., FIG. 31B). In some embodiments, the closing the nick in the first and second circular padlock probes comprises conducting an enzymatic ligation reaction thereby generating a plurality of circularized library molecules including at least first and second covalently closed circularized padlock probes. In some embodiments, closing the gap in the first and second circular padlock probes comprises conducting a polymerase-catalyzed fill-in reaction using the first or second target molecule as a template, and conducting an enzymatic ligation reaction, thereby generating a plurality of circularized library molecules including first and second covalently closed circularized padlock probes. In some embodiments, various embodiments of padlock probes carrying different adaptor sequences in their internal region can be used to generate various embodiments of covalently closed circularized padlock probes (e.g., see FIGS. 31-36).

In some embodiments, as shown in FIG. 31B, the rolling circle amplification reaction can be conducted in-solution using soluble amplification primers or on-support using capture primers immobilized to a support. In some embodiments, the first and second concatemer template molecules are subjected to a first sequencing workflow using first batch-specific sequencing primers (solid arrows), sequencing polymerases, and a plurality of nucleotide reagents to generate a plurality of first sequencing read products (dashed arrows), where the first sequencing read products include only the first batch barcode sequence (Batch BC-1). In some embodiments, the first concatemers undergo reiterative sequencing comprising up to 1000 sequencing cycles, but the second concatemers do not undergo first batch sequencing. In some embodiments, the first concatemers undergo reiterative sequencing comprising no more than 150 sequencing cycles. In some embodiments, the first and second concatemer template molecules are subjected to a second sequencing workflow using second batch-specific sequencing primers (solid arrows), sequencing polymerases, and a plurality of nucleotide reagents to generate a plurality of second sequencing read products (dashed arrows), where the second sequencing read products include only the second batch barcode sequence (Batch BC-2). In some embodiments, the second concatemers undergo reiterative sequencing comprising up to 1000 sequencing cycles, but the first concatemers do not undergo second batch sequencing. In some embodiments, the second concatemers undergo reiterative sequencing comprising no more than 150 sequencing cycles, but the first concatemers do not undergo second batch sequencing.

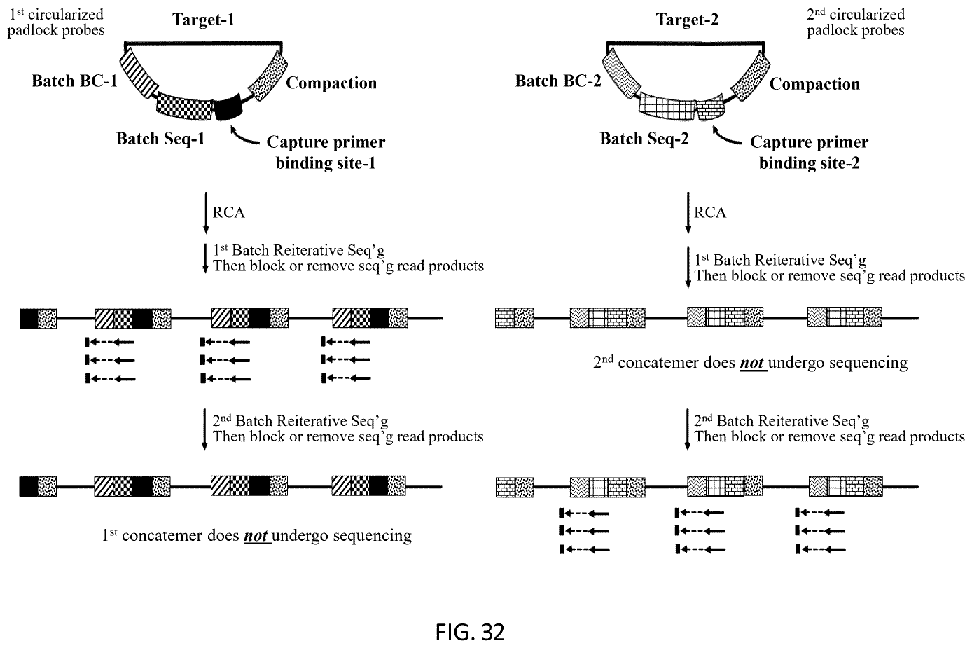

In some embodiments, the rolling circle amplification reaction can be conducted in-solution using soluble amplification primers or on-support using capture primers immobilized to a support. In some embodiments, the first circularized padlock probe (Left) comprises: (i) a batch-specific barcode sequence which corresponds to the first target sequence (Batch BC-1); (ii) a batch-specific sequencing primer binding site sequence which corresponds to the first target sequence (e.g., Batch Seq-1); (iii) a first batch capture primer binding site; and (iv) a compaction oligonucleotide binding site. In some embodiments, the second padlock probe (Right) comprises: (i) a batch-specific barcode sequence which corresponds to the second target sequence (Batch BC-2); (ii) a batch-specific sequencing primer binding site sequence which corresponds to the second target sequence (e.g., Batch Seq-2); (iii) a second batch capture primer binding site; and (iv) a compaction oligonucleotide binding site. In some embodiments, the first and second circularized padlock probes are distributed onto a support having two types of immobilized capture primers which selectively hybridize to the first or second batch capture primer binding site sequences in the first or second circularized padlock probes. In some embodiments, e.g., as shown in FIG. 32, the circularized padlock probes are subjected to rolling circle amplification (RCA) to generate first and second concatemer template molecules which are immobilized to the support. In some embodiments, the first and second circularized padlock probes can be distributed onto the support essentially simultaneously, or distributed onto the support sequentially (e.g., re-seeding the support). In some embodiments, the first and second concatemer template molecules are subjected to a first sequencing workflow using first batch-specific sequencing primers (solid arrows), sequencing polymerases, and a plurality of nucleotide reagents to generate a plurality of first sequencing read products (dashed arrows), where the first sequencing read products include only the first batch barcode sequence (Batch BC-1). In some embodiments, the first concatemers undergo reiterative sequencing comprising up to 1000 sequencing cycles, but the second concatemers do not undergo first batch sequencing. In some embodiments, the first concatemers undergo reiterative sequencing comprising no more than 150 sequencing cycles, but the second concatemers do not undergo first batch sequencing. In some embodiments, the first and second concatemer template molecules are subjected to a second sequencing workflow using second batch-specific sequencing primers (solid arrows), sequencing polymerases, and a plurality of nucleotide reagents to generate a plurality of second sequencing read products (dashed arrows), where the second sequencing read products include only the second batch barcode sequence (Batch BC-2). In some embodiments, the second concatemers undergo reiterative sequencing comprising up to 1000 sequencing cycles, but the first concatemers do not undergo second batch sequencing. In some embodiments, the second concatemers undergo reiterative sequencing comprising no more than 150 sequencing cycles, but the first concatemers do not undergo second batch sequencing.

In some embodiments, methods generating circularized library molecules further comprise step (d): conducting a rolling circle amplification reaction by hybridizing the plurality of circularized library molecules with a plurality of amplification primers and conducting rolling circle amplification reaction in a template-dependent manner, using a plurality of strand displacing polymerases and a plurality of nucleotides, thereby generating a plurality of concatemer molecules. In some embodiments, the rolling circle amplification reaction comprises hybridizing first and second covalently closed circularized padlock probes to first and second amplification primers, respectively. In some embodiments, the first and second amplification primers are immobilized to a support (e.g., first and second capture primers). In some embodiments, the first and second amplification primers are in solution. In some embodiments, the first and second amplification primers have the same sequence or have different sequences. In some embodiments, the rolling circle amplification reaction is conducted in a template-dependent manner, using a plurality of strand displacing polymerases, a plurality of nucleotides, and the first and second covalently closed circularized padlock probes, thereby generating a plurality of concatemer molecules including at least a first concatemer molecule that corresponds to a first target nucleic acid molecule, and the plurality of concatemer molecules includes at least a second concatemer molecule that corresponds to a second target nucleic acid molecule. In some embodiments, the first concatemer molecule comprises tandem repeat polynucleotide units, wherein a unit comprises the sequence of the first target molecule, the first batch barcode sequence, and the first batch sequencing primer binding site (e.g., see FIGS. 31B-36) (or a complementary sequence thereof). In some embodiments, the second concatemer molecule comprises tandem repeat polynucleotide units, wherein a unit comprises the sequence of the second target molecule, the second batch barcode sequence, and the second batch sequencing primer binding site (e.g., see FIGS. 31B-36) (or a complementary sequence thereof).

In some embodiments, when the rolling circle amplification of step (d) is conducted with amplification primers that are immobilized to a support, the covalently closed circularized padlock probes can be distributed onto the support comprising a plurality of immobilized surface primer, under a condition suitable to hybridize at least one portion of the covalently closed circularized padlock probes to the immobilized surface primers, and the rolling circle amplification reaction is conducted thereby generating concatemers immobilized to a support.

In some embodiments, when the rolling circle amplification of step (d) is conducted with amplification primers that are in solution, the covalently closed circularized padlock probes can be hybridized to amplification primers in solution, the rolling circle amplification reaction can be conducted in-solution, and the rolling circle amplification reaction and nascent concatemers can be distributed onto a support having a plurality of surface primers immobilized thereon, under a condition suitable to hybridize at least one portion of the nascent concatemers to the immobilized surface primers, and the rolling circle amplification reaction can be resumed thereby generating concatemers immobilized to a support.

In some embodiments, methods generating circularized library molecules further comprise step (e): sequencing the plurality of concatemer molecules immobilized to the support. In some embodiments, the sequencing of step (e) comprises sequencing the first batch concatemer molecules by conducting 2-1000 sequencing cycles to generate a plurality of first sequencing read products, and sequencing the second batch concatemer molecules by conducting conducting 2-1000 sequencing cycles to generate a plurality of second sequencing read products. In some embodiments, the first and second batch concatemer molecules can be sequenced essentially simultaneously using a mixture of first and second batch-specific sequencing primers. In some embodiments, the first and second batch concatemer molecules can be sequenced separately in batches using first batch-specific sequencing primers and then using second batch-specific sequencing primers.

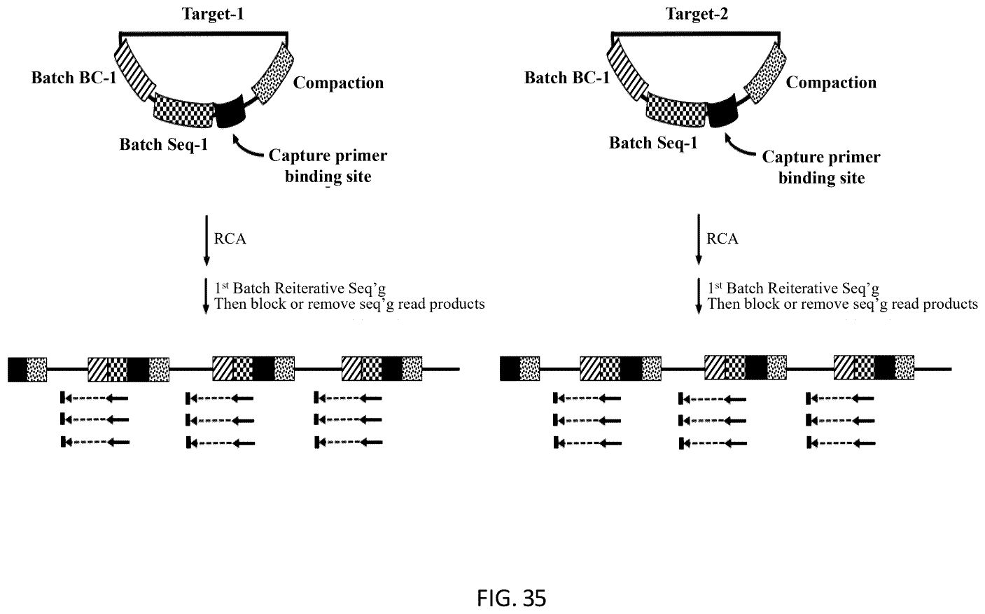

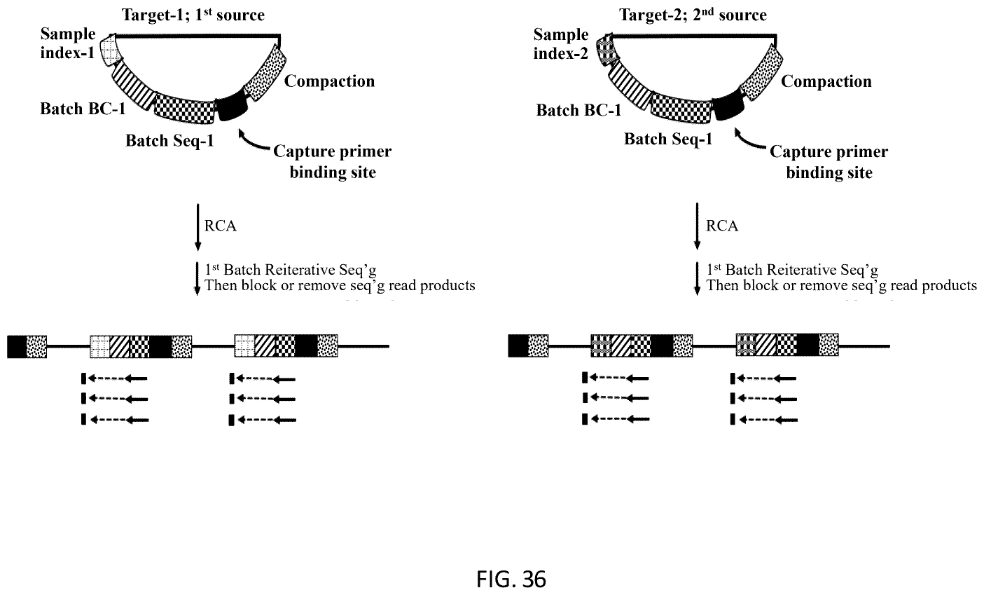

In some embodiments, only the first batch barcode region of the first concatemer molecules are sequenced (e.g., FIGS. 31B, 32, 33 and 34; left schematics). In some embodiments, the first batch barcode region and the sample index region of the first concatemer molecules are sequenced (e.g., FIG. 36; left schematics). In some embodiments, the first batch barcode is sequenced and a portion of the first sequence of interest region of the first concatemer molecules are sequenced (e.g., FIG. 35; left schematic).

In some embodiments, only the second batch barcode region of the second concatemer molecules are sequenced (e.g., FIGS. 31B, 32, 33 and 34; right schematics). In some embodiments, the second batch barcode region and the sample index region of the second concatemer molecules are sequenced (e.g., FIG. 36; right schematic). In some embodiments, the second batch barcode is sequenced and a portion of the second sequence of interest region of the second concatemer molecules are sequenced (e.g., FIG. 35; right schematic).

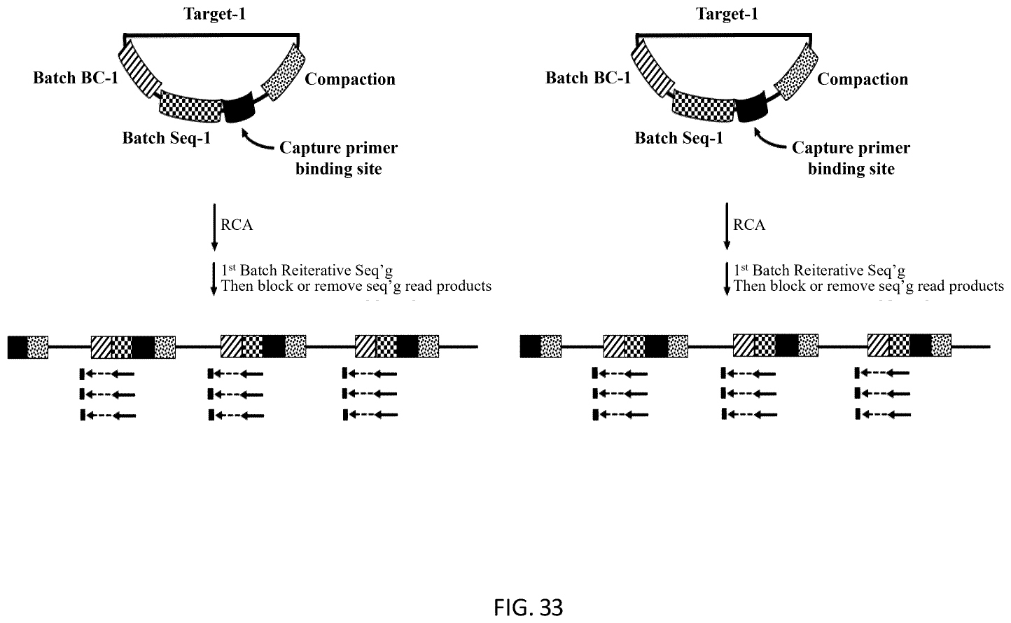

In some embodiments, e.g., as in FIG. 33, the rolling circle amplification reaction can be conducted in-solution using soluble amplification primers or on-support using capture primers immobilized to a support. In some embodiments, the first and second circularized padlock probes (Left and Right) comprises: (i) a batch-specific barcode sequence which corresponds to the first target sequence (Batch BC-1); (ii) a batch-specific sequencing primer binding site sequence which corresponds to the first target sequence (e.g., Batch Seq-1); (iii) a first batch capture primer binding site; and (iv) a compaction oligonucleotide binding site. In some embodiments, the first and second circularized padlock probes are distributed onto a support having one type of immobilized capture primers which selectively hybridize to the first batch capture primer binding site sequences in the first and second circularized padlock probes. In some embodiments, the circularized padlock probes are subjected to rolling circle amplification (RCA) to generate first and second concatemer template molecules which are immobilized to the support. In some embodiments, the first and second circularized padlock probes can be distributed onto the support essentially simultaneously, or distributed onto the support sequentially (e.g., re-seeding the support). In some embodiments, the first and second concatemer template molecules are subjected to a first sequencing workflow using first batch-specific sequencing primers (solid arrows), sequencing polymerases, and a plurality of nucleotide reagents to generate a plurality of first sequencing read products (dashed arrows), where the first sequencing read products include only the first batch barcode sequence (Batch BC-1). In some embodiments, the first and second concatemers undergo reiterative sequencing comprising up to 1000 sequencing cycles. In some embodiments, the first and second concatemers undergo reiterative sequencing comprising no more than 150 sequencing cycles.

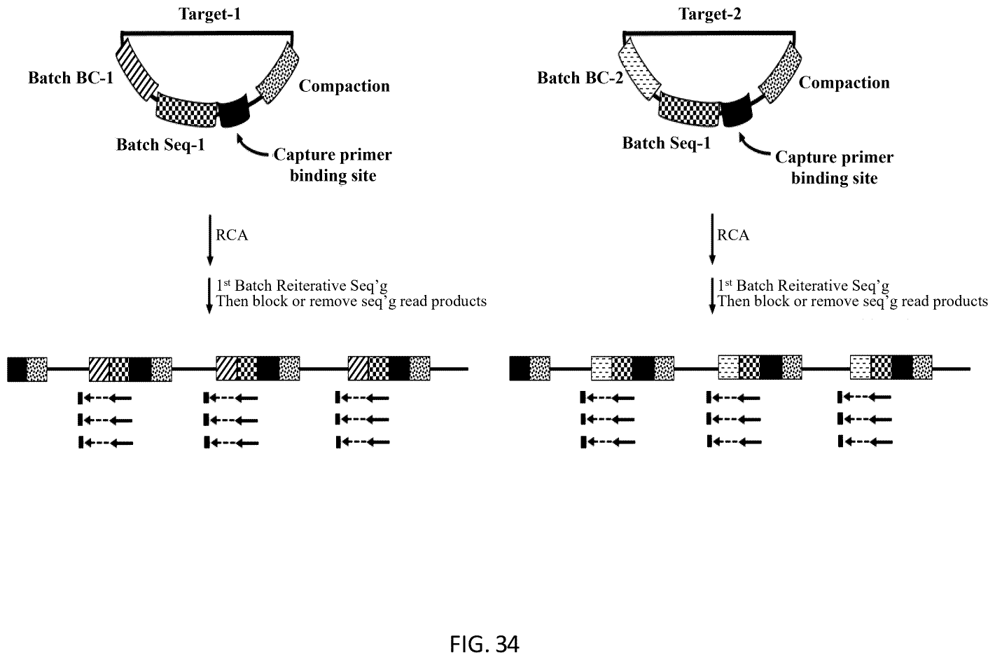

In some embodiments, e.g., as in FIG. 34, the rolling circle amplification reaction can be conducted in-solution using soluble amplification primers or on-support using capture primers immobilized to a support. In some embodiments, the first circularized padlock probe (Left) comprises: (i) a batch-specific barcode sequence which corresponds to the first target sequence (Batch BC-1); (ii) a batch-specific sequencing primer binding site sequence which corresponds to the first and second target sequence (e.g., Batch Seq-1); (iii) a first batch capture primer binding site; and (iv) a compaction oligonucleotide binding site. In some embodiments, the second padlock probe (Right) comprises: (i) a batch-specific barcode sequence which corresponds to the second target sequence (Batch BC-2); (ii) a batch-specific sequencing primer binding site sequence which corresponds to the first and second target sequence (e.g., Batch Seq-1); (iii) a first batch capture primer binding site; and (iv) a compaction oligonucleotide binding site. In some embodiments, the first and second circularized padlock probes are distributed onto a support having one type of immobilized capture primers which selectively hybridizes to the first batch capture primer binding site sequence in the first and second circularized padlock probes. In some embodiments, the circularized padlock probes are subjected to rolling circle amplification (RCA) to generate first and second concatemer template molecules which are immobilized to the support. In some embodiments, the first and second circularized padlock probes can be distributed onto the support essentially simultaneously, or distributed onto the support sequentially (e.g., re-seeding the support). In some embodiments, the first and second concatemer template molecules are subjected to a first sequencing workflow using first batch-specific sequencing primers (solid arrows), sequencing polymerases, and a plurality of nucleotide reagents to generate a plurality of first and second sequencing read products (dashed arrows). In some embodiments, the first sequencing read products include only the first batch barcode sequence (Batch BC-1). In some embodiments, the first concatemers undergo reiterative sequencing comprising up to 1000 sequencing cycles. In some embodiments, the first concatemers undergo reiterative sequencing comprising no more than 150 sequencing cycles. In some embodiments, the second sequencing read products include only the second batch barcode sequence (Batch BC-2). In some embodiments, the second concatemers undergo reiterative sequencing comprising up to 1000 sequencing cycles. In some embodiments, the second concatemers undergo reiterative sequencing comprising no more than 150 sequencing cycles.

In some embodiments, e.g., as in FIG. 35, the rolling circle amplification reaction can be conducted in-solution using soluble amplification primers or on-support using capture primers immobilized to a support. In some embodiments, the first circularized padlock probe (Left) comprises: (i) a batch-specific barcode sequence which corresponds to the first and second target sequence (Batch BC-1); (ii) a batch-specific sequencing primer binding site sequence which corresponds to the first and second target sequence (e.g., Batch Seq-1); (iii) a first batch capture primer binding site; and (iv) a compaction oligonucleotide binding site. In some embodiments, the second padlock probe (Right) comprises: (i) a batch-specific barcode sequence which corresponds to the first and second target sequence (Batch BC-1); (ii) a batch-specific sequencing primer binding site sequence which corresponds to the first and second target sequence (e.g., Batch Seq-1); (iii) a first batch capture primer binding site; and (iv) a compaction oligonucleotide binding site. In some embodiments, the first and second circularized padlock probes are distributed onto a support having one type of immobilized capture primers which selectively hybridizes to the first batch capture primer binding site sequence in the first and second circularized padlock probes. In some embodiments, the circularized padlock probes are subjected to rolling circle amplification (RCA) to generate first and second concatemer template molecules which are immobilized to the support. In some embodiments, the first and second circularized padlock probes can be distributed onto the support essentially simultaneously, or distributed onto the support sequentially (e.g., re-seeding the support). In some embodiments, the first and second concatemer template molecules are subjected to a first sequencing workflow using first batch-specific sequencing primers (solid arrows), sequencing polymerases, and a plurality of nucleotide reagents to generate a plurality of first and second sequencing read products (dashed arrows). In some embodiments, the first sequencing read products include the first batch barcode sequence (Batch BC-1) and at least a portion of the first target sequence. In some embodiments, the first concatemers undergo reiterative sequencing comprising up to 1000 sequencing cycles. In some embodiments, the first concatemers undergo reiterative sequencing comprising no more than 150 sequencing cycles. In some embodiments, the second sequencing read products include the second batch barcode sequence (Batch BC-2) and at least a portion of the second target sequence. In some embodiments, the second concatemers undergo reiterative sequencing comprising up to 1000 sequencing cycles. In some embodiments, the second concatemers undergo reiterative sequencing comprising no more than 150 sequencing cycles.

In some embodiments, e.g., in FIG. 36, the rolling circle amplification reaction can be conducted in-solution using soluble amplification primers or on-support using capture primers immobilized to a support. In some embodiments, the first circularized padlock probe (Left) comprises: (i) a first sample index which distinguish sequences of interest obtained from a first sample source (e.g., Sample index-1); (ii) a batch-specific barcode sequence which corresponds to the first target sequence (Batch BC-1); (iii) a batch-specific sequencing primer binding site sequence which corresponds to the first target sequence (e.g., Batch Seq-1); (iv) a first batch capture primer binding site; and (v) a compaction oligonucleotide binding site. In some embodiments, the second circularized padlock probe (Left) comprises: (i) a second sample index which distinguish sequences of interest obtained from a second sample source (e.g., Sample index-2); (ii) a batch-specific barcode sequence which corresponds to the first target sequence (Batch BC-1); (iii) a batch-specific sequencing primer binding site sequence which corresponds to the first target sequence (e.g., Batch Seq-1); (iv) a first batch capture primer binding site; and (v) a compaction oligonucleotide binding site. In some embodiments, the first and second circularized padlock probes are distributed onto a support having one type of immobilized capture primers which selectively hybridizes to the first batch capture primer binding site sequence in the first and second circularized padlock probes. In some embodiments, the circularized padlock probes are subjected to rolling circle amplification (RCA) to generate first and second concatemer template molecules which are immobilized to the support. In some embodiments, the first and second circularized padlock probes can be distributed onto the support essentially simultaneously, or distributed onto the support sequentially (e.g., re-seeding the support). In some embodiments, the first and second concatemer template molecules are subjected to a first sequencing workflow using first batch-specific sequencing primers (solid arrows), sequencing polymerases, and a plurality of nucleotide reagents to generate a plurality of first and second sequencing read products (dashed arrows). In some embodiments, the first sequencing read products include the first batch barcode sequence (Batch BC-1) and the first sample index sequence. In some embodiments, the first concatemers undergo reiterative sequencing comprising up to 1000 sequencing cycles. In some embodiments, the first concatemers undergo reiterative sequencing comprising no more than 150 sequencing cycles. In some embodiments, the second sequencing read products include the second batch barcode sequence (Batch BC-2) and the second sample index sequence. In some embodiments, the second concatemers undergo reiterative sequencing comprising up to 1000 sequencing cycles. In some embodiments, the second concatemers undergo reiterative sequencing comprising no more than 150 sequencing cycles.

In some embodiments, the sequencing of step (e) comprises a reiterative sequencing workflow, which comprises: step (e1) contacting the plurality of concatemer molecules with (i) a plurality of batch-specific sequencing primers, (ii) a plurality of sequencing polymerases, and (iii) a plurality of nucleotide reagents, under a condition suitable for hybridizing the plurality of batch-specific sequencing primers to their respective batch sequencing primer binding sites on the concatemers. In some embodiments, the reiterative sequencing further comprises step (e2) conducting 2-1000 sequencing cycles to generate at least a first plurality of sequencing read products and optionally a second plurality of sequencing read products. In some embodiments, the reiterative sequencing further comprises step (e3) removing the first plurality of sequencing read products from the concatemers and retaining the plurality of concatemers, and optionally removing the second plurality of sequencing read products from the concatemers and retaining the plurality of concatemers. In some embodiments, the reiterative sequencing further comprises step (e4) repeating steps (1)-(3) at least once. In some embodiments, step (4) comprises repeating steps (1)-(3) up to 100 times.

In some embodiments, the reiterative sequencing can be conducting using a sequencing-by-binding procedure, labeled and/or non-labeled chain-terminating nucleotides, or multivalent molecules. Descriptions of these three sequencing methods is described below.

In some embodiments, the plurality of batch-specific sequencing primers can be hybridized to concatemer template molecules with a hybridization reagent comprising an SSC buffer (e.g., 2× saline-sodium citrate) buffer with formamide (e.g., 10-20% formamide).

In some embodiments, the plurality of sequencing read products can be removed from the concatemers and the plurality of concatemers can be retained on the support using a de-hybridization reagent comprising an SSC buffer (e.g., saline-sodium citrate) buffer, with or without formamide, at a temperature that promotes nucleic acid denaturation.

### Generating Circularized Library Molecules Using Single-Stranded Splint Strands

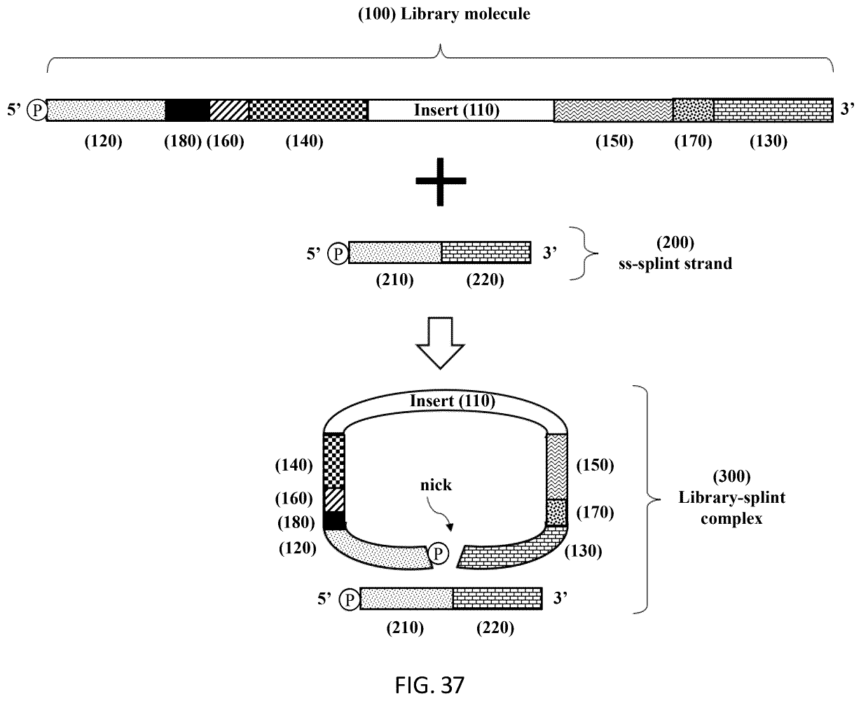

The present disclosure provides methods generating circularized library molecules comprising step (a): providing a plurality of single-stranded nucleic acid library molecules (100) wherein individual library molecules comprise the following components arranged in any order: (i) pinning primer binding site sequence (120) (e.g., batch-specific pinning primer binding site sequence); (ii) a unique identification sequence (e.g., UMI) (180); (iii) a batch-specific barcode sequence (195); (iv) a left sample index sequence (160); (v) a forward sequencing primer binding site sequence (140) (e.g., batch-specific forward sequencing primer binding site sequence (140); (vi) a sequence of interest (e.g., insert sequence) (110); (vii) a reverse sequencing primer binding site sequence (150) (e.g., batch-specific reverse sequencing primer binding site sequence (150); (viii) a right sample index sequence (170); and/or (ix) a capture primer binding site sequence (130) (e.g., batch-specific capture primer binding site sequence (130). Embodiments of various single-stranded library molecules are shown in FIGS. 37, 38, 39A, 39B, 41A and 41B.

In some embodiments, individual single-stranded library molecules (100) lacks any one or any combination of: a unique identification sequence (e.g., UMI) (180); a batch-specific barcode sequence (195); a left sample index sequence (160); a reverse sequencing primer binding site sequence (150) (e.g., batch-specific reverse sequencing primer binding site sequence (150); and/or a right sample index sequence (170).

In some embodiments, the left and right sample index sequences can be used to distinguish insert sequences that are isolated from different sample sources in a multiplex assay. The first left index sequences (160) and/or first right index sequences (170) can be employed to prepare separate sample-indexed libraries using input nucleic acids isolated from different sources. The sample-indexed libraries can be pooled together to generate a multiplex library mixture, and the pooled libraries can be circularized, amplified and/or sequenced. In some embodiments, the sequences of the left sample index (160) and the right sample index (170) are the same or different from each other. The left sample index sequence (160) can be 3-20 nucleotides in length. The right sample index sequence (170) can be 3-20 nucleotides in length. The left sample index sequence (160) and/or the right sample index sequence (170) can include a short random sequence (e.g., NNN). The short random sequence can be 3-20 nucleotides in length.

In some embodiments, the unique identification sequence (180) (e.g., a unique molecular tag) that can be used to uniquely identify individual nucleic acid library molecules to which the unique identification sequence is appended.

In some embodiments, the plurality of single-stranded nucleic acid library molecules (100) includes at least a first and second sub-population of single-stranded nucleic acid library molecules (100).

In some embodiments, the first of sub-population single-stranded library molecules comprise (i) a first batch pinning primer binding site sequence (120) (e.g., pinning primer binding site sequence-1); (ii) a unique identification sequence (e.g., UMI) (180); (iii) a first batch barcode sequence (195) (e.g., batch BC-1); (iv) a left sample index sequence (160); (v) a first batch forward sequencing primer binding site sequence (140) (e.g., batch FWD Seq-1); (vi) a first sequence of interest (e.g., first insert sequence) (110); (vii) a first batch reverse sequencing primer binding site sequence (150) (e.g., batch REV Seq-1); (viii) a right sample index sequence (170); and/or (ix) a first batch capture primer binding site sequence (130) (e.g., capture primer binding site sequence-1).

In some embodiments, the single-stranded library molecules within the first sub-population have the same first batch forward sequencing primer binding site sequence (140) (e.g., batch FWD Seq-1), and have the same or different first sequence(s) of interest.

In some embodiments, the sequence of the first batch forward sequencing primer binding site sequence (140) (e.g., batch FWD Seq-1) corresponds to the first sequence of interest, or the first batch forward sequencing primer binding site sequence (140) (e.g., batch FWD Seq-1) corresponds to one of the first sequences of interest in the first sub-population.

In some embodiments, a pre-determined first batch forward sequencing primer binding site sequence (140) (e.g., batch FWD Seq-1) can be linked to a given sequence of interest in the first sub-population (or can be linked to different sequences of interest in a first sub-population), thus the pre-determined first batch forward sequencing primer binding site sequence (140) (e.g., batch FWD Seq-1) corresponds to a given sequence of interest in the first sub-population.

In some embodiments, the single-stranded library molecules within the first sub-population have the same first batch barcode sequence (195) (e.g., batch BC-1), and have the same or different first sequence(s) of interest.

In some embodiments, the sequence of the first batch barcode sequence (195) (e.g., batch BC-1) corresponds to the first sequence of interest, or the first batch barcode sequence (195) (e.g., batch BC-1) corresponds to one of the first sequences of interest in the first sub-population.

In some embodiments, a pre-determined first batch barcode sequence (195) (e.g., batch BC-1) can be linked to a given sequence of interest in the first sub-population (or can be linked to different sequences of interest in a first sub-population), thus the pre-determined first batch barcode sequence (195) (e.g., batch BC-1) corresponds to a given sequence of interest in the first sub-population.

In some embodiments, the sequences of interest in the first sub-population are about 50-250 bases in length, or about 250-500 bases in length, or about 500-800 bases in length, or about 800-1200 bases in length, or up to 2000 bases in length.

In some embodiments, the second single-stranded library molecule comprises (i) a second batch pinning primer binding site sequence (120) (e.g., pinning primer binding site sequence-2); (ii) a unique identification sequence (e.g., UMI) (180); (iii) a second batch barcode sequence (195) (e.g., batch BC-2); (iv) a left sample index sequence (160); (v) a second batch forward sequencing primer binding site sequence (140) (e.g., batch FWD Seq2); (vi) a second sequence of interest (e.g., second insert sequence) (110); (vii) a second batch reverse sequencing primer binding site sequence (150) (e.g., batch REV Seq-2); (viii) a right sample index sequence (170); and/or (ix) a second batch capture primer binding site sequence (130) (e.g., capture primer binding site sequence-2).

In some embodiments, the single-stranded library molecules within the second sub-population have the same second batch forward sequencing primer binding site sequence (140) (e.g., batch FWD Seq-2), and have the same or different second sequence(s) of interest.

In some embodiments, the sequence of the second batch forward sequencing primer binding site sequence (140) (e.g., batch FWD Seq-2) corresponds to the second sequence of interest, or the second batch forward sequencing primer binding site sequence (140) (e.g., batch FWD Seq-2) corresponds to one of the second sequences of interest in the second sub-population.

In some embodiments, a pre-determined second batch forward sequencing primer binding site sequence (140) (e.g., batch FWD Seq-2) can be linked to a given sequence of interest in the second sub-population (or can be linked to different sequences of interest in a second sub-population), thus the pre-determined second batch forward sequencing primer binding site sequence (140) (e.g., batch FWD Seq-2) corresponds to a given sequence of interest in the second sub-population.

In some embodiments, the single-stranded library molecules within the second sub-population have the same second batch barcode sequence (195) (e.g., batch BC-2), and have the same or different second sequence(s) of interest.

In some embodiments, the sequence of the second batch barcode sequence (195) (e.g., batch BC-2) corresponds to the second sequence of interest, or the second batch barcode sequence (195) (e.g., batch BC-2) corresponds to one of the second sequences of interest in the second sub-population.

In some embodiments, a pre-determined second batch barcode sequence (195) (e.g., batch BC-2) can be linked to a given sequence of interest in the second sub-population (or can be linked to different sequences of interest in a second sub-population), thus the pre-determined second batch barcode sequence (195) (e.g., batch BC-2) corresponds to a given sequence of interest in the second sub-population.

In some embodiments, the sequences of interest in the second sub-population are about 50-250 bases in length, or about 250-500 bases in length, or about 500-800 bases in length, or about 800-1200 bases in length, or up to 2000 bases in length.

In some embodiments, the sequences of the first and second batch pinning primer binding site sequence (120) are the same or different (e.g., pinning primer binding site sequence-1 and pinning primer binding site sequence-2).

In some embodiments, the sequences of the first and second batch forward sequencing primer binding site sequence (140) are the same or different (e.g., batch FWD Seq-1 and batch FWD Seq2).

In some embodiments, the sequences of the first and second batch barcode sequence (195) are the same or different (e.g., batch BC-1 and batch BC-2).

In some embodiments, the sequences of the first and second batch reverse sequencing primer binding site sequence (150) are the same or different (e.g., batch REV Seq-1 and batch REV Seq-2).

In some embodiments, the sequences of the first and second batch capture primer binding site sequence (130) are the same or different (e.g., capture primer binding site sequence-1 and capture primer binding site sequence-2).

In some embodiments, the method for generating circularized library molecules further comprises step (b): providing a plurality of single-stranded splint strands (200) wherein individual single-stranded splint strands (200) comprises regions arranged in any order (i) a first region (210) having a sequence that hybridizes with the pinning primer binding site sequence (120) of the single stranded library molecule, and (ii) a second region (220) having a sequence that hybridizes with capture primer binding site sequence (130) of the single stranded library molecule.

In some embodiments, the method for generating circularized library molecules further comprises step (c): contacting the plurality of single-stranded nucleic acid library molecules (100) with the plurality of single-stranded splint strands (200), wherein the contacting is conducted under a condition suitable to hybridize individual library molecules (100) with individual single-stranded splint strands (200) thereby circularizing the library molecule to generate a library-splint complex (300), wherein the first region (210) of the single-stranded splint strand is hybridized to the pinning primer binding site sequence (120) of the single stranded library molecule, and wherein the second region (220) of the single-stranded splint strand is hybridized to capture primer binding site sequence (130) of the single stranded library molecule, wherein the library-splint complex (300) comprises a nick between the terminal 5′ and 3′ ends of the library molecule, and wherein the nick is enzymatically ligatable (e.g., see FIGS. 37, 38, 39A, 39B, 41A and 41B).

In some embodiments, e.g., in FIG. 37, the exemplary library molecule (100) comprises: surface pinning primer binding site sequence (120) (e.g., batch-specific surface pinning primer binding site sequence); an optional left unique identification sequence (180) (e.g., UMI); a left sample index sequence (160); a forward sequencing primer binding site sequence (140) (e.g., batch-specific forward sequencing primer binding site sequence (140); a sequence of interest (110); a reverse sequencing primer binding site sequence (150) (e.g., batch-specific reverse sequencing primer binding site sequence (150); a right sample index sequence (170); and a surface capture primer binding site sequence (130) (e.g., batch-specific surface capture primer binding site sequence (130). In some embodiments, the single-stranded splint strand (200) comprises a first region (210) that hybridizes with the surface pinning primer binding site sequence (120) of the linear single stranded library molecule (100), and a second region (220) that hybridizes with the surface capture primer binding site sequence (130) of the linear single stranded library molecule (100).

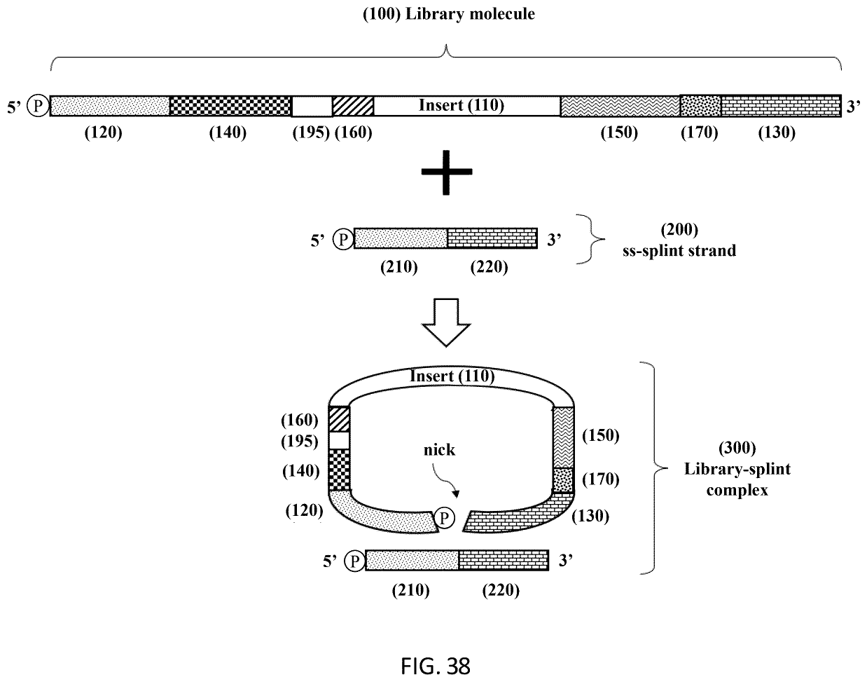

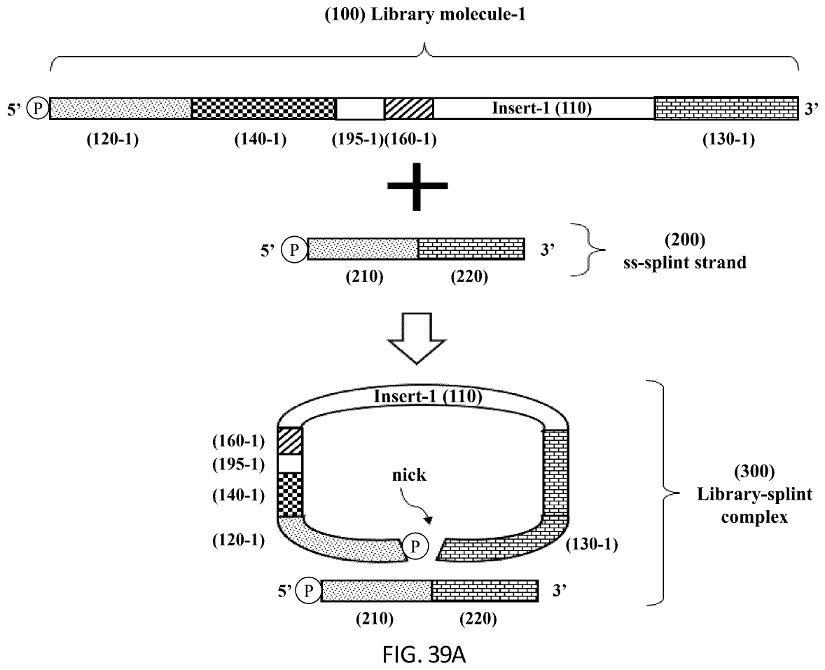

In some embodiments, e.g., in FIG. 38, the exemplary library molecule (100) comprises: surface pinning primer binding site sequence (120) (e.g., batch-specific surface pinning primer binding site sequence); a forward sequencing primer binding site sequence (140) (e.g., batch-specific forward sequencing primer binding site sequence (140); a batch-specific barcode sequence (195); a left sample index sequence (160); a sequence of interest (110); a reverse sequencing primer binding site sequence (150) (e.g., batch-specific reverse sequencing primer binding site sequence (150); a right sample index sequence (170); and a surface capture primer binding site sequence (130) (e.g., batch-specific surface capture primer binding site sequence (130). In some embodiments, the single-stranded splint strand (200) comprises a first region (210) that hybridizes with the surface pinning primer binding site sequence (120) of the linear single stranded library molecule (100), and a second region (220) that hybridizes with the surface capture primer binding site sequence (130) of the linear single stranded library molecule (100).

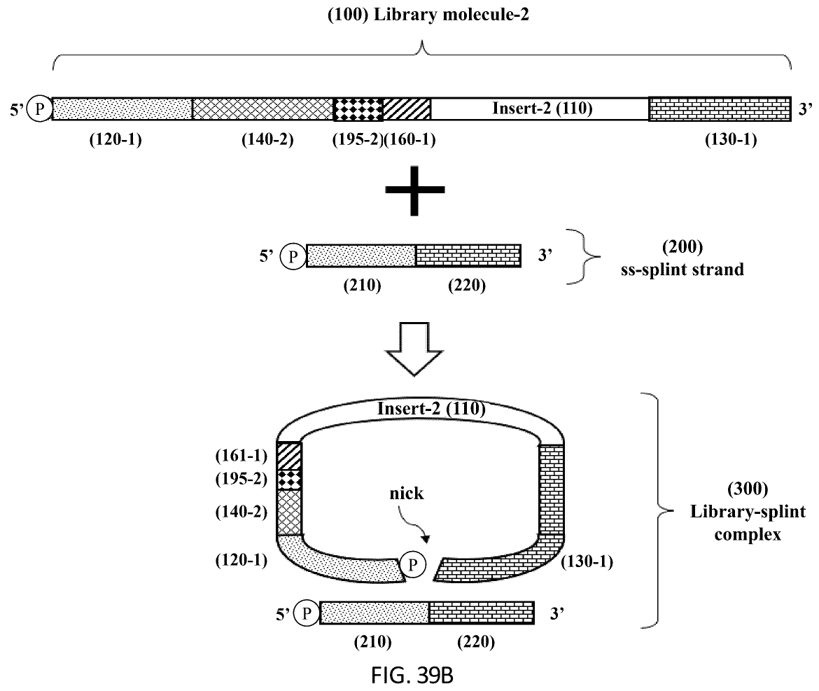

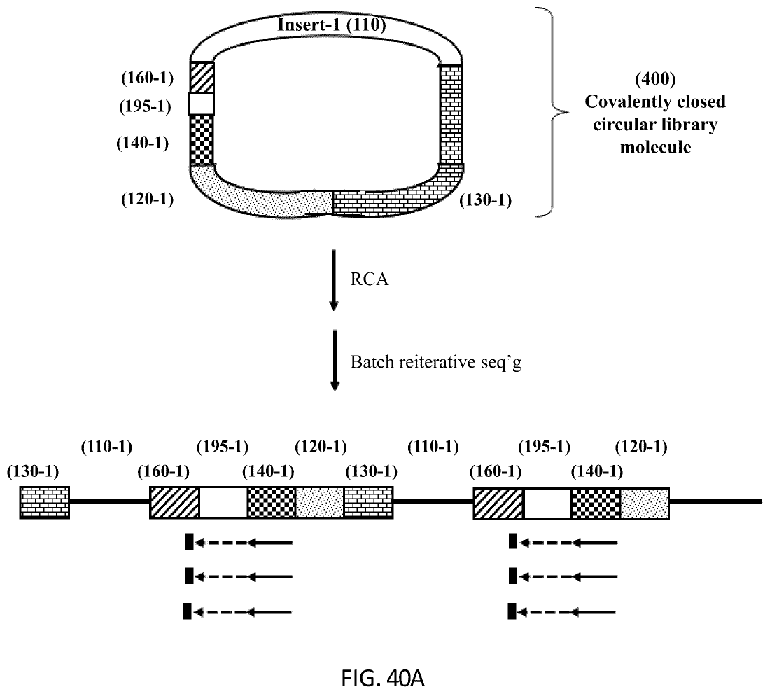

In some embodiments, e.g., in FIG. 39A, the exemplary library molecule (100) comprises: a first surface pinning primer binding site sequence (120-1); a first batch forward sequencing primer binding site sequence (140-1); a first batch barcode sequence (195-1); a first sample index sequence (160-1); a first sequence of interest (110-1); and a first surface capture primer binding site sequence (130-1). In some embodiments, the single-stranded splint strand (200) comprises a first region (210) that hybridizes with the first surface pinning primer binding site sequence (120-1) of the linear single stranded library molecule (100), and a second region (220) that hybridizes with the first surface capture primer binding site sequence (130-1) of the linear single stranded library molecule (100).

In some embodiments, e.g., in FIG. 39B, the exemplary library molecule (100) comprises: a first surface pinning primer binding site sequence (120-1); a second batch forward sequencing primer binding site sequence (140-2); a second batch barcode sequence (195-2); a first sample index sequence (160-1); a second sequence of interest (110-1); and a first surface capture primer binding site sequence (130-1). In some embodiments, the single-stranded splint strand (200) comprises a first region (210) that hybridizes with the first surface pinning primer binding site sequence (120-1) of the linear single stranded library molecule (100), and a second region (220) that hybridizes with the first surface capture primer binding site sequence (130-1) of the linear single stranded library molecule (100). In some embodiments, the first sequence of interest in the library-splint complex shown in FIG. 39A and the second sequence of interest in the library-splint complex shown in FIG. 39B have the same or different sequence.

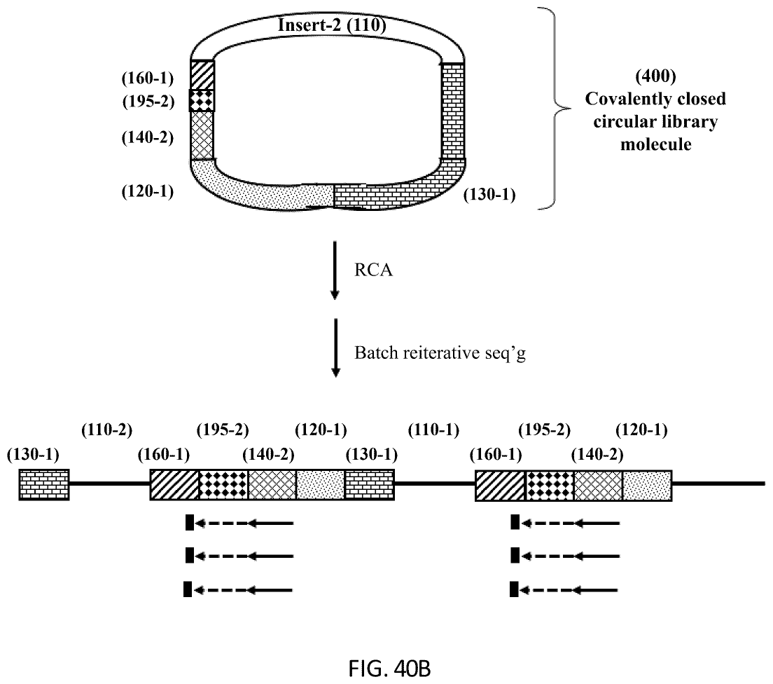

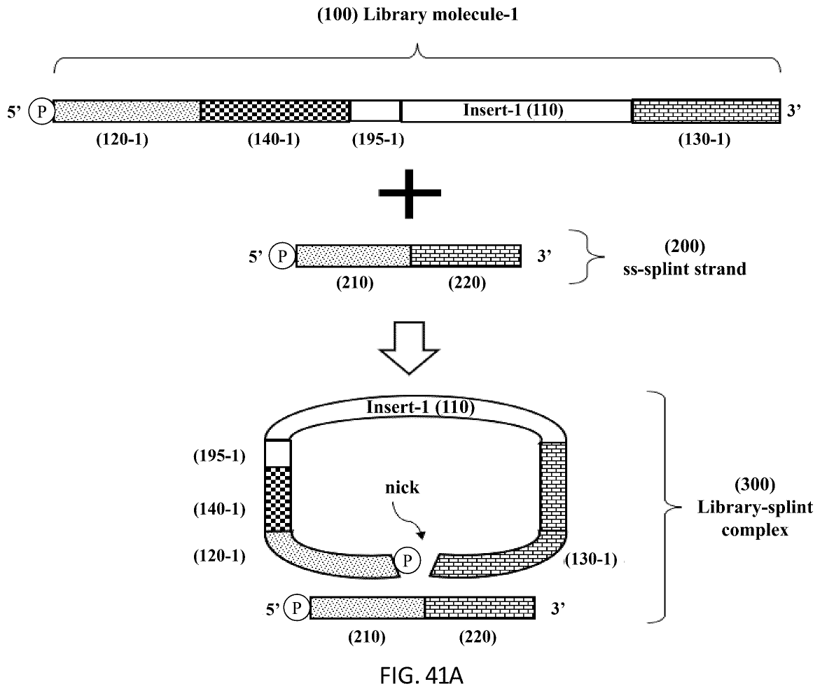

In some embodiments, e.g., FIG. 40A, the first covalently closed circular library molecule (400) is subjected to rolling circle amplification to generate a first concatemer template molecule, and the concatemer template molecule is subjected to batch reiterative sequencing. In some embodiments, the rolling circle amplification reaction can be conducted in-solution using soluble amplification primers or on-support using capture primers immobilized to a support. In some embodiments, the first covalently closed circular library molecule (400) comprises: a first surface pinning primer binding site sequence (120-1); a first batch forward sequencing primer binding site sequence (140-1) which corresponds with the first sequence of interest; a first batch barcode sequence (195-1) which corresponds with the first sequence of interest; a first sample index sequence (160-1); a first sequence of interest (110-1); and a first surface capture primer binding site sequence (130-1). In some embodiments, a plurality of the first covalently closed circular library molecule (400) shown in FIG. 40A are distributed onto a support having one type of immobilized capture primers which selectively hybridizes to the first capture primer binding site sequence (120-1) in the first covalently closed circular library molecules (400). In some embodiments, the first covalently closed circular library molecules (400) are subjected to rolling circle amplification (RCA) to generate a plurality of first concatemer template molecules which are immobilized to the support. In some embodiments, the first concatemer template molecules are subjected to a sequencing workflow using first batch-specific sequencing primers (solid arrows), sequencing polymerases, and a plurality of nucleotide reagents to generate a plurality of first sequencing read products (dashed arrows). In some embodiments, the first sequencing read products include the first batch barcode sequence (195-1) as shown in FIG. 40A. In some embodiments, the first sequencing read products include the first batch barcode sequence (195-1) and the first sample index sequence (160-1) (not shown). In some embodiments, the first sequencing read products include the first batch barcode sequence (195-1), the first sample index sequence (160-1), and at least a portion of the first sequence of interest (not shown). In some embodiments, the first concatemers undergo reiterative sequencing comprising up to 1000 sequencing cycles. In some embodiments, the first concatemers undergo reiterative sequencing comprising no more than 150 sequencing cycles.

In some embodiments, e.g., in FIG. 40B, the second covalently closed circular library molecule (400) is subjected to rolling circle amplification to generate a second concatemer template molecule, and the concatemer template molecule is subjected to batch reiterative sequencing. In some embodiments, the rolling circle amplification reaction can be conducted in-solution using soluble amplification primers or on-support using capture primers immobilized to a support. In some embodiments, the second covalently closed circular library molecule (400) comprises: a first surface pinning primer binding site sequence (120-1); a second batch forward sequencing primer binding site sequence (140-2) which corresponds with the second sequence of interest; a second batch barcode sequence (195-2) which corresponds with the second sequence of interest; a first sample index sequence (160-1); a second sequence of interest (110-2); and a first surface capture primer binding site sequence (130-1). In some embodiments, a plurality of the second covalently closed circular library molecule (400) shown in FIG. 40B are distributed onto a support having one type of immobilized capture primers which selectively hybridizes to the first capture primer binding site sequence (120-1) in the second covalently closed circular library molecules (400). In some embodiments, a plurality of the first covalently closed circular library molecule (400) shown in FIG. 40A, and a plurality of the second covalently closed circular library molecule (400) shown in FIG. 40B, are distributed onto the same support. In some embodiments, the first covalently closed circular library molecules (400) shown in FIG. 40A and the second covalently closed circular library molecules (400) shown in FIG. 40B can be distributed onto the support essentially simultaneously. In some embodiments, the first covalently closed circular library molecules (400) shown in FIG. 40A and the second covalently closed circular library molecules (400) shown in FIG. 40B can be distributed onto the support sequentially (e.g., re-seeding the support). In some embodiments, the second covalently closed circular library molecules (400) are subjected to rolling circle amplification (RCA) to generate a plurality of second concatemer template molecules which are immobilized to the support. In some embodiments, the second concatemer template molecules are subjected to a sequencing workflow using second batch sequencing primers (solid arrows), sequencing polymerases, and a plurality of nucleotide reagents to generate a plurality of second sequencing read products (dashed arrows). In some embodiments, the second concatemer template molecules are not sequenced when first batch sequencing primers are used to sequence the first concatemer template molecules. In some embodiments, the first concatemer template molecules are not sequenced when second batch sequencing primers are used to sequence the second concatemer template molecules. In some embodiments, the second sequencing read products include the second batch barcode sequence (195-2) as shown in FIG. 40B. In some embodiments, the second sequencing read products include the second batch barcode sequence (195-2) and the first sample index sequence (160-1) (not shown). In some embodiments, the second sequencing read products include the second batch barcode sequence (195-2), the first sample index sequence (160-1), and at least a portion of the second sequence of interest (not shown). In some embodiments, the second concatemer template molecules undergo reiterative sequencing comprising up to 1000 sequencing cycles. In some embodiments, the second concatemer template molecules undergo reiterative sequencing comprising no more than 150 sequencing cycles.

In some embodiments, the exemplary library molecule (100) comprises: a first surface pinning primer binding site sequence (120-1); a first batch forward sequencing primer binding site sequence (140-1); a first batch barcode sequence (195-1); a first sequence of interest (110-1); and a first surface capture primer binding site sequence (130-1). In some embodiments, the single-stranded splint strand (200) comprises a first region (210) that hybridizes with the first surface pinning primer binding site sequence (120-1) of the linear single stranded library molecule (100), and a second region (220) that hybridizes with the first surface capture primer binding site sequence (130-1) of the linear single stranded library molecule (100).

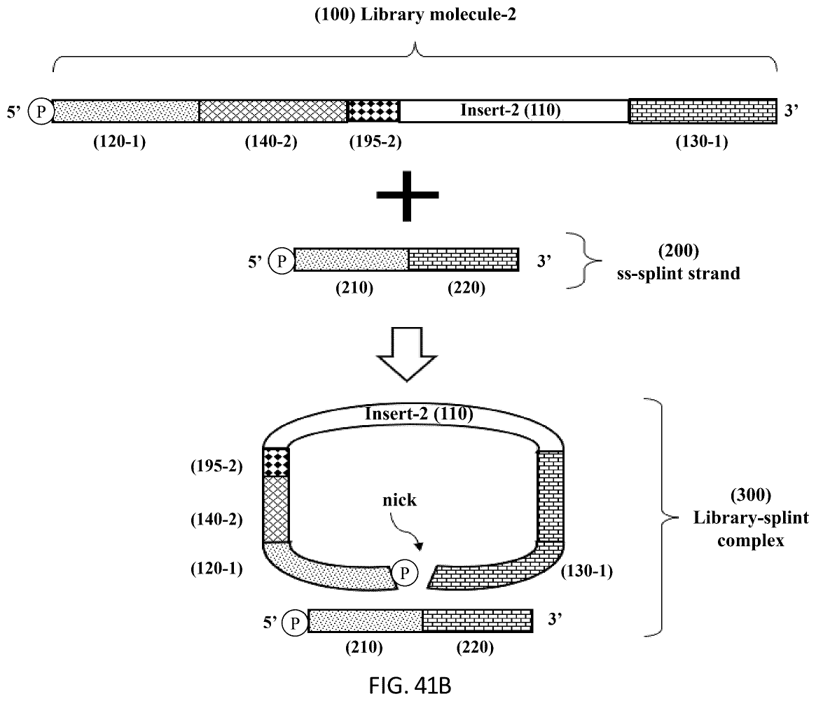

In some embodiments, e.g., in FIG. 41A, the exemplary library molecule (100) comprises: a first surface pinning primer binding site sequence (120-1); a first batch forward sequencing primer binding site sequence (140-1); a first batch barcode sequence (195-1); a first sequence of interest (110-1); and a first surface capture primer binding site sequence (130-1). In some embodiments, the single-stranded splint strand (200) comprises a first region (210) that hybridizes with the first surface pinning primer binding site sequence (120-1) of the linear single stranded library molecule (100), and a second region (220) that hybridizes with the first surface capture primer binding site sequence (130-1) of the linear single stranded library molecule (100).

In some embodiments, e.g., as in FIG. 41B, the exemplary library molecule (100) comprises: a first surface pinning primer binding site sequence (120-1); a second batch forward sequencing primer binding site sequence (140-2); a second batch barcode sequence (195-2); a second sequence of interest (110-1); and a first surface capture primer binding site sequence (130-1). In some embodiments, the single-stranded splint strand (200) comprises a first region (210) that hybridizes with the first surface pinning primer binding site sequence (120-1) of the linear single stranded library molecule (100), and a second region (220) that hybridizes with the first surface capture primer binding site sequence (130-1) of the linear single stranded library molecule (100). In some embodiments, the first sequence of interest in the library-splint complex shown in FIG. 41A and the second sequence of interest in the library-splint complex shown in FIG. 41B have the same or different sequence.

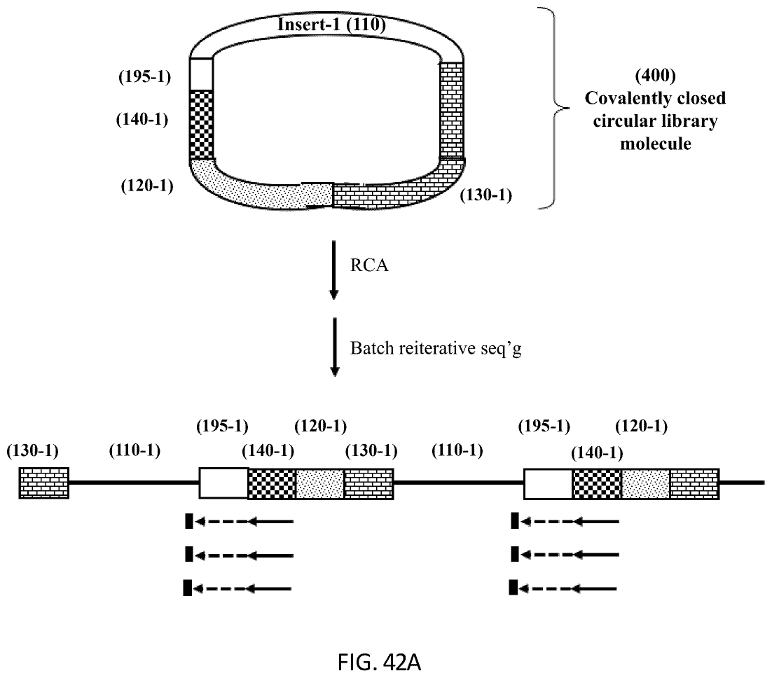

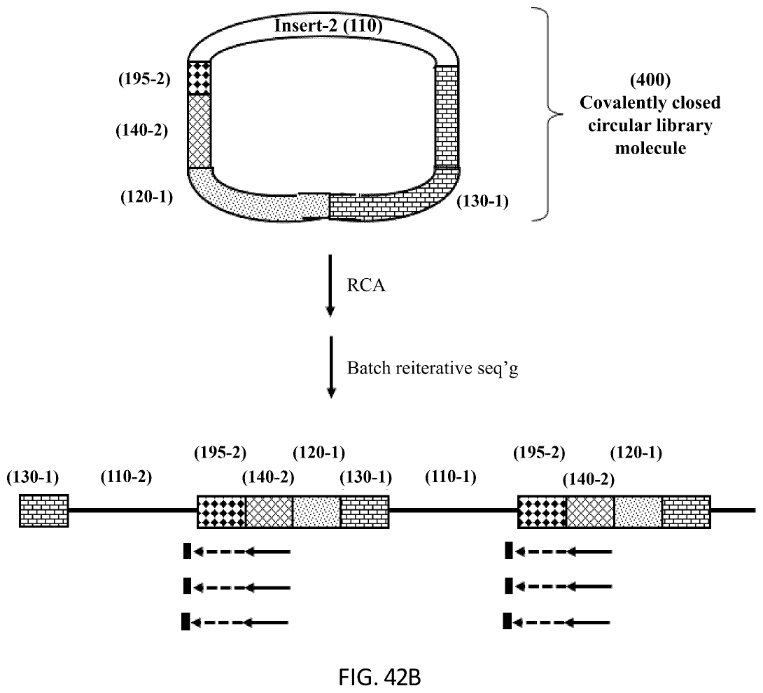

In some embodiments, e.g., as in FIG. 42A, the first covalently closed circular library molecule (400) is subjected to rolling circle amplification to generate a first concatemer template molecule, and the concatemer template molecule is subjected to batch reiterative sequencing. In some embodiments, the rolling circle amplification reaction can be conducted in-solution using soluble amplification primers or on-support using capture primers immobilized to a support. In some embodiments, the first covalently closed circular library molecule (400) comprises: a first surface pinning primer binding site sequence (120-1); a first batch forward sequencing primer binding site sequence (140-1) which corresponds with the first sequence of interest; a first batch barcode sequence (195-1) which corresponds with the first sequence of interest; a first sequence of interest (110-1); and a first surface capture primer binding site sequence (130-1). In some embodiments, a plurality of the first covalently closed circular library molecule (400) shown in FIG. 42A are distributed onto a support having one type of immobilized capture primers which selectively hybridizes to the first capture primer binding site sequence (120-1) in the first covalently closed circular library molecules (400). In some embodiments, the first covalently closed circular library molecules (400) are subjected to rolling circle amplification (RCA) to generate a plurality of first concatemer template molecules which are immobilized to the support. In some embodiments, the first concatemer template molecules are subjected to a sequencing workflow using first batch-specific sequencing primers (solid arrows), sequencing polymerases, and a plurality of nucleotide reagents to generate a plurality of first sequencing read products (dashed arrows). In some embodiments, the first sequencing read products include the first batch barcode sequence (195-1) as shown in FIG. 42A. In some embodiments, the first sequencing read products include the first batch barcode sequence (195-1) and at least a portion of the first sequence of interest (not shown). In some embodiments, the first concatemers undergo reiterative sequencing comprising up to 1000 sequencing cycles. In some embodiments, the first concatemers undergo reiterative sequencing comprising no more than 150 sequencing cycles.

In some embodiments, e.g., as in FIG. 42B, the second covalently closed circular library molecule (400) is subjected to rolling circle amplification to generate a second concatemer template molecule, and the concatemer template molecule is subjected to batch reiterative sequencing. In some embodiments, the rolling circle amplification reaction can be conducted in-solution using soluble amplification primers or on-support using capture primers immobilized to a support. In some embodiments, the second covalently closed circular library molecule (400) comprises: a first surface pinning primer binding site sequence (120-1); a second batch forward sequencing primer binding site sequence (140-2) which corresponds with the second sequence of interest; a second batch barcode sequence (195-2) which corresponds with the second sequence of interest; a second sequence of interest (110-2); and a first surface capture primer binding site sequence (130-1). In some embodiments, a plurality of the second covalently closed circular library molecule (400) shown in FIG. 42B are distributed onto a support having one type of immobilized capture primers which selectively hybridizes to the first capture primer binding site sequence (120-1) in the second covalently closed circular library molecules (400).

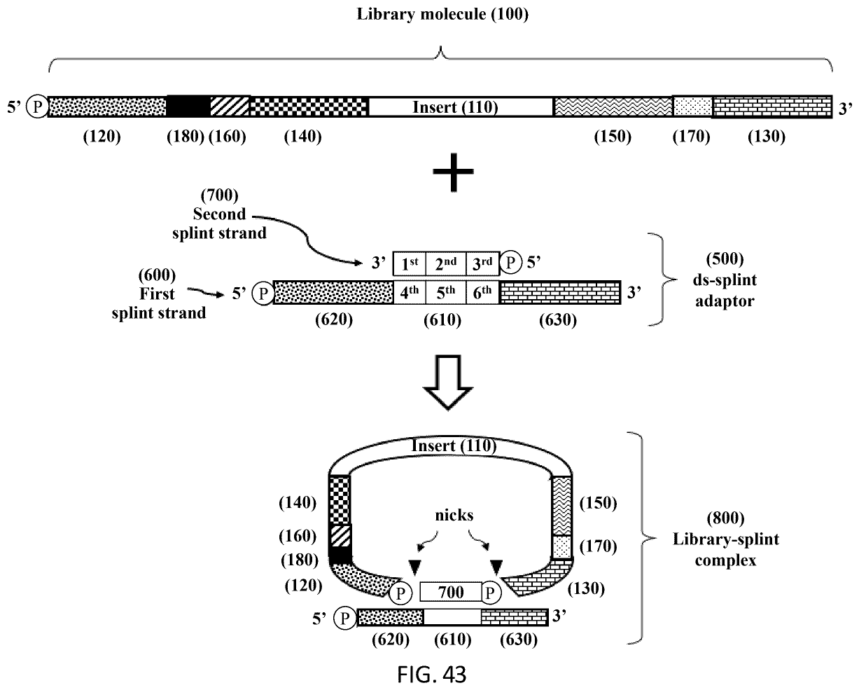

In some embodiments, a plurality of the first covalently closed circular library molecule (400) shown in FIG. 43A, and a plurality of the second covalently closed circular library molecule (400) shown in FIG. 42B, are distributed onto the same support. In some embodiments, the first covalently closed circular library molecules (400) shown in FIG. 42A and the second covalently closed circular library molecules (400) shown in FIG. 42B can be distributed onto the support essentially simultaneously. In some embodiments, the first covalently closed circular library molecules (400) shown in FIG. 42A and the second covalently closed circular library molecules (400) shown in FIG. 42B can be distributed onto the support sequentially (e.g., re-seeding the support). In some embodiments, the second covalently closed circular library molecules (400) are subjected to rolling circle amplification (RCA) to generate a plurality of second concatemer template molecules which are immobilized to the support. In some embodiments, the second concatemer template molecules are subjected to a sequencing workflow using second batch sequencing primers (solid arrows), sequencing polymerases, and a plurality of nucleotide reagents to generate a plurality of second sequencing read products (dashed arrows). In some embodiments, the second concatemer template molecules are not sequenced when first batch sequencing primers are used to sequence the first concatemer template molecules. In some embodiments, the first concatemer template molecules are not sequenced when second batch sequencing primers are used to sequence the second concatemer template molecules. In some embodiments, the second sequencing read products include the second batch barcode sequence (195-2) as shown in FIG. 42B. In some embodiments, the second sequencing read products include the second batch barcode sequence (195-2) and at least a portion of the second sequence of interest (not shown). In some embodiments, the second concatemer template molecules undergo reiterative sequencing comprising up to 1000 sequencing cycles. In some embodiments, the second concatemer template molecules undergo reiterative sequencing comprising no more than 150 sequencing cycles.

In some embodiments, the method for generating circularized library molecules further comprises step (d): ligating the nick in the plurality of library-splint complexes (300) thereby generating a plurality of a plurality of covalently closed circular library molecules (400) each hybridized to a single-stranded splint strand (200).

In some embodiments, the methods for generating circularized library molecules described herein can further comprises at least one enzymatic reaction, including a phosphorylation reaction, ligation reaction and/or exonuclease reaction. The enzymatic reactions can be conducted sequentially or essentially simultaneously. The enzymatic reactions can be conducted in a single reaction vessel. Alternatively, a first enzymatic reaction can be conducted in a first reaction vessel, then transferred to a second reaction vessel where the second enzymatic reaction is conducted, then transferred to a third reaction vessel where the third enzymatic reaction is conducted.

In some embodiments, the methods for generating circularized library molecules described herein further comprise conducting separate and sequential phosphorylation and ligation reactions which are conducted in separate reaction vessels. In some embodiments, the methods for generating circularized library molecules further comprise step (c1): contacting in a first reaction vessel the plurality of the single-stranded splint strands (200) and the plurality of the single-stranded nucleic acid library molecules (100) with a T4 polynucleotide kinase enzyme under a condition suitable to phosphorylate 5′ ends of the plurality of single-stranded splint strands (200) and/or the plurality of single-stranded nucleic acid library molecules (100); and transferring the phosphorylation reaction to a second reaction vessel. In some embodiments, the methods for generating circularized library molecules further comprise step (d1): contacting in the second reaction vessel the plurality of phosphorylated single-stranded splint strands (200) and the plurality of phosphorylated single-stranded nucleic acid library molecules (100) with a ligase, under a condition suitable to enzymatically ligate the nicks, thereby generating a plurality of covalently closed circular library molecules (400) each hybridized to a single-stranded splint strand (200). In some embodiments, the ligase enzyme comprises T7 DNA ligase, T3 ligase, T4 ligase, or Taq ligase.

In some embodiments, the methods for generating circularized library molecules described herein further comprise conducting sequential phosphorylation and ligation reactions which are conducted sequentially in the same reaction vessel. In some embodiments, the methods for generating circularized library molecules further comprise step (c2): contacting in a first reaction vessel the plurality of the single-stranded splint strands (200) and the plurality of the single-stranded nucleic acid library molecules (100) with a T4 polynucleotide kinase enzyme under a condition suitable to phosphorylate 5′ ends of the plurality of single-stranded splint strands (200) and the plurality of single-stranded nucleic acid library molecules (100). In some embodiments, the methods for generating circularized library molecules further comprise step (d2): contacting in the same first reaction vessel the phosphorylated single-stranded splint strands (200) and the phosphorylated single-stranded nucleic acid library molecules (100) with a ligase under a condition suitable to enzymatically ligate the nicks, thereby generating a plurality of covalently closed circular library molecules (400) each hybridized to a single-stranded splint strand (200). In some embodiments, the ligase enzyme comprises T7 DNA ligase, T3 ligase, T4 ligase, or Taq ligase.

In some embodiments, the methods for generating circularized library molecules described herein further comprise conducting essentially simultaneous phosphorylation and ligation reactions which are conducted together in the same reaction vessel. In some embodiments, the methods for generating circularized library molecules further comprise step (c3): contacting in a first reaction vessel the plurality of the single-stranded splint strands (200) and the plurality of the single-stranded nucleic acid library molecules (100) with a (i) T4 polynucleotide kinase enzyme and (ii) a ligase enzyme, under a condition suitable to phosphorylate 5′ ends of the plurality of single-stranded splint strands (200) and the plurality of single-stranded nucleic acid library molecules (100), and the conditions are suitable to enzymatically ligate the nicks, thereby generating a plurality of covalently closed circular library molecules (400) each hybridized to a single-stranded splint strand (200). In some embodiments, the ligase enzyme comprises T7 DNA ligase, T3 ligase, T4 ligase, or Taq ligase.

In some embodiments, the methods for generating circularized library molecules further comprise the optional step of enzymatically removing the plurality of single-stranded splint strands (200) from the plurality of covalently closed circular library molecules (400), which comprises the step: contacting the plurality of covalently closed circular library molecules (400) with at least one exonuclease enzyme to remove the plurality of single-stranded splint strands (200) and retaining the plurality of covalently closed circular library molecules (400). In some embodiments, the exonuclease reaction can be conducted in the same reaction buffer used to conduct the phosphorylation and/or ligation reactions, or in a different reaction buffer. In some embodiments, the exonuclease reaction can be conducted in a third reaction vessel after conducting the phosphorylation reaction in the first reaction vessel (step c1, see above), and conducting the ligation reaction in the second reaction vessel (step d1, see above). In some embodiments, the exonuclease reaction can be conducted in the first reaction vessel after conducting the phosphorylation reaction in the first reaction vessel (step c2, see above), and conducting the sequential ligation reaction in the first reaction vessel (step d2, see above). In some embodiments, the exonuclease reaction can be conducted in the first reaction vessel after conducting the essentially simultaneous phosphorylation and ligation reactions in the first reaction vessel (step c3, see above). In some embodiments, the at least one exonuclease enzyme comprises any combination of two or more of exonuclease I, thermolabile exonuclease I and/or T7 exonuclease.

In some embodiments, the covalently closed circular library molecules (400) can be subjected to rolling circle amplification and sequencing (e.g., batch sequencing) which are described below.

In some embodiments, the pinning primer binding site sequence (120) in the library molecules comprise the sequence 5′-CATGTAATGCACGTACTTTCAGGGT-3′ (SEQ ID NO: 1).

In some embodiments, the pinning primer binding site sequence (120) in the library molecules comprise the sequence 5′-AATGATACGGCGACCACCGA-3′ (SEQ ID NO: 2).

In some embodiments, the forward sequencing primer binding site sequence (140) in the library molecules comprise the sequence 5′-CGTGCTGGATTGGCTCACCAGACACCTTCCGACAT-3′ (SEQ ID NO: 3).

In some embodiments, the forward sequencing primer binding site sequence (140) in the library molecules comprise the sequence 5′-ACACTCTTTCCCTACACGACGCTCTTCCGATCT-3′ (SEQ ID NO: 4).

In some embodiments, the forward sequencing primer binding site sequence (140) in the library molecules comprise the sequence 5′-TCGTCGGCAGCGTCAGATGTGTATAAGAGACAG-3′ (SEQ ID NO: 5).

In some embodiments, the reverse sequencing primer binding site sequence (150) in the library molecules comprise the sequence 5′-ATGTCGGAAGGTGTGCAGGCTACCGCTTGTCAACT-3′ (SEQ ID NO: 6).

In some embodiments, the reverse sequencing primer binding site sequence (150) in the library molecules comprise the sequence 5′-AGATCGGAAGAGCACACGTCTGAACTCCAGTCAC-3′ (SEQ ID NO: 7).

In some embodiments, the reverse sequencing primer binding site sequence (150) in the library molecules comprise the sequence 5′-CTGTCTCTTATACACATCTCCGAGCCCACGAGAC-3′ (SEQ ID NO: 8).

In some embodiments, the capture primer binding site sequence (130) in the library molecules comprise the sequence 5′-AGTCGTCGCAGCCTCACCTGATC-3′ (SEQ ID NO: 9).

In some embodiments, the capture primer binding site sequence (130) in the library molecules comprise the sequence 5′-TCGTATGCCGTCTTCTGCTTG-3′ (SEQ ID NO: 10).

Generating Circularized Library Molecules using Double-Stranded Splint Adaptors

The present disclosure provides reagents, kits and methods for preparing circularized library molecules. In some embodiments, the circularized library molecules are prepared by hybridizing any of the linear library molecules described herein with a plurality of double-stranded splint strands (500) to generate a plurality of library-splint complexes (800) which includes two nicks (e.g., see FIGS. 43, 44, 45, 46A and 46B). The nicks can be enzymatically ligated to generate covalently closed circular molecules (900) in which the second splint strand (700) is covalently joined at both ends to the library molecule (100), thereby introducing the new adaptor sequences into the circularized library molecule.

The present disclosure provides methods for forming a plurality of library-splint complexes (800) comprising: (a) providing a plurality of single-stranded nucleic acid library molecule (100) wherein individual library molecules comprise: (i) a left universal adaptor sequence having a binding sequence for a first surface primer (120); (ii) a left universal adaptor sequence having a binding sequence for a first sequencing primer (140); (iii) a sequence of interest (110); (iv) a right universal adaptor sequence having a binding sequence for a second sequencing primer (150); and (v) a right universal adaptor sequence (130) having a binding sequence for a second surface primer (130). In some embodiments, the left universal adaptor sequence (120) comprises a binding sequence for a first surface primer P5. In some embodiments, the right universal adaptor sequence (130) comprises a binding sequence for a second surface primer P7. In some embodiments, the linear library further comprises a left sample index sequence (160) and/or a right sample index sequence (170). The left and right sample index sequences can be used to distinguish insert sequences that are isolated from different sample sources in a multiplex assay. The left index sequence (160) can include a random sequence (e.g., NNN) or lack a random sequence. The right index sequence (170) can include a random sequence (e.g., NNN) or lack a random sequence. Exemplary single-stranded library molecules are shown in (e.g., see FIGS. 43, 44, 45, 46A and 46B).

The methods for forming a plurality of library-splint complexes (800) further comprise step (b): hybridizing the plurality of single-stranded nucleic acid library molecules (100) with a plurality of double-stranded splint adaptors (500), wherein individual double-stranded splint adaptors (500) in the plurality comprise a first splint strand (600) hybridized to a second splint strand (700), wherein the double-stranded splint adaptor includes a double-stranded region and two flanking single-stranded regions, wherein the first splint strand comprises a first region (620), an internal region (610), and a second region (630), and wherein the internal region of the first splint strand (610) is hybridized to the second splint strand (700). In some embodiments, the first splint strand (600) comprises regions arranged in a 5′ to 3′ order a first region (620), an internal region (610), and a second region (630), and wherein the internal region of the first splint strand (610) is hybridized to the second splint strand (700). In some embodiments, the second splint strand (700) comprises regions arranged in a 5′ to 3′ order (i) a second sub-region having a universal binding sequence for a fourth surface primer, and (ii) a first sub-region having a universal binding sequence for a third surface primer. The universal binding sequences for the third surface primer do not bind the first surface primer (e.g., P5) or the second surface primer (e.g., P7). The universal binding sequences for the fourth surface primer do not bind the first surface primer (e.g., P5) or the second surface primer (e.g., P7). Exemplary double-stranded splint adaptors (500) are shown in (e.g., see FIGS. 43, 44, 45, 46A and 46B).

The hybridizing of step (b) is conducted under a condition suitable for hybridizing the first region of the first splint strand (620) to the at least first left universal adaptor sequence (120) of the library molecule, and the condition is suitable for hybridizing the second region of the first splint strand (630) to the at least first right universal sequence (130) of the library molecule, thereby circularizing the plurality of library molecules to form a plurality of library-splint complexes (800). The library-splint complex (800) comprises a first nick between 5′ end of the library molecule and 3′ end of the second splint strand (e.g., see FIGS. 43, 44, 45, 46A and 46B). The library-splint complex (800) also comprises a second nick between 5′ end of the second splint strand and 3′ end of the library molecule (e.g., see FIGS. 43, 44, 45, 46A and 47B). In some embodiments, the first and second nicks are enzymatically ligatable.

In some embodiments, e.g., as shown in FIG. 43, the exemplary library molecule (100) comprises: a pinning primer binding site sequence (120) (e.g., batch-specific pinning primer binding site sequence); an optional left unique identification sequence (180) (e.g., UMI); a left sample index sequence (160); a forward sequencing primer binding site sequence (140) (e.g., batch-specific forward sequencing primer binding site sequence (140); a sequence of interest (110); a reverse sequencing primer binding site sequence (150) (e.g., batch-specific reverse sequencing primer binding site sequence (150); a right sample index sequence (170); and a surface capture primer binding site sequence (130) (e.g., batch-specific surface capture primer binding site sequence (130). In some embodiments, the double-stranded adaptor comprises a first splint strand (600) hybridized to a second splint strand (700). In the double-stranded adaptor, the first splint strand (600) comprises a first region (620), an internal region (610), and a second region (630), wherein the internal region of the first splint strand (610) is hybridized to the second splint strand (700). In some embodiments, the first region of the first splint strand (620) can hybridize to at least a portion of the pinning primer binding site sequence (120) of a single-stranded nucleic acid library molecule (100), and the second region of the first splint strand (630) can hybridize to at least a portion of the capture primer binding site sequence (130) of the same single-stranded nucleic acid library molecule (100).

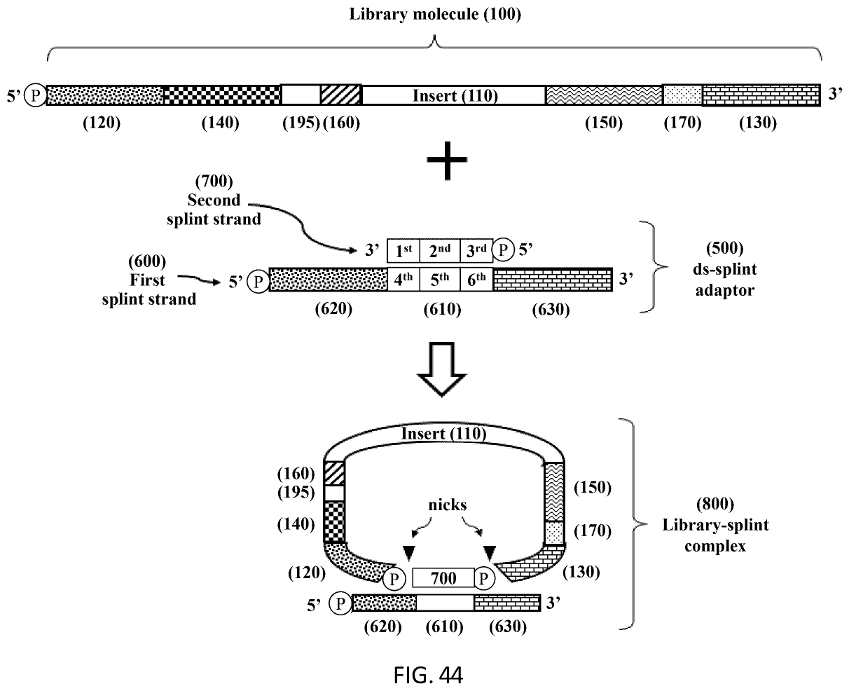

In some embodiments, e.g., in FIG. 44, the exemplary library molecule (100) comprises: a pinning primer binding site sequence (120) (e.g., batch-specific pinning primer binding site sequence); a forward sequencing primer binding site sequence (140) (e.g., batch-specific forward sequencing primer binding site sequence (140); a batch-specific barcode sequence (195); a left sample index sequence (160); a sequence of interest (110); a reverse sequencing primer binding site sequence (150) (e.g., batch-specific reverse sequencing primer binding site sequence (150); a right sample index sequence (170); and a surface capture primer binding site sequence (130) (e.g., batch-specific surface capture primer binding site sequence (130). In some embodiments, the double-stranded adaptor comprises a first splint strand (600) hybridized to a second splint strand (700). In the double-stranded adaptor, the first splint strand (600) comprises a first region (620), an internal region (610), and a second region (630), wherein the internal region of the first splint strand (610) is hybridized to the second splint strand (700). In some embodiments, the first region of the first splint strand (620) can hybridize to at least a portion of the pinning primer binding site sequence (120) of a single-stranded nucleic acid library molecule (100), and the second region of the first splint strand (630) can hybridize to at least a portion of the capture primer binding site sequence (130) of the same single-stranded nucleic acid library molecule (100)

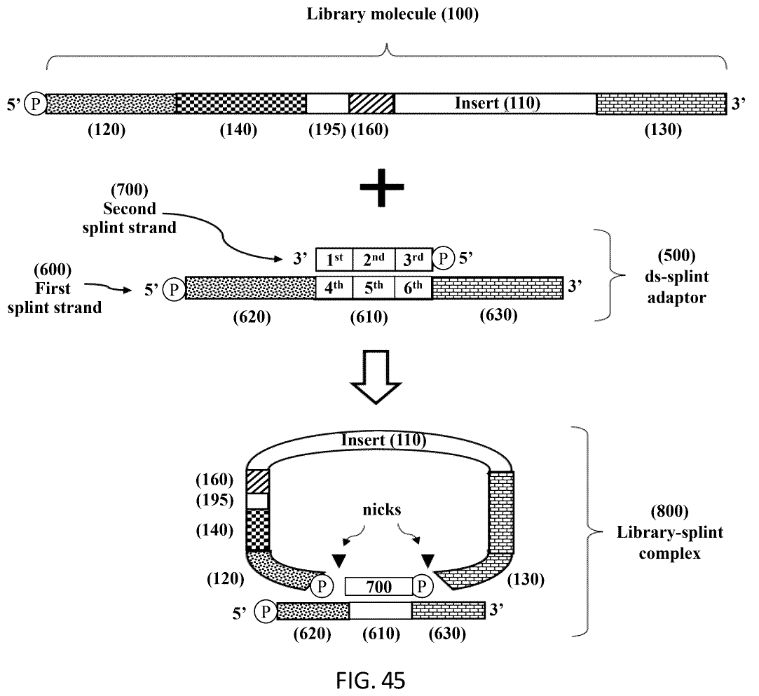

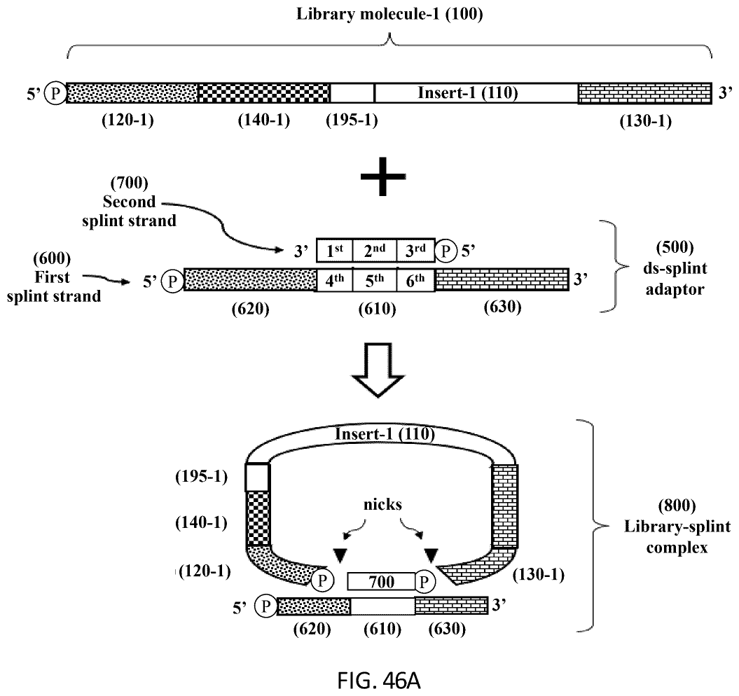

In some embodiments, e.g., as in FIG. 45, the exemplary library molecule (100) comprises: a pinning primer binding site sequence (120) (e.g., batch-specific pinning primer binding site sequence); a forward sequencing primer binding site sequence (140) (e.g., batch-specific forward sequencing primer binding site sequence (140); a batch-specific barcode sequence (195); a left sample index sequence (160); a sequence of interest (110); and a surface capture primer binding site sequence (130) (e.g., batch-specific surface capture primer binding site sequence (130). In some embodiments, the double-stranded adaptor comprises a first splint strand (600) hybridized to a second splint strand (700). In the double-stranded adaptor, the first splint strand (600) comprises a first region (620), an internal region (610), and a second region (630), wherein the internal region of the first splint strand (610) is hybridized to the second splint strand (700). In some embodiments, the first region of the first splint strand (620) can hybridize to at least a portion of the pinning primer binding site sequence (120) of a single-stranded nucleic acid library molecule (100), and the second region of the first splint strand (630) can hybridize to at least a portion of the capture primer binding site sequence (130) of the same single-stranded nucleic acid library molecule (100).

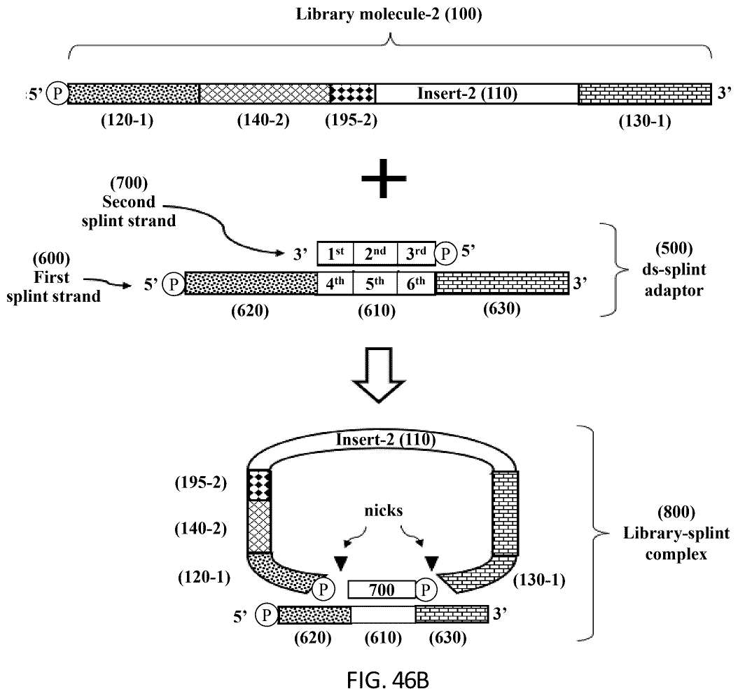

In some embodiments, e.g., in FIG. 46A, the exemplary library molecule-1 (100) comprises: a first pinning primer binding site sequence (120-1); a first batch forward sequencing primer binding site sequence (140-1); a first batch barcode sequence (195-1); a first sequence of interest (110-1); and a first surface capture primer binding site sequence (130-1). In some embodiments, the double-stranded adaptor comprises a first splint strand (600) hybridized to a second splint strand (700). In the double-stranded adaptor, the first splint strand (600) comprises a first region (620), an internal region (610), and a second region (630), wherein the internal region of the first splint strand (610) is hybridized to the second splint strand (700). In some embodiments, the first region of the first splint strand (620) can hybridize to at least a portion of the pinning primer binding site sequence (120) of a single-stranded nucleic acid library molecule (100), and the second region of the first splint strand (630) can hybridize to at least a portion of the capture primer binding site sequence (130) of the same single-stranded nucleic acid library molecule (100).

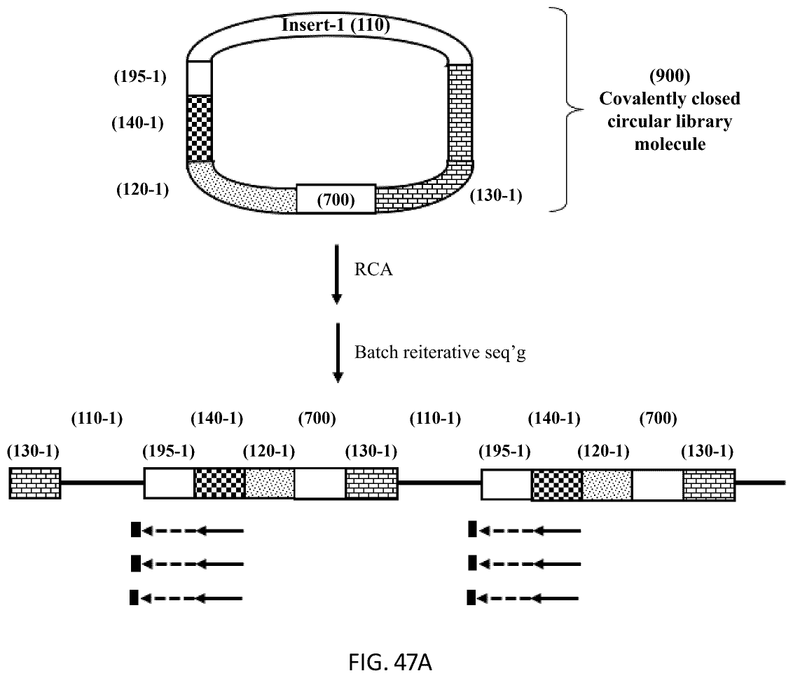

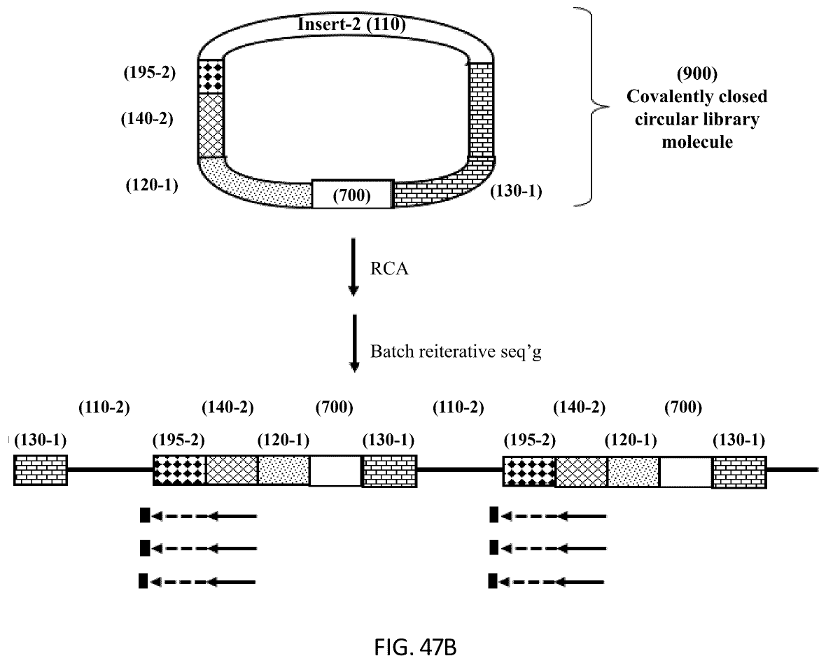

In some embodiments, e.g., as in FIG. 47A, the exemplary library molecule-2 (100) comprises: a first pinning primer binding site sequence (120-1); a second batch forward sequencing primer binding site sequence (140-2); a second batch barcode sequence (195-2); a second sequence of interest (110-2); and a first surface capture primer binding site sequence (130-1). In some embodiments, the double-stranded adaptor comprises a first splint strand (600) hybridized to a second splint strand (700). In the double-stranded adaptor, the first splint strand (600) comprises a first region (620), an internal region (610), and a second region (630), wherein the internal region of the first splint strand (610) is hybridized to the second splint strand (700). In some embodiments, the first region of the first splint strand (620) can hybridize to at least a portion of the pinning primer binding site sequence (120) of a single-stranded nucleic acid library molecule (100), and the second region of the first splint strand (630) can hybridize to at least a portion of the capture primer binding site sequence (130) of the same single-stranded nucleic acid library molecule (100). In some embodiments, the first sequence of interest in the library-splint complex shown in FIG. 46A and the second sequence of interest in the library-splint complex shown in FIG. 46B have the same or different sequence.

In some embodiments, in the methods for forming a plurality of library-splint complexes (800), 5′ end of the first splint strand (600) is phosphorylated or lacks a phosphate group. In some embodiments, 3′ end of the first splint strand (600) includes a terminal 3′ OH group or a terminal 3′ blocking group.

In some embodiments, in the methods for forming a plurality of library-splint complexes (800), 5′ end of the second splint strand (700) is phosphorylated or lacks a phosphate group. In some embodiments, 3′ end of the second splint strand (700) includes a terminal 3′ OH group or a terminal 3′ blocking group.

In some embodiment, in the methods for forming a plurality of library-splint complexes (800), the first region of the first splint strand (620) can hybridize to a sense or anti-sense strand of a double-stranded nucleic acid library molecule. In the library-splint complex (800), the second region of the first splint strand (630) can hybridize to a sense or anti-sense strand of a double-stranded nucleic acid library molecule. The double-stranded nucleic acid library molecule can be denatured to generate the single-stranded sense and anti-sense library strands.

In some embodiments, in the methods for forming a plurality of library-splint complexes (800), the second splint strand (700) does not hybridize to the sequence of interest (110), and the internal region of the first splint strand (610) does not hybridize to the sequence of interest (110).

In some embodiments, in the methods for forming a plurality of library-splint complexes (800), the first region of the first splint strand (620) does not hybridize to the sequence of interest (110), and the second region of the first splint strand (630) does not hybridize to the sequence of interest (110).

In some embodiments, in the methods for forming a plurality of library-splint complexes (800), 5′ end of the single-stranded library molecule (100) is phosphorylated or lacks a phosphate group. In some embodiments, 3′ end of the single-stranded library molecule includes a terminal 3′ OH group or a terminal 3′ blocking group.

The methods for forming a plurality of library-splint complexes (800) further comprise step (c): contacting the plurality of library-splint complexes (800) from step (b) with a ligase, under a condition suitable to enzymatically ligate the first and second nicks, thereby generating a plurality of covalently closed circular library molecules (900) each hybridized to the first splint strand (600). In some embodiments, the ligase enzyme comprises T7 DNA ligase, T3 ligase, T4 ligase, or Taq ligase.

The methods for forming a plurality of library-splint complexes (800) further comprise an optional step (d): enzymatically removing the plurality of first splint strands (600) from the plurality of covalently closed circular library molecules (900) by contacting the plurality of covalently closed circular library molecules (900) with at least one exonuclease enzyme to remove the plurality of first splint strands (600) and retaining the plurality of covalently closed circular library molecules (900). In some embodiments, the at least one exonuclease enzyme comprises any combination of two or more of exonuclease I, thermolabile exonuclease I and/or T7 exonuclease.

In some embodiments, the covalently closed circular library molecules (900) can be subjected to rolling circle amplification and sequencing (e.g., batch sequencing) which are described below.

In some embodiments, in any of the methods for forming a plurality of library-splint complexes (800) described herein, the library molecule includes a left universal binding sequence (120) which binds the first region of the first splint strand (620), where the left universal binding sequence (120) comprises the sequence 5′-AATGATACGGCGACCACCGA-3′ (SEQ ID NO: 11)

In any of the methods for forming a plurality of library-splint complexes (800) described herein, the library molecule includes a left universal binding sequence (120) comprising the sequence 5′-CATGTAATGCACGTACTTTCAGGGT-3′ (SEQ ID NO: 12).

In some embodiments, in any of the methods for forming a plurality of library-splint complexes (800) described herein, the library molecule includes a left universal binding sequence for a sequencing primer (140) where the left universal binding sequence comprises the sequence 5′-ACACTCTTTCCCTACACGACGCTCTTCCGATCT-3′ (SEQ ID NO: 13).

In some embodiments, in any of the methods for forming a plurality of library-splint complexes (800) described herein, the library molecule includes a left universal binding sequence for a sequencing primer (140) where the left universal binding sequence comprises the sequence 5′-TCGTCGGCAGCGTCAGATGTGTATAAGAGACAG-3′ (SEQ ID NO: 14).

In some embodiments, in any of the methods for forming a plurality of library-splint complexes (800) described herein, the library molecule includes a left universal binding sequence for a sequencing primer (140) where the left universal binding sequence comprises the sequence 5′-CGTGCTGGATTGGCTCACCAGACACCTTCCGACAT-3′ (SEQ ID NO: 15).

In some embodiments, in any of the methods for forming a plurality of library-splint complexes (800) described herein, the library molecule includes a left universal binding sequence for a sequencing primer (150) where the left universal binding sequence comprises the sequence 5′-AGATCGGAAGAGCACACGTCTGAACTCCAGTCAC-3′ (SEQ ID NO: 16).

In some embodiments, in any of the methods for forming a plurality of library-splint complexes (800) described herein, the library molecule includes a left universal binding sequence for a sequencing primer (150) where the left universal binding sequence comprises the sequence 5′-CTGTCTCTTATACACATCTCCGAGCCCACGAGAC-3′ (SEQ ID NO: 17).

In some embodiments, in any of the methods for forming a plurality of library-splint complexes (800) described herein, the library molecule includes a left universal binding sequence for a sequencing primer (150) where the left universal binding sequence comprises the sequence 5′-ATGTCGGAAGGTGTGCAGGCTACCGCTTGTCAACT-3′ (SEQ ID NO: 18).

In some embodiments, in any of the methods for forming a plurality of library-splint complexes (800) described herein, the library molecule includes a right universal binding sequence (130) which binds the first region of the first splint strand (630), where the right universal binding sequence (130) comprises the sequence 5′-TCGTATGCCGTCTTCTGCTTG-3′ (SEQ ID NO: 19).

In some embodiments, in any of the methods for forming a plurality of library-splint complexes (800) described herein, the library molecule includes a right universal binding sequence (130) comprising the sequence 5′-AGTCGTCGCAGCCTCACCTGATC-3′ (SEQ ID NO: 20).

In some embodiments, in any of the methods for forming a plurality of library-splint complexes (800) described herein, the first sub-region of the second splint strand (700) comprises the sequence 5′-CATGTAATGCACGTACTTTCAGGGT-3′ (SEQ ID NO: 21). In some embodiments, the second sub-region of the second splint strand (700) comprises the sequence 5′-AGTCGTCGCAGCCTCACCTGATC-3′ (SEQ ID NO: 22). In some embodiments, the second splint strand (700) comprises a first and second sub-region comprising the sequence 5′-AGTCGTCGCAGCCTCACCTGATCCATGTAATGCACGTACTTTCAGGGT-3′ (SEQ ID NO: 23).

In some embodiments, in any of the methods for forming a plurality of library-splint complexes (800) described herein, the first region of the first splint strand (620) includes a first universal adaptor sequence which comprises a universal binding sequence (or a complementary sequence thereof) for a first surface primer, where the first region (620) comprises the sequence 5′-TCGGTGGTCGCCGTATCATT-3′ (SEQ ID NO: 24). For example, the first region of the first splint strand (620) can hybridize to a P5 surface primer or a complementary sequence of the P5 surface primer. For example, the P5 surface primer comprises the sequence

5′-AATGATACGGCGACCACCGA-3′ (SEQ ID NO: 25) (short P5), or the P5 surface primer comprises the sequence 5′-AATGATACGGCGACCACCGAGATC-3′ (SEQ ID NO: 26) (long P5). In some embodiments, the second region of the first splint strand (630) includes a second universal adaptor sequence which comprises a universal binding sequence (or a complementary sequence thereof) for a second surface primer, where the second region (630) comprises the sequence 5′-CAAGCAGAAGACGGCATACGA-3′ (SEQ ID NO: 27). For example, the second region of the first splint strand (630) can hybridize to a P7 surface primer or a complementary sequence of the P7 surface primer. For example, the P7 surface primer comprises the sequence 5′-CAAGCAGAAGACGGCATACGA-3′ (SEQ ID NO: 28) (short P7), or the P7 surface primer comprises the sequence 5′-CAAGCAGAAGACGGCATACGAGAT-3′ (SEQ ID NO: 29) (long P7). In some embodiments, the first splint strand (600) includes an internal region (310) which comprises a fourth sub-region having the sequence 5′-ACCCTGAAAGTACGTGCATTACATG-3′ (SEQ ID NO: 30). In some embodiments, the first splint strand (600) includes an internal region (610) which comprises a fifth sub-region having the sequence 5′-GATCAGGTGAGGCTGCGACGACT-3′ (SEQ ID NO: 31). In some embodiments, the first splint strand (600) comprises a first region (620), an internal region (610) having a fourth and fifth sub-region, and a second region (630), having the sequence 5′-TCGGTGGTCGCCGTATCATTACCCTGAAAGTACGTGCATTACATGGATCAGGTGAGG CTGCGACGACTCAAGCAGAAGACGGCATACGA-3′ (SEQ ID NO: 32).

In some embodiments, covalently closed circular library molecules (e.g., (400) and (900)) can be generated using single-stranded library molecules (100) and either single-stranded splint strands (200) or double-stranded splint adaptors (500), as described above. In some embodiments, the covalently closed circular library molecules (e.g., (400) and (900)) can be subjected to a rolling circle amplification reaction.

In some embodiments, the method for generating circularized library molecules further comprises step (e): conducting a rolling circle amplification reaction by hybridizing the plurality of covalently closed circular library molecules (e.g., (400) or (900)) with a plurality of amplification primers and conducting rolling circle amplification reaction in a template-dependent manner, using a plurality of strand displacing polymerases and a plurality of nucleotides, thereby generating a plurality of concatemer template molecules. In some embodiments, the plurality of covalently closed circular library molecules (e.g., (400) or (900)) comprises first and second sub-population of covalently closed circular library molecules.

In some embodiments, the rolling circle amplification reaction comprises hybridizing first and second sub-populations of covalently closed circular library molecules (e.g., (400) or (900)) to first and second amplification primers, respectively. In some embodiments, the first and second amplification primers can be immobilized to a support (e.g., first and second capture primers), or the first and second amplification primers can be in solution. In some embodiments, the first and second amplification primers have the same sequence or have different sequences. In some embodiments, the first and second amplification primers having different sequences comprise first and second batch amplification primers. In some embodiments, the rolling circle amplification reaction is conducted in a template-dependent manner, using a plurality of strand displacing polymerases, a plurality of nucleotides, and the first and second sub-populations of covalently closed circular library molecules (e.g., (400) or (900)), thereby generating a plurality of concatemer template molecules including at least a first sub-population of concatemer template molecules and a second sub-population of concatemer template molecules. In some embodiments, the rolling circle amplification reaction is conducted in the presence or absence of a plurality of compaction oligonucleotides.

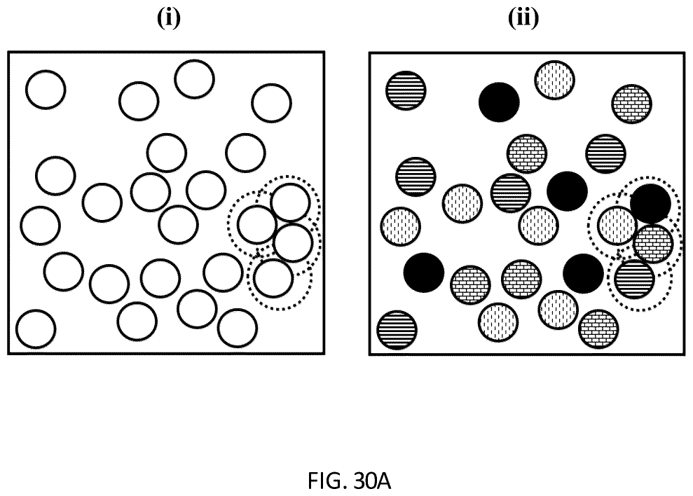

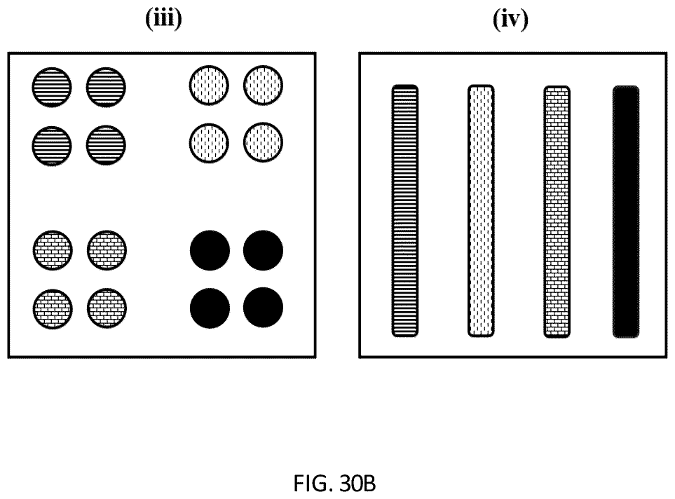

In some embodiments, individual concatemer template molecules in the first sub-population comprise tandem repeat polynucleotide units, wherein a unit comprises a first sequence of interest, the first batch barcode sequence, and a first batch sequencing primer binding site (or a complementary sequence thereof). For concatemers generated using single-stranded splint strands see FIGS. 40A, 41B, 42A, 42B; for concatemers generated using double-stranded splint adaptors see FIGS. 30A and 30B.

In some embodiments, individual concatemer template molecules in the second sub-population comprise tandem repeat polynucleotide units, wherein a unit comprises a second sequence of interest, the second batch barcode sequence, and a second batch sequencing primer binding site (or a complementary sequence thereof). For concatemers generated using single-stranded splint strands see FIGS. 40A, 40B, 42A, 42B; for concatemers generated using double-stranded splint adaptors see FIGS. 47A and 47B.

In some embodiments, e.g., in FIG. 47A, the first covalently closed circular library molecule (900) is subjected to rolling circle amplification to generate a first concatemer template molecule, and the concatemer template molecule is subjected to batch reiterative sequencing. In some embodiments, the rolling circle amplification reaction can be conducted in-solution using soluble amplification primers or on-support using capture primers immobilized to a support. In some embodiments, the first covalently closed circular library molecule (900) comprises: a first surface pinning primer binding site sequence (120-1); a first batch forward sequencing primer binding site sequence (140-1) which corresponds with the first sequence of interest; a first batch barcode sequence (195-1) which corresponds with the first sequence of interest; a first sequence of interest (110-1); and a first surface capture primer binding site sequence (130-1). In some embodiments, a plurality of the first covalently closed circular library molecule (900) shown in FIG. 47A are distributed onto a support having one type of immobilized capture primers which selectively hybridizes to the first capture primer binding site sequence (120-1) in the first covalently closed circular library molecules (900). In some embodiments, the first covalently closed circular library molecules (900) are subjected to rolling circle amplification (RCA) to generate a plurality of first concatemer template molecules which are immobilized to the support. In some embodiments, the first concatemer template molecules are subjected to a sequencing workflow using first batch-specific sequencing primers (solid arrows), sequencing polymerases, and a plurality of nucleotide reagents to generate a plurality of first sequencing read products (dashed arrows). In some embodiments, the first sequencing read products include the first batch barcode sequence (195-1) as shown in FIG. 47A. In some embodiments, the first sequencing read products include the first batch barcode sequence (195-1) and at least a portion of the first sequence of interest (not shown). In some embodiments, the first concatemers undergo reiterative sequencing comprising up to 1000 sequencing cycles.

In some embodiments, e.g., in FIG. 47B, the second covalently closed circular library molecule (900) is subjected to rolling circle amplification to generate a second concatemer template molecule, and the concatemer template molecule is subjected to batch reiterative sequencing. In some embodiments, the rolling circle amplification reaction can be conducted in-solution using soluble amplification primers or on-support using capture primers immobilized to a support. In some embodiments, the second covalently closed circular library molecule (900) comprises: a first surface pinning primer binding site sequence (120-1); a second batch forward sequencing primer binding site sequence (140-2) which corresponds with the second sequence of interest; a second batch barcode sequence (195-2) which corresponds with the second sequence of interest; a second sequence of interest (110-2); and a first surface capture primer binding site sequence (130-1). In some embodiments, a plurality of the second covalently closed circular library molecule (900) shown in FIG. 47B are distributed onto a support having one type of immobilized capture primers which selectively hybridizes to the first capture primer binding site sequence (120-1) in the second covalently closed circular library molecules (900). In some embodiments, a plurality of the first covalently closed circular library molecule (900) shown in FIG. 47A, and a plurality of the second covalently closed circular library molecule (900) shown in FIG. 47B, are distributed onto the same support. In some embodiments, the first covalently closed circular library molecules (900) shown in FIG. 47A and the second covalently closed circular library molecules (900) shown in FIG. 47B can be distributed onto the support essentially simultaneously. In some embodiments, the first covalently closed circular library molecules (900) shown in FIG. 47A and the second covalently closed circular library molecules (900) shown in FIG. 47B can be distributed onto the support sequentially (e.g., re-seeding the support). In some embodiments, the second covalently closed circular library molecules (900) are subjected to rolling circle amplification (RCA) to generate a plurality of second concatemer template molecules which are immobilized to the support. In some embodiments, the second concatemer template molecules are subjected to a sequencing workflow using second batch sequencing primers (solid arrows), sequencing polymerases, and a plurality of nucleotide reagents to generate a plurality of second sequencing read products (dashed arrows). In some embodiments, the second concatemer template molecules are not sequenced when first batch sequencing primers are used to sequence the first concatemer template molecules. In some embodiments, the first concatemer template molecules are not sequenced when second batch sequencing primers are used to sequence the second concatemer template molecules. In some embodiments, the second sequencing read products include the second batch barcode sequence (195-2) as shown in FIG. 47B. In some embodiments, the second sequencing read products include the second batch barcode sequence (195-2) and at least a portion of the second sequence of interest (not shown). In some embodiments, the second concatemer template molecules undergo reiterative sequencing comprising up to 1000 sequencing cycles.

In some embodiments, e.g., FIG. 47B, the library molecule (100) comprises: a first left junction adaptor sequence (121); an adaptor sequence for a capture primer binding site (120); a second left junction adaptor sequence (125); a left sample index sequence (160); a third left junction adaptor sequence (165); an adaptor sequence for a forward sequencing primer binding site (140); a fourth left junction adaptor sequence (145); a sequence of interest (e.g., insert; (110)); a fourth right junction adaptor sequence (155); an adaptor sequence for a reverse sequencing primer binding site (150); a third right junction adaptor sequence (175); a right sample index sequence (170); a second right junction adaptor sequence (135); an adaptor sequence for a pinning primer binding site (130); and a first right junction adaptor sequence (131).

In some embodiments, when the rolling circle amplification of step (e) is conducted with amplification primers that are immobilized to a support, the covalently closed circular library molecules (e.g., (400) or (900)) can be distributed onto the support comprising a plurality of immobilized surface primer, under a condition suitable to hybridize at least one portion of the covalently closed circular library molecules to the immobilized surface primers, and the rolling circle amplification reaction is conducted thereby generating a plurality of immobilized concatemer template molecules including at least a first sub-population of immobilized concatemer template molecules and a second sub-population of immobilized concatemer template molecules. In some embodiments, the on-support rolling circle amplification reaction is conducted in the presence or absence of a plurality of compaction oligonucleotides.

In some embodiments, when the rolling circle amplification of step (e) is conducted with amplification primers that are in solution, the covalently closed circular library molecules (e.g., (400) or (900)) can be hybridized to amplification primers in solution, and the rolling circle amplification reaction can be conducted in-solution, and the rolling circle amplification reaction and nascent concatemers can be distributed onto a support having a plurality of surface primers immobilized thereon, under a condition suitable to hybridize at least one portion of the nascent concatemers to the immobilized surface primers, and the rolling circle amplification reaction can be resumed thereby generating a plurality of immobilized concatemer template molecules including at least a first sub-population of immobilized concatemer template molecules and a second sub-population of immobilized concatemer template molecules. In some embodiments, the in-solution rolling circle amplification reaction is conducted in the presence or absence of a plurality of compaction oligonucleotides. In some embodiments, the on-support rolling circle amplification reaction is conducted in the presence or absence of a plurality of compaction oligonucleotides.

In some embodiments, methods generating circularized library molecules further comprise step (f): sequencing the first sub-population of immobilized concatemer template molecules using a plurality of first batch sequencing primers. In some embodiments, the sequencing of step (f) comprises imaging a region of the support to detect the sequencing reactions of the first sub-population of template molecules.

In some embodiments, the methods for sequencing further comprises step (f1): conducting short read sequencing by performing up to 1000 sequencing cycles of the first sub-population of concatemer template molecules to generate a plurality of first batch sequencing read products that comprise up to 1000 bases in length. In some embodiments, the first batch sequencing read products comprise the first batch barcode sequence. In some embodiments, the first batch sequencing read products comprise the first batch barcode sequence and the sample index sequence. In some embodiments, the first batch sequencing read products comprise the first batch barcode sequence and at least a portion of the first sequence of interest. In some embodiments, the first batch sequencing read products comprise the first batch barcode sequence, the sample index sequence, and at least a portion of the first sequence of interest. In some embodiments, the short read sequencing comprises hybridizing first batch sequencing primers to the first batch sequencing primer binding sites on first sub-population of concatemer template molecules and conducting up to 1000 cycles of polymerase-catalyzed sequencing reactions using nucleotide reagents.

In some embodiments, the methods for sequencing further comprises step (f2): stopping/blocking the short read sequencing of step (f1). In some embodiments, the stopping/blocking comprises incorporating a chain terminating nucleotide to 3′ terminal end of the first batch sequencing read products to inhibit further sequencing reactions. Exemplary chain terminating nucleotides include dideoxynucleotide or a nucleotide having a 2′ or 3′ chain terminating moiety.

In some embodiments, the methods for sequencing further comprise step (f3): removing the plurality of first batch sequencing read products from the concatemer template molecules of the first sub-population, and retaining the concatemer template molecules of the first sub-population.

In some embodiments, the methods for sequencing further comprise step (f4): reiteratively sequencing the concatemer template molecules of the first sub-population by repeating steps (f1)-(f3) at least once. In some embodiments, the reiterative sequencing can be conducted 1-10 times, or 10-25 times, or 25-50 times or more. For example, the reiterative sequencing can be conducted up to 100 times.

Exemplary schematics of reiterative sequencing workflows are shown in FIGS. 40A, 40B, 42A, 42B, 47A and 47B.

In some embodiments, the sequences of all of the first batch sequencing read products can be determined and aligned with a first reference sequence to confirm the presence of the first sequence of interest. The first reference sequence can be the first batch barcode and/or the first sequence of interest.

In some embodiments, hybridizing the first batch sequencing primers to the concatemer template molecules of step (f1) can be conducted with a hybridization reagent comprising an SSC buffer (e.g., 2× saline-sodium citrate) buffer with formamide (e.g., 10-20% formamide).

In some embodiments, in step (f3) the plurality of plurality of first batch sequencing read products can be removed from the concatemer template molecules and the plurality of concatemer template molecules can be retained using a de-hybridization reagent comprising an SSC buffer (e.g., saline-sodium citrate) buffer, with or without formamide, at a temperature that promotes nucleic acid denaturation.

In some embodiments, methods generating circularized library molecules further comprise step (g): sequencing the second sub-population of immobilized concatemer template molecules using a plurality of second batch sequencing primers. In some embodiments, the sequencing of step (g) comprises imaging the same region of the support to detect the sequencing reactions of the second sub-population of template molecules.

In some embodiments, the sequencing reactions of the first sub-population of template molecules is stopped before initiating the sequencing reactions of the second sub-population of template molecules.

In some embodiments, the methods for sequencing further comprises step (g1): conducting short read sequencing by performing up to 1000 sequencing cycles of the second sub-population of concatemer template molecules to generate a plurality of second batch sequencing read products that comprise up to 1000 bases in length. In some embodiments, the second batch sequencing read products comprise the second batch barcode sequence. In some embodiments, the second batch sequencing read products comprise the second batch barcode sequence and the sample index sequence. In some embodiments, the second batch sequencing read products comprise the second batch barcode sequence and at least a portion of the second sequence of interest. In some embodiments, the second batch sequencing read products comprise the second batch barcode sequence, the sample index sequence, and at least a portion of the second sequence of interest. In some embodiments, the short read sequencing comprises hybridizing second batch sequencing primers to the second batch sequencing primer binding sites on second sub-population of concatemer template molecules and conducting up to 1000 cycles of polymerase-catalyzed sequencing reactions using nucleotide reagents.

In some embodiments, the methods for sequencing further comprises step (g2): stopping/blocking the short read sequencing of step (g1). In some embodiments, the stopping/blocking comprises incorporating a chain terminating nucleotide to 3′ terminal end of the second batch sequencing read products to inhibit further sequencing reactions. Exemplary chain terminating nucleotides include dideoxynucleotide or a nucleotide having a 2′ or 3′ chain terminating moiety.

In some embodiments, the methods for sequencing further comprise step (g3): removing the plurality of second batch sequencing read products from the concatemer template molecules of the second sub-population, and retaining the concatemer template molecules of the second sub-population.

In some embodiments, the methods for sequencing further comprise step (g4): reiteratively sequencing the concatemer template molecules of the second sub-population by repeating steps (g1)-(g3) at least once. In some embodiments, the reiterative sequencing can be conducted 1-10 times, or 10-25 times, or 25-50 times or more. For example, the reiterative sequencing can be conducted up to 100 times.

Exemplary schematics of reiterative sequencing workflows are shown in FIGS. 40A, 40B, 42A, 42B, 47A and 47B.

In some embodiments, the sequences of all of the second batch sequencing read products can be determined and aligned with a second reference sequence to confirm the presence of the second sequence of interest. The second reference sequence can be the second batch barcode and/or the second sequence of interest.

In some embodiments, hybridizing the second batch sequencing primers to the concatemer template molecules of step (g1) can be conducted with a hybridization reagent comprising an SSC buffer (e.g., 2× saline-sodium citrate) buffer with formamide (e.g., 10-20% formamide).

In some embodiments, in step (g3) the plurality of plurality of second batch sequencing read products can be removed from the concatemer template molecules and the plurality of concatemer template molecules can be retained using a de-hybridization reagent comprising an SSC buffer (e.g., saline-sodium citrate) buffer, with or without formamide, at a temperature that promotes nucleic acid denaturation.

### Support

The present disclosure provides a support for use in conducting any of the batch sequencing, reiterative sequencing and/or re-seeding methods described herein. In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

The support comprises any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support can be substantially smooth and lack contours and texture. In some embodiments, the support can be regularly or irregularly contoured or textured, including protrusions, bumps, wells, etchings, pores, three-dimensional scaffolds, or any combination thereof. In some embodiments, the support comprises contours arranged in a pre-determined pattern. In some embodiments, the support comprises contours arranged in a repeating pattern. In some embodiments, the support comprises interstitial regions between the contours, where the interstial regions are arranged in a pre-determined. In some embodiments, the interstitial regions are arranged in a repeating pattern.

In some embodiments, the contours and interstial regions can be fabricated using any combination of photo-chemical, photo-lithography, electron beam lithography, micro- or nano-imprint lithography, ink-jet printing, or micron-scale printing and/or nano-scale printing.

In some embodiments, the contours can be functionalized to promote tethering/immobilizing nucleic acid molecules (e.g., capture primers, pinning primers and/or template molecules) and/or for tethering an enzyme (e.g., a polymerase). In some embodiments, the interstial regions can be modified to inhibit tethering nucleic acid molecules (e.g., capture primers, pinning primers and/or template molecules) and/or for inhibiting tethering an enzyme (e.g., a polymerase).

In some embodiments, the support comprises at least one region (e.g., a feature) which can be functionalized to tether/immobilize nucleic acid molecules and/or enzymes. In some embodiments, the features are arranged on the support in a non-predetermined manner (e.g., randomly positioned features; e.g., FIG. 30A(i)). In some embodiments, the features are arranged on the support in a predetermined manner (e.g., patterned features; e.g., FIGS. 30B(iii) and 30B(iv)). In some embodiments, the features are arranged on the support in repeating pattern (e.g., FIGS. 30B(iii) and 30B(iv)).

In some embodiments, a support comprises a plurality of features located at random and non-predetermined positions on the support, where individual features can attach to a nucleic acid molecule (e.g., capture primer, pinning primer or template molecule). Each of the features on the support can be functionalized with a chemical compound to attach to a nucleic acid molecule.

For example, the features on the support can attach to nucleic acid capture primers (e.g., see FIG. 30A(i)). In some embodiments, the capture primers can be attached to the support such that some of the nearest neighbor capture primers touch each other and/or overlap each other when viewed from any angle of the support including above, below or side views of the support. The dotted lines that surround the four capture primers represents nearest neighbor capture primers that touch each other (e.g., FIG. 30A(i)).

In some embodiments, e.g. FIG. 30A(i), the capture primers can be attached to the support such that some of the nearest neighbor capture primers touch each other and/or overlap each other when viewed from any angle of the support including above, below or side views of the support. The dotted lines that surround the four capture primers represents nearest neighbor capture primers that touch each other.

In some embodiments, e.g., FIG. 30A(ii), the template molecules can attach to the support (e.g., via attachment to the capture primers) such that some of the nearest neighbor template molecules touch each other and/or overlap each other when viewed from any angle of the support including above, below or side views of the support. The dotted lines that surround the four template molecules represent nearest neighbor template molecules that touch each other.

In some embodiments, e.g., FIG. 30B(iii), the template molecule comprise one of four different batch sequences. The different batch sequences of the template molecules are represented by horizontal stripes, vertical dashed, brick or solid black. For example, the template molecules can be immobilized to the support to form spots arranged in row and columns. In some embodiments, e.g., FIG. 30B(iv), The template molecule comprise one of four different batch sequences. The different batch sequences of the template molecules are represented by horizontal stripes, vertical dashed, brick or solid black. For example, the template molecules can be immobilized to the support to form stripes.

In some embodiments, the capture primers on the support can attach to nucleic acid template molecules having one of four different batch sequences (e.g., see FIG. 30).A (ii) In some embodiments, the template molecules can attach to the support (via attachment to the capture primers) such that some of the nearest neighbor template molecules touch each other and/or overlap each other when viewed from any angle of the support including above, below or side views of the support. The dotted lines that surround the four template molecules represent nearest neighbor template molecules that touch each other (e.g., FIG. 30A(ii)).

In some embodiments, the support comprises a contour and at least one feature on or near the contour for tethering nucleic acid molecules. For example, one or more wells (e.g., a plurality of contours) can be fabricated on the support where the bottom of individual wells include a feature having a chemical modification for tethering one or more nucleic acid molecules. The skilled artisan will recognize that the support can be fabricated with any type of contour(s) and feature(s) that are on or near the contour(s), where the features are designed to tether at least one nucleic acid molecule.

In some embodiments, the support lacks contours. In some embodiments, the support lacks features arranged in a pre-determined pattern where the features have a chemical functionality for tethering nucleic acid molecules and/or enzymes to the support. In some embodiments, the support comprises features positioned at random non-predetermined locations on the support. In some embodiments, the support lacks interstitial regions arranged in a pre-determined pattern where the interstial regions are sites designed to inhibit tethering nucleic acid molecules or enzymes.

In some embodiments, any of the features for tethering nucleic acids and/or enzymes can be positioned on the support using ink-jet printing, or micron-scale or nano-scale printing. In some embodiments, the features can be made in any shape including for example, circular, square, triangular or rectangular (e.g., FIGS. 30A(i) and 30B(iii)).

In some embodiments, at least one surface of the support can be modified with a chemical compound that enables attachment of a polymer coating to the support. For example, the support can be modified with a silane compound. In some embodiments, the silane compound can bind a polymer coating. In some embodiments, at least one surface of the support is passivated with at least one polymer coating layer (e.g., FIG. 28). In some embodiments, the support is passivated with 1, 2, 3, 4, 5, 6, 7, 8, 9, 10 or more polymer coating layers. In some embodiments, the coating forms a continuous layer on the support wherein the coating forms no pre-determined pattern.

In some embodiments, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, the coating is distributed on the support in a pre-determined pattern, for example the pre-determined pattern comprises or spots arranged in rows and/or columns or other pre-determined patterns. In some embodiments, the coating having a pre-determined pattern comprises at least one interstitial region that lacks a polymer coating. In some embodiments, the passivated layer forms a porous or semi-porous layer.

In some embodiments, at least one of the polymer coating layers comprises a hydrophilic polymer layer. In some embodiments, at least one polymer coating layer comprises polymer molecules having a molecular weight of at least 1000 Daltons. The hydrophilic polymer coating layer can comprise polyethylene glycol (PEG). The hydrophilic polymer layer can comprise unbranched PEG. The hydrophilic polymer layer can comprise branched PEG having at least 4 branches, for example the branched PEG comprises 4-16 branches. In some embodiments, the hydrophilic polymer layer comprises cross-linking or lacks cross-linking. In some embodiments, the hydrophilic polymer layer comprises cross-linking to form a hydrogel.

In some embodiments, the hydrophilic polymer layer comprises a monolayer having unbranched polymers which can form a brush monolayer. In some embodiments, the brush monolayer can form an extended brush monolayer. In some embodiments, the brush monolayer comprises a plurality of unbranched polymers where one end of a given unbranched polymer is attached to the support and the other end of the same given unbranched polymer is attached to an oligonucleotide primer (e.g., capture primer or pinning primer). In some embodiments, the density of the plurality of oligonucleotide primers attached to the brush monolayer is about 10²-10¹⁵ per um².

In some embodiments, the coating layer has a degree of hydrophilicity which can be measured as a water contact angle, where the water contact angle is no more than 45 degrees.

In some embodiments, any layer of the polymer coating includes a plurality of oligonucleotide primers covalently tethered to the polymer layer. In some embodiments, the plurality of oligonucleotide primers are distributed at a plurality of depths throughout any of the polymer layers. In some embodiments, the density of the plurality of oligonucleotide primers in any of the polymer layers is about 10²-10¹⁵ per um². In some embodiments, individual oligonucleotide primers comprise nucleic acid molecules comprising DNA, RNA, DNA/RNA chimeric or analogs thereof. In some embodiments, the plurality of oligonucleotide primers are about 10-100 nucleotides in length. In some embodiments, individual oligonucleotide primers in the plurality comprise 3′ extendible ends or 3′ non-extendible ends. In some embodiments, the 3′ non-extendible ends comprise a 3′ chain terminating moiety. In some embodiments, individual oligonucleotide primers have their 5′ or 3′ ends or an internal region attached to the polymer layer. In some embodiments, 5′ ends of the plurality of oligonucleotide primers are attached to the polymer layer. In some embodiments, the plurality of oligonucleotide primer are randomly distributed throughout and embedded within at least one of the polymer layers. In some embodiments, the plurality of oligonucleotide primer are distributed in or on at least one of the polymer layers in a random manner or a pre-determined pattern. In some embodiments, the plurality of oligonucleotide primers are distributed in or on at least one of the polymer layers in a non-random pre-determined pattern, for example the pre-determined pattern comprises stripes or spots arranged in rows and/or columns or other pre-determined patterns.

In some embodiments, the support comprises a first layer comprising a first monolayer having hydrophilic polymer molecules tethered to the support. In some embodiments, at least some of the polymer molecules in the first layer are covalently tethered to oligonucleotide primers. In some embodiments, the tethered oligonucleotide primers in the first monolayer are arranged in a random manner or in a pre-determined pattern. In some embodiments, the polymer molecules in the first layer are not tethered to oligonucleotide primers.

In some embodiments, the support further comprises a second layer comprising a second monolayer having hydrophilic polymer molecules tethered to the first monolayer. In some embodiments, at least some of the polymer molecules in the second layer are covalently tethered to oligonucleotide primers. In some embodiments, the tethered oligonucleotide primers in the second monolayer are arranged in a random manner or in a pre-determined pattern. In some embodiments, the polymer molecules in the second layer are not tethered to oligonucleotide primers.

In some embodiments, the support further comprises a third layer comprising a third monolayer having hydrophilic polymer molecules tethered to the second monolayer. In some embodiments, at least some of the polymer molecules in the third layer are covalently tethered to oligonucleotide primers. In some embodiments, the tethered oligonucleotide primers in the third monolayer are arranged in a random manner or in a pre-determined pattern. In some embodiments, the polymer molecules in the third layer are not tethered to oligonucleotide primers.

In some embodiments, the support comprises a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl) acrylamide-co-acrylamide (PAZAM).

In some embodiments, at least one of the polymer layers comprise oligonucleotide primers including capture primers, pinning primers, or a mixture of capture and pinning primers. In some embodiments, the plurality of oligonucleotide primers comprise one type of capture primer (e.g., having that same batch capture primer sequence) or a mixture of 2-100 different types of capture primers (e.g., having 2-100 different batch capture primer sequences). In some embodiments, the plurality of oligonucleotide primers comprise one type of pinning primer (e.g., having that same batch pinning primer sequence) or a mixture of 2-100 different types of pinning primers (e.g., having 2-100 different batch pinning primer sequences).

In some embodiments, individual capture primers (e.g., which are tethered to and/or embedded in a polymer layer) can be used in an on-support amplification reaction wherein individual capture primers hybridize to a capture primer binding site in a circularized library molecule, and rolling circle amplification can be conducted to generate a concatemer template molecule which is tethered and/or embedded in the polymer layer.

In some embodiments, individual capture primers (e.g., which are tethered to and/or embedded in a polymer layer) can be used in an in-solution amplification workflow wherein individual capture primers can hybridize to a capture primer binding site in a nascent concatemer molecule, and rolling circle amplification can continue on the polymer layer to generate a concatemer template molecule which is tethered and/or embedded in the polymer layer.

In some embodiments, the density of the capture primers in a polymer layer can be modulated (e.g., increased or decreased) to achieve a desired density of immobilized concatemer template molecules on a support. Generally, a polymer layer having a high density of capture primers will generate concatemer template molecules that are tightly packed and immobilized to the support at a density of about 10⁵-10¹⁵ per mm² which cannot be achieved using supports fabricated to include nano-scale features for attachment of template molecules.

In some embodiments, a single pinning primer (e.g., which is tethered to or embedded in a polymer layer) can hybridize to a pinning primer binding site in a concatemer molecule to generate a concatemer template molecule which is tethered or embedded (e.g., pinned down) in the polymer layer.

In some embodiments, at least one of the polymer layers comprise a plurality of capture primers and/or pinning primers having a cleavable region that is cleavable with a restriction endonuclease enzyme. For example, the cleavable region comprises a recognition site for a type I, type II, type IIs, type IIB, type III, or type IV restriction enzyme. In some embodiments, the plurality of capture primers and/or pinning primers include a cleavable region that is cleavable with an enzyme that generates an abasic site. For example, the cleavable region comprises at least one nucleotide having a scissile moiety including uridine, 8-oxo-7,8-dihydrogunine or deoxyinosine. In some embodiments, the plurality of capture primers and/or pinning primers lack a cleavable region.

In some embodiments, the support comprises at least one partition/barrier that creates separate regions of the support. For example, the partition/barrier can prevent fluid flow on one portion of the support. The partition/barrier can inhibit nucleic acid and/or enzyme reactions on a portion of the support. In some embodiments, the partition/barrier can be placed on the support. In some embodiments, the partition/barrier is not placed on the support but is positioned to block fluid flow onto the support.

In some embodiments, the support lacks partitions/barriers that would create separate regions of the support. For example, the support is passivated with at least one polymer coating formed as a continuous layer, and at least one of the polymer layers comprise a plurality of capture primers that are randomly distributed throughout and on the polymer layer. The capture primers can be used to generate immobilized concatemer template molecules. Thus, the immobilized template molecules are in fluid communication with each other in a massively parallel manner with no barriers to physically separate different batches of template molecules. Instead, sub-populations of template molecules carrying different batch sequencing primer binding sites which enables batch sequencing. Asynchronous sequencing is achieved using concatemer template molecules in fluid communication with each other on the same non-partitioned support.

### Fragmenting Nucleic Acids

The present disclosure provides methods for preparing nucleic acid library molecules for use in any of the methods described including batch sequencing, re-seeding, re-iterative sequencing, padlock probe workflows, single-stranded splint workflows and/or double-stranded splint workflow.

In some embodiments, the insert region of a nucleic acid library molecule comprises a sequence of interest extracted from any source. The insert region can be prepared using recombinant nucleic acid technology including but not limited to any combination of vector cloning, transgenic host cell preparation, host cell culturing and/or PCR amplification.

In some embodiments, the insert region can be in fragmented or un-fragmented form, and can be used to prepare linear nucleic acid library molecules. Fragmented forms of the insert region can be obtained by mechanical force, enzymatic or chemical fragmentation methods. The fragmented insert regions can be generated using procedures that yield a population of fragments having overlapping sequences or non-overlapping sequences.

Mechanical fragmentation typically generates randomly fragmented nucleic acid molecules. Mechanical fragmentation methods include mechanical shearing such as fluid shear, constant shear and pulsatile shear. Mechanical fragmentation methods also include mechanical stress including sonication, nebulization and acoustic cavitation. In some embodiments focused acoustic energy can be used to randomly fragment nucleic acid molecules. A commercially-available apparatus (e.g., Covaris) can be used to fragment nucleic acid molecules using focused acoustic energy.

Enzymatic fragmentation procedures can be conducted under conditions suitable to generate randomly or non-randomly fragmented nucleic acid molecules. For example, restriction endonuclease enzyme digestion can be conducted to completion to generate non-randomly fragmented nucleic acid molecule. Alternatively, partial or incomplete restriction enzyme digestion can be conducted to generate randomly-fragmented nucleic acid molecules. Enzymatic fragmentation using restriction endonuclease enzymes includes any one or any combination of two or more restriction enzymes selected from a group consisting of type I, type II, type IIs, type IIB, type III, or type IV restriction enzymes. Enzymatic fragmentation includes digestion of the nucleic acid with a rare-cutting restriction enzyme, comprising Not I, Asc I, Bae I, AspC I, Pac I, Fse I, Sap I, Sfi I or Psr I. Enzymatic fragmentation include use of any combination of a nicking restriction endonuclease, endonuclease and/or exonuclease. Enzymatic fragmentation can be achieved by conducting a nick translation reaction.

In some embodiments, enzymatic fragmentation can be achieved by reacting nucleic acids with an enzyme mixture, for example an enzyme that generates single-stranded nicks and another enzyme that catalyzes double-stranded cleavage. An exemplary enzyme mixture is FRAGMENTASE (e.g., from New England Biolabs).

Fragments of the insert region can be generated with PCR using sequence-specific primers that hybridize to target regions in genomic DNA samples to generate insert regions having known fragment lengths and sequences.

Targeted genome fragmentation methods using CRISPR/Cas9 can be used to generate fragmented insert regions.

Fragments of the insert portion can also be generated using a transposase-based tagmentation method using NEXTERA (from Epicentre).

The insert region can be single-stranded or double-stranded. The ends of the double-stranded insert region can be blunt-ended, or have a 5′ overhang or a 3′ overhang end, or any combination thereof. One or both ends of the insert region can be subjected to an enzymatic tailing reaction to generate a non-template poly-A tail by employing a terminal transferase reaction. The ends of the insert region can be compatible for joining to at least one adaptor sequence (e.g., universal adaptor sequence or batch-specific adaptor sequence).

The insert region can be any length, for example the insert region can be about 50-250, or about 250-500, or about 500-750, or about 750-1000, or about 100-2000 bases or base pairs in length. The fragments containing the insert region can be subjected to a size selection process, or the fragments are not size selected. For example, the fragments can be size selected by gel electrophoresis and gel slice extraction. The fragments can be size selected using a solid phase adherence/immobilization method which typically employs micro paramagnetic beads coated with a chemical functional group that interacts with nucleic acids under certain ionic strength conditions with or without polyethylene glycol or polyalkylene glycol. Commercially-available solid phase adherence beads include SPRI (Solid Phase Reversible Immobilization) beads from Beckman Coulter (AMPUR XP paramagnetic beads, catalog No. B23318), MAGNA PURE magnetic glass particles (Roche Diagnostics, catalog No. 03003990001), MAGNASIL paramagnetic beads from Promega (catalog No. MD1360), MAGTRATION paramagnetic beads and system from Precision System Science (catalog Nos. A1120 and A1060), MAG-BIND from Omega Bio-Tek (catalog No. M1378-01), MAGPREP silica from Millapore (catalog No. 101193), SNARE DNA purification systems from Bangs Laboratories (catalog Nos. BP691, BP692 and BP693), and CHEMAGEN M-PVA beads from Perkin Elmer (catalog No. CMG-200).

In some embodiments, the fragmented nucleic acids can be subjected to enzymatic reactions for end-repair and/or A-tailing. The fragmented nucleic acids can be contacted with a plurality of enzymes under a condition suitable to generate nucleic acid fragments having blunt-ended 5′ phosphorylated ends. In some embodiments, the plurality of enzymes generates blunt-ended fragment having a non-template A-tail at their 3′ ends. The plurality of enzymes comprise two or more enzymes that can catalyze nucleic acid end-repair, phosphorylation and/or A-tailing. The end-repair enzymes include a DNA polymerase (e.g., T4 DNA polymerase) and Klenow fragment. The 5′ end phosphorylation enzyme comprises T4 polynucleotide kinase. The A-tailing enzyme includes a Taq polymerase (e.g., non-proof-reading polymerase) and dATP. In some embodiments, the fragmenting, end-repair, phosphorylation and A-tailing can be conducted in a one-pot reaction using a mixture of enzymes.

### Appending Adaptors to Fragmented or Unfragmented Nucleic Acids

In some embodiments, individual fragmented (or unfragmented) nucleic acids can be covalently joined to at least one adaptor sequence for library preparation. In general, a nucleic acid fragment is covalently joined at both ends to one or more adaptors to generate a linear library molecule having the arrangement left adaptor-insert-right adaptor. In some embodiments, at least one fragment in the population of fragmented nucleic acids comprises a sequence-of-interest. Individual library molecules in the population of library molecules can have an insert region that is the same or different as other library molecules in the population. In some embodiments, about 1-10 ng, or about 10-50 ng, or about 50-100 ng of input fragmented nucleic acids can be appended to one or more adaptors to generate a linear library.

Individual nucleic acid fragments can be appended on one or both ends to at least one adaptor sequence to form a recombinant nucleic acid linear library molecule having the general arrangement left adaptor-insert-right adaptor.

In some embodiments, the nucleic acid fragments can be appended with any one or any combination of two or more adaptors, and arranged in any order, where the adaptors comprise an adaptor having a binding sequence for a pinning primer (120), an adaptor having a binding sequence for a capture primer (130), an adaptor having a binding sequence for a forward sequencing primer (140), an adaptor having a binding sequence for a reverse sequencing primer (150), a left sample index sequence (160), a right sample index sequence (170), a left unique identification sequence (180) and/or, an adaptor sequence for binding a compaction oligonucleotide.

In some embodiments, any of the adaptors comprise universal adaptor sequences or batch-specific adaptor sequences.

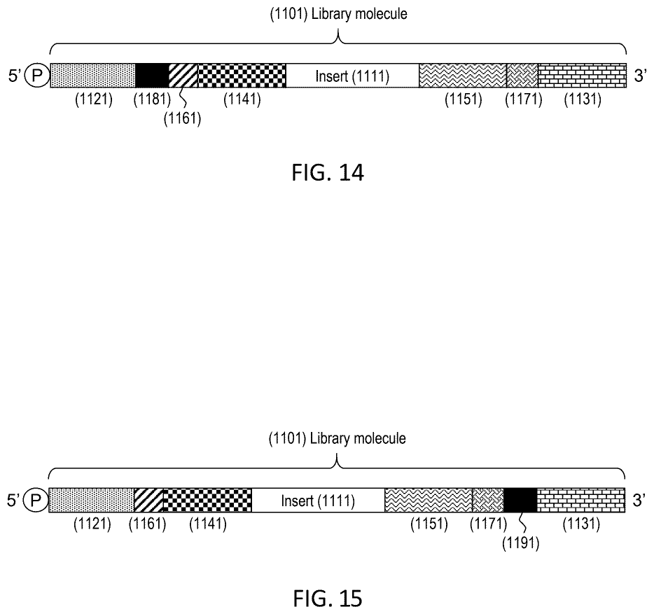

Exemplary linear library molecules are shown in FIGS. 14-15. The skilled artisan appreciates that many other embodiments of linear library molecules comprising adaptor sequences with other arrangements are possible.

The adaptors can be prepared using chemical synthesis procedures using native nucleotides with or without nucleotide analogs or modified nucleotide linkages that confer certain properties, including resistance to enzymatic digestion, or increased thermal stability. Examples of nucleotide analogs and modified nucleotide linkages that inhibit nuclease digestion include phosphorothioate, 2′-O-methyl RNA, inverted dT, and 2′ 3′ dideoxy-dT. Insert regions that include locked nucleic acids (LNA) have increased thermal stability.

The insert region can be joined at one or both ends to at least one adaptor sequence using a ligase enzyme and/or primer extension reaction to generate a linear library molecule. Covalent linkage between an insert region and the adaptor(s) can be achieved with a DNA or RNA ligase. Exemplary DNA ligases that can ligate double-stranded DNA molecules include T4 DNA ligase and T7 DNA ligase. An adaptor sequence can be appended to an insert sequence by PCR using a tailed primer having 5′ region carrying a adaptor sequence and a 3′ region that is complementary to a portion of the insert sequence. A adaptor sequence can be appended to an insert sequence which is flanked one side or both sides with first and second adaptor sequences by PCR using a tailed primer having 5′ region carrying a third adaptor sequence and a 3′ region that is complementary to a portion of the first or second adaptor sequence.

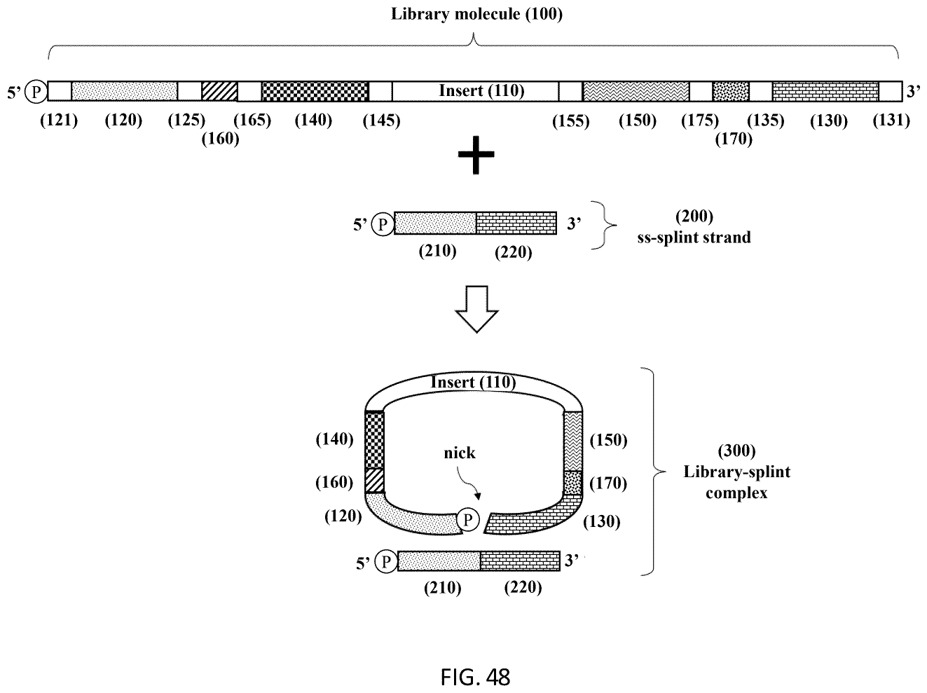

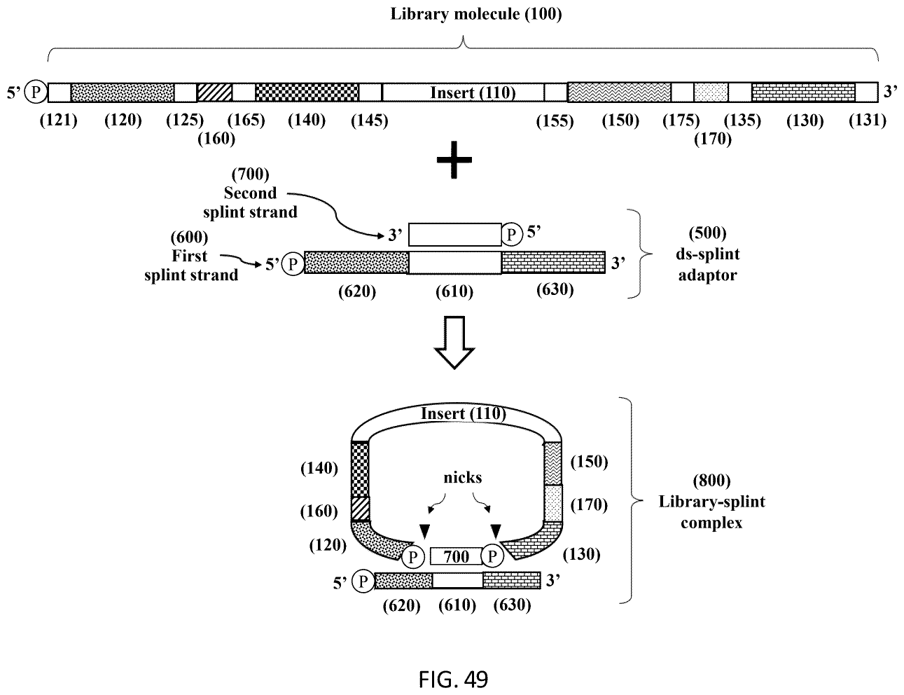

In some embodiments, the nucleic acid library molecule (100) further comprises at least one junction adaptor sequence located between any of the adaptor sequences described herein (e.g., see FIGS. 48 and 49). For example, a first left junction adaptor sequence (121) can be located upstream (e.g., located 5′) of the adaptor sequence for a pinning primer binding site (120). In some embodiments, a second left junction adaptor sequence (125) can be located between the adaptor sequence for a pinning primer binding site (120) and the left sample index sequence (160). In some embodiments, a third left junction adaptor sequence (165) can be located between the left sample index sequence (160) and the adaptor sequence for the forward sequencing primer binding site (140). In some embodiments, s fourth junction adaptor sequence (145) can be located between the adaptor sequence for the forward sequencing primer binding site (140) and the sequence-of-interest (e.g., insert region (110)). In some embodiments, a first right junction adaptor sequence (131) can be located downstream (e.g., located 3′) of the adaptor sequence for a capture primer binding site (130). In some embodiments, a second right junction adaptor sequence (135) can be located between the adaptor sequence for a capture primer binding site (130) and the right sample index sequence (170). In some embodiments, a third right junction adaptor sequence (175) can be located between the right sample index sequence (170) and the adaptor sequence for a reverse sequencing primer binding site (150). In some embodiments, a fourth right junction adaptor sequence (155) can be located between the adaptor sequence for a reverse sequencing primer binding site (150) and the sequence-of-interest (e.g., insert (110)).

Any of the junction adaptor sequences comprise any sequence and can be 3-60 nucleotides in length. Any of the junction adaptor sequences comprise a universal sequence, a batch-specific sequence, or a unique sequence. Any of the junction adaptor sequences comprise a random sequence (e.g., NNN) having 3-20 nucleotides. Any of the junction adaptor sequences comprise a binding sequence for an amplification primer, a sequencing primer or a compaction oligonucleotide. Any of the junction adaptor sequences comprise a binding sequence for an immobilized capture primer. Any of the junction adaptor sequences comprise a sample index sequence. Any of the junction adaptor sequences comprise a unique identification sequence (e.g., UMI). Any of the junction adaptor sequences, particularly junction adaptor sequence (145) comprises a Tn5 transposon-end sequence, for example 5′-AGATGTGTATAAGAGACAG-3′ (SEQ ID NO: 37). Any of the junction adaptor sequences, particularly junction adaptor sequence (155) comprises a Tn5 transposon-end sequence, for example 5′-CTGTCTCTTATACACATCT-3′ (SEQ ID NO: 36). The Tn5 transposon-end sequences can be introduced into the library molecule (100) via a transposase-mediated reaction which includes contacting double-stranded input DNA (e.g., genomic DNA) with a Tn-5 type transposase enzyme, and a double-stranded oligonucleotide comprising the Tn transposon-end sequence linked to an adaptor sequence or a sample index sequence under a condition that is suitable to form a transposon synaptic complex. In the double-stranded oligonucleotide, the Tn transposon-end sequence can be located 5′ or 3′ relative to an adaptor sequence or a sample index sequence.

In some embodiments, e.g., FIG. 48, the single-stranded splint strand (200) comprises a first region (210) that hybridizes with one end of the linear single stranded library molecule (100) including at least a portion of the adaptor sequence for a capture primer binding site (120) and/or at least a portion of the first left junction adaptor sequence (121). The single-stranded splint strand (200) comprises a second region (220) that hybridizes with the other end of the linear single stranded library molecule (100) including at least a portion of the adaptor sequence for a pinning primer binding site (120) and/or at least a portion of the first right junction adaptor sequence (131). For the sake of simplicity, the library-splint complex (300) does not show any of the junction adaptors. The skilled artisan will recognize that the library-splint complex (300) can include any one or any combination of two or more of the junction adaptors that are present in the library molecule (100).

In some embodiments, e.g., in FIG. 49, the library molecule (100) comprises: a first left junction adaptor sequence (121); an adaptor sequence for a capture primer binding site (120); a second left junction adaptor sequence (125); a left sample index sequence (160); a third left junction adaptor sequence (165); an adaptor sequence for a forward sequencing primer binding site (140); a fourth left junction adaptor sequence (145); a sequence of interest (e.g., insert; (110)); a fourth right junction adaptor sequence (155); an adaptor sequence for a reverse sequencing primer binding site (150); a third right junction adaptor sequence (175); a right sample index sequence (170); a second right junction adaptor sequence (135); an adaptor sequence for a pinning primer binding site (130); and a first right junction adaptor sequence (131). The double-stranded splint adaptor (500) comprises a first splint strand (600) having a first region (620) that hybridizes with one end of the linear single stranded library molecule (100) including at least a portion of the adaptor sequence for a pinning primer binding site (120) and/or at least a portion of the first left junction adaptor sequence (121). The double-stranded splint adaptor (500) comprises a first splint strand (600) having a second region (630) that hybridizes with the other end of the linear single stranded library molecule (100) including at least a portion of the adaptor sequence for a capture primer binding site (130) and/or at least a portion of the first right junction adaptor sequence (131). For the sake of simplicity, the library-splint complex (300) does not show any of the junction adaptors. The skilled artisan will recognize that the library-splint complex (300) can include any one or any combination of two or more of the junction adaptors that are present in the library molecule (100).

In some embodiments, a linear library molecule (100) can be generated by employing a ligation reaction and an optional primer extension reaction. The library molecule can be generated by joining the first end of a double-stranded insert region (110) to a first double-stranded adaptor, and joining the second end of the double-stranded insert region (110) to a second double-stranded adaptor. The first and second double-stranded adaptors each comprise two nucleic acid strands that are fully complementary along their lengths.

In some embodiments, individual double-stranded insert regions (110) can be joined to a first and a second double-stranded adaptor using a DNA ligase enzyme to generate a double-stranded recombinant molecule. In some embodiments the first and second double-stranded adaptors carry the same adaptor sequences. In some embodiments the first and second double-stranded adaptors carry different adaptor sequences.

In some embodiments, the library molecule can be generated by joining the first end of a double-stranded insert region (110) to a first double-stranded adaptor having a having a binding sequence for a forward sequencing primer (140), and joining the second end of the double-stranded insert region (110) to a second double-stranded adaptor having a binding sequence for a reverse sequencing primer (150), wherein the joining is conducted using a DNA ligase enzyme to generate a double-stranded recombinant molecule. In some embodiments, the first double-stranded adaptor further comprises a left sample index sequence (160) and/or a binding sequence for a pinning primer (120). In some embodiments, the second double-stranded adaptor further comprises a right sample index sequence (170) and/or a binding sequence for a capture primer (130).

In some embodiments, the ligating end of the first and/or the second double-stranded adaptors comprise a blunt end, or an overhang end (e.g., 5′ or 3′ overhang end).

In some embodiments, a linear library molecule (100) can be generated by employing a ligation reaction and primer extension reaction. The library molecule can be generated by joining the first end of a double-stranded insert region (110) to a first double-stranded Y-shaped adaptor (e.g., a first forked adaptor), and joining the second end of a double-stranded insert region (110) to a second double-stranded Y-shaped adaptor (e.g., a second forked adaptor). The first and second Y-shaped adaptors each comprise two nucleic acid strands, where a portion of the two strands are fully complementary to each other and are annealed together and another portion of the two strands are not complementary to each other and are mismatched. In some embodiments, the ligating end of the first and second Y-shaped adaptors comprise an annealed portion that forms a blunt end or an overhang end (e.g., 5′ or 3′ overhang end).

In some embodiments the first and second Y-shaped adaptors carry the same adaptor sequences. In some embodiments the first and second Y-shaped adaptors carry different adaptor sequences.

In some embodiments, the first strand of the annealed portion and/or the mismatched portion of the Y-shaped adaptor can include at least a portion of an adaptor sequence having a binding sequence for a forward sequencing primer (140) (or a complementary sequence thereof). In some embodiments, the first strand of the annealed portion and/or the mismatched portion of the Y-shaped adaptor can further include a left sample index sequence (160). In some embodiments, the first strand of the annealed portion and/or the mismatched portion of the Y-shaped adaptor can further include an adaptor sequence having a binding sequence for a pinning primer (120).

In some embodiments, the second strand of the annealed portion and/or the mismatched portion of the Y-shaped adaptor can include at least a portion of an adaptor sequence having a binding sequence for a reverse sequencing primer (150) (or a complementary sequence thereof). In some embodiments, the second strand of the annealed portion and/or the mismatched portion of the Y-shaped adaptor can further include a right sample index sequence (170). In some embodiments, the second strand of the annealed portion and/or the mismatched portion of the Y-shaped adaptor can further include an adaptor sequence having a binding sequence for a capture primer (130).

The double-stranded insert region (110) can be joined to the first and second double-stranded Y-shaped adaptors using a DNA ligase enzyme to generate a double-stranded recombinant molecule.

In some embodiments, the double-stranded recombinant molecules which are generated by ligating the insert region (110) to double-stranded adaptors or Y-shaped adaptors can be subjected to a denaturing condition to generate single-stranded recombinant molecules, and then a primer extension reaction. At least one additional adaptor sequence can be appended to the recombinant molecules by conducting a primer extension reaction using tailed primers (e.g., tailed PCR primers), by contacting/hybridizing the single-stranded recombinant molecules with a plurality of first tailed primers and conducting at least one primer extension reaction to generate a first double-stranded tailed extension product.

In some embodiments, an additional adaptor sequence can be appended to the first double-stranded tailed extension product by conducting a primer extension reaction using tailed primers (e.g., tailed PCR primers), by contacting/hybridizing the first double-stranded tailed extension product with a plurality of second tailed primers and conducting at least one primer extension reaction to generate a second double-stranded tailed extension product.

In some embodiments, the plurality of first tailed primers each comprise a 5′ region carrying an adaptor sequence having a binding sequence for a capture surface primer (130), and a 3′ region that is complementary to at least a portion of the adaptor sequence having a binding sequence for a reverse sequencing primer (150) of the single-stranded recombinant molecules.

In some embodiments, the plurality of first tailed primers each comprise a 5′ region carrying an adaptor sequence having a binding sequence for a capture surface primer (130), an internal region comprising a right sample index sequence (170), and a 3′ region that is complementary to at least a portion of the adaptor sequence having a binding sequence for a reverse sequencing primer (150) of the single-stranded recombinant molecules.

In some embodiments, the plurality of second tailed primers each comprise a 5′ region carrying an adaptor sequence having a binding sequence for a pinning surface primer (120), and a 3′ region that is complementary to at least a portion of the adaptor sequence having a binding sequence for a forward sequencing primer (140) of the first double-stranded tailed extension product.

In some embodiments, the plurality of second tailed primers each comprise a 5′ region carrying an adaptor sequence having a binding sequence for a pinning surface primer (120), an internal region comprising a left sample index sequence (160), and a 3′ region that is complementary to at least a portion of the adaptor sequence having a binding sequence for a forward sequencing primer (140) of the first double-stranded tailed extension product.

In some embodiments, the first tailed PCR primers can be used to conduct a first primer extension reaction and the second tailed PCR primers can be used conduct a second primer extension to generate library molecules comprising an insert region appended on both sides with at least one adaptor. In some embodiments, the first and second tailed PCR primers can be used to conduct multiple PCR cycles (e.g., about 5-20 PCR cycles) to generate library molecules comprising an insert region appended on both sides with at least one adaptor.

### Template Molecules

The present disclosure provides a plurality of nucleic acid template molecules for use in conducting any of the batch sequencing, reiterative sequencing and/or re-seeding methods described herein. In some embodiments, the plurality of template molecules are immobilized to a support. In some embodiments, the plurality of template molecules comprise single-stranded or double-stranded nucleic acid molecules, or a mixture of single-stranded and double-stranded nucleic acid molecules. In some embodiments, the plurality of template molecules comprise nucleic acid molecules comprising DNA, RNA, DNA/RNA chimeric or analogs thereof. In some embodiments, the plurality of template molecules are immobilized to the support at a density of about 10²-10¹⁵ template molecules per mm².

In some embodiments, the plurality of template molecules comprises at least one nucleotide having a scissile moiety that can be cleaved to generate an abasic site in the template molecule. Exemplary nucleotides having a scissile moiety include uridine, 8-oxo-7,8-dihydrogunine and deoxyinosine. In some embodiments, the plurality of template molecules lack a nucleotide having a scissile moiety. In some embodiments, the plurality of template molecules comprise a mixture of template molecules that either lack a nucleotide having a scissile moiety or include at least one nucleotide having a scissile moiety. In some embodiments, the plurality of template molecules lack a scissile moiety.

In some embodiments, the plurality of template molecules comprise at least one recognition site for a restriction endonuclease enzyme, including a type I, type II, type IIs, type IIB, type III, or type IV restriction enzymes. In some embodiments, the plurality of template molecules comprise the same restriction enzyme site. In some embodiments, the plurality of template molecules comprise a mixture of template molecules having different restriction enzyme sites, or a mixture of template molecules lacking a restriction enzyme site and template molecules having a restriction enzyme site. In some embodiments, the plurality of template molecules lack a recognition site for a restriction endonuclease enzyme.

In some embodiments, individual template molecules in the plurality of template molecules comprise nucleic acid concatemer template molecules. In some embodiments, the concatemer template molecules can be generated by conducting rolling circle amplification using circularized library molecules and amplification primers. In some embodiments, a concatemer molecule comprises a single-stranded nucleic acid strand carrying numerous tandem copies of a polynucleotide unit, where each polynucleotide unit comprises a sequence of interest and at least one sequencing primer binding site. In some embodiments, the sequence of interest of one of the concatemer template molecules in the plurality and the sequence of interest of a different concatemer template molecule are the same or different.

In some embodiments, concatemer template molecules immobilized to a support can be generated using circularized library molecules and conducting rolling circle amplification. In some embodiments, the circularized library molecules can be generated using padlock probes, single-stranded splint strands, or double-stranded adaptors. Methods for generating circularized library molecules are described herein.

In some embodiments, the at least one sequencing primer binding site sequence comprises a pre-determined batch sequencing primer binding site sequence. In some embodiments, a pre-determined batch sequencing primer binding site sequence can be linked to a given sequence of interest, thus the pre-determined batch sequencing primer binding site sequence corresponds to a given sequence of interest. In some embodiments, in a batch-specific sequencing workflow, a batch sequencing primer can be used to selectively sequence at least a portion of a polynucleotide unit having a cognate batch sequencing primer binding site sequence.

In some embodiments, the polynucleotide unit of a concatemer molecule further comprises at least one barcode sequence. In some embodiments, a pre-determined batch barcode sequence can be linked to a given sequence of interest, thus the pre-determined batch barcode sequence corresponds to a given sequence of interest. In some embodiments, in a batch-specific sequencing workflow, the batch barcode sequence can be sequenced and the sequence of interest need not be sequenced. Thus, the batch barcode sequence serves as a surrogate for the sequence of interest that is linked to the batch barcode sequence.

In some embodiments, a polynucleotide unit further comprises at least one sample index sequence that can be used in a multiplex assay to distinguish sequences of interest obtained from different sample sources.

In some embodiments, a polynucleotide unit further comprises a capture primer binding site. In some embodiments, a capture primer serves as an amplification primer for a circularized library molecule in a rolling circle amplification reaction. In some embodiments, the capture primer binding site of the circularized library molecule can hybridize to a capture primer which is immobilized to a support thereby immobilizing the circularized library molecule to the support. In some embodiments, an immobilized concatemer molecule can be generated by hybridizing a single capture primer to a single circularized library molecule and conducting rolling circle amplification to generate an immobilized concatemer molecule.

In some embodiments, a polynucleotide unit further comprises a surface pinning binding site. In some embodiments, in a concatemer template molecule, the surface pinning binding site can hybridize to a pinning primer which is immobilized to a support thereby pinning a portion of the concatemer template molecule to the support.

In some embodiments, a polynucleotide unit further comprises a compaction oligonucleotide binding site. In some embodiments, in a concatemer template molecule, the compaction oligonucleotide binding site binds a compaction oligonucleotide to cause compaction of the concatemer template molecule into a DNA nanoball.

In some embodiments, the plurality of nucleic acid template molecules comprises a plurality of sub-populations of nucleic acid template molecules including at least a first sub-population and a second sub-population. In some embodiments, the plurality of nucleic acid template molecules comprises 2-100 or more sub-populations of template molecules. In some embodiments, individual template molecules in a given sub-population comprise a sequence of interest, a sequencing primer binding site sequence that corresponds to the sequence of interest, and optionally a barcode sequence that corresponds to the sequence of interest. In some embodiments, the template molecules of a given sub-population have a sequencing primer binding site that differs from the sequencing primer binding site in the other sub-populations. Thus, the different sequencing primer binding sites of the different sub-populations enable batch sequencing of the template molecules.

In some embodiments, the plurality of nucleic acid template molecules further comprise any combination of a sample index sequence, a capture primer binding site, a surface pinning primer binding site and/or a compaction oligonucleotide binding site.

In some embodiments, at least one of the template molecules in the plurality comprises a concatemer molecule which includes a plurality of tandem copies of a polynucleotide unit, where each polynucleotide unit comprises (i) a sequence of interest; (ii) a sequencing primer binding site sequence which corresponds to the sequence of interest; and (iii) optionally a barcode sequence which corresponds to the sequence of interest. In some embodiments, the polynucleotide unit of the at least one concatemer molecule further comprises any combination of (iv) a sample index sequence that can be used in a multiplex assay to distinguish sequences of interest obtained from different sample sources; (v) a capture primer binding site; (vi) a surface pinning primer binding site; and/or (vii) a compaction oligonucleotide binding site.

In some embodiments, individual template molecules in the first sub-population comprise a first sequence of interest, a first batch sequencing primer binding site sequence that corresponds to the first sequence of interest, and optionally a first batch barcode sequence that corresponds to the first sequence of interest. In some embodiments, template molecules in the first sub-population have the same sequence of interest or different sequences of interest. In some embodiments, template molecules in the first sub-population have the same first batch sequencing primer binding site sequence which corresponds to the first sequence of interest or corresponds to one of the first sequence of interest. In some embodiments, template molecules in the first sub-population have the same first batch barcode sequence or different first batch barcode sequences. In some embodiments, a first barcode sequence corresponds to a first sequence of interest, or corresponds to one of the first sequences of interest.

In some embodiments, individual template molecules in the first sub-population comprise the same first sequence of interest, the same first batch sequencing primer binding site sequence that corresponds to the first sequence of interest, and the same first batch barcode sequence that corresponds to the first sequence of interest.

In some embodiments, individual template molecules in the first sub-population comprise at least two different first sequences of interest, the same first batch sequencing primer binding site sequence that corresponds to the different first sequences of interest, and at least two different first batch barcode sequences where each first batch barcode sequence corresponds to a particular first sequence of interest.

In some embodiments, individual template molecules in the first sub-population comprise at least two different first sequences of interest, the same first batch sequencing primer binding site sequence that corresponds to the different first sequences of interest, and one first batch barcode sequence that corresponds to the different first sequences of interest.

In some embodiments, the template molecules in the first sub-population further comprise a sample index sequence that can be used in a multiplex assay to distinguish sequences of interest obtained from different sample sources. In some embodiments, template molecules in the first sub-population have the same sample index sequences. In some embodiments, the first sub-population comprises a mixture of template molecules having different sample index sequences. For example, some of the template molecules in the first sub-population comprises a first batch first sample index sequence, and some of the template molecules in the first sub-population comprises a first batch second sample index sequence.

In some embodiments, the first sub-population comprising a mixture of template molecules having different sample index sequences can be generated by conducting separate library prep workflows to generate: (i) a first set of library molecules comprising a first sequence of interest from a first source, a first batch barcode sequence that corresponds to the first sequence of interest, a first batch sequencing primer binding site sequence that corresponds to the first sequence of interest, and a first sample index that corresponds to the first source of the first sequence of interest, and (ii) a second set of library molecules comprising the first sequence of interest from a second source, a first batch barcode sequence that corresponds to the first sequence of interest, a first batch sequencing primer binding site sequence that corresponds to the first sequence of interest, and a second sample index that corresponds to the second source of the first sequence of interest. The resulting first and second library preps can be mixed together to generate a mixture of template molecules in the first sub-population having a mixture of different sample index sequences.

In some embodiments, the template molecules in the first sub-population further comprise at least one binding site for a compaction oligonucleotide (e.g., a universal binding site for a compaction oligonucleotide). The compaction oligonucleotide can hybridize to the template molecule to pull together distal portions of the template molecule causing compaction of the template molecule to form a DNA nanoball.

In some embodiments, the template molecules in the first sub-population further comprise a first batch capture primer binding site. In some embodiments, template molecules in the first sub-population have the same first batch capture primer binding site.

In some embodiments, the template molecules in the first sub-population further comprise a first batch surface pinning binding site which can hybridize to a first surface pinning primer which is immobilized to a support thereby pinning a portion of the template molecules of the first sub-population to the support. In some embodiments, template molecules in the first sub-population have the same first batch surface pinning binding site.

In some embodiments, individual template molecules in the first sub-population of template molecules comprise first sub-population concatemer template molecules. In some embodiments, individual concatemer molecule in the first sub-population comprise a single-stranded nucleic acid strand carrying a plurality of tandem copies of a polynucleotide unit, where each polynucleotide unit comprises (i) a first sequence of interest; and (ii) a first batch sequencing primer binding site sequence which corresponds to the first sequence of interest. In some embodiments, the polynucleotide unit of individual concatemer molecules in the first sub-population further comprise any combination of (iii) a first batch barcode sequence which corresponds to the first sequence of interest; (iv) a sample index sequence that can be used in a multiplex assay to distinguish sequences of interest obtained from different sample sources; (v) a first batch capture primer binding site; (vi) a first batch surface pinning primer binding site; and/or (vii) a compaction oligonucleotide binding site.

In some embodiments, individual template molecules in the second sub-population comprise a second sequence of interest, a second batch sequencing primer binding site sequence that corresponds to the second sequence of interest, and optionally a second batch barcode sequence that corresponds to the second sequence of interest. In some embodiments, template molecules in the second sub-population have the same sequence of interest or different sequences of interest. In some embodiments, template molecules in the second sub-population have the same second batch sequencing primer binding site sequence which corresponds to the second sequence of interest or corresponds to one of the second sequence of interest. In some embodiments, the first and second batch sequencing primer binding sites have different sequences. In some embodiments, template molecules in the second sub-population have the same second batch barcode sequence or different second batch barcode sequences. In some embodiments, a second barcode sequence corresponds to a second sequence of interest, or corresponds to one of the second sequences of interest.

In some embodiments, individual template molecules in the second sub-population comprise the same second sequence of interest, the same second batch sequencing primer binding site sequence that corresponds to the second sequence of interest, and the same second batch barcode sequence that corresponds to the second sequence of interest.

In some embodiments, individual template molecules in the second sub-population comprise at least two different second sequences of interest, the same second batch sequencing primer binding site sequence that corresponds to the different second sequences of interest, and at least two different second batch barcode sequences where each second batch barcode sequence corresponds to a particular second sequence of interest.

In some embodiments, individual template molecules in the second sub-population comprise at least two different second sequences of interest, the same second batch sequencing primer binding site sequence that corresponds to the different second sequences of interest, and one second batch barcode sequence that corresponds to the different second sequences of interest.

In some embodiments, the template molecules in the second sub-population further comprise a sample index sequence that can be used in a multiplex assay to distinguish sequences of interest obtained from different sample sources. In some embodiments, template molecules in the second sub-population have the same sample index sequences. In some embodiments, the second sub-population comprises a mixture of template molecules having different sample index sequences. For example, some of the template molecules in the second sub-population comprises a second batch first sample index sequence, and some of the template molecules in the second sub-population comprises a second batch second sample index sequence.

In some embodiments, the second sub-population comprising a mixture of template molecules having different sample index sequences can be generated by conducting separate library prep workflows to generate: (i) a first set of library molecules comprising a second sequence of interest from a first source, a second batch barcode sequence that corresponds to the second sequence of interest, a second batch sequencing primer binding site sequence that corresponds to the second sequence of interest, and a first sample index that corresponds to the first source of the second sequence of interest, and (ii) a second set of library molecules comprising the second sequence of interest from a second source, a second batch barcode sequence that corresponds to the second sequence of interest, a second batch sequencing primer binding site sequence that corresponds to the second sequence of interest, and a second sample index that corresponds to the second source of the second sequence of interest. The resulting first and second library preps can be mixed together to generate a mixture of template molecules in the second sub-population having a mixture of different sample index sequences.

In some embodiments, the template molecules in the second sub-population further comprise at least one binding site for a compaction oligonucleotide (e.g., a universal binding site for a compaction oligonucleotide). The compaction oligonucleotide can hybridize to the template molecule to pull together distal portions of the template molecule causing compaction of the template molecule to form a DNA nanoball.

In some embodiments, the template molecules in the second sub-population further comprise a second batch capture primer binding site. In some embodiments, template molecules in the second sub-population have the same second batch capture primer binding site.

In some embodiments, the template molecules in the second sub-population further comprise a second batch surface pinning binding site which can hybridize to a second surface pinning primer which is immobilized to a support thereby pinning a portion of the template molecules of the second sub-population to the support. In some embodiments, template molecules in the second sub-population have the same second batch surface pinning binding site.

In some embodiments, individual template molecules in the second sub-population of template molecules comprise second sub-population concatemer template molecules. In some embodiments, individual concatemer molecule in the second sub-population comprise a single-stranded nucleic acid strand carrying a plurality of tandem copies of a polynucleotide unit, where each polynucleotide unit comprises (i) a second sequence of interest; and (ii) a second batch sequencing primer binding site sequence which corresponds to the second sequence of interest. In some embodiments, the polynucleotide unit of individual concatemer molecules in the second sub-population further comprise any combination of (iii) a second batch barcode sequence which corresponds to the second sequence of interest; (iv) a sample index sequence that can be used in a multiplex assay to distinguish sequences of interest obtained from different sample sources; (v) a second batch capture primer binding site; (vi) a second batch surface pinning primer binding site; and/or (vii) a compaction oligonucleotide binding site.

In some embodiments, the plurality of nucleic acid template molecules are immobilized to a support at a density of about 10²-10¹⁵ template molecules per mm². In some embodiments, the immobilized template molecules comprise one population or a mixture of at least two sub-populations of template molecules including at least a first and second sub-population of template molecules.

In some embodiments, the plurality of template molecules are immobilized to the support at a density where at least some of the immobilized template molecules comprise nearest neighbor template molecules that do not touch each other and/or do not overlap each other when viewed from any angle of the support including above, below or side views of the support. In some embodiments, the immobilized template molecules have visible interstial space between the immobilized template molecules at a given field of view (FOV) of the support.

In some embodiments, the plurality of template molecules are immobilized to the support at a high density where at least some of the immobilized template molecules comprise nearest neighbor template molecules that touch each other and/or overlap each other when viewed from any angle of the support including above, below or side views of the support. In some embodiments, the high density immobilized template molecules have little or no visible interstial space between the immobilized template molecules at a given field of view (FOV) of the support.

In some embodiments, the immobilized template molecules are optically resolvable as discrete spots. In some embodiments, the immobilized template molecules are not optically resolvable as spots. In some embodiments, the immobilized template molecules comprise a mixture of template molecules that are, or are not, optically resolvable as discrete spots.

In some embodiments, about 20-75%, or about 25-65%, or about 30-55%, or about 35-45% of the immobilized template molecules are optically resolvable as a discrete spot when viewed from any angle above, below or side view of the support.

In some embodiments, about 20-75%, or about 25-65%, or about 30-55%, or about 35-45% of the immobilized template molecules have a nearest neighbor distance of 15-10 nm.

In some embodiments, about 20-75%, or about 25-65%, or about 30-55%, or about 35-45% of the immobilized template molecules have a nearest neighbor distance of 10-5 nm.

In some embodiments, about 20-75%, or about 25-65%, or about 30-55%, or about 35-45% of the immobilized template molecules have a nearest neighbor distance of 5-1 nm or smaller nearest neighbor distance.

In some embodiments, interstitial space between the immobilized template molecules is about 15-10 nm, or about 10-5 nm, or about 5-1 nm, or smaller.

In some embodiments, the support comprises a plurality of template molecules immobilized at random (e.g., random and non-repeating positions) and non-pre-determined positions on the support. In some embodiments, the plurality of template molecules includes one population of template molecules, or a mixture of at least two sub-populations of template molecules including at least a first and second sub-population of template molecules. In some embodiments, the support comprises features on the support that are located in a random and non-pre-determined manner, where the features are sites for attachment of the template molecules. In some embodiments, the support lacks any contours (e.g., wells, protrusions, and the like) arranged in a pre-determined pattern where the contours have features that are sites for attachment of the nucleic acid template molecules. In some embodiments, the support lacks features arranged in a pre-determined pattern. In some embodiments, the support lacks features arranged in a pre-determined pattern where the feature have a chemical functionality for tethering a nucleic acid template molecule to the support. In some embodiments, the support lacks interstitial regions arranged in a pre-determined pattern where the interstial regions are sites designed to have no attached template molecules.

In some embodiments, the support comprises a plurality of template molecules immobilized at pre-determined positions on the support. For example the template molecules can be immobilized on the support in a pre-determined pattern comprises stripes or spots arranged in rows and/or columns or other pre-determined patterns. In some embodiments, the pre-determined pattern has a repeating pattern. In some embodiments, the plurality of template molecules includes one population of template molecules, or a mixture of at least two sub-populations of template molecules including at least a first and second sub-population of template molecules. In some embodiments, the support comprises features on the support that are located in a pre-determined manner, where the features are sites for attachment of the template molecules. In some embodiments, the support includes contours (e.g., wells, protrusions, and the like) arranged in a pre-determined pattern where the contours have features that are sites for attachment of the nucleic acid template molecules. In some embodiments, the support includes features arranged in a pre-determined pattern where the features can be fabricated using photo-chemical, photo-lithography, electron beam lithography, micro- or nano-imprint lithography, ink-jet printing, or micron-scale or nano-scale printing. In some embodiments, the support includes features arranged in a pre-determined pattern where the feature have a chemical functionality for tethering a nucleic acid template molecule to the support. In some embodiments, the support includes interstitial regions arranged in a pre-determined pattern where the interstial regions are sites designed to have no attached template molecules.

The present disclosure provides methods for sequencing any of the immobilized concatemer molecules described herein. Any of the methods for conducting rolling circle amplification reaction described herein can be used to generate a plurality of concatemer molecules immobilized to a support, and the immobilized concatemers can be subjected to sequencing reactions using sequencing polymerases and nucleotide reagents which include nucleotides, nucleotide analogs and/or multivalent molecules. In some embodiments, the sequencing reactions employ nucleotide reagents comprising detectably labeled nucleotide analogs. In some embodiments, the sequencing reactions employ a two-stage sequencing reaction comprising binding detectably labeled multivalent molecules, and incorporating nucleotide analogs. In some embodiments, the sequencing reactions employ non-labeled nucleotide analogs. The terms concatemer molecule and template molecule are used interchangeably.

### Methods for Sequencing Using Nucleotide Analogs

The present disclosure provides methods for sequencing any of the immobilized concatemer molecules described herein, the methods comprising step (a): contacting a sequencing polymerase to (i) a nucleic acid concatemer molecule and (ii) a nucleic acid sequencing primer, wherein the contacting is conducted under a condition suitable to bind the sequencing polymerase to the nucleic acid concatemer molecule which is hybridized to the nucleic acid primer, wherein the nucleic acid concatemer molecule hybridized to the nucleic acid primer forms the nucleic acid duplex. In some embodiments, the sequencing polymerase comprises a recombinant mutant sequencing polymerase that can bind and incorporate nucleotide analogs. In some embodiments, the sequencing primer comprises a 3′ extendible end.

In some embodiments, the sequencing primer comprises a 3′ extendible end or a 3′ non-extendible end. In some embodiments, the plurality of nucleic acid concatemer molecules comprise amplified template molecules (e.g., clonally amplified template molecules). In some embodiments, the plurality of nucleic acid concatemer molecules comprise one copy of a target sequence of interest. In some embodiments, the plurality of nucleic acid molecules comprise two or more tandem copies of a target sequence of interest (e.g., concatemers). In some embodiments, the nucleic acid concatemer molecules in the plurality of nucleic acid concatemer molecules comprise the same target sequence of interest or different target sequences of interest. In some embodiments, the plurality of nucleic acid concatemer molecules and/or the plurality of nucleic acid primers are in solution or are immobilized to a support. In some embodiments, when the plurality of nucleic acid concatemer molecules and/or the plurality of nucleic acid primers are immobilized to a support, the binding with the first sequencing polymerase generates a plurality of immobilized first complexed polymerases. In some embodiments, the plurality of nucleic acid concatemer molecules and/or nucleic acid primers are immobilized to 10²-10¹⁵ different sites on a support. In some embodiments, the binding of the plurality of concatemer molecules and nucleic acid primers with the plurality of first sequencing polymerases generates a plurality of first complexed polymerases immobilized to 10²-10¹⁵ different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., fluorophore). In some embodiments, step (b) further comprises removing the chain terminating moiety from the incorporated chain terminating nucleotide to generate an extendible 3′OH group. In some embodiments, the sequencing of step (b) further comprises repeating at least once the steps of: (i) incorporating a detectably labeled chain terminating nucleotide into the terminal 3′ end of a hybridized first sequencing primer; (ii) detecting and identifying the incorporated chain terminating nucleotide; and (iii) removing the chain terminating moiety and/or the detectable label from the incorporated chain terminating nucleotide to generate an extendible 3′OH sugar group on the incorporated chain terminating nucleotide.

In some embodiments, the methods for sequencing further comprise step (c): incorporating at least one nucleotide into the 3′ end of the extendible primer under a condition suitable for incorporating the at least one nucleotide. In some embodiments, the suitable conditions for nucleotide binding the polymerase and for incorporation the nucleotide can be the same or different. In some embodiments, conditions suitable for incorporating the nucleotide comprise inclusion of at least one catalytic cation comprising magnesium and/or manganese. In some embodiments, the at least one nucleotide binds the sequencing polymerase and incorporates into the 3′ end of the extendible primer. In some embodiments, the incorporating the nucleotide into the 3′ end of the primer in step (c) comprises a primer extension reaction.

In some embodiments, the methods for sequencing further comprise step (d): repeating the incorporating at least one nucleotide into the 3′ end of the extendible primer of steps (b) and (c) at least once. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base. In some embodiments, the method further comprises detecting the at least one incorporated nucleotide at step (c) and/or (d). In some embodiments, the method further comprises identifying the at least one incorporated nucleotide at step (c) and/or (d). In some embodiments, the sequence of the nucleic acid concatemer molecule can be determined by detecting and identifying the nucleotide that binds the sequencing polymerase, thereby determining the sequence of the concatemer molecule. In some embodiments, the sequence of the nucleic acid concatemer molecule can be determined by detecting and identifying the nucleotide that incorporates into the 3′ end of the primer, thereby determining the sequence of the concatemer molecule.

In some embodiments, in the methods for sequencing, the plurality of sequencing polymerases that are bound to the nucleic acid duplexes comprise a plurality of complexed polymerases, having at least a first and second complexed polymerase, wherein (a) the first complexed polymerases comprises a first sequencing polymerase bound to a first nucleic acid duplex comprising a first nucleic acid template sequence which is hybridized to a first nucleic acid primer, (b) the second complexed polymerases comprises a second sequencing polymerase bound to a second nucleic acid duplex comprising a second nucleic acid template sequence which is hybridized to a second nucleic acid primer, (c) the first and second nucleic acid template sequences comprise the same or different sequences, (d) the first and second nucleic acid concatemers are clonally-amplified, (e) the first and second primers comprise extendible 3′ ends or non-extendible 3′ ends, and (f) the plurality of complexed polymerases are immobilized to a support. In some embodiments, the density of the plurality of complexed polymerases is about 10²-10¹⁵ complexed polymerases per mm² that are immobilized to the support.

### Sequencing-by-Binding

The present disclosure provides methods for sequencing any of the immobilized concatemer molecules described herein, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

### Sequencing Polymerases

The present disclosure provides methods for sequencing any of the immobilized concatemer molecules described herein, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

### Nucleotides

The present disclosure provides methods for sequencing any of the immobilized concatemer molecules described herein, where any of the sequencing methods described herein employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride. In some embodiments, the chain terminating moiety may be cleavable/removable with nitrous acid. In some embodiments, a chain terminating moiety may be cleavable/removable using a solution comprising nitrite, such as, for example, a combination of nitrite with an acid such as acetic acid, sulfuric acid, or nitric acid. In some further embodiments, said solution may comprise an organic acid.

In some embodiments, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as, for example, formic acid, acetic acid, propionic acid, butyric acid, isobutyric acid, or the like.

In some embodiments, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

Supports with Low Non-Specific Binding Coatings

The present disclosure provides compositions and methods for use of a support having a plurality of surface primers immobilized thereon, for preparing any of the immobilized concatemers described herein. In some embodiments, the support is passivated with a low non-specific binding coating (e.g., FIG. 27). The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

In general, the supports comprise a substrate (or support structure), one or more layers of a covalently or non-covalently attached low-binding, chemical modification layers, e.g., silane layers, polymer films, and one or more covalently or non-covalently attached primer sequences that may be used for tethering single-stranded target nucleic acid(s) to the support surface. In some embodiments, the formulation of the surface, e.g., the chemical composition of one or more layers, the coupling chemistry used to cross-link the one or more layers to the support surface and/or to each other, and the total number of layers, may be varied such that non-specific binding of proteins, nucleic acid molecules, and other hybridization and amplification reaction components to the support surface is minimized or reduced relative to a comparable monolayer. Often, the formulation of the surface may be varied such that non-specific hybridization on the support surface is minimized or reduced relative to a comparable monolayer. The formulation of the surface may be varied such that non-specific amplification on the support surface is minimized or reduced relative to a comparable monolayer. The formulation of the surface may be varied such that specific amplification rates and/or yields on the support surface are maximized. Amplification levels suitable for detection are achieved in no more than 2, 3, 4, 5, 6, 7, 8, 9, 10, 15, 20, 25, 30, or more than 30 amplification cycles in some cases disclosed herein. Amplification levels suitable for detection are achieved in no 30, 40, 50, 60, 70, 80, 90, 100, 110, 120, 130, 140, 150, or more than 150 amplification cycles in some cases.

The substrate or support structure that comprises one or more chemically-modified layers, e.g., layers of a low non-specific binding polymer, may be independent or integrated into another structure or assembly. For example, in some embodiments, the substrate or support structure may comprise one or more surfaces within an integrated or assembled microfluidic flow cell. The substrate or support structure may comprise one or more surfaces within a microplate format, e.g., the bottom surface of the wells in a microplate. As noted above, in some preferred embodiments, the substrate or support structure comprises the interior surface (such as the lumen surface) of a capillary. In alternate preferred embodiments the substrate or support structure comprises the interior surface (such as the lumen surface) of a capillary etched into a planar chip.

The attachment chemistry used to graft a first chemically-modified layer to a surface will generally be dependent on both the material from which the surface is fabricated and the chemical nature of the layer. In some embodiments, the first layer may be covalently attached to the surface. In some embodiments, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H2O₂)), base treatment in KOH and NaOH, and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl)triethoxysilane (APTES), any of a variety of PEG-silanes (e.g., comprising molecular weights of 1K, 2K, 5K, 10 K, 20 K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of preferred polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the substrate. Alternately, the surface coating may be patterned, such that the chemical modification layers are confined to one or more discrete regions of the substrate. For example, the surface may be patterned using photolithographic techniques to create an ordered array or random pattern of chemically-modified regions on the surface. Alternately or in combination, the substrate surface may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In order to achieve low nonspecific binding surfaces, hydrophilic polymers may be nonspecifically adsorbed or covalently grafted to the surface. Typically, passivation is performed utilizing poly(ethylene glycol) (PEG, also known as polyethylene oxide (PEO) or polyoxyethylene) or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting surface. In some embodiments, oligonucleotide primers with different base sequences and base modifications (or other biomolecules, e.g., enzymes or antibodies) may be tethered to the resulting surface layer at various surface densities. In some embodiments, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Examples of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

As noted, the low non-specific binding coatings of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, in some embodiments, exposure of the surface to fluorescent dyes (e.g., cyanine dyes such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under a condition where fluorescence signal is linearly related (or related in a predictable manner) to the number of fluorophores on the support surface (e.g., under a condition where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some embodiments, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or other molecules) per unit area. In some embodiments, the low-binding supports of the present disclosure may exhibit non-specific protein binding (or non-specific binding of other specified molecules, (e.g., cyanine dyes such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 M solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rho11 (Jena Biosciences), 10 μM Aminoallyl-dUTP-ATTO-Rho11 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding substrates at 37° C. for 15 minutes in a 384 well plate format. Each well was rinsed 2-3× with 50 uL deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (Olympus Corp., Center Valley, PA) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, New York), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm².

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaces disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, in some embodiments adequate wash steps may be performed in less than 30 seconds.

The low-binding surfaces of the present disclosure exhibit significant improvement in stability or durability to prolonged exposure to solvents and elevated temperatures, or to repeated cycles of solvent exposure or changes in temperature. For example, in some embodiments, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer (e.g., capture primers) may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers on the low binding support surfaces of the present disclosure may range from about 100 primer molecules per μm² to about 100,000 primer molecules per μm². In some embodiments, the resultant surface density of primers on the low binding support surfaces of the present disclosure may range from about 100,000 primer molecules per μm² to about 10¹⁵ primer molecules per μm². In some embodiments, the surface density of primers may be at least 1,000, at least 10,000, at least 100,000, or at least 10¹⁵ primer molecules per μm². In some embodiments, the surface density of primers may be at most 10,000, at most 100,000, at most 1,000,000, or at most 10¹⁵ primer molecules per μm². Any of the lower and upper values described in this paragraph may be combined to form a range included within the present disclosure, for example, in some embodiments the surface density of primers may range from about 10,000 molecules per μm² to about 10¹⁵ molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000 per μm², while also comprising at least a second region having a substantially different local density.

The low non-specific binding coating comprise one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule to about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

Fluorescence imaging may be performed using any of a variety of fluorophores, fluorescence imaging techniques, and fluorescence imaging instruments known to those of skill in the art. Examples of suitable fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), Cyanine dye-7 (Cy7), etc. Examples of fluorescence imaging techniques that may be used include, but are not limited to, fluorescence microscopy imaging, fluorescence confocal imaging, two-photon fluorescence, and the like. Examples of fluorescence imaging instruments that may be used include, but are not limited to, fluorescence microscopes equipped with an image sensor or camera, confocal fluorescence microscopes, two-photon fluorescence microscopes, or custom instruments that comprise a suitable selection of light sources, lenses, mirrors, prisms, dichroic reflectors, apertures, and image sensors or cameras, etc. A non-limiting example of a fluorescence microscope equipped for acquiring images of the disclosed low-binding support surfaces and clonally-amplified colonies (polonies) of template nucleic acid sequences hybridized thereon is the Olympus IX83 inverted fluorescence microscope equipped with) 20×, 0.75 NA, a 532 nm light source, a bandpass and dichroic mirror filter set optimized for 532 nm long-pass excitation and Cy3 fluorescence emission filter, a Semrock 532 nm dichroic reflector, and a camera (Andor sCMOS, Zyla 4.2) where the excitation light intensity is adjusted to avoid signal saturation. Often, the support surface may be immersed in a buffer (e.g., 25 mM ACES, pH 7.4 buffer) while the image is acquired.

In some instances, the performance of nucleic acid hybridization and/or amplification reactions using the disclosed reaction formulations and low non-specific binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. The surfaces of the instant disclosure are also provided in co-pending International Application Serial No. PCT/US2019/061556, which is hereby incorporated by reference in its entirety.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (S)—Bᵢₙₜₑᵣ in the FOV) yields a discernable feature that can be classified. In some instances, the intrastitial background (Bᵢₙₜᵣₐ) can contribute a confounding fluorescence signal that is not specific to the target of interest, but is present in the same ROI thus making it far more difficult to average and subtract.

The implementation of nucleic acid amplification on the low-binding substrates of the present disclosure may decrease the Bᵢₙₜₑᵣ background signal by reducing non-specific binding, may lead to improvements in specific nucleic acid amplification, and may lead to a decrease in non-specific amplification that can impact the background signal arising from both the interstitial and intrastitial regions. In some instances, the disclosed low-binding support surfaces, optionally used in combination with the disclosed hybridization buffer formulations, may lead to improvements in CNR by a factor of 2, 5, 10, 100, or 1000-fold over those achieved using conventional supports and hybridization, amplification, and/or sequencing protocols. Although described here in the context of using fluorescence imaging as the read-out or detection mode, the same principles apply to the use of the disclosed low non-specific binding supports and nucleic acid hybridization and amplification formulations for other detection modes as well, including both optical and non-optical detection modes.

The disclosed low-binding supports, optionally used in combination with the disclosed hybridization and/or amplification protocols, yield solid-phase reactions that exhibit: (i) negligible non-specific binding of protein and other reaction components (thus minimizing substrate background), (ii) negligible non-specific nucleic acid amplification product, and (iii) provide tunable nucleic acid amplification reactions.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

In some embodiments, a fluorescence image of the surface exhibits a contrast-to-noise ratio (CNR) of at least 20 when a sample nucleic acid molecule or complementary sequences thereof are labeled with a Cyanine dye-3 (Cy3) fluorophore, and when the fluorescence image is acquired using an inverted fluorescence microscope (e.g., Olympus IX83) with a 20×0.75 NA objective, a 532 nm light source, a bandpass and dichroic mirror filter set optimized for 532 nm excitation and Cy3 fluorescence emission, and a camera (e.g., Andor sCMOS, Zyla 4.2) under non-signal saturating conditions while the surface is immersed in a buffer (e.g., 25 mM ACES, pH 7.4 buffer).

### EXAMPLES

The following examples are meant to be illustrative and can be used to further understand embodiments of the present disclosure and should not be construed as limiting the scope of the present teachings in any way.

### Example 1: Two-Plex Batch Sequencing Template Concatemers Prepared with Single-Stranded Splint or Double-Stranded Adaptors

Covalently closed circular libraries containing phiX insert regions were prepared by hybridizing linear library molecules to either single-stranded splints (e.g., FIG. 37) or double-stranded adaptors (e.g., FIG. 43).

The single-stranded splint workflow: single-stranded nucleic acid library molecules (100) included the following components (e.g., FIG. 37): (i) pinning primer binding site sequence (120); (ii) a left sample index sequence (160); (iii) a forward sequencing primer binding site sequence (140) (Element Biosciences' ss-Splint sequencing primer); (iv) a sequence of interest (e.g., insert sequence) (110); (v) a reverse sequencing primer binding site sequence (150); (vi) a right sample index sequence (170); and (vii) a capture primer binding site sequence (130). The single-stranded library molecules were hybridized with single-stranded splint strands (200) to generate library-splint complexes (300) having one nick. The nick was ligated to generate covalently closed circular library molecules (400). The single stranded splint strand (200) was removed enzymatically. The forward sequencing primer binding site sequence (140) was Element Biosciences' ss-Splint sequencing primer having the sequence 5′-CGTGCTGGATTGGCTCACCAGACACCTTCCGACAT-3′ (SEQ ID NO: 33).

The double-stranded splint workflow: single-stranded nucleic acid library molecules (100) included the following components (e.g., FIG. 43): (i) pinning primer binding site sequence (120); (ii) a left sample index sequence (160); (iii) a forward sequencing primer binding site sequence (140) (Element Biosciences' ss-Splint sequencing primer); (iv) a sequence of interest (e.g., insert sequence) (110); (v) a reverse sequencing primer binding site sequence (150); (vi) a right sample index sequence (170); and (vii) a capture primer binding site sequence (130). The single-stranded library molecules were hybridized with double-stranded splint adaptors (500) to generate library-splint complexes (800) having two nicks. The nicks were ligated to generate covalently closed circular library molecules (900). The single stranded splint strand (600) was removed enzymatically. The forward sequencing primer binding site sequence (140) was TruSeq (HP10) having the sequence 5′-ACACTCTTTCCCTACACGACGCTCTTCCGATCT-3′ (SEQ ID NO: 34).

The two types of covalently closed circular library molecules (400) and (900) were mixed at 1:1 ration and 20 pM was distributed onto a flowcell having a plurality of capture and pinning primers immobilized thereon. The capture primer was designed to capture both types of covalently closed circular library molecules (e.g., (400) and (900)). The loaded covalently closed circular library molecules (400) and (900) were subjected to on-support rolling circle amplification using the immobilized capture primers as amplification primers, thereby generating two type of concatemer template molecules. The rolling circle amplification reaction was conducted in the presence of compaction oligonucleotides to generated compact DNA nanoballs. The pinning primer was designed to pin down both types of concatemer template molecules resulting from rolling circle amplification. In other experiments, 30 and 40 pM of covalently closed circular library molecules (400) and (900) were loaded onto a flowcell to increase the density of immobilized concatemer molecules.

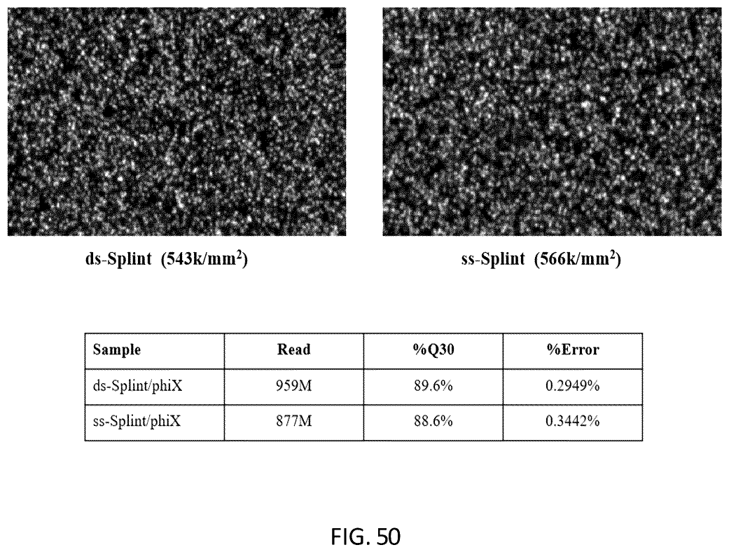

A first batch sequencing reaction was conducted using the TruSeq (HP10) sequencing primer and the two-stage sequencing reaction. 31 sequencing cycles were conducted and fluorescent images were acquired after reacting the concatemer template molecules with labeled multivalent molecules (FIG. 50, left). After 31 sequencing cycles, the first batch sequencing read products were removed.

A second batch sequencing reaction was conducted using Element Biosciences' ss-Splint sequencing primer and the two-stage sequencing reaction. 31 sequencing cycles were conducted and fluorescent images were acquired after reacting the concatemer template molecules with labeled multivalent molecules (FIG. 50, right). After 31 sequencing cycles, the second batch sequencing read products were removed.

In some embodiments, e.g., in FIG. 50, the support (e.g., flowcell) was loaded with 20 pM of a 1:1 mixture of covalently closed circular library molecules generated from either single-stranded splint strands or double-stranded adaptors. The loaded covalently closed circular library molecules were subjected to rolling circle amplification to generate immobilized concatemer template molecules. 31 cycles of first batch sequencing was conducted using first batch sequencing primers (e.g., TruSeq sequencing primers) that selectively hybridized to the concatemer template molecules generated from double-stranded splint adaptors (left image was obtained at one of the 31 sequencing cycles). The first batch sequencing read products were removed. 31 cycles of second batch sequencing was conducted using second batch sequencing primers (e.g., Element Biosciences' ss-Splint sequencing primers) that selectively hybridized to the concatemer template molecules generated from single-stranded splint strands (right image was obtained at one of the 31 sequencing cycles). Other loading concentrations were tested including 30 PM and 40 pM.

The Table in FIG. 50 shows the number of millions of reads, quality scores (% Q30), and percent error.

### Example 2: Four-Plex and Eight-Plex Batch Sequencing Template Concatemers Prepared with Single-Stranded Splints

Four sub-populations of library molecules (100) were prepared having PhiX sequence of interest where individual library molecules included: (i) a universal surface pinning primer binding site sequence (120); (ii) a left sample index sequence (160) (e.g., one of four different sample indexes 9 bases in length); (iii) a forward sequencing primer binding site sequence (140) (e.g., one of four different batch-specific forward sequencing primer binding site sequence (140)); (iv) a sequence of interest (110) (e.g., PhiX); (v) a reverse sequencing primer binding site sequence (150) (e.g., batch-specific reverse sequencing primer binding site sequence (150); (vi) a right sample index sequence (170) (e.g., one of four different sample indexes having a random sequence 3-mer (e.g., NNN) and a 9 base sample index sequence); and (vii) a universal surface capture primer binding site sequence (130). The library molecules did not include a unique molecular index (UMI). For example, see FIG. 37.

Universal single-stranded splint strands (200) were prepared having: (i) a first region (210) that hybridizes with the surface pinning primer binding site sequence (120) of the single stranded library molecule (100), and a second region (220) that hybridizes with the surface capture primer binding site sequence (130) of the single stranded library molecule (100). For example, see FIG. 37.

In four separate reactions, the library molecules (100) were hybridized with universal single-stranded splint strands (200) to generate four sub-populations of library-splint complexes (300) with a nick (e.g., see FIG. 37). The library-splint complexes (300) in the four sub-populations carried one of four different forward sequencing primer binding site sequences.

The library-splint complexes (300) were subjected to separate ligation reaction to generate four sub-populations of covalently closed circular library molecules (400) where individual covalently closed circular library molecules included: (i) a universal surface pinning primer binding site sequence (120); (ii) a left sample index sequence (160) (e.g., one of four different sample indexes 9 bases in length); (iii) a forward sequencing primer binding site sequence (140) (e.g., one of four different batch-specific forward sequencing primer binding site sequence (140)); (iv) a sequence of interest (110) (e.g., PhiX); (v) a reverse sequencing primer binding site sequence (150) (e.g., batch-specific reverse sequencing primer binding site sequence (150); (vi) a right sample index sequence (170) (e.g., one of four different sample indexes having a random sequence 3-mer (e.g., NNN) and a 9 base sample index sequence); and (vii) a universal surface capture primer binding site sequence (130).

The four sub-populations of covalently closed circular library molecules (400) were mixed at 1:1:1:1 ratio and 200 pM of the mixture was distributed onto a flowcell having a plurality of universal capture and pinning primers immobilized thereon. The loaded covalently closed circular library molecules (400) were subjected to on-support rolling circle amplification using the immobilized capture primers as amplification primers, thereby generating four sub-populations of concatemer template molecules, where the concatemers in the different sub-populations carried one of four different forward sequencing primer binding site sequence. The rolling circle amplification reaction was conducted in the presence of compaction oligonucleotides to generate compact concatemers (e.g., DNA nanoballs; polonies).

The density of each sub-population of polonies on the flowcell was measured to be about 400 K/mm² to about 450 K/mm², resulting in a total polony density of about 1600 K/mm².

A first batch sequencing reaction was conducted using a first batch forward sequencing primer and the two-stage sequencing reaction to sequence the PhiX insert region. 31 sequencing cycles were conducted and fluorescent images were acquired after reacting the concatemer template molecules with labeled multivalent molecules (FIG. 50, left). After 31 sequencing cycles, the first batch sequencing read products were removed.

A second batch sequencing reaction was conducted using a second batch forward sequencing primer and the two-stage sequencing reaction to sequence the PhiX insert region. 31 sequencing cycles were conducted and fluorescent images were acquired after reacting the concatemer template molecules with labeled multivalent molecules (FIG. 50, left). After 31 sequencing cycles, the second batch sequencing read products were removed.

The sequencing reactions were repeated using the third and fourth batch forward sequencing primers as described above. The quality scores of the sequencing reads were determined to be about 96% at Q30 and 85% at Q40.

In a similar manner, an 8-plex library prep, circularization and sequencing workflow was conducted using eight sub-populations of libraries that were prepared using eight different batch-specific forward sequencing primer binding site sequences (140) and eight different batch-specific reverse sequencing primer binding site sequence (150).

The eight sub-populations of covalently closed circular library molecules (400) were mixed at equal ratio (e.g., each at 8.5 pM, 12.5 pM or 25 pM) and the mixture was distributed onto a flowcell having a plurality of universal capture and pinning primers immobilized thereon. Thus, 68 pM, 100 pM or 200 pM of the covalently closed circular library molecules were loaded onto a flowcell. Rolling circle amplification reaction was conducted. The density of each sub-population of polonies on the flowcell was measured to be about 270 K/mm² to about 290 K/mm², resulting in a total polony density of about 2100 K/mm². Eight rounds of batch sequencing (31 cycles) were conducted in a manner similar to that described above. The quality scores of the sequencing reads of the eight different sub-populations are listed in Table 1 below.

TABLE 1

| % Q30 | % Q40 |
|---|---|
| 93.3 | 72.2 |
| 91.7 | 67.5 |
| 97 | 88 |
| 96 | 80.9 |
| 91.6 | 66.3 |
| 91.2 | 66.3 |
| 88.6 | 55.7 |
| 97.2 | 85.9 |

It is to be appreciated that the Detailed Description section, and not any other section, is intended to be used to interpret the claims. Other sections may set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

Additionally, some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

While preferred embodiments of the present invention have been shown and described herein, it will be obvious to those skilled in the art that such embodiments are provided by way of example only. The breadth and scope of this disclosure should not be limited by any of the above-described exemplary embodiments. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A computer-implemented method for sequencing three-dimensional samples, comprising:
obtaining, by a processor, a first plurality of flow cell images of a sample in a first plurality of sequencing cycles from a first subset of channels of a sequencing system;
obtaining, by the processor, a second plurality of flow cell images of the sample in a second plurality of sequencing cycles from the first subset of the channels of the sequencing system;
generating, by the processor, a first set of base calls for a first subset of polonies of the sample based on the first plurality of flow cell images; and
generating, by the processor, a second set of base calls for a second subset of polonies of the sample based on the second plurality of flow cell images,
wherein the first subset of channel comprises only some of the channels of the sequencing system, and wherein the second plurality of sequencing cycles are after the first plurality of sequencing cycles.

2. (canceled)

3. (canceled)

4. The computer-implemented method of claim 1, wherein image intensities of the second subset of polonies are below a predetermined threshold in the first plurality of flow cell images.

5. (canceled)

6. The computer-implemented method of claim 1, wherein the second subset of polonies are at least 5×, 10×, 15×, or 20× darker than the first subset of polonies in the first plurality of flow cell images.

7. The computer-implemented method of claim 1, wherein image intensities of the first subset of polonies are below a predetermined threshold in the second plurality of flow cell images.

8. The computer-implemented method of claim 1, wherein the first subset of polonies appear dark in the second plurality of flow cell images.

9. (canceled)

10. The computer-implemented method of claim 1, wherein the first subset of polonies is different from the second subset of polonies.

11. The computer-implemented method of claim 1, wherein the first subset of polonies and the second subset of polonies at least partly overlap spatially in 2D or 3D.

12. The computer-implemented method of claim 1, wherein the first subset of polonies and the second subset of polonies comprise identical batch-specific sequencing binding sites configured to bind to identical sequencing primers.

13. The computer-implemented method of claim 1, wherein each polony of the first subset of polonies and the second subset of polonies comprises an identical batch-specific sequencing binding sites configured to bind to identical sequencing primers.

14. The computer-implemented method of claim 1, wherein each of the first subset of polonies is configured to bind to a first sequencing primer, and each of the second subset of polonies is configured to bind to a second sequencing primer.

15. The computer-implemented method of claim 1, wherein obtaining the first plurality of flow cell images of the sample in the first plurality of sequencing cycles from the first subset of channels comprises:
acquiring, by an optical system of the sequencing system, the first plurality of flow cell images of the sample in the first plurality of sequencing cycles only from the first subset of channels but not from a dark channel of the sequencing system.

16. The computer-implemented method of claim 1, wherein obtaining the first plurality of flow cell images of the sample in the first plurality of sequencing cycles from the one or more channels comprises:
acquiring, by an optical system of the sequencing system, the first plurality of flow cell images of the sample in the first plurality of sequencing cycles from the one or more channels.

17. The computer-implemented method of claim 1, wherein obtaining the first plurality of flow cell images of the sample in the first plurality of sequencing cycles from the first subset of channels comprises:
controlling, by the processor, an optical system to avoid collecting data from one or more image sensors of a dark channel in the first plurality of sequencing cycle.

18. (canceled)

19. (canceled)

20. The computer-implemented method of claim 1, wherein obtaining the second plurality of flow cell images of the sample in the second plurality of sequencing cycles from the one or more channels comprises:
acquiring, by an optical system of the sequencing system, the second plurality of flow cell images of the sample in the second plurality of sequencing cycles from the one or more channels.

21. (canceled)

22. (canceled)

23. The computer-implemented method of claim 1, wherein obtaining the second plurality of flow cell images of the sample in the second plurality of sequencing cycles from the first subset of channels comprises:
controlling, by the processor, an optical system to avoid collecting any data from one or more image sensors of a dark channel in the second plurality of sequencing cycle.

24. The computer-implemented method of claim 1, wherein obtaining the second plurality of flow cell images of the sample in the second plurality of sequencing cycles from the first subset of channels comprises:
controlling, by the processor, an optical system to avoid illuminating light within a predetermined frequency range to the sample in the second plurality of sequencing cycle, the predetermined frequency range corresponding to a dark channel.

25. The computer-implemented method of claim 1, wherein the first set of base calls comprises only one, two, three, or four types of nucleotide bases.

26. (canceled)

27. The computer-implemented method of claim 1, wherein the second set of base calls comprises only one, two, three, or four types of nucleotide bases.

28-150. (canceled)

151. A computer-implemented system sequencing three-dimensional samples, comprising:
one or more hardware processors;
one or more data storage devices storing instructions executable by the one or more hardware processors to cause the one or more hardware processors to perform operations, the operations comprising:
obtaining, by a processor, a first plurality of flow cell images of a sample in a first plurality of sequencing cycles from a first subset of channels;
obtaining, by the processor, a second plurality of flow cell images of the sample in a second plurality of sequencing cycles from the first subset of the channels;
generating, by the processor, a first set of base calls for a first subset of polonies of the sample based on the first plurality of flow cell images; and
generating, by the processor, a second set of base calls for a second subset of polonies of the sample based on the second plurality of flow cell images,
wherein the first subset of channel comprises only some of the channels, and wherein the second plurality of sequencing cycles are after the first plurality of sequencing cycles.

152-154. (canceled)

155. A computer-implemented method for base calling in sequencing data analysis, comprising:
obtaining, by a processor, a first plurality of flow cell images of a sample, wherein each of the first plurality of flow cell images is acquired at a corresponding location along an axial axis;
generating, by the processor, a first plurality of processed images, the first plurality of processed images corresponding to the first plurality of flow cell images;
filtering, by the processor, the first plurality of flow cell images based on the first plurality of processed images thereby generating a first plurality of filtered images;
obtaining, by the processor, a 3D polony map;
extracting, by the processor, image intensity of polonies based on the 3D polony map from:
a second plurality of flow cell images;
a second plurality of processed images;
a second plurality of filtered images; or
their combinations; and
performing, by the processor, base callings based on the extracted image intensity of the polonies.

156-231. (canceled)
